(19) United States
(12) Patent Application Publication
(10) Pub. No.: US 2012/0095211 A1
(43) Pub. Date: Apr. 19, 2012

(54) SUBSTITUTED PROLINE INHIBITORS OF HEPATITIS C VIRUS REPLICATION

(75) Inventors: Brad Buckman, Oakland, CA (US); John B. Nicholas, Redwood City, CA (US); Vladimir Serebryany, Burlingame, CA (US); Scott D. Seiwert, Halfmoon Bay, CA (US)

(73) Assignee: InterMune, Inc., Brisbane, CA (US)

(21) Appl. No.: 13/237,843

(22) Filed: Sep. 20, 2011

Related U.S. Application Data
(60) Provisional application No. 61/385,515, filed on Sep. 22, 2010.
(60) Provisional application No. 61/385,525, filed on Sep. 22, 2010.
(60) Provisional application No. 61/437,570, filed on Jan. 28, 2011.
(60) Provisional application No. 61/446,419, filed on Feb. 24, 2011.

(51) Int. Cl. C07D 487/04

(52) U.S. Cl. 540/460

(57) ABSTRACT

The embodiments provide compounds of the general Formulae I, Ia, II, IIa, III, IIIa, IIIb, IV, IVa, IVb, V, Va, Vb, VI, VIa, VIb, and VIc, as well as compositions, including pharmaceutical compositions, comprising a subject compound. The embodiments further provide treatment methods, including methods of treating a hepatitis C virus infection and methods of treating liver fibrosis, the methods generally involving administering to an individual in need thereof an effective amount of a subject compound or composition. The embodiments also provide methods for the synthesis of subject compounds and intermediates in the synthetic methods.

### RELATED APPLICATIONS

This application claims the benefit of U.S. Provisional Application Nos. 61/385,515, filed Sep. 22, 2010; 61/385,525, filed Sep. 22, 2010; 61/437,570, filed Jan. 28, 2011; and 61/446,419, filed Feb. 24, 2011; all of which are incorporated herein by reference in their entirety.

### BACKGROUND OF THE INVENTION

1. Field of the Invention

The present invention relates to compounds, processes for their synthesis, compositions and methods for the treatment of hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders (NR) or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A is necessary for N53-mediated processing events and enhances proteolytic efficiency at all sites recognized by NS3. The NS3 protease also exhibits nucleoside triphosphatase and RNA helicase activities. NS5B is an RNA-dependent RNA polymerase involved in the replication of HCV RNA.

### SUMMARY OF THE INVENTION

Some embodiments disclosed herein include a compound having a formula I:

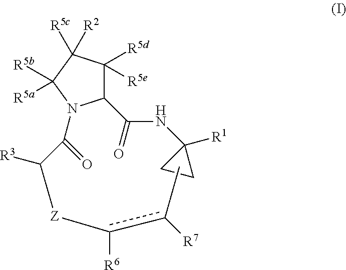

or a pharmaceutically acceptable salt or prodrug thereof, wherein:
(a) R¹ is —C(O)NHS(O)₂R^{1a}, —C(O)NHS(O)₂NR^{1b}R^{1c}, —C(O)NHS(O)R^{1a}, —C(O)NHS(O)NR^{1b}R^{1c}, —C(O)NHC(O)R^{1a}, —C(O)NHOR^{1d}, —C(O)NR^{1b}R^{1c}, —C(O)R^{1a}, —C(O)OR^{1d}, or —C(O)C(O)NR^{1b}R^{1c}, —C(O)C(O)OH, or —P(O)R¹ⁱR^{1j};
   R^{1a} is selected from the group consisting of —H, —C(O)NHO(CH₂)ₘR^{1e}, optionally substituted C₁₋₆ alkyl, optionally substituted —(CH₂)ₘC₃₋₇ cycloalkyl, optionally substituted —(CH₂)ₘaryl, optionally substituted —(CH₂)ₘheterocyclyl, and optionally substituted —(CH₂)ₘheteroaryl;
   R^{1b}, R^{1c}, and R^{1d} are independently selected from the group consisting of —H, optionally substituted C₁₋₆ alkyl, optionally substituted —(CH₂)ₘC₃₋₇ cycloalkyl, optionally substituted —(CH₂)ₘaryl, optionally substituted —(CH₂)ₘheterocyclyl, and optionally substituted —(CH₂)ₘheteroaryl;
   or R^{1b} and R^{1c} are taken together with the nitrogen to which they are attached to form optionally substituted heteroaryl or heterocyclyl, each optionally substituted with 1-3 R^{1f};
   R^{1e} is selected from the group consisting of C₁₋₆ alkyl, —(CH₂)ₘC₃₋₇ cycloalkyl, optionally substituted aryl, and optionally substituted heteroaryl;
   R^{1f} is each independently selected from the group consisting of halo, cyano, amido, phenyl, heteroaryl, heterocyclyl, C₁₋₆ alkyl, C₃₋₇ cycloalkyl, C₁₋₆ alkoxy, C₃₋₇ cycloalkyloxy, —NO₂, —N(R^{1g})₂, —NHC(O)R^{1g}, —NHC(O)NHR^{1g}, and —NHC(O)OR^{1h};
   R^{1g} is —H, C₁₋₆ alkyl, or C₃₋₇ cycloalkyl;
   R^{1h} is C₁₋₆ alkyl or C₃₋₇ cycloalkyl;
   R¹ⁱ and R^{1j} are each separately selected from the group consisting of hydroxy, —(O)ₜ—C₁₋₆ alkyl, —(O)ₜ—(CH₂)ₘC₃₋₇cycloalkyl, —(O)ₜ-aryl, and —(O)ₜ-heteroaryl, each optionally substituted with one or more substituents each independently selected from the group consisting of halo, cyano, nitro, hydroxy, —C(O)OH, C₁₋₆ alkyl, —(CH₂)ₘC₃₋₇cycloalkyl, C₂₋₆ alkenyl, C₁₋₆ alkoxy, hydroxy-C₁₋₆ alkyl, C₁₋₆ alkyl optionally substituted with up to 5 fluoro, and C₁₋₆ alkoxy optionally substituted with up to 5 fluoro;
(b) R² is

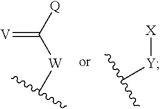

V is Selected from O, S, or NH; when V is O or S, W is O, —NR^{2k}—, or —CR^{2k}—; when V is NH, W is —NR^{2k}— or —CR^{2k}—; where R^{2k} is —H, optionally substituted C₁₋₆ alkyl or optionally substituted C₃₋₇ cycloalkyl;
Y is —O—, —S—, —S(O)—, —S(O)₂—, —OCH₂—, —CH₂O—, or a bond;
X is —(CH₂)ₚR^{2b};
Q is —(CH₂)ₚR^{2b} or —O(CH₂)ₚR^{2b};
R^{2b} is selected from the group consisting hydrogen, alkyl, aryl, heterocyclyl, or heteroaryl; each optionally substituted with one or more substituents selected from the group consisting of halo, cyano, nitro, hydroxy, cyanoamino, optionally substituted C₁₋₆ alkyl, optionally substituted C₃₋₇ cycloalkyl, alkylcycloalkyl, C₂₋₆ alkenyl, C₂₋₆ alkynyl, optionally substituted heterocyclyl, optionally substituted C₁₋₆ alkoxy, optionally substituted aryl, optionally substituted heteroaryl, arylthio, ester, sulfonamide, urea, thiourea, amido, thioamide, carboxyl, carbamyl, carbamate, sulfide, sulfoxide, sulfonyl, amino, alkoxyamino, aminoalkoxy, aminoalkylthio, aminoalkyl, C₁₋₆ alkylthio, alkoxyheterocyclyl, alkylamino, hydroxyalkylamino, alkylcarboxy, carbonyl, spirocyclic cyclopropyl, spirocyclic cyclobutyl, spirocyclic cyclopentyl, spirocyclic cyclohexyl, and —NR^{2c}R^{2d};
R^{2c} and R^{2d} are each independently —H, or independently selected from the group consisting of optionally substituted C₁₋₆ alkyl, optionally substituted C₃₋₇ cycloalkyl, and optionally substituted phenyl; or R² and R^{2d} are taken together with the nitrogen to which they are attached to form heterocyclyl or heteroaryl;
(c) R³ is —NR^{3a}R^{3b} or optionally substituted aryl;
   R^{3a} is selected from the group consisting of —H, optionally substituted C₁₋₆ alkyl, optionally substituted C₃₋₇ cycloalkyl, optionally substituted C₄₋₁₀ alkylcycloalkyl, aryl, heteroaryl, arylalkyl, heteroarylalkyl; wherein said aryl, said heteroaryl, said arylalkyl, and said heteroarylalkyl are each optionally substituted with one or more substituents selected from the group consisting of halo, —CF₃, nitro, cyano, hydroxy, cyanoamino, —SH, optionally substituted C₁₋₆ alkyl, optionally substituted C₃₋₇ cycloalkyl, optionally substituted C₁₋₆ alkoxy, optionally substituted C₂₋₆ alkenyl, optionally substituted C₂₋₆ alkynyl, optionally substituted aryl, optionally substituted heteroaryl, optionally substituted heterocycl, aryloxy, arylthio, C₁₋₆ alkylthio, —N[(CH₂)_{q}OH][(CH₂)_{q}OH], —S(O)₂NR^{3c}R^{3d}, —NHC(O)NR^{3c}R^{3d}, —NHC(S)NR^{3c}R^{3d}, —C(O)NR^{3c}R^{3d}, —NR^{3c}R^{3d}, —C(O)R^{3e}, —C(O)OR^{3e}, —NHC(O)R^{3e}, —NHC(O)OR^{3e}, —S(O)ₘR^{3e}, —NHS(O)₂R^{3e}, —NR^{3e}[(CH₂)_{q}OH], —O[(CH₂)_{q}NR^{3c}R^{3d}], and —S[(CH₂)_{q}NR^{3c}R^{3d}];
   R^{3b} is selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted aryl, optionally substituted heteroaryl, optionally substituted heterocyclyl, —C(O)R^{3e}, —C(O)OR^{3e}, —C(O)NR^{3c}R^{3d}, —C(S)NR^{3c}R^{3d}, —S(O)ₘR^{3e}, —S(O)₂OR^{3e}, —S(O)₂NR^{3c}R^{3d}, —C(O)CHR^{3f}(CH₂)ₙC(O)R^{3g}, —C(O)CHR^{3f}NHC(O)R^{3g};
   or R^{3a} and R^{3b} are taken together with the nitrogen to which they are attached to form optionally substituted heterocyclyl or optionally substituted heteroaryl;
   R^{3c} and R^{3d} are each independently selected from the group consisting of —H, optionally substituted C₁₋₆ alkyl, optionally substituted C₁₋₆ alkoxy, C₂₋₆ alkenyl, C₂₋₆ alkynyl, carboxyl, halo, hydroxyl, amino, amido, —OC(O)—C₁₋₆ alkyl, optionally substituted C₃₋₇ cycloalkyl, optionally substituted C₃₋₇ cycloalkyloxy, optionally substituted C₄₋₁₀ alkylcycloalkyl, optionally substituted C₄₋₁₀ cycloalkyl-alkyl, optionally substituted aryl, optionally substituted C₇₋₁₀ arylalkyl, optionally substituted heteroaryl, optionally substituted C₆₋₁₂ heteroarylalkyl and optionally substituted heterocyclyl; or R^{3c} and R^{3d} are taken together with the nitrogen to which they are attached to form optionally substituted heterocyclyl or optionally substituted heteroaryl;
   R^{3e} is selected from the group consisting of optionally substituted C₁₋₆ alkyl, optionally substituted C₃₋₇ cycloalkyl, optionally substituted C₂₋₆ alkenyl, optionally substituted C₂₋₆ alkynyl, optionally substituted C₆₋₁₀ aryl, optionally substituted heterocyclyl, optionally substituted heteroaryl, and optionally substituted bicycloalkyl;
   R^{3f} is optionally substituted C₁₋₆ alkyl, optionally substituted C₃₋₇ cycloalkyl, optionally substituted C₆₋₁₀ aryl, optionally substituted heteroaryl, optionally substituted heterocyclyl, aryloxy and heteroaryloxy;
   R^{3g} is C₁₋₆ alkyl, C₃₋₇ cycloalkyl, or C₄₋₁₀ alkylcycloalkyl, which are all optionally substituted from one to three times with halo, cyano, nitro, hydroxy, C₁₋₆ alkyl optionally substituted with up to 5 fluoro, or phenyl;
(d) R^{5a}, R^{5b}, R^{5c}, R^{5d}, and R^{5e} are each independently selected from —H, optionally substituted C₁₋₆ alkyl, optionally substituted C₂₋₆ alkenyl, optionally substituted C₂₋₆ alkynyl, or optionally substituted C₁₋₆ alkoxy;
   provided that at least one of R^{5c}, R^{5d}, and R^{5e} is not —H or at least one of R^{5a} and R^{5b} is methyl;
   or R^{5a} and R^{5b} together with the carbon atom to which they are attached to form a C₃₋₆ cycloalkyl or C₃₋₆ cycloalkoxy, and R^{5c}, R^{5d}, and R^{5e} are —H;
   or R^{5d} and R^{5e} together with the carbon atom to which they are attached to form a C₃₋₆ cycloalkyl or C₃₋₆ cycloalkoxy, and R^{5a}, R^{5b}, and R^{5c} are —H;
(e) R⁶ and R⁷ are each independently hydrogen, halo, or together with the carbon atoms to which they are attached to form an optionally substituted cycloalkyl;
(f) Z is C₃₋₆ alkyl or three- to seven-membered heteroalkyl containing 1-2 heteroatoms selected from O or N, wherein each said alkylene and said heteroalkylene is optionally substituted by 1-3 R⁸;
   wherein R⁸ is —OH, —F, C₁₋₆ alkyl optionally substituted with up to 5 fluoro, or —SOₘR^{8a};
   R^{8a} is selected from the group consisting of C₁₋₆ alkyl, C₃₋₇ cycloalkyl, and C₆₋₁₀ aryl, each optionally substituted with one or more substituents each independently selected from the group consisting of halo, cyano, nitro, hydroxy, optionally substituted C₁₋₆ alkyl, C₂₋₆ alkenyl, C₁₋₆ alkoxy, and phenyl;
(g) each m is independently 0, 1 or 2;
(h) each n is independently 1, 2, or 3;
(i) each p is independently 0, 1, 2, 3, 4, 5, or 6;
(j) each q is independently 1, 2, 3, 4, 5, or 6;
(k) each t is independently 0 or 1; and
(l) the dashed line represents an optional double bond.

Other embodiments disclosed herein include a compound having a formula II:

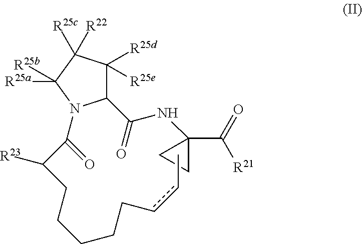

or a pharmaceutically acceptable salt or prodrug thereof, wherein:
(a) R²¹ is selected from hydroxy, —NHS(O)₂R^{21a}, —NHS(O)₂NR^{21b}R^{21c} or —NR^{21b}R^{21c}; wherein
   R^{21a} is selected from the group consisting of optionally substituted C₁₋₆ alkyl, optionally substituted C₃₋₇ cycloalkyl, optionally substituted aryl, and optionally substituted heterocyclyl;
   R^{21b} and R^{21c} are each independently selected from —H, optionally substituted C₁₋₆ alkyl, optionally substituted C₃₋₇ cycloalkyl, optionally substituted aryl, optionally substituted heterocyclyl, and arylalkyl; or R^{21b} and R^{21c} together with the nitrogen to which they are attached to form an optionally substituted 3-7 membered heterocyclyl ring;
(b) R²² is selected from

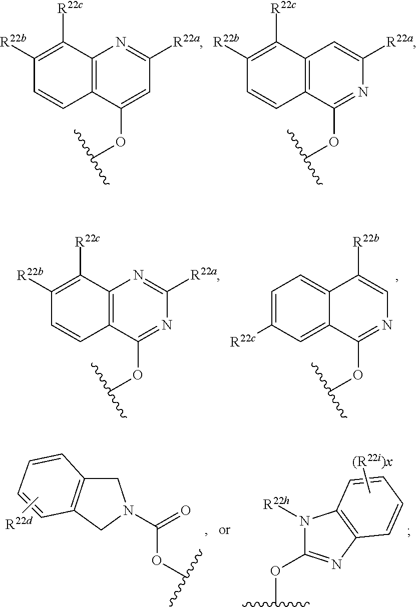

wherein
   R^{22a} is selected from the group consisting of —H, halo, C₁₋₆ alkyl, C₃₋₇ cycloalkyl, C₁₋₆ haloalkyl, C₁₋₆ thioalkyl, C₁₋₆ alkoxy, C₃₋₇ cycloalkyloxy, C₂₋₇ alkoxyalkyl, aryl, heterocyclyl, and heteroaryl; wherein said C₃₋₇ cycloalkyl, said aryl, said heterocyclyl and said heteroaryl are each substituted with 1-3 R^{22e};
   R^{22b} is selected from the group consisting of —H, halo, C₁₋₆ alkyl, C₃₋₇ cycloalkyl, C₁₋₆ haloalkyl, C₁₋₆ alkoxy, C₃₋₇ cycloalkyloxy, hydroxy, phenyl, heterocyclyl, heteroaryl, and —NR^{22f};
   R^{22c} is —H, optionally substituted C₁₋₆ alkyl, optionally substituted C₁₋₆ alkoxy, C₁₋₆ alkylamino or halo;
   R^{22d} is selected from the group consisting of —H, halo, cyano, hydroxy, optionally substituted C₁₋₆ alkyl, and optionally substituted C₁₋₆ alkoxy;
   R^{22e} is selected from the group consisting of —H, halo, C₁₋₆ alkyl, C₃₋₇ cycloalkyl, C₁₋₆ alkoxy, C₃₋₇ cycloalkyloxy, —NO₂, —NR^{22f}R^{22g}, NHC(O)R^{22f}, —NHC(O)OR^{22g}, and —NHC(O)NHR^{22f};
   R^{22f} and R^{22g} are each independently —H, C₁₋₆ alkyl, or C₃₋₇ cycloalkyl;
   R^{22h} is —H or C₁₋₆ alkyl optionally substituted with up to 5 fluoro;
   R²²ⁱ is selected from the group consisting of halo, cyano, nitro, hydroxy, cyanoamino, —SH, optionally substituted C₁₋₆ alkyl, optionally substituted C₁₋₆ alkoxy, C₂₋₆ alkenyl, C₂₋₆ alkynyl, heterocyclyl, optionally substituted aryl, optionally substituted heteroaryl, aryloxy, arylthio, C₁₋₆ alkylthio, —N[(CH₂)_{q}OH][(CH₂)_{q}OH], —S(O)₂NR^{22j}R^{22k}, NHC(O)NR^{22j}R^{22k}, —NHC(S)NR^{22j}R^{22k}, C(O)NR^{22j}R^{22k}, NR^{22j}R^{22k}, C(O)R^{22l}, —C(O)OR^{22l}, —NHC(O)R^{22l}, —NHC(O)OR^{22l}, —SOₘR^{22l}, —NHS(O)₂R^{22l}, —NR^{22l}[(CH₂)_{q}OH], —O[(CH₂)_{q}NR^{22m}R²²ⁿ], —S[(CH₂)_{q}NR^{22m}R²²ⁿ], —(CH₂)_{q}NR^{22m}R²²ⁿ, —(CH₂)_{q}R^{22p} and —O(CH₂)ₚR^{22p};
   R²²ⁱ and R^{22k} are each separately a —H, or separately selected from the group consisting of C₁₋₆ alkyl, —(CH₂)ₘC₃₋₇ cycloalkyl, and phenyl, each optionally substituted with one or more substituents each independently selected from the group consisting of halo, cyano, nitro, hydroxy, C₁₋₆ alkyl, C₃₋₇ cycloalkyl, C₄₋₁₀ alkylcycloalkyl, C₂₋₆ alkenyl, hydroxy-C₁₋₆ alkyl, C₁₋₆ alkyl optionally substituted with up to 5 fluoro, and C₁₋₆ alkoxy optionally substituted with up to 5 fluoro; or R²²ⁱ and R^{22k} are taken together with the nitrogen to which they are attached to form a heterocyclyl;
   R^{22l} is selected from the group consisting of C₁₋₆ alkyl, C₃₋₇ cycloalkyl, heterocyclyl, and C₆₋₁₀ aryl, each optionally substituted with one or more substituents each independently selected from the group consisting of halo, cyano, nitro, hydroxy, C₁₋₆ alkyl, C₂₋₆ alkenyl, —(CH₂)ₘC₃₋₇ cycloalkyl, C₁₋₆ alkoxy, phenyl, and hydroxy-C₁₋₆ alkyl;
   R^{22m} and R²²ⁿ are each separately —H or C₁₋₆ alkyl; or R^{22m} and R²²ⁿ are taken together with the nitrogen to which they are attached to form a heterocyclyl;
   each R^{22p} is heteroaryl;
   each p is separately 0, 1, 2, 3, 4, 5, or 6;
   each q is separately 1, 2, 3, 4, 5, or 6;
   each m is separately 0, 1 or 2;
   x is 0, 1, 2 or 3;
(c) R²³ is —NR^{23a}R^{23b} or aryl optionally substituted with 1-3 substituents independently selected from halo, C₁₋₆ alkyl, or C₁₋₆ haloalkyl;
   R^{23a} is selected from the group consisting of —H, optionally substituted C₁₋₆ alkyl, optionally substituted C₃₋₇ cycloalkyl, optionally substituted C₄₋₁₀ alkylcycloalkyl, aryl, heteroaryl, arylalkyl, heteroarylalkyl; wherein said aryl, said heteroaryl, said arylalkyl, and said heteroarylalkyl are each optionally substituted with one or more substituents selected from the group consisting of halo, nitro, cyano, hydroxy, cyanoamino, —SH, optionally substituted C₁₋₆ alkyl, optionally substituted C₃₋₇ cycloalkyl, optionally substituted C₁₋₆ alkoxy, optionally substituted C₂₋₆ alkenyl, optionally substituted C₂₋₆ alkynyl, optionally substituted aryl, optionally substituted heteroaryl, aryloxy, arylthio, C₁₋₆ alkylthio, —N[(CH₂)_{q}OH][(CH₂)_{q}OH], —S(O)₂NR^{23c}R^{23d}, —NHC(O)NR^{23c}R^{23d}, NHC(S)NR^{23c}R^{23d}, C(O)NR^{23c}R^{23d}, NR^{23c}R^{23d}, C(O)R^{23e}, —C(O)OR^{23e}, —NHC(O)R^{23e}, —NHC(O)OR^{23e}, —S(O)ₘR^{23e}, —NHS(O)₂R^{23e}, —NR^{23e}[(CH₂)_{q}OH], —O[(CH₂)_{q}NR^{23c}R^{23d}], and —S[(CH₂)_{q}NR^{23c}R^{23d}];
   R^{23b} is selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted aryl, optionally substituted heteroaryl, —C(O)R^{23e}, —C(O)OR^{23e}, —C(O)NR^{23c}R^{23d}, —C(S)NR^{23c}R^{23d}, S(O)ₘR^{23e}, S(O)₂OR^{23e}, —S(O)₂NR^{3c}R^{3d}, —C(O)CHR^{23f}(CH₂)ₙC(O)R^{23g}, —C(O)CHR^{23f}NHC(O)R^{23g};
   R^{23c} and R^{23d} are each independently selected from the group consisting of —H, optionally substituted C₁₋₆ alkyl, optionally substituted C₁₋₆ alkoxy, C₂₋₆ alkenyl, C₂₋₆ alkynyl, carboxyl, halo, hydroxyl, amino, amido, —OC(O)—C₁₋₆ alkyl, optionally substituted C₃₋₇ cycloalkyl, optionally substituted C₃₋₇ cycloalkyloxy, optionally substituted C₄₋₁₀ alkylcycloalkyl, optionally substituted C₄₋₁₀ cycloalkyl-alkyl, optionally substituted aryl, optionally substituted C₇₋₁₀ arylalkyl, optionally substituted heteroaryl, optionally substituted C₆₋₁₂ heteroarylalkyl and optionally substituted heterocyclyl; or R^{23c} and R^{23d} are taken together with the nitrogen to which they are attached to form optionally substituted heterocyclyl or optionally substituted heteroaryl;
   R^{23e} is selected from the group consisting of optionally substituted C₁₋₆ alkyl, optionally substituted C₃₋₇ cycloalkyl, optionally substituted C₂₋₆ alkenyl, optionally substituted C₂₋₆ alkynyl, optionally substituted C₆₋₁₀ aryl, and optionally substituted heterocyclyl;
   R^{23f} is optionally substituted C₁₋₆ alkyl, optionally substituted C₃₋₇ cycloalkyl, optionally substituted C₆₋₁₀ aryl, optionally substituted heteroaryl, optionally substituted heterocyclyl, aryloxy and heteroaryloxy;
   R^{23g} is C₁₋₆ alkyl, C₃₋₇ cycloalkyl, or C₄₋₁₀ alkylcycloalkyl, which are all optionally substituted from one to three times with halo, cyano, nitro, hydroxy, C₁₋₆ alkyl optionally substituted with up to 5 fluoro, or phenyl;
(d) R^{25a}, R^{25b}, R^{25c}, R^{25d} and R^{25e} are each independently selected from —H, optionally substituted C₁₋₆ alkyl, optionally substituted C₂₋₆ alkenyl, optionally substituted C₂₋₆ alkynyl or optionally substituted C₁₋₆ alkoxy;
   provided that at least one of R^{25c}, R^{25d}, and R^{25e} is not —H or at least one of R^{25a} and R^{25b} is methyl;
   or R^{25a} and R^{25b} together with the carbon atom to which they are attached to form a C₃₋₇ cycloalkyl, and R^{25c}, R^{25d} and R^{25e} are —H;
   or R^{25d} and R^{25e} together with the carbon atom to which they are attached to form a C₃₋₇ cycloalkyl, and R^{25a}, R^{25b}, and R^{25c} are —H; and
(e) the dashed line represents an optional double bond.

Other embodiments disclosed herein include a compound having the structure of Formula III:

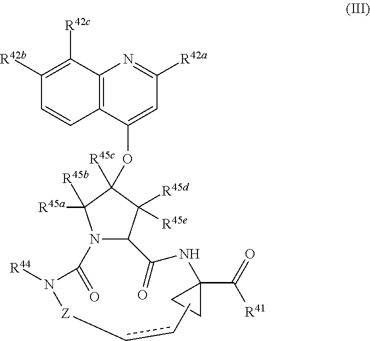

or a pharmaceutically acceptable salt or prodrug thereof, wherein:
(a) R⁴¹ is selected from —OR^{41a}, —NHS(O)₂R^{41b}, —NHS(O)₂NR^{41c}R^{41d}, or —NR^{41c}R^{41d}; wherein
   R^{41a} is selected from the group consisting of —H, optionally substituted C₁₋₆ alkyl, optionally substituted C₃₋₇ cycloalkyl, optionally substituted aryl, optionally substituted heteroaryl, and optionally substituted heterocyclyl;
   R^{41b} is selected from the group consisting of optionally substituted C₁₋₆ alkyl, optionally substituted C₃₋₇ cycloalkyl, optionally substituted aryl, optionally substituted heteroaryl, and optionally substituted heterocyclyl;
   R^{41c} and R^{41d} are each independently selected from hydrogen, optionally substituted C₁₋₆ alkyl, optionally substituted C₃₋₇ cycloalkyl, optionally substituted aryl, optionally substituted heterocyclyl, and arylalkyl; or R^{41c} and R^{41d} together with the N to which they are attached to form an optionally substituted 3-7 membered heterocyclyl ring;
(b) R^{42a} is selected from the group consisting of —H, halo, C₁₋₆ alkyl, C₃₋₇ cycloalkyl, C₁₋₆ haloalkyl, C₁₋₆ thioalkyl, C₁₋₆ alkoxy, C₃₋₇ cycloalkyloxy, C₂₋₇ alkoxyalkyl, C₆₋₁₀ aryl, heterocyclyl, and heteroaryl; wherein said cycloalkyl, aryl, heterocyclyl and heteroaryl are each substituted with R^{42d};
   R^{42d} is selected from the group consisting of —H, halo, C₁₋₆ alkyl, C₃₋₇ cycloalkyl, C₁₋₆ alkoxy, C₃₋₇ cycloalkyloxy, —NO₂, —NR^{42e}R^{42f}, —NHC(O)R^{42e}, —NHC(O)OR^{42f}, and —NHC(O)NHR^{42e}; wherein R^{42e} and R^{42f} are each independently —H, C₁₋₆ alkyl, or C₃₋₇ cycloalkyl;
(c) R^{42b} is selected from the group consisting of —H, halo, C₁₋₆ alkyl, hydroxy, C₁₋₆ alkoxy, C₃₋₇ cycloalkyl, C₁₋₆ haloalkyl, C₃₋₇ cycloalkyloxy, phenyl, heterocyclyl, heteroaryl, and NR^{42e};
(d) R^{42c} is selected from —H, halo, optionally substituted C₁₋₆ alkyl, optionally substituted C₁₋₆ alkoxy, C₁₋₆ alkylamino;
(e) R⁴⁴ is selected from —H, optionally substituted C₁₋₆ alkyl, optionally substituted C₁₋₆ alkoxy-C₁₋₆ alkyl, or optionally substituted C₃₋₇ cycloalkyl;
(f) R^{45a}, R^{45b}, R^{45c}, R^{45d}, and R^{45e} are each independently selected from —H, optionally substituted C₁₋₆ alkyl, optionally substituted C₂₋₆ alkenyl, optionally substituted C₂₋₆ alkynyl or optionally substituted C₁₋₆ alkoxy; and
(g) the dashed line represents an optional double bond.

Some embodiments disclosed herein include a compound having the structure of Formula IV:

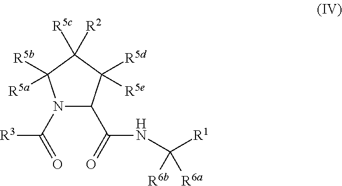

or a pharmaceutically acceptable salt or prodrug thereof, wherein:
(a) R¹ is —C(O)NHS(O)₂R^{1a}, —C(O)NHS(O)₂NR^{1b}R^{1c}, —C(O)NHS(O)R^{1a}, —C(O)NHS(O)NR^{1b}R^{1c}, —C(O)NHC(O)R^{1a}, —C(O)NHOR^{1d}, —C(O)NR^{1b}R^{1c}, —C(O)R^{1a}, —C(O)OR^{1d}, or —C(O)C(O)NR^{1b}R^{1c}, —C(O)C(O)OH, or —P(O)R¹ⁱR^{1j};
   R^{1a} is selected from the group consisting of —H, —C(O)NHO(CH₂)ₘR^{1e}, optionally substituted C₁₋₆ alkyl, optionally substituted —(CH₂)ₘC₃₋₇ cycloalkyl, optionally substituted —(CH₂)ₘaryl, optionally substituted —(CH₂)ₘheterocyclyl, and optionally substituted —(CH₂)ₘheteroaryl;
   R^{1b}, R^{1c}, and R^{1d} are independently selected from the group consisting of —H, optionally substituted C₁₋₆ alkyl, optionally substituted —(CH₂)ₘC₃₋₇ cycloalkyl, optionally substituted —(CH₂)ₘaryl, optionally substituted —(CH₂)ₘheterocyclyl, and optionally substituted —(CH₂)ₘheteroaryl;
   or R^{1b} and R^{1c} are taken together with the nitrogen to which they are attached to form optionally substituted heteroaryl or heterocyclyl, each optionally substituted with 1-3 R^{1f};
   R^{1e} is selected from the group consisting of C₁₋₆ alkyl, —(CH₂)ₘC₃₋₇ cycloalkyl, optionally substituted aryl, and optionally substituted heteroaryl;
   R^{1f} is each independently selected from the group consisting of halo, cyano, amido, phenyl, heteroaryl, heterocyclyl, C₁₋₆ alkyl, C₃₋₇ cycloalkyl, C₁₋₆ alkoxy, C₃₋₇ cycloalkyloxy, —NO₂, —N(R^{1g})₂, —NHC(O)R^{1g}, —NHC(O)NHR^{1g}, and —NHC(O)OR^{1h};
   R^{1g} is —H, C₁₋₆ alkyl, or C₃₋₇ cycloalkyl;
   R^{1h} is C₁₋₆ alkyl or C₃₋₇ cycloalkyl;
   R¹ⁱ and R^{1j} are each separately selected from the group consisting of hydroxy, —(O)ₜ—C₁₋₆ alkyl, —(O)ₜ—(CH₂)ₘC₃₋₇cycloalkyl, —(O)ₜ-aryl, and —(O)ₜ-heteroaryl, each optionally substituted with one or more substituents each independently selected from the group consisting of halo, cyano, nitro, hydroxy, —C(O)OH, C₁₋₆ alkyl, —(CH₂)ₘC₃₋₇cycloalkyl, C₂₋₆ alkenyl, C₁₋₆ alkoxy, hydroxy-C₁₋₆ alkyl, C₁₋₆ alkyl optionally substituted with up to 5 fluoro, and C₁₋₆ alkoxy optionally substituted with up to 5 fluoro;
(b) R² is

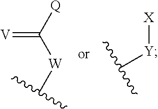

V is selected from O, S, or NH; when V is O or S, W is O, —NR^{2k}—, or —CR^{2k}—; when V is NH, W is —NR^{2k}— or —CR^{2k}—; where R^{2k} is —H, optionally substituted C₁₋₆ alkyl or optionally substituted C₃₋₇ cycloalkyl;
Y is —O—, —S—, —S(O)—, —S(O)₂—, —OCH₂—, —CH₂O—, or a bond;
X is —(CH₂)ₚR^{2b};
Q is —(CH₂)ₚR^{2b} or —O(CH₂)ₚR^{2b};
R^{2b} is selected from the group consisting hydrogen, alkyl, aryl, heterocyclyl, or heteroaryl; each optionally substituted with one or more substituents selected from the group consisting of halo, cyano, nitro, hydroxy, cyanoamino, optionally substituted C₁₋₆ alkyl, optionally substituted C₃₋₇ cycloalkyl, alkylcycloalkyl, C₂₋₆ alkenyl, C₂₋₆ alkynyl, optionally substituted heterocyclyl, optionally substituted C₁₋₆ alkoxy, optionally substituted aryl, optionally substituted heteroaryl, arylthio, ester, sulfonamide, urea, thiourea, amido, thioamide, carboxyl, carbamyl, carbamate, sulfide, sulfoxide, sulfonyl, amino, alkoxyamino, aminoalkoxy, aminoalkylthio, aminoalkyl, C₁₋₆ alkylthio, alkoxyheterocyclyl, alkylamino, hydroxyalkylamino, alkylcarboxy, carbonyl, spirocyclic cyclopropyl, spirocyclic cyclobutyl, spirocyclic cyclopentyl, spirocyclic cyclohexyl, and —NR^{2c}R^{2d};
R^{2c} and R^{2d} are each independently —H, or independently selected from the group consisting of optionally substituted C₁₋₆ alkyl, optionally substituted C₃₋₇ cycloalkyl, and optionally substituted phenyl; or R^{2c} and R^{2d} are taken together with the nitrogen to which they are attached to form heterocyclyl or heteroaryl;
(c) R³ is —H, —R⁷, or —R⁷—NR^{3a}R^{3b}; wherein
   R⁷ is selected from the group consisting of optionally substituted C₁₋₆ alkyl, optionally substituted C₃₋₇ cycloalkyl, optionally substituted aryl, optionally substituted heteroaryl, optionally substituted C₄₋₁₀ cycloalkyl-alkyl, optionally substituted C₇₋₁₂ arylalkyl, and optionally substituted C₆₋₁₂ heteroarylalkyl;
   R^{3a} is selected from the group consisting of —H, optionally substituted C₁₋₆ alkyl, optionally substituted C₃₋₇ cycloalkyl, optionally substituted C₄₋₁₀ alkylcycloalkyl, aryl, heteroaryl, arylalkyl, heteroarylalkyl; wherein said aryl, said heteroaryl, said arylalkyl, and said heteroarylalkyl are each optionally substituted with one or more substituents selected from the group consisting of halo, CF₃, nitro, cyano, hydroxy, cyanoamino, —SH, optionally substituted C₁₋₆ alkyl, optionally substituted C₃₋₇ cycloalkyl, optionally substituted C₁₋₆ alkoxy, optionally substituted C₂₋₆ alkenyl, optionally substituted C₂₋₆ alkynyl, optionally substituted aryl, optionally substituted heteroaryl, optionally substituted heterocyclyl, aryloxy, arylthio, C₁₋₆ alkylthio, —N[(CH₂)_{q}OH][(CH₂)_{q}OH], —S(O)₂NR^{3c}R^{3d}, —NHC(O)NR^{3c}R^{3d}, —NHC(S)NR^{3c}R^{3d}, —C(O)NR^{3c}R^{3d}, —NR^{3c}R^{3d}, —C(O)R^{3e}, —C(O)OR^{3e}, —NHC(O)R^{3e}, —NHC(O)OR^{3e}, —S(O)ₘR^{3e}, —NHS(O)₂R^{3e}, —NR^{3e}[(CH₂)_{q}OH], —O[(CH₂)_{q}NR^{3c}R^{3d}], and —S[(CH₂)_{q}NR^{3c}R^{3d}];
   R^{3b} is selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted aryl, optionally substituted heteroaryl, optionally substituted heterocyclyl, —C(O)R^{3e}, —C(O)OR^{3e}, —C(O)NR^{3c}R^{3d}, —C(S)NR^{3c}R^{3d}, —S(O)ₘR^{3e}, —S(O)₂OR^{3e}, —S(O)₂NR^{3c}R^{3d}, —C(O)CHR^{3f}(CH₂)ₙC(O)R^{3g}, and —C(O)CHR^{3f}NHC(O)R^{3g};
   or R^{3a} and R^{3b} are taken together with the nitrogen to which they are attached to form optionally substituted heterocyclyl or optionally substituted heteroaryl;
   R^{3c} and R^{3d} are each independently selected from the group consisting of —H, optionally substituted C₁₋₆ alkyl, optionally substituted C₁₋₆ alkoxy, C₂₋₆ alkenyl, C₂₋₆ alkynyl, carboxyl, halo, hydroxyl, amino, amido, —OC(O)—C₁₋₆ alkyl, optionally substituted C₃₋₇ cycloalkyl, optionally substituted C₃₋₇ cycloalkyloxy, optionally substituted C₄₋₁₀ alkylcycloalkyl, optionally substituted C₄₋₁₀ cycloalkyl-alkyl, optionally substituted aryl, optionally substituted C₇₋₁₀ arylalkyl, optionally substituted heteroaryl, optionally substituted C₆₋₁₂ heteroarylalkyl and optionally substituted heterocyclyl; or R^{3c} and R^{3d} are taken together with the nitrogen to which they are attached to form optionally substituted heterocyclyl or optionally substituted heteroaryl;
   R^{3e} is selected from the group consisting of —H, optionally substituted C₁₋₆ alkyl, optionally substituted C₃₋₇ cycloalkyl, optionally substituted C₂₋₆ alkenyl, optionally substituted C₂₋₆ alkynyl, optionally substituted C₆₋₁₀ aryl, optionally substituted heterocyclyl, optionally substituted heteroaryl, and optionally substituted bicycloalkyl;
   R^{3f} is optionally substituted C₁₋₆ alkyl, optionally substituted C₃₋₇ cycloalkyl, optionally substituted C₆₋₁₀ aryl, optionally substituted heteroaryl, optionally substituted heterocyclyl, aryloxy and heteroaryloxy;
   R^{3g} is C₁₋₆ alkyl, C₃₋₇ cycloalkyl, or C₄₋₁₀ alkylcycloalkyl, which are all optionally substituted from one to three times with halo, cyano, nitro, hydroxy, C₁₋₆ alkyl optionally substituted with up to 5 fluoro, or phenyl;
(d) R^{5a}, R^{5b}, R^{5c}, R^{5d} and R^{5e} are each independently selected from —H, optionally substituted C₁₋₆ alkyl, optionally substituted C₂₋₆ alkenyl, optionally substituted C₂₋₆ alkynyl or optionally substituted C₁₋₆ alkoxy;
   provided that at least one of R^{5a}, R^{5b}, R^{5c}, R^{5d} and R^{5e} is not —H;
   or R^{5a} and R^{5b} together with the carbon atom to which they are attached to form a C₃₋₆ cycloalkyl or C₃₋₆ cycloalkoxy, and R^{5c}, R^{5d} and R^{5e} are —H;
   or R^{5d} and R^{5e} together with the carbon atom to which they are attached to form a C₃₋₆ cycloalkyl or C₃₋₆ cycloalkoxy, and R^{5a}, R^{5b} and R^{5c} are —H;
(e) R^{6a} and R^{6b} are independently —H or an optionally substituted moiety selected from the group consisting of C₁₋₆ alkyl, C₄₋₁₀ cycloalkyl-alkyl, C₂₋₆ alkenyl, C₃₋7 cycloalkyl, C₇₋₁₀ arylalkyl, C₆₋₁₂ heteroaryl-alkyl, aryl, and heteroaryl; or R^{6a} and R^{6b} are taken together to form C₃₋₇ cycloalkyl or three to seven-membered heterocyclyl, each optionally substituted by 1-3 substituents selected from the group consisting of C₁₋₆ alkyl, C₂₋₆ alkenyl, and optionally substituted C₃₋₇ cycloalkyl;
(f) each m is independently 0, 1 or 2;
(g) each n is independently 1, 2, or 3;
(h) each p is independently 0, 1, 2, 3, 4, 5, or 6;
(i) each q is independently 1, 2, 3, 4, 5, or 6; and,
(k) each t is independently 0 or 1.

Some embodiments disclosed herein include compounds having the structure of Formula V:

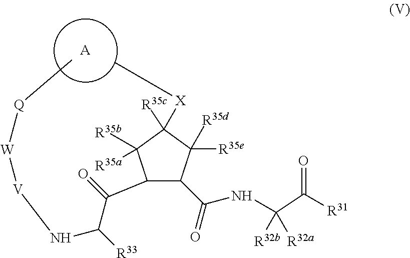

or a pharmaceutically acceptable salt or prodrug thereof, wherein:
(a) A is an optionally substituted moiety selected from the group consisting of aryl, heteroaryl, and heterocyclyl;
(b) X is a bond or selected from the group consisting of —O—, —S—, —S(O)—, —S(O)₂—, —OCH₂—, —CH₂O—, —OC(O)—, —NHC(O)—, and —NH—;
(c) Q is C₅₋₈ alkylene, C₅₋₈ alkenylene, or C₅₋₈ heteroalkylene; each optionally substituted with 1-3 substituents selected from C₁₋₆ alkyl or halo;
(d) V is —C(O)—, —S(O)₂—, or —CR^{34a}R^{34b} wherein R^{34a} and R^{34b} are independently selected from C₁₋₆ alkyl, C₂₋₆ alkenyl, or halo;
(e) W is —O—, —NH—, or a bond;
(e) R³¹ is —NHS(O)₂R^{31a}, —NHS(O)₂NR^{31b}R^{31c}, NHS(O)R^{31a}, —NHS(O)NR^{31b}R^{31c}, —NHC(O)R^{31a}, —NHOR^{31d}, —NR^{31b}R^{31c}, —R^{31a}, OR^{31d}, or —C(O)NR^{31b}R^{31c}, —C(O)OH, or —P(O)R¹ⁱR^{1j}; wherein
   R^{31a} is selected from the group consisting of —H, —C(O)NHO(CH₂)ₘR^{31e}, optionally substituted C₁₋₆ alkyl, optionally substituted —(CH₂)ₘC₃₋₇ cycloalkyl, optionally substituted —(CH₂)ₘaryl, optionally substituted —(CH₂)ₘheterocyclyl, and optionally substituted —(CH₂)ₘheteroaryl;
   R^{31b}, R^{31c}, and R^{31d} are independently selected from the group consisting of —H, optionally substituted C₁₋₆ alkyl, optionally substituted —(CH₂)ₘC₃₋₇ cycloalkyl, optionally substituted —(CH₂)ₘaryl, optionally substituted —(CH₂)ₘheterocyclyl, and optionally substituted —(CH₂)ₘheteroaryl;
   or R^{31b} and R^{31c} are taken together with the nitrogen to which they are attached to form optionally substituted heteroaryl or heterocyclyl, each optionally substituted with 1-3 R^{31f};
   R^{31e} is selected from the group consisting of C₁₋₆ alkyl, —(CH₂)ₘC₃₋₇ cycloalkyl, optionally substituted aryl, and optionally substituted heteroaryl;
   R^{31f} is each independently selected from the group consisting of halo, cyano, amido, phenyl, heteroaryl, heterocyclyl, C₁₋₆ alkyl, C₃₋₇ cycloalkyl, C₁₋₆ alkoxy, C₃₋₇ cycloalkoxy, —NO₂, —N(R^{31g})₂, —NHC(O)R^{31g}, —NHC(O)NHR^{31g}, and —NHC(O)OR^{31h};
   R^{31g} is —H, C₁₋₆ alkyl, or C₃₋₇ cycloalkyl;
   R^{31h} is C₁₋₆ alkyl or C₃₋₇ cycloalkyl;
(f) R^{32a} and R^{32b} are independently —H or an optionally substituted moiety selected from the group consisting of C₁₋₆ alkyl, C₄₋₁₀ cycloalkyl-alkyl, C₂₋₆ alkenyl, C₃₋₇ cycloalkyl, C₇₋₁₀ arylalkyl, C₆₋₁₂ heteroaryl-alkyl, aryl, and heteroaryl; alternatively R^{32a} and R^{32b} are taken together to form C₃₋₇ cycloalkyl or three to seven-membered heterocyclyl, each optionally substituted by 1-3 R^{32c}; wherein R^{32c} is selected from the group consisting of C₁₋₆ alkyl, C₂₋₆ alkenyl, and optionally substituted C₃₋₇ cycloalkyl;
(g) R³³ is selected from the group consisting of —H, C₁₋₆ alkyl optionally substituted with up to 5 fluoro, C₂₋₆ alkenyl, C₃₋₇ cycloalkyl optionally substituted with up to 5 fluoro, C₄₋₁₀ cycloalkyl-alkyl, optionally substituted aryl, optionally substituted heteroaryl, optionally substituted C₇₋₁₀ arylalkyl, and optionally substituted C₆₋₁₂ heteroaryl-alkyl; and
(h) R^{35a}, R^{35b}, R^{35c}, R^{35d}, and R^{35e} are each independently selected from —H, optionally substituted C₁₋₆ alkyl, optionally substituted C₂₋₆ alkenyl, optionally substituted C₂₋₆ alkynyl or optionally substituted C₁₋₆ alkoxy;
   provided that at least one of R^{35a}, R^{35b}, R^{35c}, R^{35d}, and R^{35e} is not —H;
   or R^{35a} and R^{35b} together with the carbon atom to which they are attached to form a C₃₋₆ cycloalkyl, and R^{35c}, R^{35d} and R^{35e} are —H;
   or R^{5d} and R^{5e} together with the carbon atom to which they are attached to form a C₃₋₆ cycloalkyl, and R^{35a}, R^{35b} and R^{35c} are —H; and
(i) each m is independently 0, 1 or 2.

Some embodiments disclosed herein include compounds of Formula VI:

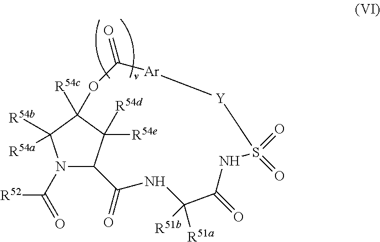

or a pharmaceutically acceptable salt or prodrug thereof, wherein:
(a) Ar is optionally substituted heteroaryl, optionally substituted aryl, or optionally substituted heterocyclyl;
(b) Y is (L)ᵣ;
   r is an integer from 5 to 12;
   each L is separately selected, where L is selected from the group consisting of C(R⁵⁸)₂, NR⁵⁹, —C(═O)NR⁵⁹—, O (oxygen), —(R⁵⁸)C═C(R⁵⁸)—, C(═O), C₃₋₇ cycloalkyl, optionally substituted aryl, optionally substituted heterocycle, and optionally substituted heteroaryl;
   each R⁵⁸ is separately selected, where R⁵⁸ is selected from the group consisting of H (hydrogen), C₁₋₆alkoxy, C₁₋₆alkyl, aryl, halo, hydroxy, R^{a}R^{b}N—, C₁₋₆alkyl optionally substituted with up to 5 halo, and C₁₋₆alkoxy optionally substituted with up to 5 halo, or optionally two vicinal R⁵⁸ and the carbons to which they are attached are together a fused three- to six-membered carbocyclic ring optionally substituted with up to two C₁₋₆alkyl groups, or optionally two geminal R⁵⁸ and the carbon to which they are attached are together a fused three- to six-membered carbocyclic ring optionally substituted with up to two C₁₋₆alkyl groups;
   each R^{a}R^{b}N is separately selected, wherein R^{a} and R^{b} are each separately selected from the group consisting of hydrogen, C₂₋₆alkenyl, and C₁₋₆alkyl;
   each R⁵⁹ is separately selected, where R⁵⁹ is selected from the group consisting of hydrogen, optionally substituted aryl, optionally substituted heteroaryl, optionally substituted C₁₋₆alkyl, and C₁₋₆alkyl optionally substituted with up to 5 halo;
(c) v is 0 or 1;
(d) R^{51a} and R^{51b} are independently —H or an optionally substituted moiety selected from the group consisting of C₁₋₆ alkyl, C₄₋₁₀ cycloalkyl-alkyl, C₂₋₆ alkenyl, C₃₋₇ cycloalkyl, C₇₋₁₀ arylalkyl, C₆₋₁₂ heteroaryl-alkyl, aryl, and heteroaryl; alternatively R^{51a} and R^{51b} are taken together to form C₃₋₇ cycloalkyl or three to seven-membered heterocyclyl, each optionally substituted by 1-3 R⁵¹, wherein R^{51c} is selected from the group consisting of C₁₋₆ alkyl, C₂₋₆ alkenyl, C₂₋₆ alkynyl and optionally substituted C₃₋₇ cycloalkyl;
(e) R⁵² is —H, —R⁵⁷, or —R⁵⁷—NR^{53a}R^{53b}; wherein
   R⁵⁷ is selected from the group consisting of optionally substituted C₁₋₆ alkyl, optionally substituted C₃₋₇ cycloalkyl, optionally substituted aryl, optionally substituted heteroaryl, optionally substituted C₄₋₁₀ cycloalkyl-alkyl, optionally substituted C₇₋₁₂ arylalkyl, and optionally substituted C₆₋₁₂ heteroarylalkyl;
   R^{53a} is selected from the group consisting of —H, optionally substituted C₁₋₆ alkyl, optionally substituted C₃₋₇ cycloalkyl, optionally substituted C₄₋₁₀ alkylcycloalkyl, aryl, heteroaryl, arylalkyl, heteroarylalkyl; wherein said aryl, said heteroaryl, said arylalkyl, and said heteroarylalkyl are each optionally substituted with one or more substituents selected from the group consisting of halo, nitro, cyano, hydroxy, cyanoamino, —SH, optionally substituted C₁₋₆ alkyl, optionally substituted C₃₋₇ cycloalkyl, optionally substituted C₁₋₆ alkoxy, optionally substituted C₂₋₆ alkenyl, optionally substituted C₂₋₆ alkynyl, optionally substituted aryl, optionally substituted heteroaryl, aryloxy, arylthio, C₁₋₆ alkylthio, —N [(CH₂)_{q}OH][(CH₂)_{q}OH], —S(O)₂NR^{53c}R^{53d}, —NHC(O)NR^{53c}R^{53d}, —NHC(S)NR^{3c}R^{3d}, —C(O)NR^{3c}R^{3d}, —NR^{3c}R^{53d}, —C(O)R^{53e}, —C(O)OR^{53e}, —NHC(O)R^{53e}, —NHC(O)OR^{53e}, —S(O)ₘR^{53e}, —NH S(O)₂R^{53e}, —NR^{53e}[(CH₂)_{q}OH], —O[(CH₂)_{q}NR^{53c}R^{53d}], and —S[(CH₂)_{q}NR^{53c}R^{53d}];
   R^{53b} is selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted aryl, optionally substituted heteroaryl, —C(O)R^{53e}, —C(O)OR^{53e}, —C(O)NR^{53c}R^{53d}, —C(S)NR^{53c}R^{53d}, —S(O)ₘR^{53e}, —S(O)₂OR^{53e}, —S(O)₂NR^{53c}R^{53d}, —C(O)CHR^{53f}(CH₂)ₙC(O)R^{53g}, and —C(O)CHR^{53f}NHC(O)R^{53g};
   R^{53c} and R^{53d} are each independently selected from the group consisting of —H, optionally substituted C₁₋₆ alkyl, optionally substituted C₁₋₆ alkoxy, C₂₋₆ alkenyl, C₂₋₆ alkynyl, carboxyl, halo, hydroxyl, amino, amido, —OC(O)—C₁₋₆ alkyl, optionally substituted C₃₋₇ cycloalkyl, optionally substituted C₃₋₇ cycloalkoxy, optionally substituted C₄₋₁₀ alkylcycloalkyl, optionally substituted C₄₋₁₀ cycloalkyl-alkyl, optionally substituted aryl, optionally substituted C₇₋₁₀ arylalkyl, optionally substituted heteroaryl, optionally substituted C₆₋₁₂ heteroarylalkyl and optionally substituted heterocyclyl; or R^{53c} and R^{53d} are taken together with the nitrogen to which they are attached to form optionally substituted heterocyclyl or optionally substituted heteroaryl;
   R^{53e} is selected from the group consisting of —H, optionally substituted C₁₋₆ alkyl, optionally substituted C₃₋₇ cycloalkyl, optionally substituted C₂₋₆ alkenyl, optionally substituted C₂₋₆ alkynyl, optionally substituted C₆₋₁₀ aryl, and optionally substituted heterocyclyl;
   R^{53f} is optionally substituted C₁₋₆ alkyl, optionally substituted C₃₋₇ cycloalkyl, optionally substituted C₆₋₁₀ aryl, optionally substituted heteroaryl, optionally substituted heterocyclyl, aryloxy and heteroaryloxy;
   R^{53g} is C₁₋₆ alkyl, C₃₋₇ cycloalkyl, or C₄₋₁₀ alkylcycloalkyl, which are all optionally substituted from one to three times with halo, cyano, nitro, hydroxy, C₁₋₆ alkyl optionally substituted with up to 5 fluoro, or phenyl;
(f) R^{54a}, R^{54b}, R^{54c}, R^{54d}, and R^{54e} are each independently selected from —H, optionally substituted C₁₋₆ alkyl, optionally substituted C₁₋₆ alkenyl, optionally substituted C₁₋₆ alkynyl or optionally substituted C₁₋₆ alkoxy;
   provided that at least one of R^{54a}, R^{54b}, R^{54c}, R^{54d}, and R^{54e} is not —H;
   or R^{54a} and R^{54b} together with the carbon atom to which they are attached to form a C₃₋₆ cycloalkyl, and R^{54e}, R^{54d}, and R^{54e} are —H;
   or R^{54d} and R^{54e} together with the carbon atom to which they are attached to form a C₃₋₆ cycloalkyl, and R^{54a}, R^{54b} and R^{54c} are —H;
(g) each m is independently 0, 1 or 2;
(h) each n is independently 1, 2, or 3; and
(i) each q is independently 1, 2, 3, 4, 5, or 6.

Other embodiments disclosed herein include pharmaceutical compositions comprising a pharmaceutically acceptable excipient and a compound described above.

Other embodiments disclosed herein include methods of inhibiting NS3/NS4 protease activity comprising contacting a NS3/NS4 protease with a compound of any one the compounds or compositions described above.

Other embodiments disclosed herein include methods of treating HCV infection in an individual comprising administering to the individual an effective amount of any one the compounds or compositions described above.

Other embodiments disclosed herein include methods of treating liver fibrosis in an individual comprising administering to the individual an effective amount of any one the compounds or compositions described above.

Other embodiments disclosed herein include increasing liver function in an individual having a hepatitis C virus infection comprising administering to the individual an effective amount of any one the compounds or compositions described above.

Still other embodiments comprise methods for synthesizing the compounds described above and intermediates in such synthetic methods.

### DETAILED DESCRIPTION OF THE PREFERRED EMBODIMENT

As used herein, common organic abbreviations are defined as follows:
* Indicates a chiral center that is optionally of the R or S configuration
Ac Acetyl
aq. Aqueous
Bn Benzyl
Bz Benzoyl
BOC or Boc tert-Butoxycarbonyl
BOC₂O di-tert-butyl dicarbonate
CBz Carbobenzyloxy
CDI 1,1′-carbonyldiimidazole
° C. Temperature in degrees Centigrade
DBU 1,8-Diazabicyclo[5.4.0]undec-7-ene
DCC N,N-Dicyclohexylcarbodiimide
DCM methylene chloride
DIC N,N-Diisopropylcarbodiimide
DIBALH Diisobutylaluminium hydride
DIEA Diisopropylethylamine
DMAP 4-Dimethylaminopyridine
DMF N,N′-Dimethylformamide
DMSO Dimethylsulfoxide
EDAC N-(3-Dimethylaminopropyl)-N′-ethylcarbodiimide hydrochloride, also abbreviated as EDAC hydrochloride, EDC, EDC hydrochloride, WSC and WSC hydrochloride
Et Ethyl
EtOAc Ethyl acetate
EtOH Ethanol
Fmoc Fluorenylmethyloxycarbonyl
g Gram(s)
h Hour (hours)
HATU 2-(1H-7-azabenzotriazol-1-yl)-1,1,3,3-tetramethyl uranium hexafluorophosphate
HBTU (o-Benzotriazol-1-yl N,N,N′,N′-tetramethyluronium hexafluorophosphate)
HOBT 1-Hydroxybenzotriazole
iPr Isopropyl
mCPBA meta-Chloroperoxybenzoic Acid
MeOH Methanol
mg milligram
mL Milliliter(s)
NaHMDS sodium bis(trimethylsilyl)amide or sodium hexamethyldisilazide,
NBS N-Bromosuccinimide
NCS N-Chlorosuccinimide
PFP pentafluorophenyl
P Protecting group for an amine
P′ Protecting group for a carboxylic acid
P″ Protecting group for an alcohol
Pd/C Palladium on activated carbon
ppt Precipitate
rt Room temperature
sec, s secondary
TBAF Tetra-n-butylammonium fluoride
TB TU (o-Benzotriazol-1-yl N,N, N′,N′-tetramethyluronium tetrafluoroborate)
TBS t-butyldimethylsilyl
TBDPS t-butyldiphenylsilyl
TCDI 1,1′-Thiocarbonyl diimidazole
TEA Triethylamine
Tert, t tertiary
TFA Trifluoracetic acid
TMS Trimethylsilyl

As used herein, the term “hepatic fibrosis,” used interchangeably herein with “liver fibrosis,” refers to the growth of scar tissue in the liver that can occur in the context of a chronic hepatitis infection.

The terms “individual,” “host,” “subject,” and “patient” are used interchangeably herein, and refer to a mammal, including, but not limited to, primates, including simians and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, murines, simians, humans, mammalian farm animals, mammalian sport animals, and mammalian pets.

The term “alkyl” as used herein refers to a radical of a fully saturated hydrocarbon, including, but not limited to, methyl, ethyl, n-propyl, isopropyl (or i-propyl), n-butyl, isobutyl, tert-butyl (or t-butyl), n-hexyl,

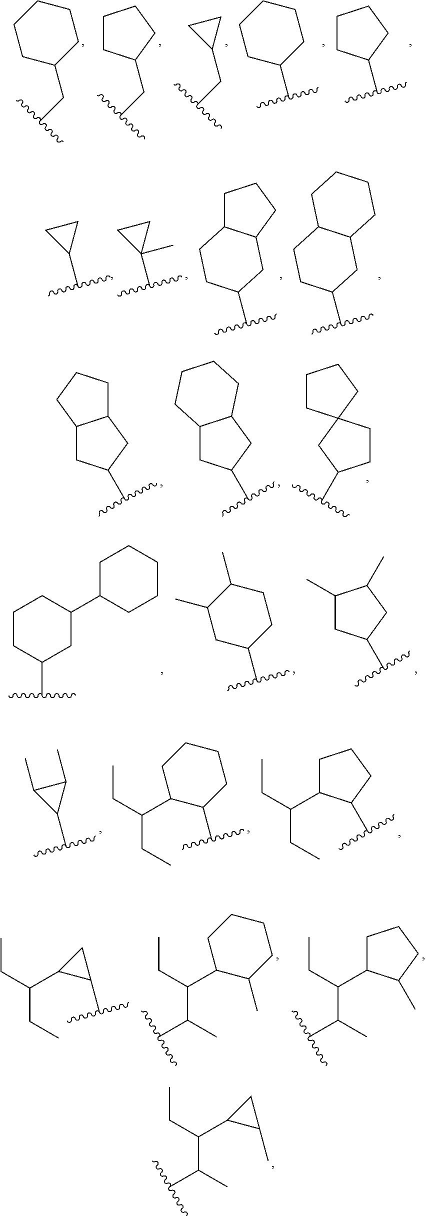

and the like. For example, the term “alkyl” as used herein includes radicals of fully saturated hydrocarbons defined by the following general formula's: the general formula for linear or branched fully saturated hydrocarbons not containing a cyclic structure is CₙH₂ₙ₊₂; the general formula for a fully saturated hydrocarbon containing one ring is CₙH₂ₙ; the general formula for a fully saturated hydrocarbon containing two rings is CₙH₂₍ₙ₋₁₎; the general formula for a saturated hydrocarbon containing three rings is CₙH₂₍ₙ₋₂₎. When the term “alkyl” and a more specific term for alkyl (such as propyl, butyl, etc.) is used without specifying linear or branched, the term is to be interpreted to include linear and branched alkyl.

The term “halo” used herein refers to fluoro, chloro, bromo, or iodo.

The term “alkoxy” used herein refers to straight or branched chain alkyl radical covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like. When the term “alkoxy” and a more specific term for alkoxy (such as propoxy, butaoxy, etc.) is used without specifying linear or branched, the term is to be interpreted to include linear and branched alkoxy.

The term “alkenyl” used herein refers to a monovalent straight or branched chain radical of from two to twenty carbon atoms containing a carbon double bond including, but not limited to, 1-propenyl, 2-propenyl, 2-methyl-1-propenyl, 1-butenyl, 2-butenyl, and the like.

The term “alkynyl” used herein refers to a monovalent straight or branched chain radical of from two to twenty carbon atoms containing a carbon triple bond including, but not limited to, 1-propynyl, 1-butynyl, 2-butynyl, and the like.

The term “alkylene” used herein refers to a linker formed from a straight-chained unsaturated aliphatic hydrocarbon, which links together molecular fragments via their terminal carbon atoms. Examples include, but not limited to, methylene (—CH₂—), ethylene (—CH₂CH₂—), propylene (—CH₂CH₂CH₂—), butylene (—(CH₂)₄—), pentylene (—(CH₂)₅—), hexylene (—(CH₂)₆—), and the like.

The term “heteroalkylene” used herein refers to an alkylene group in which one or more of the carbon atoms are independently replaced with the same or different heteroatoms selected from oxygen, sulfur and nitrogen. “X- to Y-membered” heteroalkylene indicates that the heteroalkylene contains total of X to Y atoms, including carbon atoms and heteroatoms. For example, 4- to 10-membered heteroalkylene contains 4, 5, 6, 7, 8, 9 or 10 atoms in the linker group. The heteroalkylene may have 1-4 heteroatoms, 1-3 heteroatoms, 1-2 heteroatoms or 1 heteroatom. Examples of heteroalkylene include, but not limited to, —CH₂—O—, —CH₂—CH₂—O—, —CH₂—CH₂—CH₂—O—, —CH₂—NH—, —CH₂—CH₂—NH—, —CH₂—CH₂—CH₂—NH—, —CH₂—CH₂—NH—CH₂—, —O—CH₂—CH₂—O—CH₂—CH₂—O—, —O—CH₂—CH₂—O—CH₂—CH₂—, and the like

The term “alkenylene” used herein refers to a linker formed from an alkenyl moiety (as defined above) in which a hydrogen atom has been removed to yield a divalent radical, which links together molecular fragments via their terminal carbon atoms. Examples include, but not limited to, butenylene (—CH₂CH═CHCH₂—), pentenylene (—CH₂CH₂CH═CHCH₂—), hexenylene (—CH₂CH₂CH₂CH═CHCH₂—). The double bond may be between any two carbon atoms, thus the location of the double bond on the linker is not limited.

The term “aryl” used herein refers to homocyclic aromatic radical whether one ring or multiple fused rings. Examples of aryl groups include, but are not limited to, phenyl, naphthyl, phenanthrenyl, naphthacenyl, and the like.

The term “cycloalkyl” used herein refers to saturated aliphatic ring system radical having three to twenty carbon atoms including, but not limited to, cyclopropyl, cyclopentyl, cyclohexyl, cycloheptyl, and the like. The cycloalkyl may be monocycloalkyl or polycycloalkyl, such as bicycloalkyl and tricycloalkyl, etc.

The term “cycloalkenyl” used herein refers to aliphatic ring system radical having three to twenty carbon atoms having at least one carbon-carbon double bond in the ring. Examples of cycloalkenyl groups include, but are not limited to, cyclopropenyl, cyclopentenyl, cyclohexenyl, cycloheptenyl, bicyclo[3.1.0]hexyl, and the like. The cycloalkenyl may include monocycloalkenyl or polycycloalkenyl.

The term “cycloalkoxy” used herein refers to a cycloalkyl ring system wherein one or more carbon atom in the ring system is replaced by an oxygen atom.

The term “cycloalkyloxy” used herein refers to cycloalkyl radical covalently bonded to the parent molecule through an —O— linkage.

The term “polycycloalkyl” used herein refers to saturated aliphatic ring system radical having at least two rings that are fused with or without bridgehead carbons. Examples of polycycloalkyl groups include, but are not limited to, bicyclo[4.4.0]decanyl, bicyclo[2.2.1]heptanyl, adamantyl, norbornyl, and the like.

The term “polycycloalkenyl” used herein refers to aliphatic ring system radical having at least two rings that are fused with or without bridgehead carbons in which at least one of the rings has a carbon-carbon double bond. Examples of polycycloalkenyl groups include, but are not limited to, norbornylenyl, 1,1′-bicyclopentenyl, bicycle[3.1.0]hexyl and the like.

The term “polycyclic hydrocarbon” used herein refers to a ring system radical in which all of the ring members are carbon atoms. Polycyclic hydrocarbons can be aromatic (e.g., aryl group) or can contain less than the maximum number of non-cumulative double bonds (e.g., cycloalkyl and cycloalkenyl). Examples of polycyclic hydrocarbon include, but are not limited to, naphthyl, dihydronaphthyl, indenyl, fluorenyl, and the like.

The term “heterocyclic” or “heterocyclyl” or “heterocycloalkyl” used herein refers to cyclic non-aromatic ring system radical having at least one ring in which one or more ring atoms are not carbon, namely heteroatom. The cyclic non-aromatic ring system may contain one or more rings that are aromatic provided that the system as a whole is not aromatic (i.e., it contains at least one non-aromatic ring). The cyclic non-aromatic ring system may contain 1, 2, 3, or 4 heteroatom(s) independently selected from N, S or O. The cyclic non-aromatic ring system also includes polycyclic moieties containing one or more heteroatoms. In some fused ring systems, the one or more heteroatoms may be present in only one of the rings. Examples of heterocyclic groups include, but are not limited to, morpholinyl, tetrahydrofuranyl, dioxolanyl, imidazolidinyl, thiomorpholinyl, thiazolidinyl, oxazolidinyl, oxathiolanyl, tetrahydrothiophenyl, pyrazolidinyl, dioxolanyl, pyrrolidinyl, pyranyl, piperidyl, piperazyl, piperidinyl, piperazinyl, oxetanyl, indolinyl, isoindolinyl, thienylene, 4H-quinolizinyl and the like.

The term “heterocyclylalkyl” used herein refers to one or more heterocyclyl groups appended to an alkyl radical. Examples of heterocyclylalkyl include, but are not limited to, morpholinylmethyl, morpholinylethyl, morpholinylpropyl, tetrahydrofuranylmethyl, pyrrolidinylpropyl, and the like.

The term “polycyclic ring system” or “polycyclic moiety” used herein refers to a bicyclic moiety or tricyclic moiety optionally containing one or more heteroatoms, wherein said ring system or moiety may be aromatic or non-aromatic. Non-aromatic polycyclic moiety includes a bicyclic or tricyclic moiety optionally containing one or more heteroatoms wherein at least one of the rings is not an aryl or heteroaryl. The bicyclic moiety contains two rings wherein the rings are fused, the bicyclic moiety can be appended at any position of the two rings. For example, bicyclic moiety may refer to a radical including but not limited to:

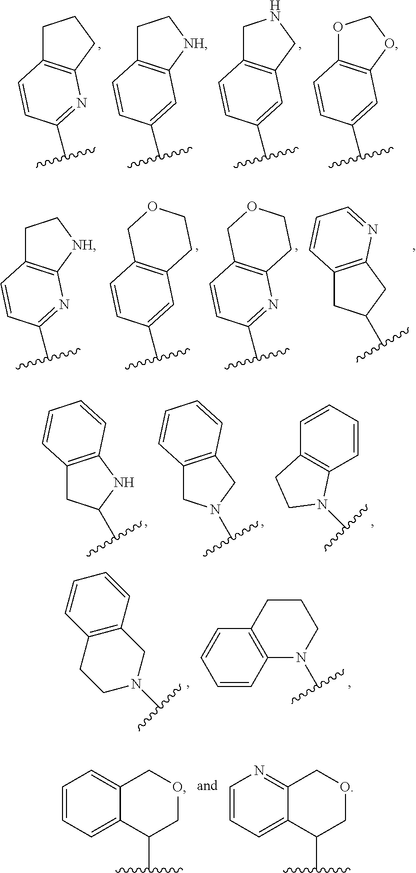

The tricyclic moiety contains a bicyclic moiety with an additional fused ring, the tricyclic moiety can be appended at any position of the three rings. For example, tricyclic moiety may refer to a radical including but not limited to:

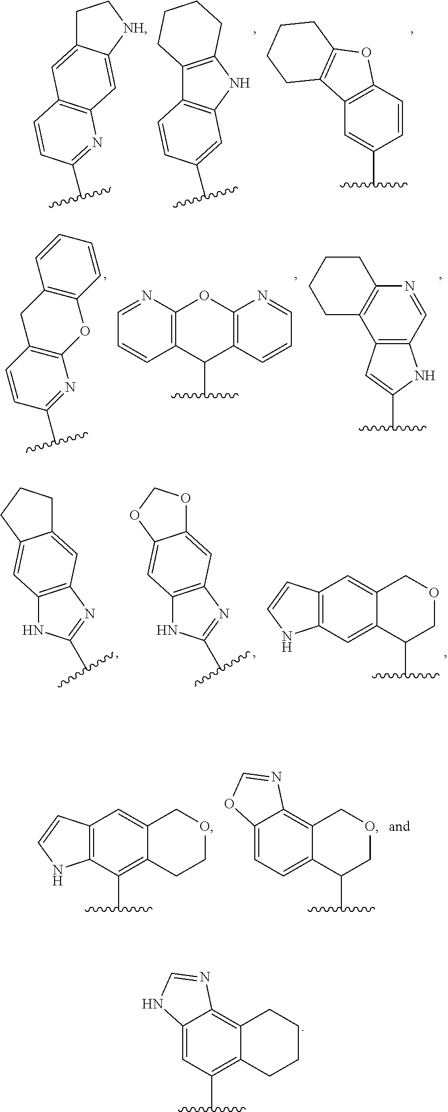

The term “heteroaryl” used herein refers to an aromatic heterocyclic group containing 1-4, 1-3, 1-2 or 1 heteroatom(s) independently selected from N, S or O, whether one ring or multiple fused rings. In some fused ring systems, the one or more heteroatoms may be present in only one of the rings. Examples of heteroaryl groups include, but are not limited to, furan, thiophene (thienyl), pyrrolyl, imidazolyl, pyrazolyl, isoxazolyl, oxazolyl, triazolyl, pyridinyl, pyridazinyl, pyrimidinyl, pyrazinyl, indolyl, isoindolyl, indazolyl, purinyl, quinolinyl, isoquinolinyl, quinazolinyl, quinoxalinyl, phthalazinyl, thiadiazolyl, isothiazolyl, benzothiazyl, benzoxazolyl, thiazyl, benzofuran, benzopyridinyl, benzimidazolyl, benzothiophene and the like.

The term “alkylcycloalkyl” used herein refers to one or more straight or branched chain alkyl radical appended to a cycloalkyl radical. Examples of alkylcycloalkyl include, but are not limited to, methylcyclohexyl, ethylcyclohexyl, methylcyclopentyl, ethylcyclopentyl, and the like.

The term “cycloalkylalkyl” used herein refers to one or more cycloalkyl groups appended to an alkyl radical. Examples of cycloalkylalkyl include, but are not limited to, cyclohexylmethyl, cyclohexylethyl, cyclopentylmethyl, cyclopentylethyl, and the like.

The term “heteroarylalkyl” used herein refers to one or more heteroaryl groups appended to an alkyl radical. Examples of heteroarylalkyl include, but are not limited to, pyridylmethyl, furanylmethyl, thiopheneylethyl, and the like.

The term “aryloxy” used herein refers to an aryl radical covalently bonded to the parent molecule through an —O— linkage.

The term “heteroaryloxy” used herein refers to a heteroaryl radical covalently bonded to the parent molecule through an —O— linkage.

The term “alkylthio” used herein refers to straight or branched chain alkyl radical covalently bonded to the parent molecule through an —S— linkage. Examples of alkoxy groups include, but are not limited to, methoxy, ethoxy, propoxy, isopropoxy, butoxy, n-butoxy, sec-butoxy, t-butoxy and the like.

The term “arylthio” used herein refers to an aryl radical covalently bonded to the parent molecule through an —S— linkage.

The term “alkylamino” used herein refers to nitrogen radical with one or more alkyl groups attached thereto. Thus, monoalkylamino refers to nitrogen radical with one alkyl group attached thereto and dialkylamino refers to nitrogen radical with two alkyl groups attached thereto.

The term “cyanoamino” used herein refers to nitrogen radical with nitrile group attached thereto.

The term “hydroxyalkyl” used herein refers to one or more hydroxy groups appended to an alkyl radical.

The term “aminoalkyl” used herein refers to one or more amino groups appended to an alkyl radical.

The term “arylalkyl” used herein refers to one or more aryl groups appended to an alkyl radical. Examples of arylalkyl groups include, but are not limited to, benzyl, phenethyl, phenpropyl, phenbutyl, and the like.

The term “amido” or “amide” used herein refers to —NR^{A}C(O)R or —C(O)NR^{A}R^{B} group. Unless specifically indicated or defined, R, R^{A} and R^{B} may be independently selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted three- to ten-membered heterocycloalkyl (e.g., tetrahydrofuryl), optionally substituted C₆₋₁₀ aryl, optionally substituted three- to ten-membered heteroaryl, halo (e.g., chloro, bromo, iodo and fluoro), cyano, hydroxy, optionally substituted C₁₋₆ alkoxy, aryloxy, heteroaryloxy, sulfhydryl (mercapto), C₁₋₆ alkylthio, arylthio, mono- and di-(C₁₋₆)alkyl amino, quaternary ammonium salts, amino(C₁₋₆)alkoxy, hydroxy(C₁₋₆)alkylamino, amino(C₁₋₆)alkylthio, cyanoamino, nitro, carbamyl, keto (oxo), carbonyl, carboxy, glycolyl, glycyl, hydrazino, guanyl, sulfamyl, sulfonyl, sulfinyl, thiocarbonyl, and thiocarboxy. R, R^{A}, and R^{B} can be the same or different.

The term “amino” or “amine” used herein refers to —NR^{A}R^{B}. Unless specifically indicated or defined, R^{A} and R^{B} may be as defined above, and can be the same or different.

The term “thioamide” used herein refers to —C(S)NR^{A}R^{B} or —NR^{A}C(S)R group. Unless specifically indicated or defined, R, R^{A}, and R^{B} may be as defined above, and can be the same or different.

The term “carbamate” used herein refers to —NR^{A}C(O)OR, —OC(O)NR^{A}R^{B} group. Unless specifically indicated or defined, R, R^{A}, and R^{B} may be as defined above, and can be the same or different.

The term “thiocarbamate” used herein refers to —NR^{A}C(S)OR, —OC(S)NR^{A}R^{B} group. Unless specifically indicated or defined, R, R^{A}, and R^{B} may be as defined above, and can be the same or different.

The term “carbamoyl” used herein refers to —C(O)NH₂.

The term “urea” or “carbamide” used herein refers to —NRC(O)NR^{A}R^{B} group. Unless specifically indicated or defined, R, R^{A}, and R^{B} may be as defined above, and can be the same or different.

The term “thiourea” or “thiocarbamide” used herein refers to —NRC(S)NR^{A}R^{B} group. Unless specifically indicated or defined, R, R^{A}, and R^{B} may be as defined above, and can be the same or different.

The term “keto” and “carbonyl” used herein refers to C═O.

The term “carboxy” used herein refers to —C(O)OH.

The term “ester” used herein refers to —C(O)OR group.

The term “cyano” used herein refers to —CN.

The term “sulfide” used herein refers to —SH.

The term “sulfamyl” used herein refers to —S(O)₂NH₂.

The term “sulfonamide” used herein refers to —S(O)₂NR^{A}R^{B} or —NHS(O)₂R group. Unless specifically indicated or defined, R, R^{A}, and R^{B} may be as defined above, and can be the same or different.

The term “sulfamide” used herein refers to —NRS(O)₂NR^{A}R^{B} group. Unless specifically indicated or defined, R, R^{A}, and R^{B} may be as defined above, and can be the same or different.

The term “sulfonyl” used herein refers to —SO₂R. Unless specifically indicated or defined, R may be selected from the group consisting of optionally substituted C₁₋₆ alkyl, optionally substituted C₃₋₇ cycloalkyl, optionally substituted C₂₋₆ alkenyl, optionally substituted C₂₋₆ alkynyl, and optionally substituted C₆₋₁₀ aryl.

The term “sulfinyl” or “sulfoxide” used herein refers to —SOR. Unless specifically indicated or defined, R may be as defined above.

The term “thiocarbonyl” used herein refers to C═S.

The term “thiocarboxy” used herein refers to —C(S)OH.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the terms “group” and “moiety.”

As used herein, when a group is described to be optionally substituted, the group may be unsubstituted or substituted.

As used herein, a substituted group is derived from the unsubstituted parent structure in which there has been an exchange of one or more hydrogen atoms for another atom or group. Unless otherwise indicated, when substituted, the substituent group(s) is (are) one or more group(s) individually and independently selected from C₁₋₆ alkyl, C₂₋₆ alkenyl, C₂₋₆ alkynyl, C₃₋₇ cycloalkyl, three- to ten-membered heterocycloalkyl (e.g., tetrahydrofuryl), C₆₋₁₀ aryl, three- to ten-membered heteroaryl, halo (e.g., chloro, bromo, iodo and fluoro), cyano, hydroxy, C₁₋₆ alkoxy, aryloxy, heteroaryloxy, sulfhydryl (mercapto), C₁₋₆ alkylthio, arylthio, mono- and di-(C₁₋₆)alkyl amino, quaternary ammonium salts, amino(C₁₋₆)alkoxy, hydroxy(C₁₋₆)alkylamino, amino(C₁₋₆)alkylthio, cyanoamino, nitro, carbamyl, keto (oxo), carbonyl, carboxy, glycolyl, glycyl, hydrazino, guanyl, sulfamyl, sulfonyl, sulfinyl, thiocarbonyl, thiocarboxy, and combinations thereof. Each of said C₁₋₆ alkyl, said C₁₋₆ alkoxy, said C₁₋₆ alkenyl, said mono- and di-(C₁₋₆)alkyl amino, and said C₁₋₆ alkylthio may be further substituted with one or more substituents selected from the group consisting of halo, hydroxy, nitro, cyano, aryl, cycloalkyl, and carboxyl. Each of said C₃₋₇ cycloalkyl, said three- to ten-membered heterocyclyl, said C₆₋₁₀ aryl, said three- to ten-membered heteroaryl, said aryloxy, and said arylthio may be further substituted with one or more substituents selected from the group consisting of alkyl, alkeny, alkynyl, alkoxy, cycloalkyl, heterocyclyl, halo, hydroxy, carboxyl, nitro, cyano, amino, amido, alkylamino, alkylthio, —SO₂-alkyl, haloalkyl, haloalkoxy, aryl and heteroaryl. The protecting groups that can form the protective derivatives of the above substituents are known to those of skill in the art and can be found in references such as Greene and Wuts Protective Groups in Organic Synthesis; John Wiley and Sons: New York, 1999. Wherever a substituent is described as “optionally substituted” that substituent can be substituted with the above substituents unless the context clearly dictates otherwise.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

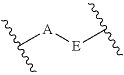

includes the substituent being oriented such that the A is attached at the leftmost attachment point of the molecule as well as attached at the rightmost attachment point of the molecule.

It is to be understood that certain radical naming conventions can include either a mono-radical or a di-radical, depending on the context. For example, where a substituent requires two points of attachment to the rest of the molecule, it is understood that the substituent is a di-radical. A substituent identified as alkyl, that requires two points of attachment, includes di-radicals such as —CH₂—, —CH₂CH₂—, —CH₂CH(CH₃)CH₂—, and the like; a substituent depicted as alkoxy that requires two points of attachment, includes di-radicals such as —OCH₂—, —OCH₂CH₂—, —OCH₂CH(CH₃)CH₂—, and the like: and a substituent depicted as arylC(O)— that requires two points of attachment, includes di-radicals such as

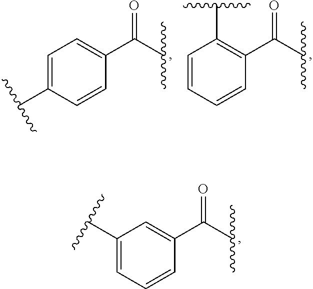

and the like.

Asymmetric carbon atoms may be present in the compounds described. All such isomers, including diastereomers and enantiomers, as well as the mixtures thereof are intended to be included in the scope of the recited compound. In certain cases, compounds can exist in tautomeric forms. All tautomeric forms are intended to be included in the scope. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Various forms are included in the embodiments, including polymorphs, solvates, hydrates, conformers, salts, and prodrug derivatives. A polymorph is a composition having the same chemical formula, but a different structure. A solvate is a composition formed by solvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The present embodiments provide compounds of Formulae I, Ia, II, IIa, III, IIIa, IIIb, IV, IVa, IVb, V, Va, Vb, VI, VIa, VIb, and VIc, as well as pharmaceutical compositions and formulations comprising any compound of Formulae I, Ia, II, IIa, III, IIIa, IIIb, IV, IVa, IVb, V, Va, Vb, VI, VIa, VIb, and VIc. A subject compound is useful for treating HCV infection and other disorders, as discussed below.

### Formula I

Some embodiments include a compound having a formula I:

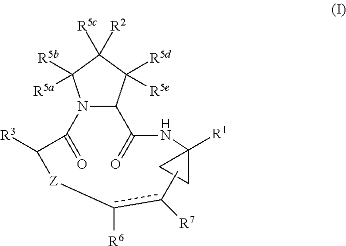

or a pharmaceutically acceptable salt or prodrug thereof, where the variables are as defined above for Formula I.

In some embodiments, the compound of Formula I has the structure of Formula Ia:

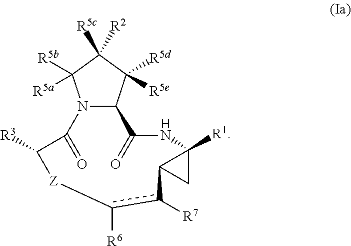

In some embodiments,
R² is

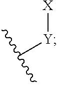

Y is —O— or a bond;
X is

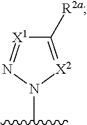

X¹ and X² are each independently selected from —CR^{2e}— or —N—;
R^{2a} and R^{2e} are each selected from the group consisting of —H, halo, optionally substituted aryl, optionally substituted heteroaryl; or R^{2a} and R^{2e} together form an aryl ring optionally substituted by 1-3 R^{2f};
R^{2f} is selected from the group consisting of halo, —C(O)OR^{2g}, —C(O)NR^{2h}R²ⁱ, —NR^{2h}R²ⁱ, —NHC(O)NR^{2h}R²ⁱ, —NHC(O)OR^{2g}, —NHS(O)₂R^{2g}, C₁₋₆ alkyl optionally substituted with up to 5 fluoro, C₂₋₆ alkenyl, C₃₋₇ cycloalkyl, optionally substituted C₁₋₆ alkoxy, optionally substituted aryl, optionally substituted heteroaryl, and optionally substituted heterocyclyl;
R^{2g} is selected from the group consisting of —H, C₁₋₆ alkoxy, C₁₋₆ alkyl, C₃₋₇ cycloalkyl, aryl, arylalkyl heteroaryl, optionally substituted heterocyclyl; and
R^{2h} and R²ⁱ are each independently selected from the group consisting of —H, optionally substituted C₁₋₆ alkyl, optionally substituted C₂₋₆ alkenyl, optionally substituted aryl, optionally substituted arylalkyl optionally substituted heteroaryl, and optionally substituted heterocyclyl.

In some embodiments,
R² is

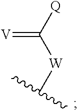

V and W are each 0; and
Q is selected from the group consisting of:

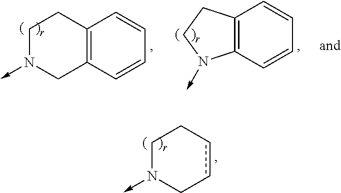

each is optionally substituted with one or more substituents selected from the group consisting of halo, cyano, nitro, hydroxy, cyanoamino, optionally substituted C₁₋₆ alkyl, optionally substituted C₃₋₇ cycloalkyl, C₂₋₆ alkenyl, C₂₋₆ alkynyl, optionally substituted heterocyclyl, optionally substituted C₁₋₆ alkoxy, optionally substituted aryl, optionally substituted heteroaryl, arylthio, ester, sulfonamide, urea, thiourea, amido, thioamide, carboxyl, carbamyl, carbamate, sulfide, sulfoxide, sulfonyl, amino, alkoxyamino, aminoalkoxy, aminoalkylthio, aminoalkyl, C₁₋₆ alkylthio, alkoxyheterocyclyl, alkylamino, hydroxyalkylamino, alkylcarboxy, carbonyl, spirocyclic cyclopropyl, spirocyclic cyclobutyl, spirocyclic cyclopentyl, and spirocyclic cyclohexyl; and wherein r=0 or 1.

In some embodiments, Q is

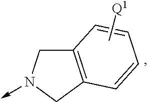

where Q′ is selected from the group consisting of —H, halo, methyl, and C₁₋₆ alkoxy optionally substituted with up to 5 fluoro. In some embodiments, Q¹ is —H, —Cl or —F.

In some embodiments,
R² is

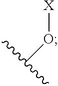

X is selected from

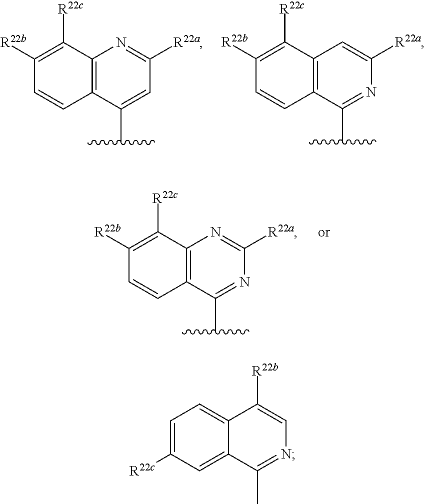

R^{22a} is selected from the group consisting of aryl, heterocyclyl and heteroaryl, each substituted with R^{22e};
R^{22b} is selected from the group consisting of —H, halo, C₁₋₆ alkoxy, C₃₋₇ cycloalkyloxy, and hydroxy;
R^{22c} is —H, optionally substituted C₁₋₆ alkyl or halo;
R^{22e} is selected from the group consisting of —H, C₁₋₆ alkyl, C₃₋₇ cycloalkyl, and —NR^{22f}R^{22g}; wherein R^{22f} and R^{22g} are each independently —H, C₁₋₆ alkyl, or C₃₋₇ cycloalkyl;
R³ is —NR^{3a}R^{3b} or aryl optionally substituted with 1-3 substituents independently selected from halo or C₁₋₆ haloalkyl;
R^{3a} is selected from the group consisting of —H, C₁₋₆ alkyl, and C₃₋₇ cycloalkyl; and
R^{3b} is selected from the group consisting of —H, —C(O)OR^{3e}, —C(O)NR^{3c}R^{3d}, and aryl optionally substituted with 1-3 substituents selected from the group consisting of halo, —CF₃, hydroxy, nitro, amino, optionally substituted C₁₋₆ alkyl, optionally substituted C₁₋₆ alkoxy, optionally substituted heterocyclyl, and optionally substituted heteroaryl.

In some embodiments, R¹ is —C(O)NHS(O)₂R^{1a}, —C(O)NHS(O)₂NR^{1b}R^{1c}, —C(O)NR^{1b}R^{1c}, or —C(O)OH.

In some embodiments,
R¹ is —C(O)NHS(O)₂R^{1a}, —C(O)NHS(O)₂NR^{1b}R^{1c}, —C(O)NR^{1b}R^{1c}, or —C(O)OH;
R² is;

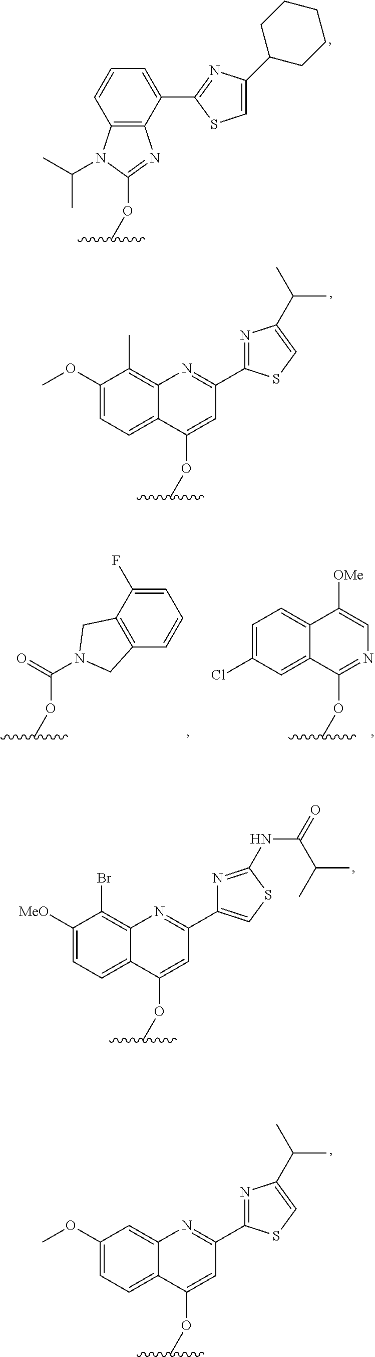

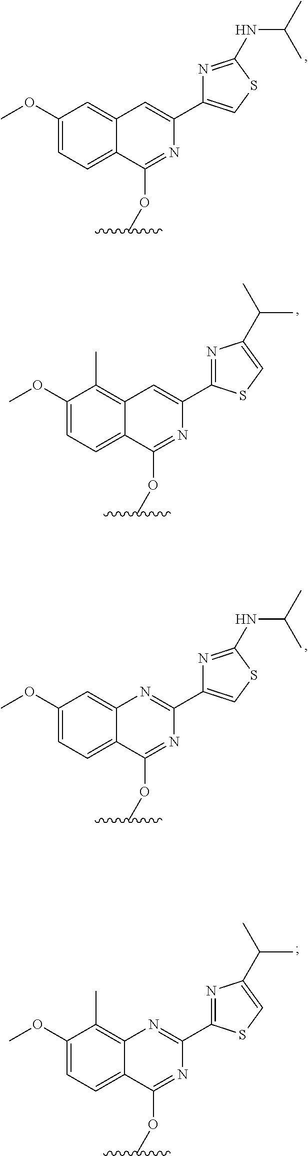

and
R³ is —NR^{3a}R^{3b} or aryl optionally substituted with 1-3 substituents independently selected from halo, C₁₋₆ alkyl, or C₁₋₆ haloalkyl.

In some embodiments, R^{1a} is optionally substituted C₃₋₇ cycloalkyl; and R^{1b} and R^{1c} are independently selected from optionally substituted C₁₋₆ alkyl or optionally substituted heterocyclyl.

In some embodiments, R³ is —NR^{3a}R^{3b} or aryl optionally substituted with 1-3 substituents independently selected from halo or C₁₋₆ haloalkyl, where R^{3a} is selected from the group consisting of —H, optionally substituted C₁₋₆ alkyl, C₄₋₁₀ alkylcycloalkyl, and C₃₋₇ cycloalkyl; and R^{3b} is —H, —C(O)OR^{3e}, —C(O)NR^{3c}R^{3d}, heteroaryl, or aryl, wherein the heteroaryl or aryl of R³¹) is optionally substituted with 1-3 substituents each independently selected from the group consisting of halo, —C(O)OH, nitro, amino, optionally substituted C₁₋₆ alkyl, optionally substituted C₁₋₆ alkoxy.

In some embodiments,
R³ is —NR^{3a}R^{3b} or aryl optionally substituted with 1-3 substituents independently selected from halo or C₁₋₆ haloalkyl;
   R^{3a} is selected from the group consisting of —H, C₁₋₆ alkyl, and C₃₋₇ cycloalkyl;
   R^{3b} is selected from the group consisting of —H, —C(O)OR^{3e}, —C(O)NR^{3c}R^{3d}, heteroaryl, and aryl, wherein the heteroaryl or aryl of R³¹) is optionally substituted with halo or C₁₋₆ haloalkyl; and
   R^{3c} and R^{3d} are taken together with the nitrogen to which they are attached to form optionally substituted heterocyclyl or optionally substituted heteroaryl; and
   R^{3e} is C₁₋₆ alkyl, C₃₋₇ cycloalkyl, or heterocyclyl; each optionally substituted with one or more substituents each independently selected from the group consisting of halo, cyano, nitro, hydroxy, C₁₋₆ alkyl optionally substituted with up to 5 fluoro, C₂₋₆ alkenyl, —(CH₂)ₚC₃₋₇ cycloalkyl, C₁₋₆ alkoxy optionally substituted with up to 5 fluoro, phenyl, and hydroxy-C₁₋₆ alkyl.

In some embodiments, R^{5a}, R^{5b}, R^{5c}, R^{5d}, and R^{5e} are each independently selected from —H, optionally substituted C₁₋₆ alkyl, optionally substituted C₂₋₆ alkenyl, optionally substituted C₂₋₆ alkynyl, or optionally substituted C₁₋₆ alkoxy; provided that at least one of R^{5c}, R^{5d}, and R^{5e} is not —H or at least one of R^{5a} and R^{5b} is methyl.

In some embodiments, R^{5a}, R^{5b}, R^{5c}, R^{5d}, and R^{5e} are each independently selected from —H or optionally substituted C₁₋₆ alkyl; provided that at least one of R^{5c}, R^{5d}, and R^{5e} is not —H or at least one of R^{5a} and R^{5b} is methyl.

In some embodiments, at least one of R^{5c}, R^{5d}, and R^{5e} is not hydrogen.

In some embodiments, at least one of R^{5a}, R^{5b}, R^{5c}, R^{5d}, and R^{5e} is optionally substituted C₁₋₃ alkyl.

In some embodiments, at least one of R^{5a}, R^{5b}, R^{5c}, R^{5d}, and R^{5e} is methyl.

In some embodiments, R^{5e} is not hydrogen. In one embodiments, R^{5e} is methyl.

### Formula II

Some embodiments include a compound having a formula II:

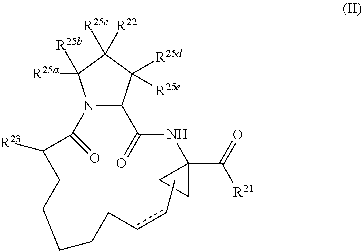

or a pharmaceutically acceptable salt or prodrug thereof, where the variables are as defined above for Formula II.

In some embodiments, compounds of Formula II have the structure of formula IIa:

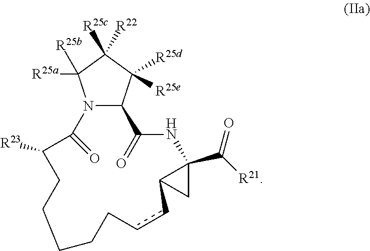

In some embodiments,
R²² is

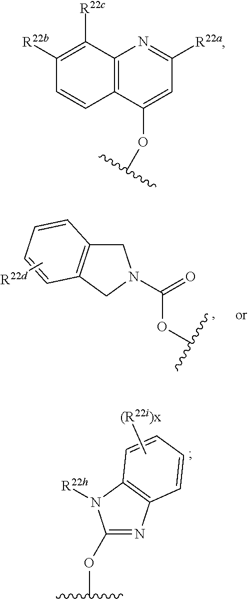

R^{22a} is thiazole optionally substituted by C₁₋₆ alkyl or thiazole optionally substituted by —NH—C₁₋₆ alkyl;
R^{22b} is C₁₋₆ alkoxy or C₃₋₇ cycloalkyloxy; and
R²³ is —NHR^{23b} or C₆₋₁₀ aryl optionally substituted with 1-3 substituents independently selected from halo, C₁₋₆ alkyl, or C₁₋₆ haloalkyl; where R^{23b} is selected from the group consisting of —H, —C(O)OR^{23c}, —C(O)R^{23c} or —C(O)NR^{23c}R^{23d}.

In some embodiments, R^{22b} is C₁₋₆ alkoxy; and R^{22c} is —H, —Br, or optionally substituted C₁₋₆ alkyl. In some embodiments, R^{22a} is thiazole optionally substituted by propyl or thiazole optionally substituted with —NH-propyl; R^{22b} is methoxy; and R^{22c} is —H or methyl.

In some embodiments, R²¹ is hydroxyl, —NHS(O)₂R^{21a} or —NR^{21b}R^{21c}; where R^{21a} is optionally substituted C₁₋₆ alkyl or optionally substituted C₃₋₇cycloalkyl; and R^{21b} and R^{21c} are each independently optionally substituted C₁₋₆ alkyl or optionally substituted C₃₋₇ cycloalkyl.

In some embodiments,
R²² is

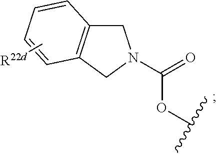

R^{22d} is —H, halo, or C₁₋₆ haloalkyl;
R²³ is —NHR^{23b} or C₆₋₁₀ aryl optionally substituted with 1-3 substituents independently selected from halo, C₁₋₆ alkyl, or C₁₋₆ haloalkyl; where R^{23b} is selected from the group consisting of —H, —C(O)OR^{23e}, —C(O)R^{23e}, or —C(O)NR^{23c}R^{23d}.

In some embodiments, R²³ is —NHR^{23b} or phenyl optionally substituted with 1-3 substituents independently selected from halo or C₁₋₆ haloalkyl; where R^{23b} is selected from the group consisting of —H, —C(O)OR^{23e}, or —C(O)NR^{23c}R^{23d}, where R^{23e} and R^{23d} are taken together with the nitrogen to which they are attached to form optionally substituted heterocyclyl.

In some embodiments, R^{25a}, R^{25b}, R^{25c}, R^{25d} and R^{25e} are each independently selected from —H, optionally substituted C₁₋₆ alkyl, optionally substituted C₂₋₆ alkenyl, optionally substituted C₂₋₆ alkynyl or optionally substituted C₁₋₆ alkoxy, provided that at least one of R^{25c}, R^{25d} and R^{25e} is not —H or at least one of R^{25a} and R^{25b} is methyl.

In some embodiments, R^{25a}, R^{25b}, R^{25c}, R^{25d} and R^{25e} are each independently selected from —H or optionally substituted C₁₋₆ alkyl, provided that at least one of R^{25e}, R^{25d} and R^{25e} is not —H or at least one of R^{25a} and R^{25b} is methyl.

In some embodiments, at least one of R^{25c}, R^{25d} and R^{25e} is not —H.

In some embodiments, at least one of R^{25a}, R^{25b}, R^{25c}, R^{25d} and R^{25e} is optionally substituted C₁₋₃ alkyl.

In some embodiments, at least one of R^{25a}, R^{25b}, R^{25c}, R^{25d} and R^{25e} is methyl.

In some embodiments, R^{25e} is not hydrogen. In one embodiment, R^{25e} is methyl.

In some embodiments, R²² is selected from

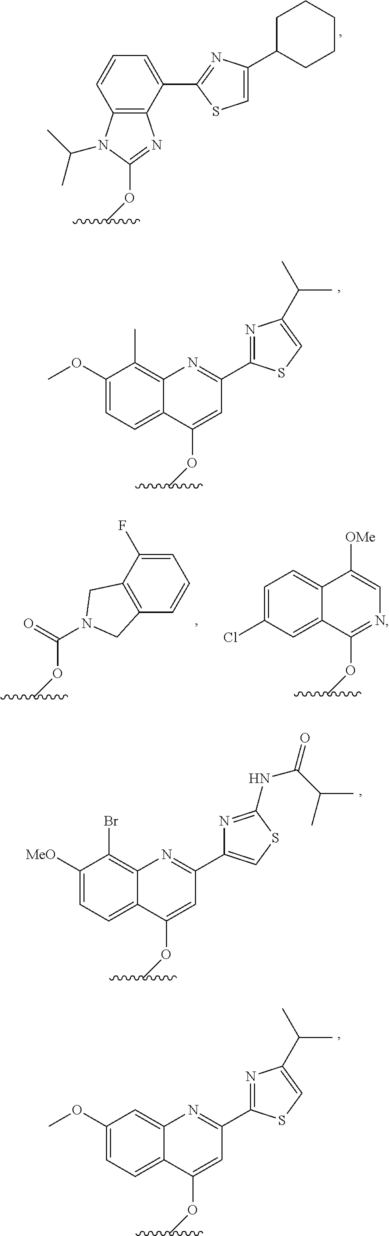

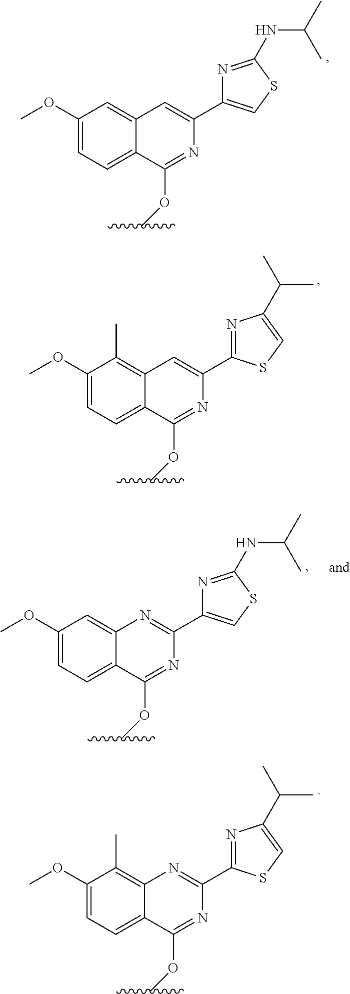

Examples of compounds of Formula I or II include, but are not limited to, the following:

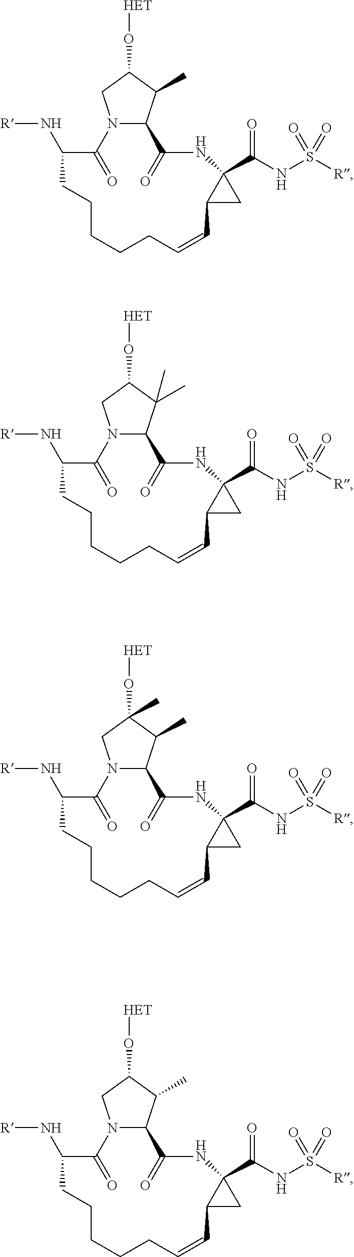

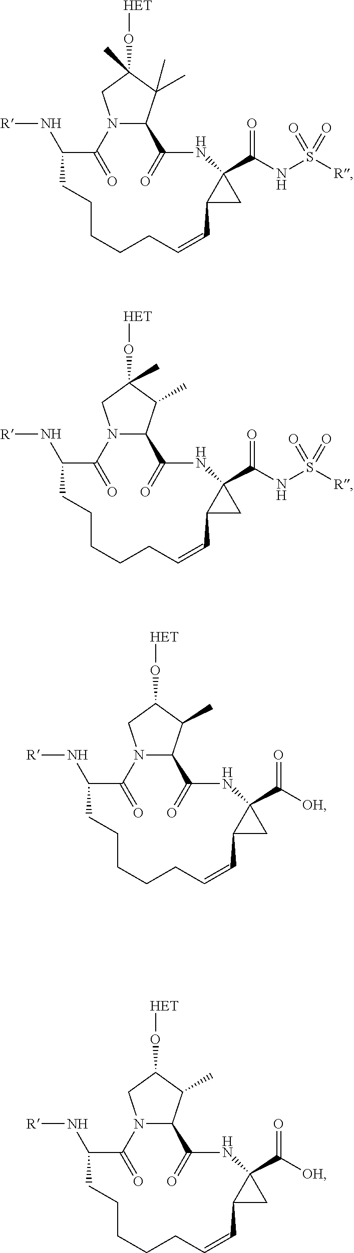

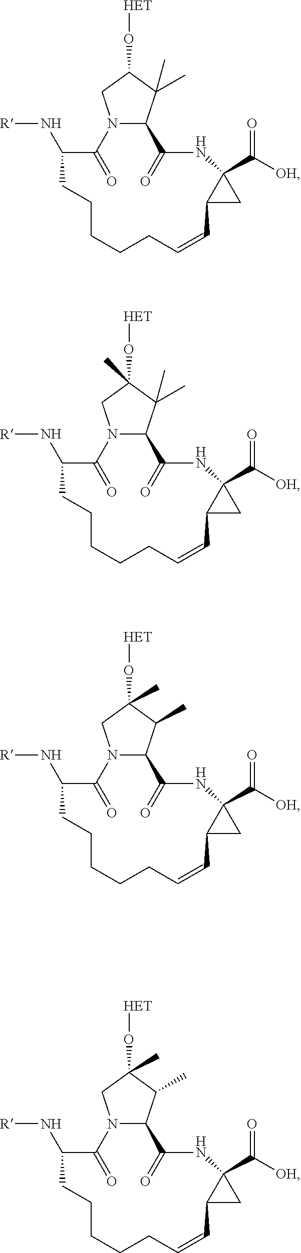

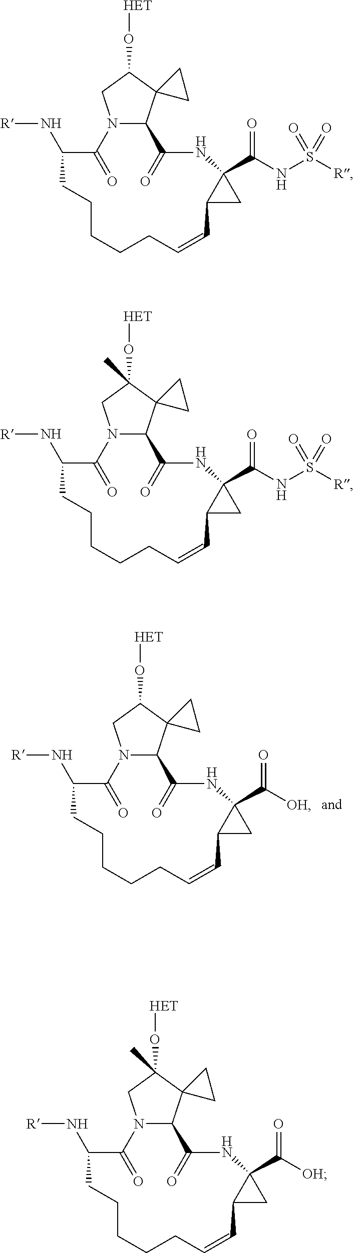

where non-limiting examples of R′ include hydrogen or a carbamate such as

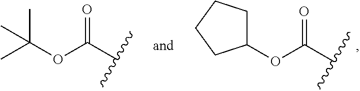

non-limiting examples of R″ include alkyl groups and cycloalkyl groups such as

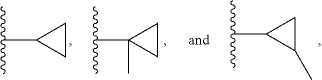

and non-limiting examples of HET include the following:

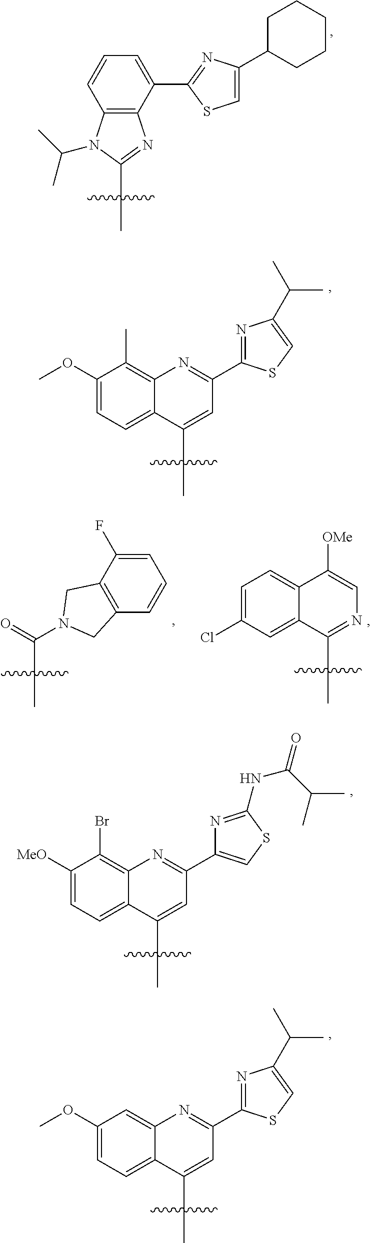

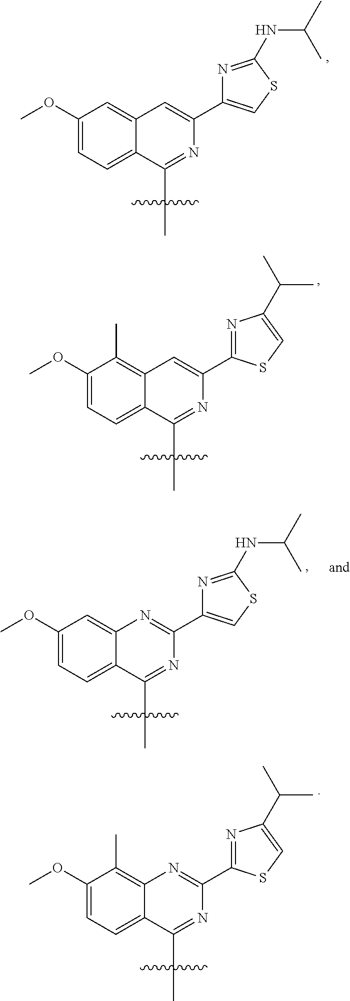

Examples of Formula I or II include, but are not limited to:

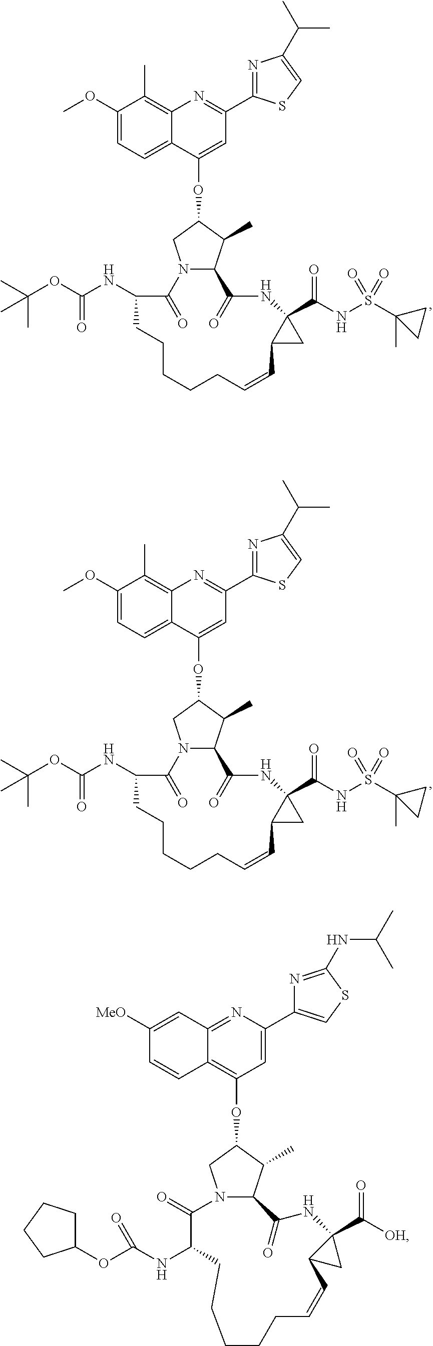

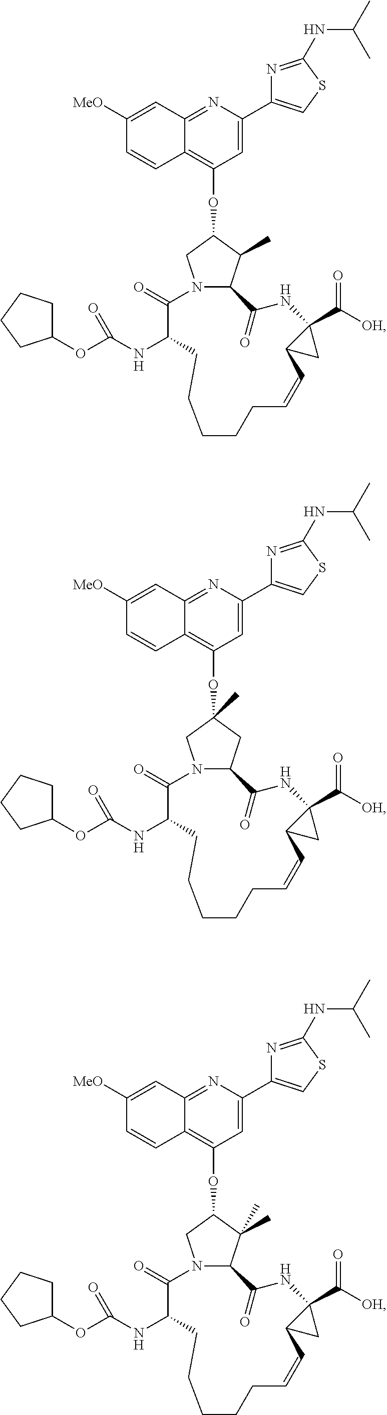

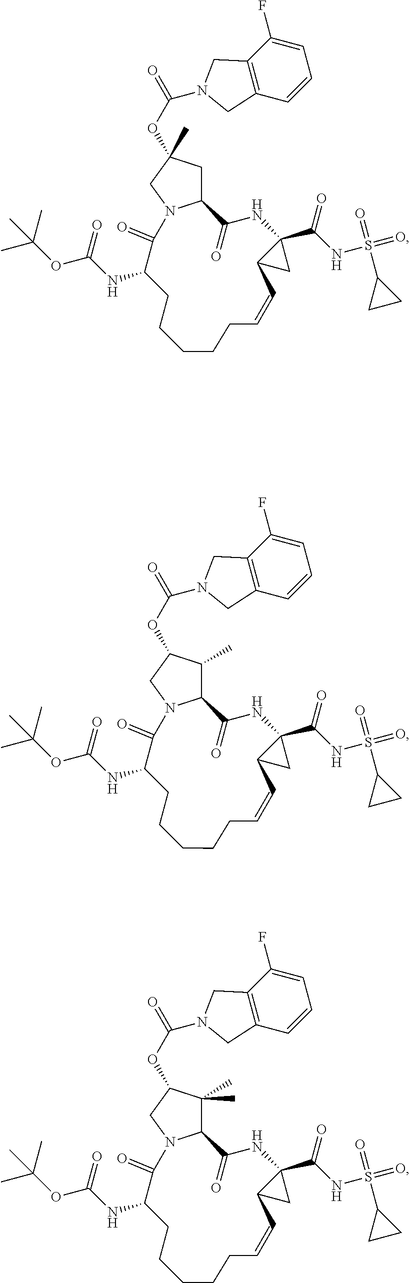

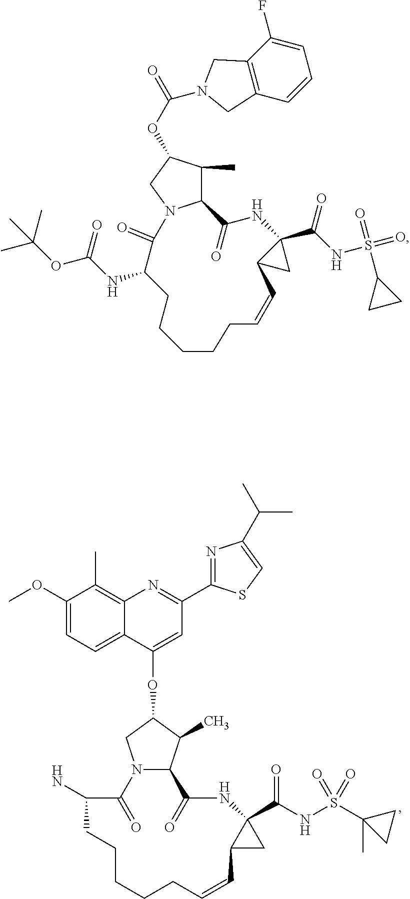

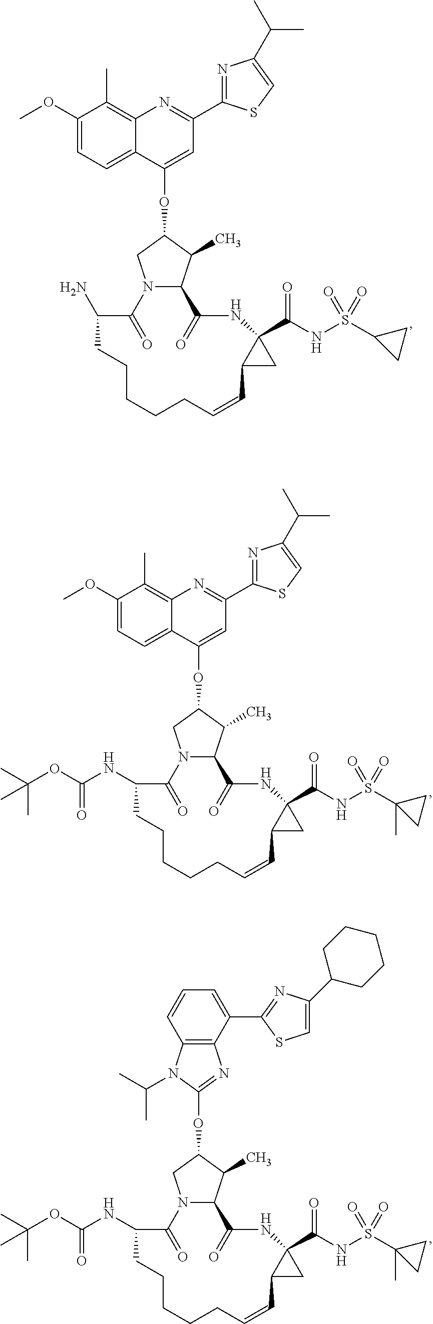

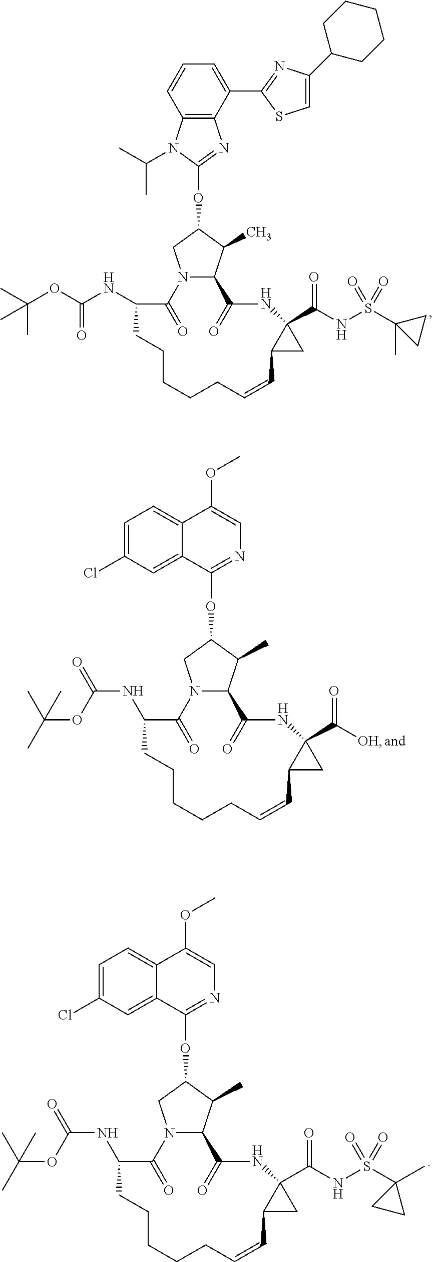

### Formula III

Some embodiments include a compound having the structure of Formula III:

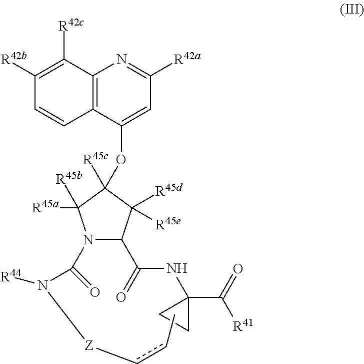

or a pharmaceutically acceptable salt or prodrug thereof, where the variables are as defined above for Formula III.

In some embodiments, compound of Formula III have the structure of Formula IIIa:

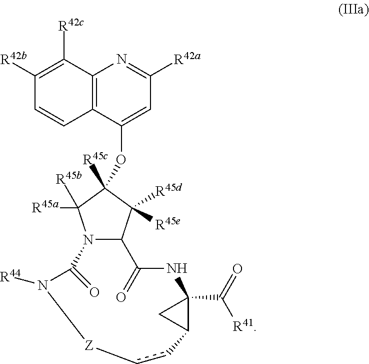

In some embodiments, compound of Formula III have the structure of Formula IIIb:

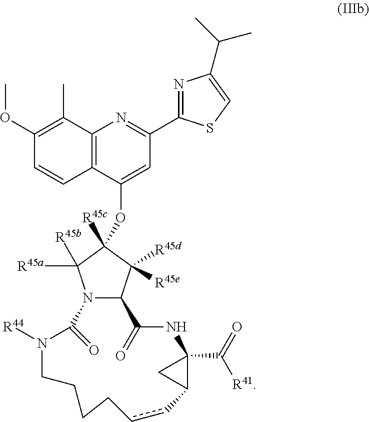

In some embodiments, at least one of R^{45a}, R^{45b}, R^{45c}, R^{45d}, and R^{45e} is not —H; or R^{45a} and R^{45b} together with the carbon atom to which they are attached to form a C₃₋₆ cycloalkyl, and R^{45c}, R^{45d} and R^{45e} are —H; or R^{45d} and R^{45e} together with the carbon atom to which they are attached to form a C₃₋₆ cycloalkyl, and R^{45a}, R^{45b} and R^{45c} are —H.

In some embodiments,
R⁴¹ is hydroxy, —NHS(O)₂R^{41b}, or —NR^{41c}R^{41d}.
R^{42a} is thiazole optionally substituted by C₁₋₆ alkyl or thiazole optionally substituted by —NH—C₁₋₆ alkyl;
R^{42b} is C₁₋₆ alkoxy;
R^{42c} is —H, optionally substituted C₁₋₆ alkyl or —Br; and
R⁴⁴ is optionally substituted C₁₋₆ alkyl.

In some embodiments,
R^{42a} is thiazole optionally substituted by C₁₋₆ alkyl;
R^{42b} is methyl; and
R^{42c} is methoxy.

In some embodiments, R^{45a}, R^{45b}, R^{45c}, R^{45d}, and R^{45e} are each independently selected from —H, optionally substituted C₁₋₆ alkyl, optionally substituted C₂₋₆ alkenyl, optionally substituted C₂₋₆ alkynyl or optionally substituted C₁₋₆ alkoxy, provided that at least one of R^{45a}, R^{45b}, R^{45c}, R^{45d}, and R^{45e} is not —H.

In some embodiments, R^{45a}, R^{45b}, R^{45c}, R^{45d}, and R^{45e} are each independently selected from —H, optionally substituted C₁₋₆ alkyl, provided that at least one of R^{45a}, R^{45b}, R^{45c}, R^{45d}, and R^{45e} is not —H.

In some embodiments, at least one of R^{45c}, R^{45d}, and R^{45e} is not —H.

In some embodiments, at least one of R^{45a}, R^{45b}, R^{45c}, R^{45d}, and R^{45e} is optionally substituted C₁₋₃ alkyl.

In some embodiments, at least one of R^{45a}, R^{45b}, R^{45c}, R^{45d}, and R^{45e} is methyl.

In some embodiments, R^{45e} is not hydrogen. In one embodiment, R^{45e} is methyl.

Some non-limiting embodiments of Formula III are selected from the group consisting of:

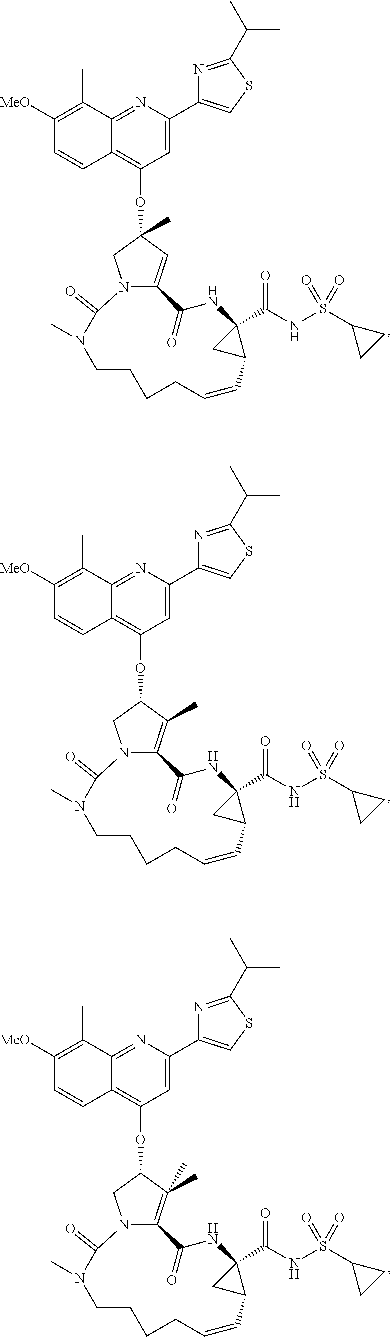

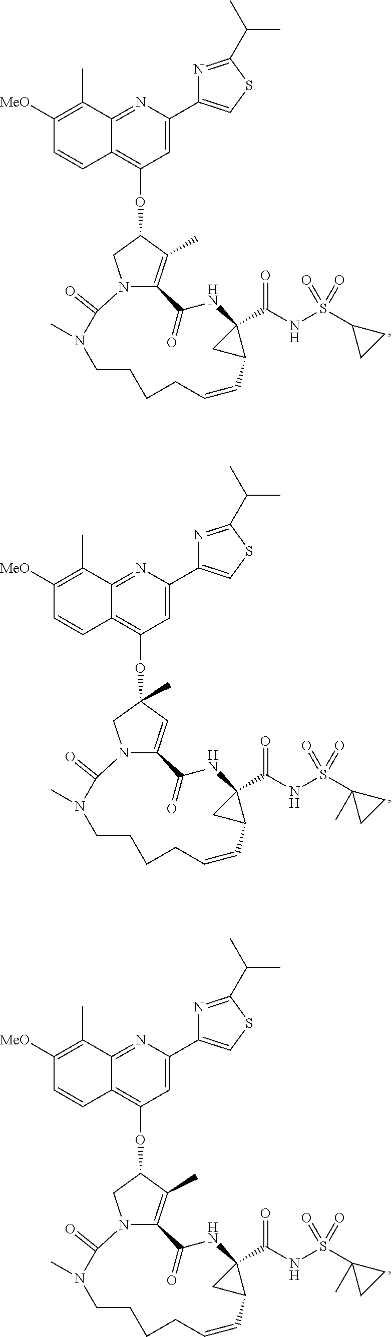

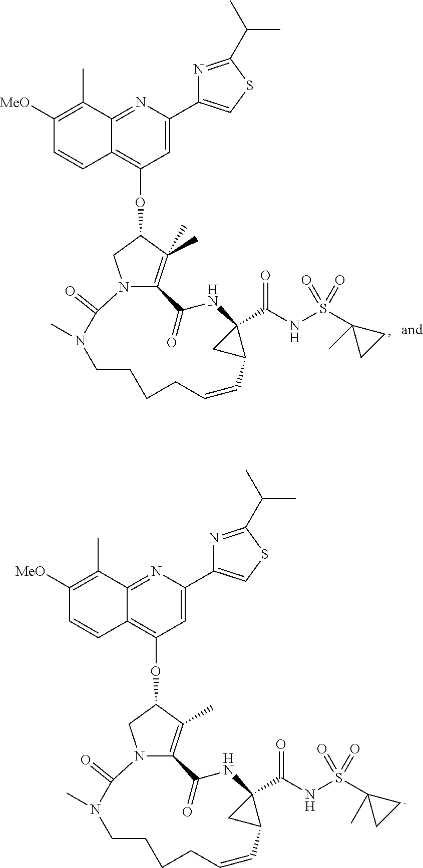

### Formula IV

Other embodiments disclosed herein include compounds have the structure of Formula IV:

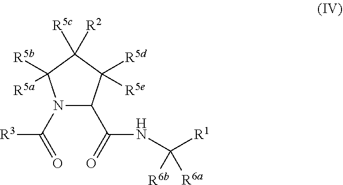

or a pharmaceutically acceptable salt or prodrug thereof, where the variables are as defined above for Formula IV.

Some embodiments of compounds of Formula IV have the structure of Formula IVa:

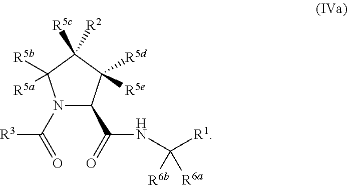

In some embodiments,
(a) R¹ is —C(O)NHS(O)₂R^{1a}, —C(O)NHS(O)NR^{1b}R^{1c}, —C(O)NR^{1b}R^{1c} or —C(O)OH;
(b) R² is

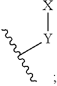

Y is —O—;
X is

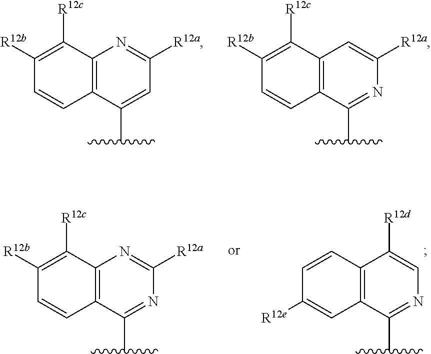

R^{12a} is selected from the group consisting of aryl, heterocyclyl and heteroaryl, each substituted with R^{12f}; wherein R^{12f} is selected from the group consisting of —H, C₁₋₆ alkyl, C₃₋₇ cycloalkyl, and —NR^{12g}R^{12h}; wherein R^{12g} and R^{12h} are each independently —H, C₁₋₆ alkyl, C₃₋₇ cycloalkyl or —C(O)—C₁₋₆ alkyl;
R^{12b} and R^{12d} are independently selected from —H, halo, C₁₋₆ alkoxy, C₃₋₇ cycloalkyloxy, or hydroxy;
R^{12c} and R^{12e} are independently selected from —H, halo, or optionally substituted C₁₋₆ alkyl;
(c) R³ is —CR^{4a}R^{4b}NR^{3a}R^{3b}, —CR^{4a}R^{4b}, or optionally substituted C₆₋₁₀ aryl; wherein
   R^{3a} is selected from the group consisting of —H, C₁₋₆ alkyl, and C₃₋₇ cycloalkyl;
   R^{3b} is selected from the group consisting of —H, —C(O)NR^{3c}R^{3d}, —C(O)OR^{3e}, heteroaryl, and aryl, wherein said heteroary and said aryl of R^{3b} are each independently optionally substituted with 1-3 substituents each independently selected from the group consisting of halo, —C(O)OH, nitro, amino, optionally substituted C₁₋₆ alkyl, and optionally substituted C₁₋₆ alkoxy; and
   R^{4a} and R^{4b} are independently selected from the group consisting of —H, optionally substituted C₁₋₆ alkyl, C₂₋₆ alkenyl, optionally substituted C₃₋₇ cycloalkyl, optionally substituted aryl, optionally substituted heteroaryl, optionally substituted C₇₋₁₀ arylalkyl, and optionally substituted C₆₋₁₂ heteroaryl-alkyl; and
(d) R^{6a} and R^{6b} are taken together to form C₃₋₇ cycloalkyl optionally substituted with C₁₋₆ alkyl or C₂₋₆ alkenyl.

In some embodiments,
(a) R¹ is —C(O)NHS(O)₂R^{1a}, —C(O)NHS(O)NR^{1a}R^{1b}, C(O)NR^{1b}R^{1c}, or —C(O)OH;

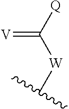

(b) R² is
   V is O or S; W is —O— or —NH—;
   Q is

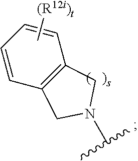

s is 1 or 2; t is 0, 1, 2, 3, or 4; and each R¹²ⁱ is independently selected from the group consisting of halo, methyl, and C₁₋₆ alkoxy optionally substituted with up to 5 fluoro;
(d) R³ is —CR^{4a}R^{4b}NR^{3a}R^{3b} or —CR^{4a}R^{4b}-optionally substituted aryl; wherein
   R^{3a} is selected from the group consisting of —H, C₁₋₆ alkyl, and C₃₋₇ cycloalkyl;
   R^{3b} is selected from the group consisting of —H, —C(O)OR^{3e}, —C(O)NR^{3c}R^{3d}, heteroaryl, and aryl, wherein said heteroaryl or said aryl of R^{3b} is optionally substituted with 1-3 substituents selected from the group consisting of halo, —C(O)OH, nitro, amino, optionally substituted C₁₋₆ alkyl, and optionally substituted C₁₋₆ alkoxy;
   R^{4a} and R^{4b} are independently selected from the group consisting of —H, optionally substituted C₁₋₆ alkyl, C₂₋₆ alkenyl, optionally substituted C₃₋₇ cycloalkyl, optionally substituted aryl, optionally substituted heteroaryl, optionally substituted C₇₋₁₀ arylalkyl, and optionally substituted C₆₋₁₂ heteroaryl-alkyl; and
(e) R^{6a} and R^{6b} are taken together to form C₃₋₇ cycloalkyl optionally substituted with C₁₋₆ alkyl or C₂₋₆ alkenyl.

In some embodiments, each R¹²ⁱ is independently —CF₃, —Cl or —F.

In some embodiments, R³ is —CR^{4a}R^{4b}NR^{3a}R^{3b} or —CR^{4a}R^{4b}-aryl optionally substituted with 1-3 substituents independently selected from halo or C₁₋₆ haloalkyl; wherein
R^{3a} is selected from the group consisting of —H, C₁₋₆ alkyl, and C₃₋₇ cycloalkyl;
R^{3b} is selected from the group consisting of —H, —C(O)OR^{3e}, —C(O)NR^{3c}R^{3d}, heteroaryl, and aryl, wherein said heteroaryl or said aryl of R^{3b} is optionally substituted with halo or C₁₋₆ haloalkyl; and
R^{3c} and R^{4d} are taken together with the nitrogen to which they are attached to form optionally substituted heterocyclyl or optionally substituted heteroaryl;
R^{3e} is optionally substituted C₁₋₆ alkyl, C₃₋₇ cycloalkyl, or heterocyclyl; wherein said C₃₋₇ cycloalkyl and said heterocyclyl are each optionally substituted with one or more substituents each independently selected from the group consisting of halo, cyano, nitro, hydroxy, C₁₋₆ alkyl optionally substituted with up to 5 fluoro, C₂₋₆ alkenyl, C₃₋₇ cycloalkyl-alkyl, C₁₋₆ alkoxy optionally substituted with up to 5 fluoro, phenyl, and hydroxy-C₁₋₆ alkyl; and
R^{4a} and R^{4b} are independently selected from the group consisting of —H, optionally substituted C₁₋₆ alkyl, C₂₋₆ alkenyl, optionally substituted C₃₋₇ cycloalkyl, optionally substituted aryl, optionally substituted heteroaryl, optionally substituted C₇₋₁₀ arylalkyl, and optionally substituted C₆₋₁₂ heteroaryl-alkyl.

In some embodiments, R^{4a} and R^{4b} are independently —H, C₁₋₆ alkyl optionally substituted with up to 5 fluoro, or C₂₋₆ alkenyl.

In some embodiments, R¹ is —C(O)NHS(O)₂R^{1a}, —C(O)NHS(O)NR^{1b}R^{1c}, C(O)NR^{1b}R^{1c}, or —C(O)OH; wherein
R^{1a} is —(CH₂)ₘC₃₋₇ cycloalkyl optionally substituted with C₁₋₆ alkyl optionally substituted with up to 5 fluoro; and
R^{1b} and R^{1c} are independently —H, optionally substituted C₁₋₆ alkyl, optionally substituted C₃₋₇ cycloalkyl, optionally substituted aryl, optionally substituted heterocyclyl, or R^{1b} and R^{1c} are taken together with the nitrogen to which they are attached to form three- to seven-membered heterocyclyl optionally substituted with 1-3 R^{1f}.

Some embodiments of compounds of Formula IV have the structure of Formula IVb:

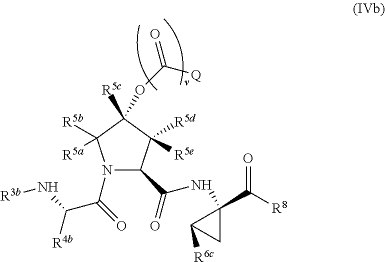

(a) R⁸ is —NHS(O)₂R^{1a}, —NHS(O)₂NR^{1b}R^{1c}, —NR^{1b}R^{1c}, —OR^{1d};
   R^{1b}, R^{1c}, and R^{1d} are independently selected from the group consisting of —H, C₁₋₆ alkyl, C₃₋₇ cycloalkyl, C₆₋₁₀ aryl, heteroaryl, and heterocyclyl; each optionally substituted with one or more substituents independently selected from the group consisting of halo, cyano, nitro, hydroxyl, optionally substituted C₁₋₆ alkyl, —(CH₂)ₘC₃₋₇ cycloalkyl, C₂₋₆ alkenyl, C₁₋₆ alkoxy optionally substituted with up to 5 fluoro, C₄₋₁₁ alkylcycloalkyl, and phenyl;
   or R^{1b} and R^{1c} are taken together with the nitrogen to which they are attached to form heteroaryl or three- to seven-membered heterocyclyl, each optionally substituted with 1-3 R^{1f};
(b) Q is heteroaryl or heterocyclyl; each optionally substituted with one or more substituents each independently selected from the group consisting of halo, cyano, nitro, hydroxy, C₁₋₆ alkyl optionally substituted with up to 5 fluoro, C₁₋₆ alkoxy optionally substituted with up to 5 fluoro, C₃₋₇ cycloalkoxy, optionally substituted aryl, and optionally substituted heteroaryl;
(c) R^{4b} is selected from the group consisting of —H, C₁₋₆ alkyl optionally substituted with up to 5 fluoro, C₂₋₆ alkenyl, C₃₋₇ cycloalkyl optionally substituted with up to 5 fluoro, and C₄₋₇ cycloalkyl-alkyl; and
(d) R^{3b} is —H, —C(O)OR^{3e}, —C(O)NR^{3c}R^{3d}, aryl, or heteroaryl; wherein said aryl and said heteroaryl are each independently optionally substituted by 1-3 substituents selected from the group consisting of halo, —C(O)OH, —C(O)NR^{3cc}R^{3cc}, —NR^{3dd}R^{3dd}, nitro, amino, cyano, C₁₋₆ alkyl optionally substituted with up to 5 fluoro or cyano, C₁₋₆ alkoxy optionally substituted with up to 5 fluoro, C₁₋₆ alkylthio group optionally substituted with up to 5 fluoro, C₁₋₆ alkylamino optionally substituted with up to 5 fluoro, three- to seven-membered heterocyclyl, and heteroaryl containing 1-3 heteroatoms independently selected from N or O;
   each R^{3cc} is independently selected from the group consisting of hydrogen, —C(O)NR^{A}R^{B}, and C(O)OR;
   each R^{3dd} is independently selected from the group consisting of hydrogen, C(O)R, —C(O)NR^{A}R^{B}, and C(O)OR, —S(O)₂NR^{A}R^{B}, and —S(O)₂R;
   R, R^{A} and R^{B} are each independently selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted three- to ten-membered heterocycloalkyl, optionally substituted C₆₋₁₀ aryl, optionally substituted three- to ten-membered heteroaryl, halo, cyano, hydroxy, optionally substituted C₁₋₆ alkoxy, aryloxy, heteroaryloxy, sulfhydryl, C₁₋₆ alkylthio, arylthio, mono- and di-(C₁₋₆)alkyl amino, quaternary ammonium salts, amino(C₁₋₆)alkoxy, hydroxy(C₁₋₆)alkylamino, amino(C₁₋₆)alkylthio, cyanoamino, nitro, carbamyl, keto (oxo), carbonyl, carboxy, glycolyl, glycyl, hydrazino, guanyl, sulfamyl, sulfonyl, sulfinyl, thiocarbonyl, and thiocarboxy;
R^{3e} is —H, optionally substituted C₁₋₆ alkyl, optionally substituted C₆₋₁₀ aryl, optionally substituted heteroaryl, or optionally substituted three- to seven-membered heterocyclyl; and
(e) R^{6c} is optionally substituted C₁₋₆ alkyl, optionally substituted C₂₋₆ alkenyl, or optionally substituted C₃₋₇ cycloalkyl; and
(f) v is 0 or 1.

In some embodiments,
Q is

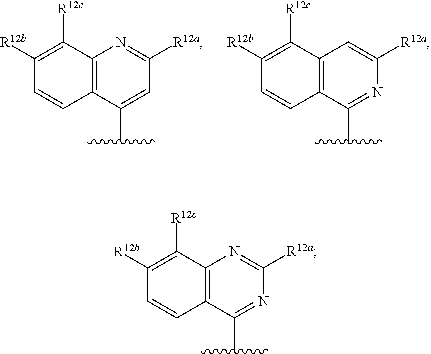

v is 0;
   R^{12a} is a thiazole optionally substituted with a moiety selected from —C₁₋₆ alkyl, —NH—C₁₋₆ alkyl, or —NHC(O)—C₁₋₆ alkyl;
   R^{12b} is C₁₋₆ alkoxy or C₃₋₇ cycloalkyloxy;
   R^{12c} is H halo, or optionally substituted C₁₋₆ alkyl; and
   R^{3b} is —H, —C(O)NR^{3c}R^{3d}, —C(O)OR^{3e}, heteroaryl or aryl, wherein the heteroaryl or aryl of R³″ is optionally substituted by 1-3 R³″; wherein
   R^{1c} and R^{ad} are taken together with the nitrogen to which they are attached to form optionally substituted heterocyclyl or optionally substituted heteroaryl;
   R^{3e} is optionally substituted C₁₋₆ alkyl, optionally substituted C₃₋₇ cycloalkyl or optionally substituted C₃₋₇ heterocyclyl containing 1-3 O atoms;
   R^{3h} is halo, amino, nitro, —C(O)OH, CONR^{3cc}R^{3cc}, —NR^{3dd}R^{3dd}, alkyl optionally substituted with up to 5 fluoro, or C₁₋₈ alkoxy optionally substituted with up to 5 fluoro;
   each R^{3 cc} is independently selected from the group consisting of hydrogen, —C(O)NR^{A}R^{B}, and C(O)OR; and
   each R^{3dd} is independently selected from the group consisting of hydrogen, C(O)R, —C(O)NR^{A}R^{B}, and C(O)OR, —S(O)₂NR^{A}R^{B}, and —S(O)₂R;
   R, R^{A} and R^{B} are each independently selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted three- to ten-membered heterocycloalkyl, optionally substituted C₆₋₁₀ aryl, optionally substituted three- to ten-membered heteroaryl, halo, cyano, hydroxy, optionally substituted C₁₋₆ alkoxy, aryloxy, heteroaryloxy, sulfhydryl, C₁₋₆ alkylthio, arylthio, mono- and di-(C₁₋₆)alkyl amino, quaternary ammonium salts, amino(C₁₋₆)alkoxy, hydroxy(C₁₋₆)alkylamino, amino(C₁₋₆)alkylthio, cyanoamino, nitro, carbamyl, keto (oxo), carbonyl, carboxy, glycolyl, glycyl, hydrazino, guanyl, sulfamyl, sulfonyl, sulfinyl, thiocarbonyl, and thiocarboxy.

In some embodiments,
Q is

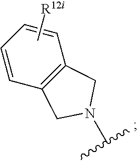

v is 1;
R¹²ⁱ is —H, halo, or C₁₋₆ haloalkyl; and
R^{3b} is —H, —C(O)NR^{3c}R^{3d}, —C(O)OR^{3ea}, heteroaryl, and aryl, wherein said heteroary and said aryl of R^{3b} are each independently optionally substituted by 1-3 R^{3h}; wherein
   R^{3c} and R^{3d} are taken together with the nitrogen to which they are attached to form optionally substituted heterocyclyl or optionally substituted heteroaryl;
   R^{3e} is optionally substituted C₁₋₆ alkyl, optionally substituted C₃₋₇ cycloalkyl or optionally substituted C₃₋₇ heterocyclyl containing 1-3 O atom; and
   R^{3h} is halo, amino, nitro, —C(O)OH, C₁₋₈ alkyl optionally substituted with up to 5 fluoro, or C₁₋₈ alkoxy optionally substituted with up to 5 fluoro.

In some embodiments, R¹²ⁱ is —F or —CF₃.

In some embodiments, R^{1b}, R^{1c}, and R^{1d} are independently selected from the group consisting of —H, C₁₋₆ alkyl, C₃₋₇ cycloalkyl, C₆₋₁₀ aryl, heteroaryl, and heterocyclyl; each optionally substituted with one or more substituents independently selected from the group consisting of halo, cyano, nitro, hydroxyl, C₁₋₆ alkyl optionally substituted with up to 5 fluoro, —(CH₂)ₘC₃₋₇ cycloalkyl, C₂₋₆ alkenyl, C₁₋₆ alkoxy optionally substituted with up to 5 fluoro, C₄₋₁₁ alkylcycloalkyl, and phenyl.

In some embodiments, R⁸ is hydroxy or —NHS(O)₂R^{1a}; wherein R^{1a} is C₁₋₆ alkyl or C₃₋₇ cycloalkyl; each optionally substituted with one or more substituents each independently selected from the group consisting of halo, C₁₋₆ alkyl optionally substituted with up to 5 fluoro, hydroxy-C₁₋₆ alkyl, C₂₋₆ alkenyl, and C₁₋₆ alkoxy optionally substituted with up to 5 fluoro.

In some embodiments, R^{3b} is —H, —C(O)NR^{3c}R^{3d}, —C(O)OR^{3e}, heteroaryl or aryl, wherein the heteroaryl or aryl of R^{3b} is optionally substituted by 1-3 R^{3h}; wherein R^{3e} is optionally substituted C₁₋₆ alkyl or optionally substituted C₃₋₇ cycloalkyl, and R^{3h} is halo or C₁₋₆ alkyl optionally substituted with up to 5 fluoro.

In some embodiments, at least one of R^{5c}, R^{5d}, and R^{5e} is not hydrogen.

In some embodiments, at least one of R^{5a}, R^{5b}, R^{5c}, R^{5d} and R^{5e} is optionally substituted C₁₋₆ alkyl.

In some embodiments, at least one of R^{5a}, R^{5b}, R^{5c}, R^{5d} and R^{5e} is methyl.

In some embodiments, R^{5d} or R^{5e} is not hydrogen. In one embodiment, at least one of R^{5d} and R^{5e} is methyl.

Examples of compounds of Formula IV, IVa and/or IVb include, but are not limited to, the following:

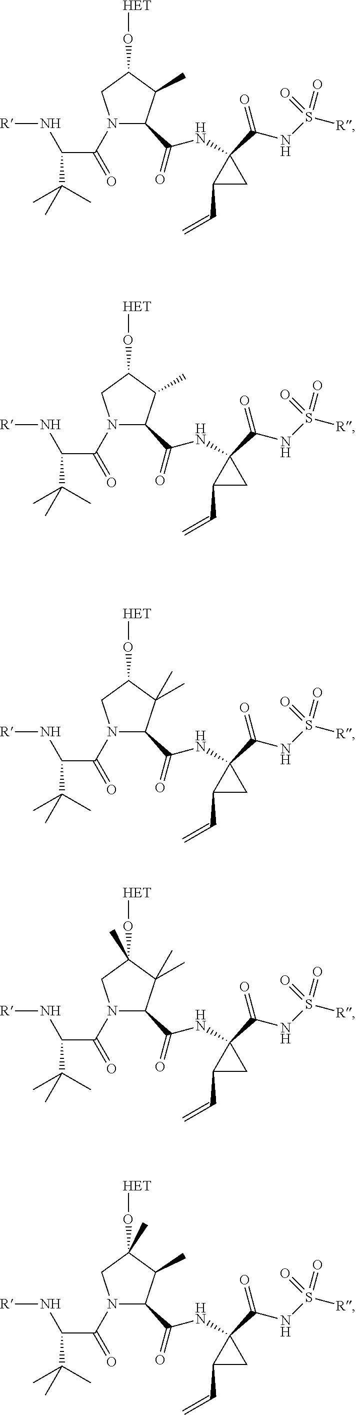

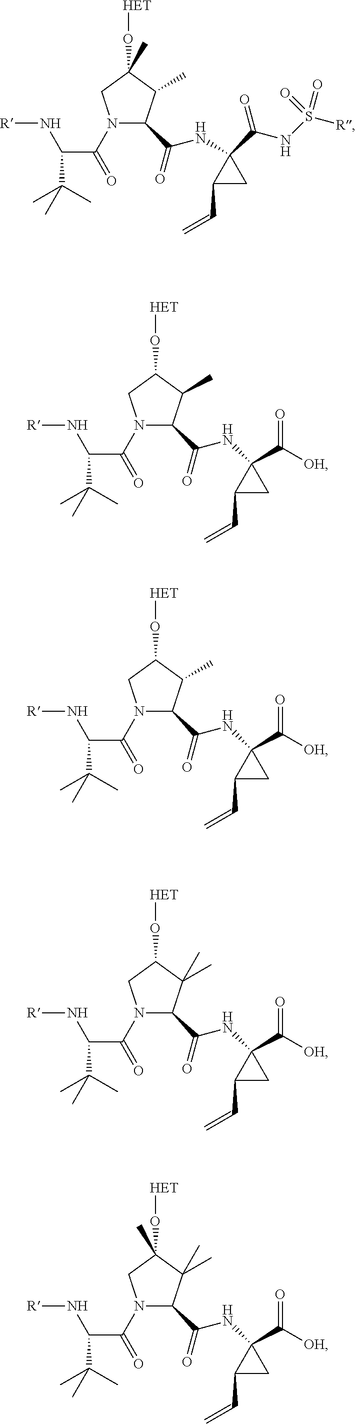

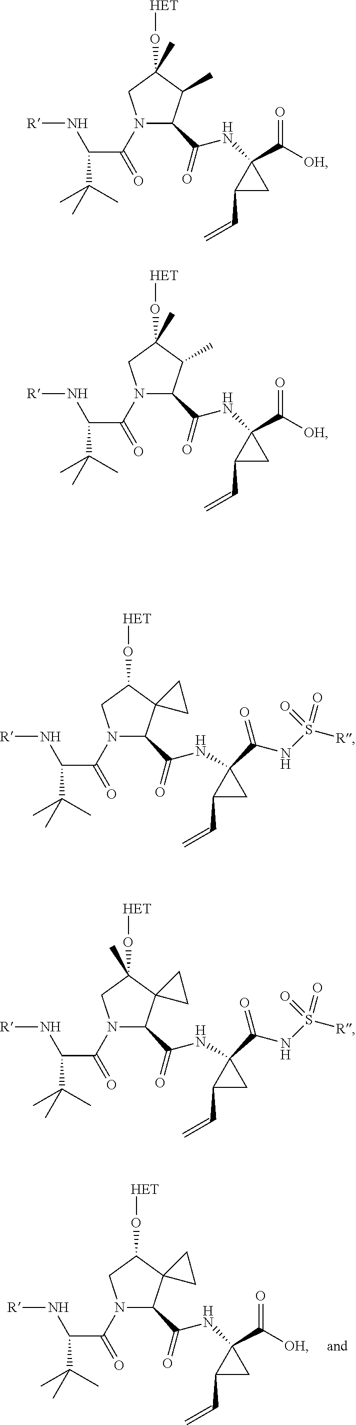

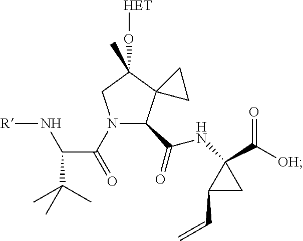

wherein non-limiting examples of R′ include hydrogen or a carbamate such as

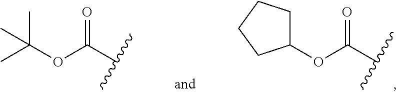

non-limiting examples of R″ include alkyl groups and cycloalkyl groups such as

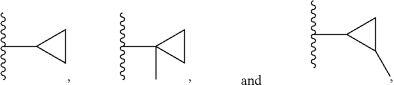

and non-limiting examples of HET include the following:

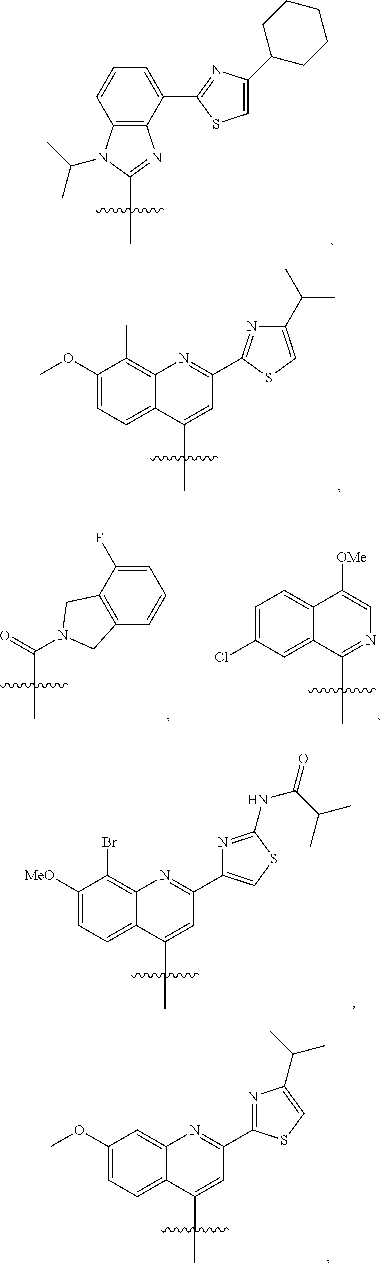

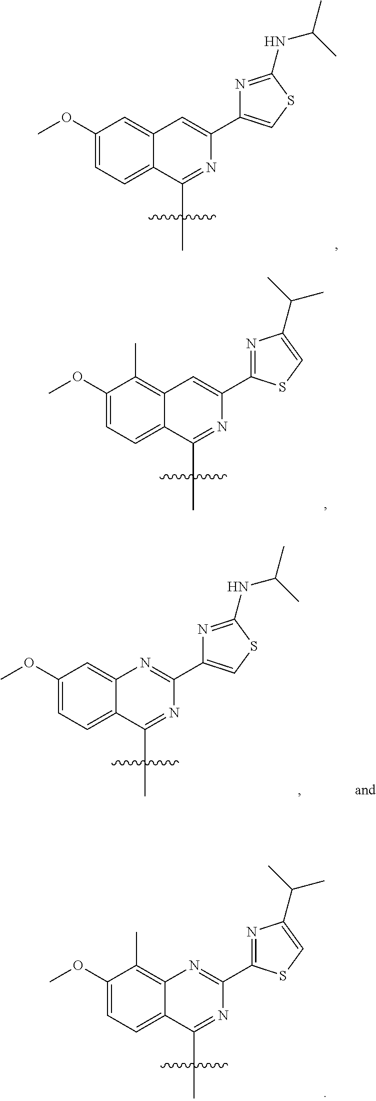

Examples of compounds of Formula IV, IVa or IVb include, but not limited to:

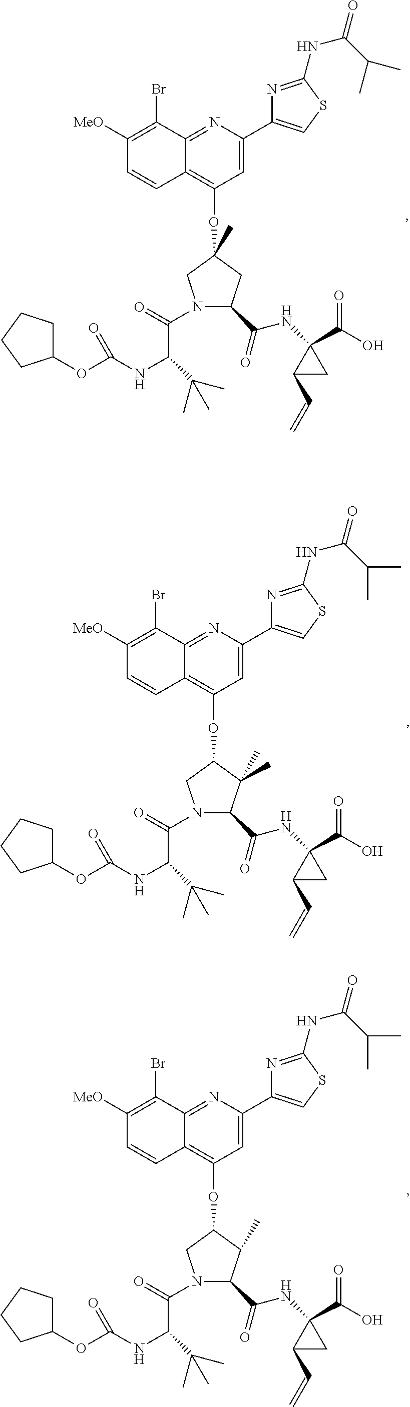

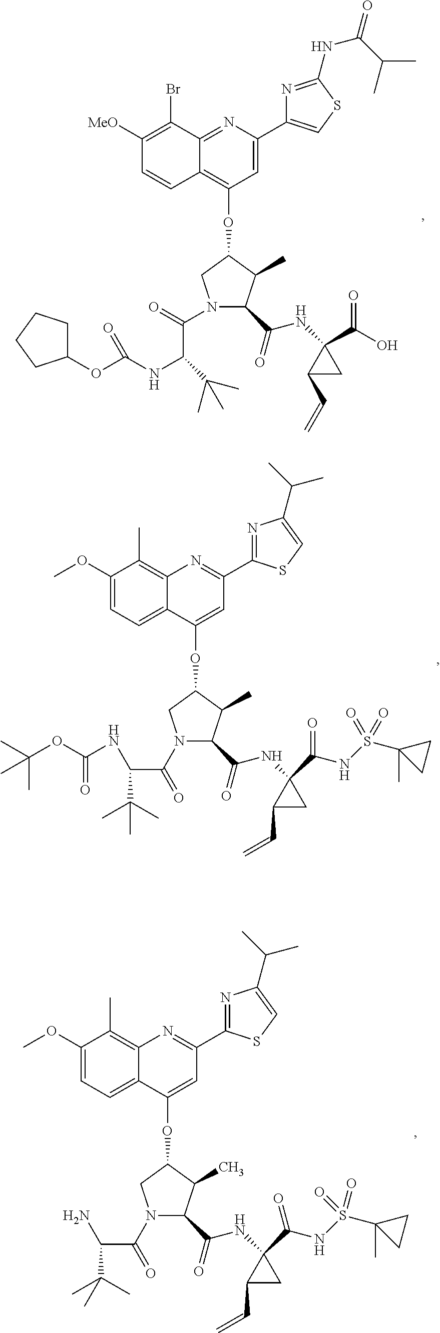

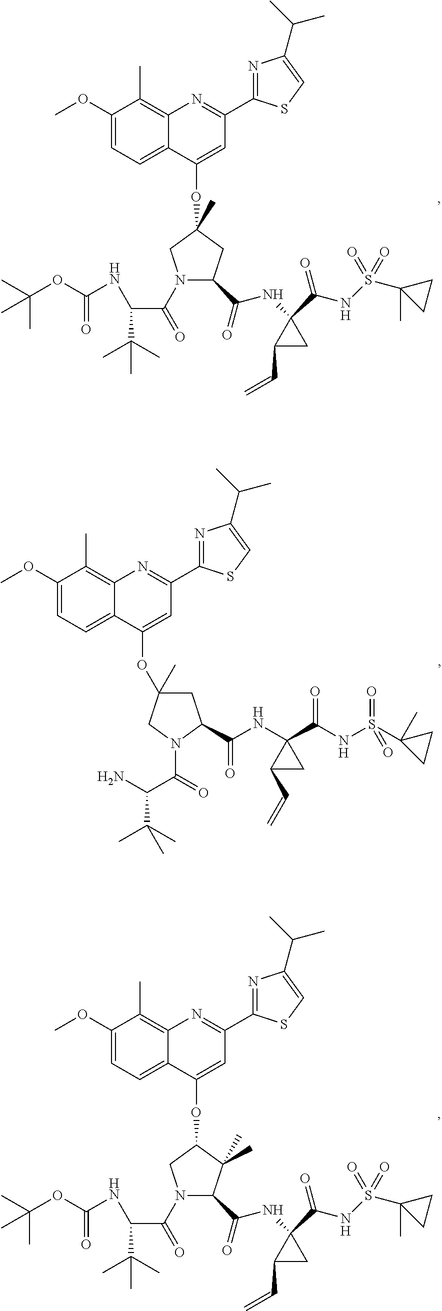

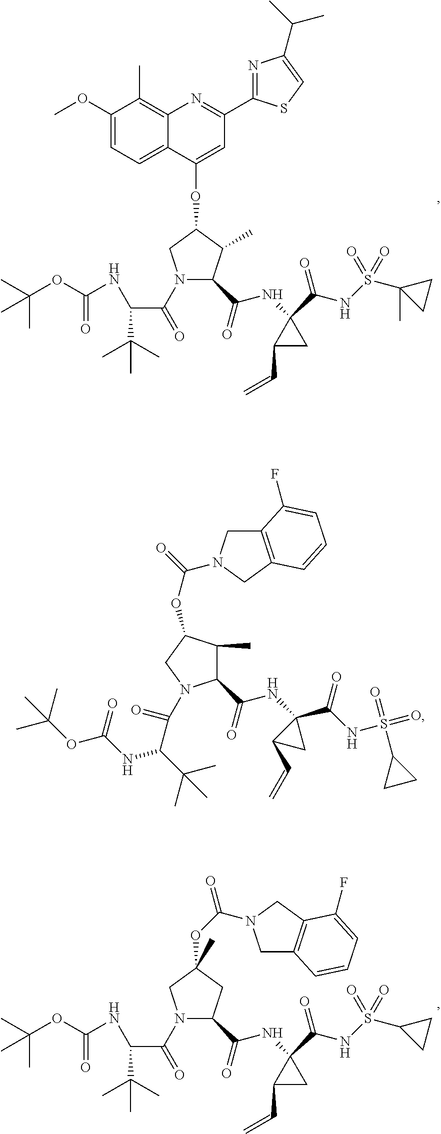

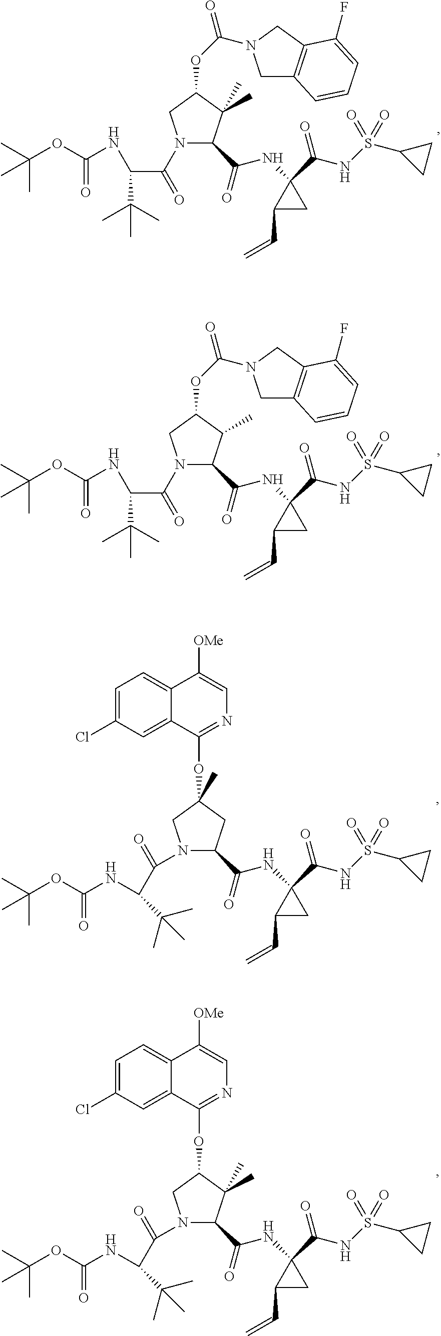

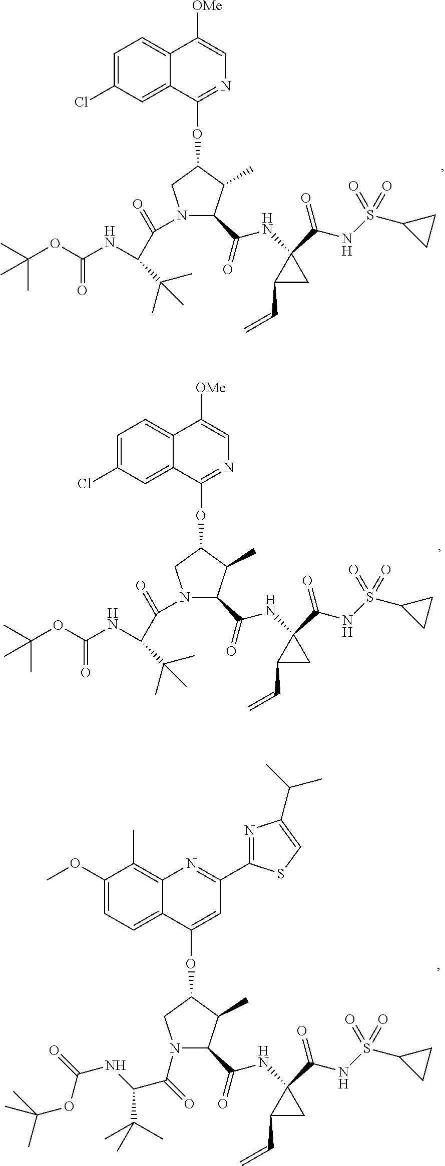

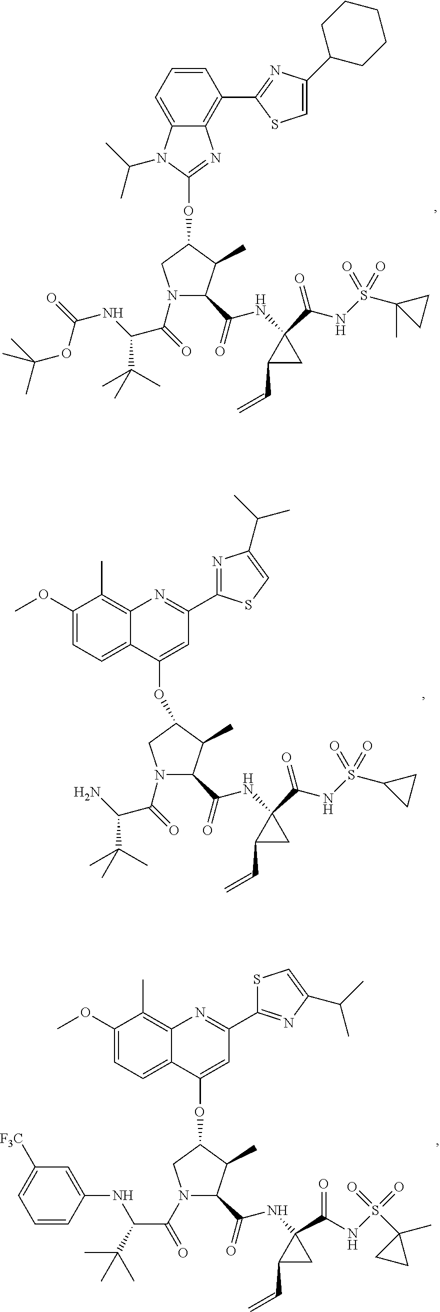

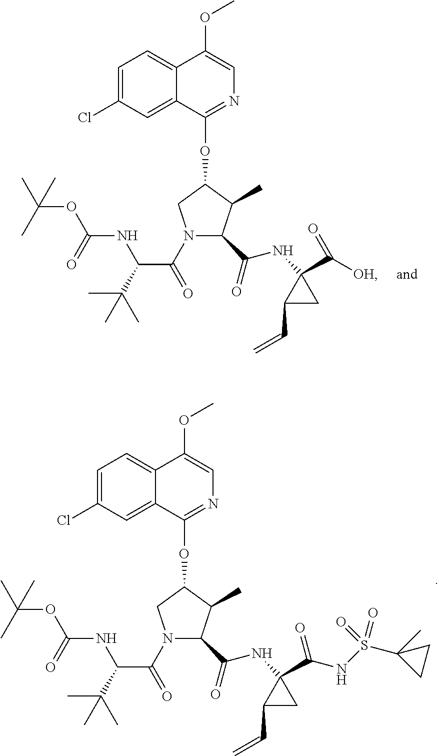

### Formula V

Some embodiments include compounds having the structure of Formula V:

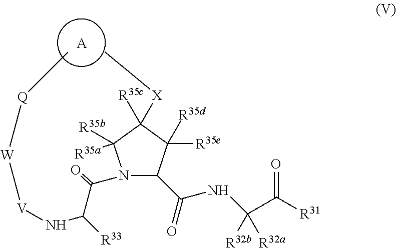

or a pharmaceutically acceptable salt or prodrug thereof, where the variables are as defined above for Formula V.

Some embodiments of Formula V include compounds having the structure of Formula Va:

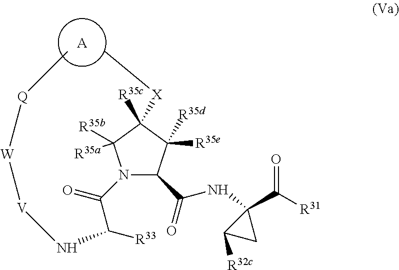

wherein R^{32c} is —H, C₁₋₆ alkyl, C₂₋₆ alkenyl, and optionally substituted C₃₋₇ cycloalkyl.

In some embodiments,
A is heteroaryl or heterocyclyl; each optionally substituted with halo;
X is a bond or selected from the group consisting of —OCH₂—, —CH₂O—, —OC(O)—, and —NHC(O)—;
V is —C(O)—;
W is —O— or —NH—;
R³¹ is selected from —NHS(O)₂R^{31a}, —NHS(O)₂NR^{31b}R^{31c}, NR^{31b}R^{31c}, —OR^{31d},
R^{32c} is C₁₋₆ alkyl or C₂₋₆ alkenyl; and
R³³ is C₁₋₆ alkyl optionally substituted with up to 5 fluoro, C₃₋₇ cycloalkyl optionally substituted with up to 5 fluoro, or C₄₋₁₀ cycloalkyl-alkyl.

Some embodiments of Formula V include compounds having the structure of Formula Vb:

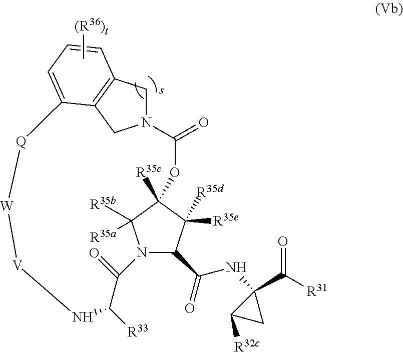

wherein s is 1 or 2; t is 1, 2, 3 or 4; and each R³⁶ is independently —Cl or —F.

In some embodiments, Q is C₅₋₈ alkylene optionally substituted with 1-3 C₁₋₆ alkyl.

In some embodiments, at least one of R^{35a}, R^{35b}, and R^{35c} is not —H.

In some embodiments, at least one of R^{35a}, R^{35b}, R^{35c}, R^{35d}, and R^{35e} is optionally substituted C₁₋₆ alkyl.

In some embodiments, at least one of R^{35a}, R^{35b}, R^{35c}, R^{35d}, and R^{35e} is methyl.

In some embodiments, R^{35d} or R^{35e} is not hydrogen. In one embodiment, at least one of R^{35d} and R^{35e} is methyl.

Examples of compounds of Formula V, Va or Vb include, but are not limited to:

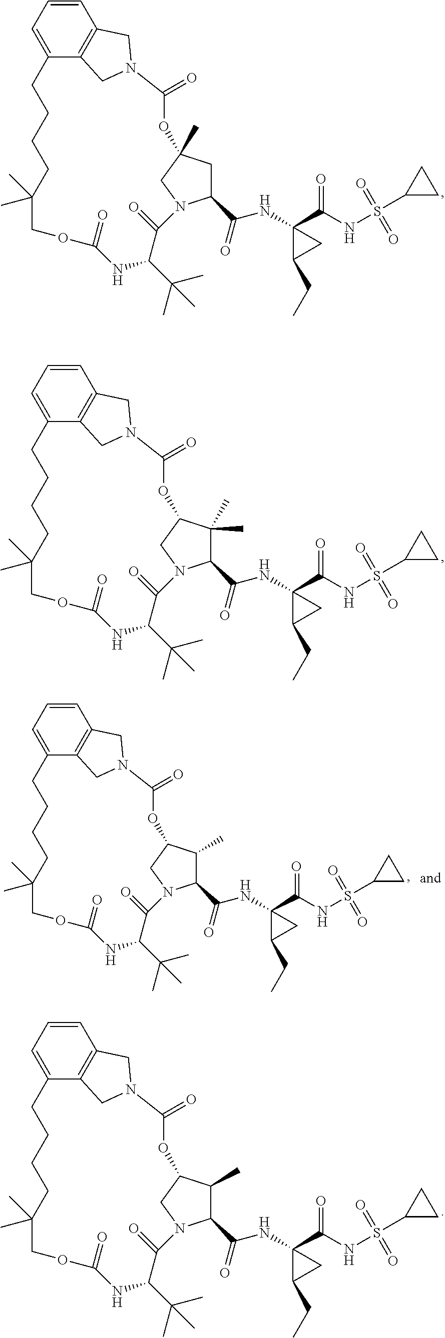

### Formula VI

Some embodiments include compounds of Formula VI:

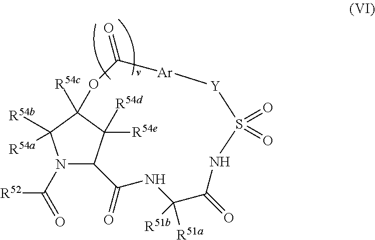

or a pharmaceutically acceptable salt or prodrug thereof, where the variables are as defined above for Formula VI.

Some embodiments of Formula VI include compounds having the structure of Formula VIa:

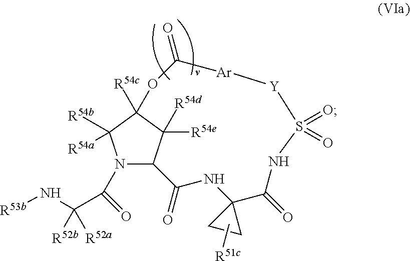

wherein:
Ar is optionally substituted C₅₋₁₀ fused bicyclic heteroaryl, optionally substituted C₆₋₁₀ aryl, or optionally substituted isoindolinyl;
R^{51c} is —H or C₁₋₆ alkyl, C₂₋₆ alkenyl, or C₂₋₆ alkynyl;
R^{52a} and R^{52b} are independently selected from the group consisting of —H, optionally substituted C₁₋₆ alkyl, C₂₋₆ alkenyl, optionally substituted C₃₋₇ cycloalkyl, optionally substituted aryl, optionally substituted heteroaryl, optionally substituted C₇₋₁₀ arylalkyl, and optionally substituted C₆₋₁₂ heteroaryl-alkyl;
R^{53b} is selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, —C(O)OR^{53e}, —C(O)R^{53e}, —C(O)NR^{53c}R^{53d}, —S(O)₂R^{53e}, —S(O)₂NR^{53c}R^{53d}, —S(O)₂OR^{53e}, heteroaryl optionally substituted by 1-3 R^{53h}, and aryl optionally substituted by 1-3 R^{53h}; wherein
   R^{53e} is selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, and optionally substituted heterocyclyl; and
   R^{53h} is selected from the group consisting of halo, CF₃, heterocyclyl, amino, —NO₂, —NR^{3dd}R^{3dd}, C(═O)NR^{3dd}R^{3dd}, C(═O)OR^{53hh}, optionally substituted C₁₋₆ alkyl, optionally substituted C₁₋₆ alkoxy, C₂₋₆ alkenyl, C₂₋₆ alkynyl, and heteroaryl containing 1-3 heteroatoms independently selected from N or O;
   each R^{53hh} independently selected from the group consisting of hydrogen, and C₁₋₆ alkyl; and
   each R^{3dd} is independently selected from the group consisting of hydrogen, C(O)R, —C(O)NR^{A}R^{B}, and C(O)OR, —S(O)₂NR^{A}R^{B}, and —S(O)₂R;
   R, R^{A} and R^{B} are each independently selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted three- to ten-membered heterocycloalkyl, optionally substituted C₆₋₁₀ aryl, optionally substituted three- to ten-membered heteroaryl, halo, cyano, hydroxy, optionally substituted C₁₋₆ alkoxy, aryloxy, heteroaryloxy, sulfhydryl, C₁₋₆ alkylthio, arylthio, mono- and di-(C₁₋₆)alkyl amino, quaternary ammonium salts, amino(C₁₋₆)alkoxy, hydroxy(C₁₋₆)alkylamino, amino(C₁₋₆)alkylthio, cyanoamino, nitro, carbamyl, keto (oxo), carbonyl, carboxy, glycolyl, glycyl, hydrazino, guanyl, sulfamyl, sulfonyl, sulfinyl, thiocarbonyl, and thiocarboxy.

Some embodiments of Formula VI include compounds having the structure of Formula VIb:

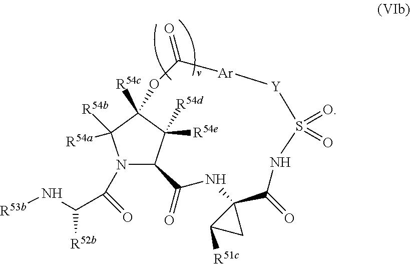

In some embodiments, v is 0.

In some embodiments, Ar is optionally substituted benzoimidazolen-1,2-yl.

In some embodiments, Y is represented by:

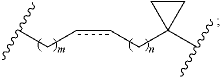

wherein m and n are independently 0, 1, 2, 3, 4, 5, or 6; and the dashed line represents an optional double bond.

Some embodiments of Formula VI include compounds having the structure of Formula VIc:

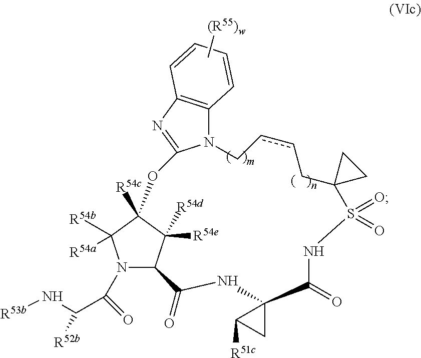

wherein R⁵⁵ is independently —H, halo, C₁₋₆ alkyl optionally substituted with up to 5 fluoro, or C₁₋₆ alkyl optionally substituted with up to 5 fluoro; m and n are independently 0, 1, 2, 3, 4, 5, or 6; w is 0, 1, 2, 3 or 4; and the dashed line represents an optional double bond.

In some embodiments, the sum of m and n is 2, 3, 4, 5, or 6.

In some embodiments, at least one of R^{54c}, R^{54d}, and R^{54e} is not hydrogen.

In some embodiments, at least one of R^{54a}, R^{54b}, R^{54c}, R^{54d}, and R^{54e} is optionally substituted C₁₋₆ alkyl.

In some embodiments, at least one of R^{54a}, R^{54b}, R^{54c}, R^{54d}, and R^{54e} is methyl.

In some embodiments, R^{54d} or R^{54e} is not hydrogen. In one embodiment, at least one of R^{54d} and R^{54e} is methyl.

### Compositions

The present embodiments further provide compositions, including pharmaceutical compositions, comprising compounds of the general Formulae I, Ia, II, IIa, III, IIIa, IIIb, IV, IVa, IVb, V, Va, Vb, VI, VIa, VIb, and VIc or any compounds disclosed herein.

A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The present embodiments provide for a method of inhibiting NS3/NS4 protease activity comprising contacting a NS3/NS4 protease with a compound disclosed herein.

The present embodiments provide for a method of treating hepatitis by modulating NS3/NS4 protease comprising contacting a NS3/NS4 protease with a compound disclosed herein.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

In many embodiments, a subject compound inhibits the enzymatic activity of a hepatitis virus C (HCV) NS3 protease. Whether a subject compound inhibits HCV NS3 protease can be readily determined using any known method. Typical methods involve a determination of whether an HCV polyprotein or other polypeptide comprising an NS3 recognition site is cleaved by NS3 in the presence of the agent. In many embodiments, a subject compound inhibits NS3 enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound inhibits enzymatic activity of an HCV NS3 protease with an IC₅₀ of less than about 50 μM, e.g., a subject compound inhibits an HCV NS3 protease with an IC₅₀ of less than about 40 μM, less than about 25 μM, less than about 10 μM, less than about 1 μM, less than about 100 nM, less than about 80 nM, less than about 60 nM, less than about 50 nM, less than about 25 nM, less than about 10 nM, less than about 5 nM, less than about 1 nM, or less than about 0.5 nM, or less.

In many embodiments, a subject compound inhibits the enzymatic activity of a hepatitis virus C (HCV) NS3 helicase. Whether a subject compound inhibits HCV NS3 helicase can be readily determined using any known method. In many embodiments, a subject compound inhibits NS3 enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Treating a Hepatitis Virus Infection

The methods and compositions described herein are generally useful in treatment of an of HCV infection.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

The method involves administering an effective amount of a compound of Formulae I, Ia, II, IIa, III, IIIa, IIIb, IV, IVa, IVb, V, Va, Vb, VI, VIa, VIb, and VIc, or any compounds disclosed herein, optionally in combination with an effective amount of one or more additional antiviral agents. In some embodiments, an effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective to reduce viral load to lower than 100 genome copies/mL serum.

In some embodiments, an effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective to achieve a 1.5-log, a 2-log, a 2.5-log, a 3-log, a 3.5-log, a 4-log, a 4.5-log, or a S-log reduction in viral titer in the serum of the individual.

In many embodiments, an effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount effective to reduce ALT levels to less than about 45 IU/mL serum.

A therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound of Formulae I, Ia, II, IIa, III, IIIa, IIIb, IV, IVa, IVb, V, Va, Vb, VI, VIa, VIb, and VIc, or any compounds disclosed herein and an additional antiviral agent is a synergistic amount. The additional antiviral agent may itself be a combination of antiviral agents, e.g., a combination of pegylated interferon-alfa and ribavirin. As used herein, a “synergistic combination” or a “synergistic amount” of a compound of Formulae I, Ia, II, IIa, III, IIIa, IIIb, IV, IVa, IVb, V, Va, Vb, VI, VIa, VIb, and VIc, or any compounds disclosed herein and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of the compound of Formulae I, Ia, II, IIa, III, IIIa, IIIb, IV, IVa, IVb, V, Va, Vb, VI, VIa, VIb, and VIc, or any compounds disclosed herein when administered at that same dosage as a monotherapy and (ii) the therapeutic or prophylactic benefit of the additional antiviral agent when administered at the same dosage as a monotherapy.

In some embodiments, a selected amount of a compound of Formulae I, Ia, II, IIa, III, IIIa, IIIb, IV, IVa, IVb, V, Va, Vb, VI, VIa, VIb, and VIc, or any compounds disclosed herein and a selected amount of an additional antiviral agent are effective when used in combination therapy for a disease, but the selected amount of the compound of Formulae I, Ia, II, IIa, III, IIIa, IIIb, IV, IVa, IVb, V, Va, Vb, VI, VIa, VIb, and VIc, or any compounds disclosed herein and/or the selected amount of the additional antiviral agent is ineffective when used in monotherapy for the disease. Thus, the embodiments encompass (1) regimens in which a selected amount of the additional antiviral agent enhances the therapeutic benefit of a selected amount of the compound of Formulae I, Ia, II, IIa, III, IIIa, IIIb, IV, IVa, IVb, V, Va, Vb, VI, VIa, VIb, and VIc, or any compounds disclosed herein when used in combination therapy for a disease, where the selected amount of the additional antiviral agent provides no therapeutic benefit when used in monotherapy for the disease (2) regimens in which a selected amount of the compound of Formulae I, Ia, II, IIa, III, IIIa, IIIb, IV, IVa, IVb, V, Va, Vb, VI, VIa, VIb, and VIc, or any compounds disclosed herein enhances the therapeutic benefit of a selected amount of the additional antiviral agent when used in combination therapy for a disease, where the selected amount of the compound of Formulae I, Ia, II, IIa, III, IIIa, IIIb, IV, IVa, IVb, V, Va, Vb, VI, VIa, VIb, and VIc, or any compounds disclosed herein provides no therapeutic benefit when used in monotherapy for the disease and (3) regimens in which a selected amount of the compound of Formulae I, Ia, II, IIa, III, IIIa, IIIb, IV, IVa, IVb, V, Va, Vb, VI, VIa, VIb, and VIc, or any compounds disclosed herein and a selected amount of the additional antiviral agent provide a therapeutic benefit when used in combination therapy for a disease, where each of the selected amounts of the compound of Formulae I, Ia, II, IIa, III, IIIa, IIIb, IV, IVa, IVb, V, Va, Vb, VI, VIa, VIb, and VIc, or any compounds disclosed herein and the additional antiviral agent, respectively, provides no therapeutic benefit when used in monotherapy for the disease. As used herein, a “synergistically effective amount” of a compound of Formulae I, Ia, II, IIa, III, IIIa, IIIb, IV, IVa, IVb, V, Va, Vb, VI, VIa, VIb, and VIc, or any compounds disclosed herein and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents. Effective amounts of compounds of Formulae I, Ia, II, IIa, III, IIIa, IIIb, IV, IVa, IVb, V, Va, Vb, VI, VIa, VIb, and VIc, or any compounds disclosed herein, with and without one or more additional antiviral agents, as well as dosing regimens, are as discussed below.

Whether treatment with a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that effects a change of one unit or more in the fibrosis stage based on pre- and post-therapy liver biopsies. In particular embodiments, a therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound of Formulae I, Ia, II, IIa, III, IIIa, IIIb, IV, IVa, IVb, V, Va, Vb, VI, VIa, VIb, and VIc, or any compounds disclosed herein. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with an interferon receptor agonist and pirfenidone (or a pirfenidone analog). These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is effective in reducing the incidence of a disorder associated with cirrhosis of the liver can readily be determined by those skilled in the art.

Reduction in liver fibrosis increases liver function. Thus, the embodiments provide methods for increasing liver function, generally involving administering a therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Formulations

The above-discussed active agent(s) can be formulated using well-known reagents and methods. Compositions are provided in formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., a Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an agent is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the agents can be achieved in various ways, including oral, buccal, rectal, parenteral, intraperitoneal, intradermal, subcutaneous, intramuscular, transdermal, intratracheal, etc., administration. In many embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Other Antiviral or Antifibrotic Agents

As discussed above, a subject method will in some embodiments be carried out by administering an NS3 inhibitor that is a compound of Formulae I, Ia, II, IIa, III, IIIa, IIIb, IV, IVa, IVb, V, Va, Vb, VI, VIa, VIb, and VIc, or any compounds disclosed herein, and optionally one or more additional antiviral agent(s).

In some embodiments, the method further includes administration of one or more interferon receptor agonist(s).

In other embodiments, the method further includes administration of pirfenidone or a pirfenidone analog.

Additional antiviral agents that are suitable for use in combination therapy include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′3,′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β-D-ribofuranosyl-1H-1,2,4-triazole-3-carb oxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid, 5-thiazolylmethyl ester[5S-(5R*,8R*,10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man. Because of its strong inhibitory effect on cytochrome P450 3A and the inhibitory effect on cytochrome P450 2D6, ritonavir at doses below the normal therapeutic dosage may be combined with other protease inhibitors to achieve therapeutic levels of the second protease inhibitor while reducing the number of dosage units required, the dosing frequency, or both.

In some embodiments, the method further includes administration of another protease inhibitor. In some embodiments, the method further includes administration of a NS5A inhibitor. In some embodiments, the method further includes administration of a helicase inhibitor. In some embodiments, the method further includes administration of a polymerase inhibitor.

In some embodiments, an additional antiviral agent is administered during the entire course of NS3 inhibitor compound treatment. In other embodiments, an additional antiviral agent is administered for a period of time that is overlapping with that of the NS3 inhibitor compound treatment, e.g., the additional antiviral agent treatment can begin before the NS3 inhibitor compound treatment begins and end before the NS3 inhibitor compound treatment ends; the additional antiviral agent treatment can begin after the NS3 inhibitor compound treatment begins and end after the NS3 inhibitor compound treatment ends; the additional antiviral agent treatment can begin after the NS3 inhibitor compound treatment begins and end before the NS3 inhibitor compound treatment ends; or the additional antiviral agent treatment can begin before the NS3 inhibitor compound treatment begins and end after the NS3 inhibitor compound treatment ends.

### Methods of Treatment

### Monotherapies

The NS3 inhibitor compounds described herein may be used in acute or chronic therapy for HCV disease. In many embodiments, the NS3 inhibitor compound is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The NS3 inhibitor compound can be administered 5 times per day, 4 times per day, tid, bid, qd, qod, biw, tiw, qw, qow, three times per month, or once monthly. In other embodiments, the NS3 inhibitor compound is administered as a continuous infusion.

In many embodiments, an NS3 inhibitor compound of the embodiments is administered orally.

In connection with the above-described methods for the treatment of HCV disease in a patient, an NS3 inhibitor compound as described herein may be administered to the patient at a dosage from about 0.01 mg to about 100 mg/kg patient bodyweight per day, in 1 to 5 divided doses per day. In some embodiments, the NS3 inhibitor compound is administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific NS3 inhibitor compound, the severity of the symptoms and the susceptibility of the subject to side effects. Preferred dosages for a given NS3 inhibitor compound are readily determinable by those of skill in the art by a variety of means. A preferred means is to measure the physiological potency of a given interferon receptor agonist.

In many embodiments, multiple doses of NS3 inhibitor compound are administered. For example, an NS3 inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with a TNF-α Antagonist and an Interferon

Some embodiments provide a method of treating an HCV infection in an individual having an HCV infection, the method comprising administering an effective amount of an NS3 inhibitor, and effective amount of a TNF-α antagonist, and an effective amount of one or more interferons.

### Subjects Suitable for Treatment

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Any of the above treatment regimens can be administered to individuals who have been diagnosed with an HCV infection. Any of the above treatment regimens can be administered to individuals having advanced or severe stage liver fibrosis as measured by a Knodell score of 3 or 4 or no or early stage liver fibrosis as measured by a Knodell score of 0, 1, or 2. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (MBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system.).

### Synthetic Methods

The compounds described herein can be prepared according to the procedures and schemes shown below. The numberings in each of the following schemes, are meant for that specific scheme only, and should not be construed or confused with the same numberings, if any, in other schemes.

Methods for preparing the final compounds described herein utilize a substituted proline precursor. Once the substituted proline precursor is obtained, the rest of the molecule may be prepared using a variety of synthetic approaches that add the remaining pieces in any suitable order. As a non-limiting illustration of the flexibility provided to the skilled artisan upon obtaining the substituted proline precursor, Formula IV-1 depicted below is a typical subgenus of compounds to Formula IV and identifies the variety of attachment points that may be utilized to produce the final compound.

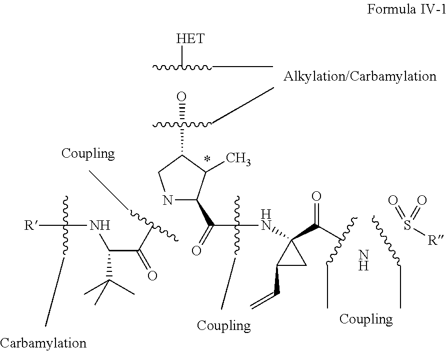

A compound of Formula IV-1, wherein R′ includes, but is not limited to, hydrogen or a carbamate such as t-butyl or cyclopentyl carbamate, R″ includes, but is not limited to alkyl and cycloalkyl groups such as cyclopropyl and methyl-substituted cyclopropyl, the “*” indicates that the carbon atom may optionally be of an R or S configuration, and HET includes, but is not limited to, the following:

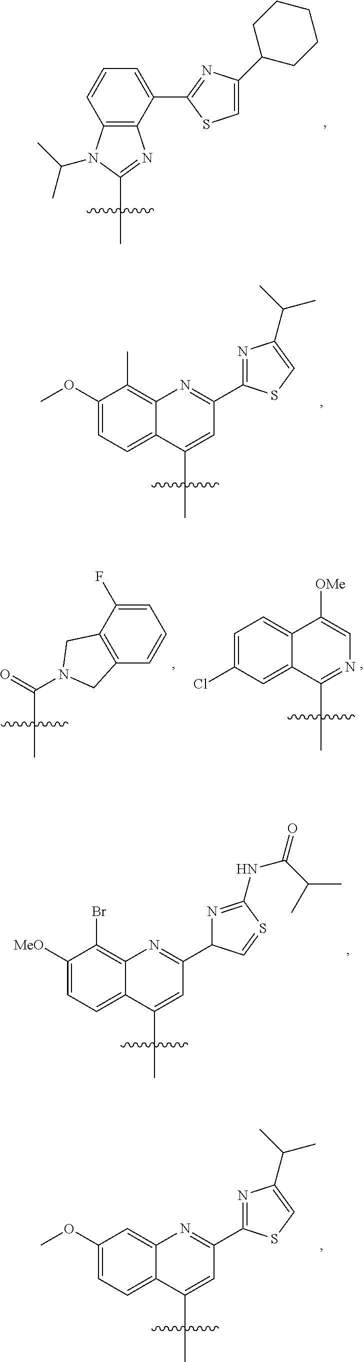

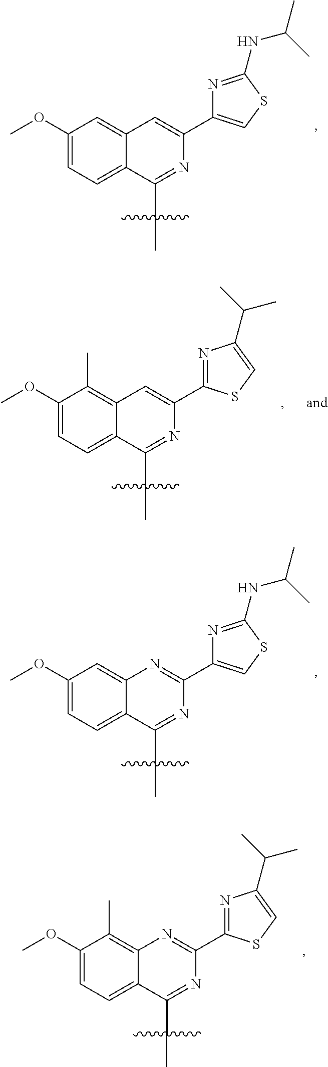

may be prepared using several synthetic approaches. One such approach may utilize a proline core derivatized with a HET moiety, such as compounds of Formulas A to H, or salts thereof, and rely on the introduction of peptide fragments via coupling steps.

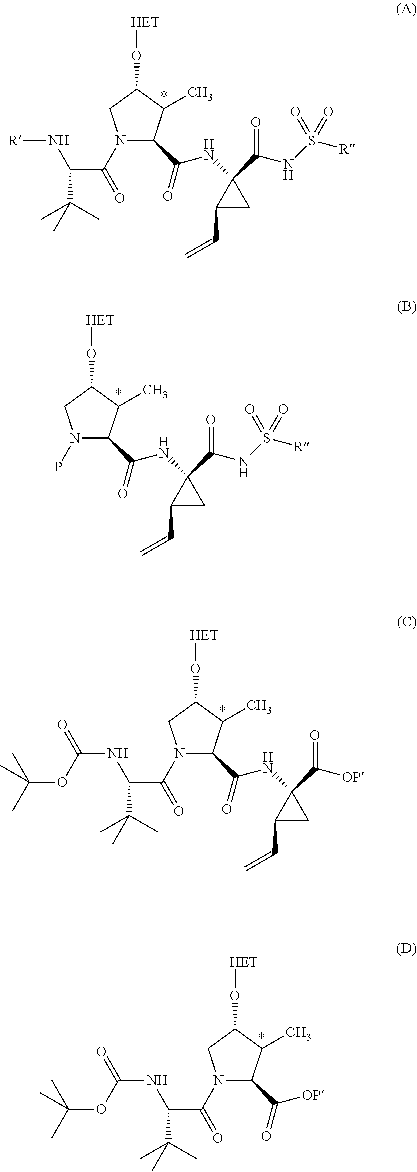

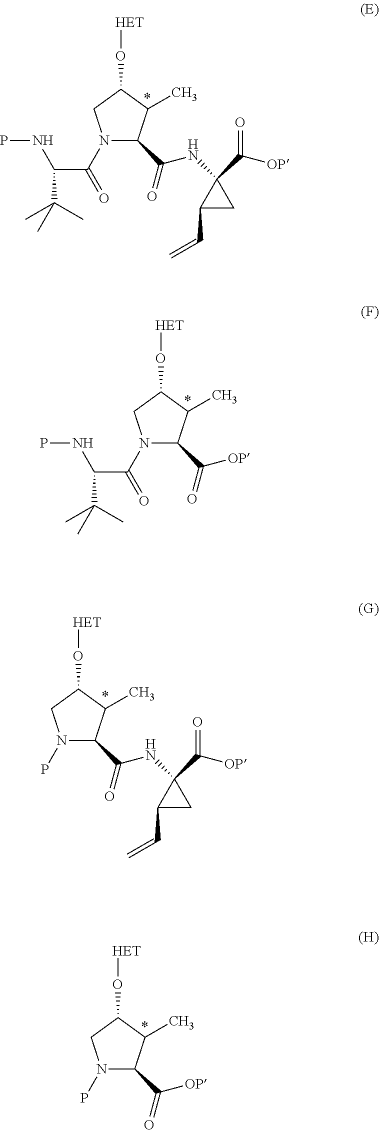

In Formulas A to H, P and P′ are any orthogonal protecting groups, and HET and R″ are as described above. It is readily envisioned that protection and/or deprotection of the respective carboxylic acid and amino moieties may be necessary to accomplish the desired coupling. Furthermore, it is readily envisioned that the requisite peptide fragments may be introduced in any desired stepwise manner.

One non-limiting example of the preparation of HET derivatized proline cores such as Compounds A to H is described in Schemes A-1 and A-2.

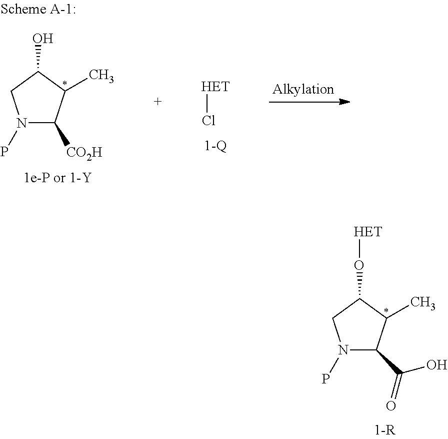

Scheme A-1 illustrates the reaction of Compound 1e-P or 1-Y with Compound 1-Q to afford Compound 1-R. In Scheme A-1, HET is as described above. As a non-limiting example, treatment of Compound 1e-P or 1-Y with a suitable base, such as potassium t-butoxide, in a suitable solvent, such as DMSO, in the presence of Compound 1-Q readily affords Compound 1-R. Surprisingly Compound 1-R can be obtained as a crystalline intermediate.

Peptide coupling to Compound 1-R may proceed in any order. One embodiment of peptide coupling is described in Scheme A-2.

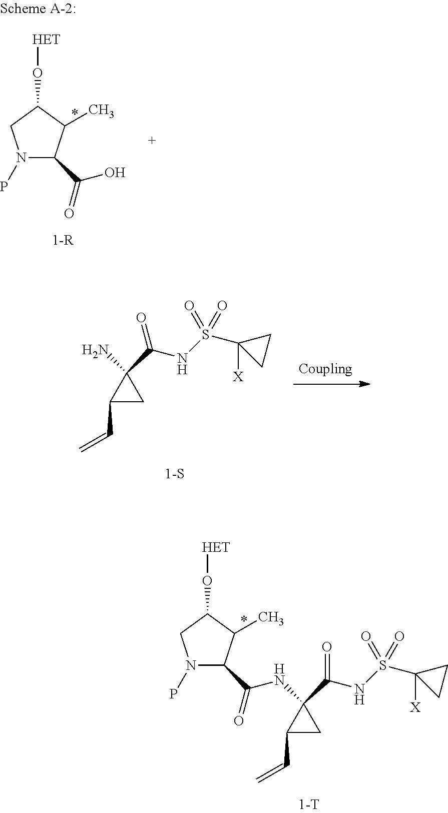

Scheme A-2 illustrates the coupling of Compound 1-R with Compound 1-S to afford Compound 1-T. In Compound 1-R, HET is as previously described. In Compound 1-S, X is hydrogen or methyl.

Many reaction conditions are known to effectuate peptide coupling and are described in more detail below. As a non-limiting example, treatment of Compound 1-R with HATU will afford an activated carbonyl containing intermediate, such that the primary amine of Compound 1-S will readily react to form an amide bond. Optionally, a suitable base may be used, such as DIEA.

Compound 1-T may be used to prepare a final compound via the peptide coupling described in Scheme A-3.

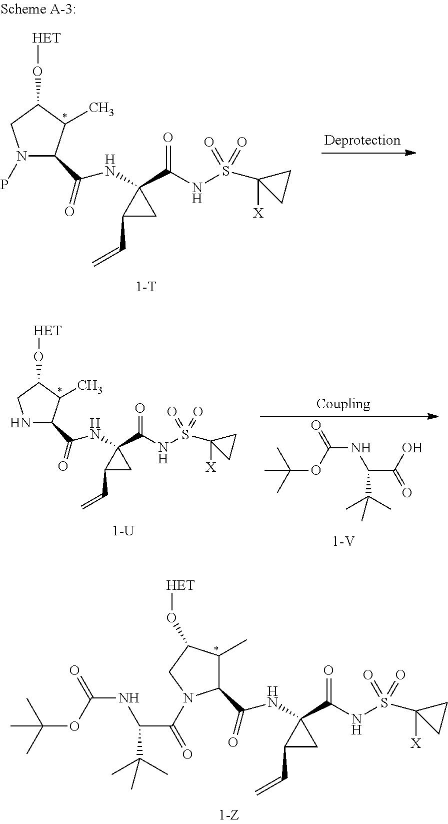

Scheme A-3 illustrates the final manipulations of Compound 1-T to afford a compound of Compound 1-Z for one embodiment. In Scheme A-3, HET and X are as previously described.

Deprotection of the amino protecting group, P, in Compound 1-T readily affords Compound 1-U. Depending upon the protecting group for the amine, the deprotection may readily occur under any of several conditions. As a non-limiting example, when P is a carbamate protecting group, such as BOC, deprotection may readily proceed upon exposure to an acid source, such as hydrochloric acid, in a suitable solvent, such as dioxane. Coupling Compound 1-U with Compound 1-V will readily afford the compound of Compound 1-Z, or a salt thereof. Many reaction conditions are known to effectuate peptide coupling and are described in more detail below. As a non-limiting example, treatment of Compound 1-V with HATU will afford an activated carbonyl containing intermediate, such that the primary amine of Compound 1-U will readily react to form an amide bond. Optionally, a suitable base may be used, such as DIEA.

An alternative peptide coupling method is described in Scheme A-4.

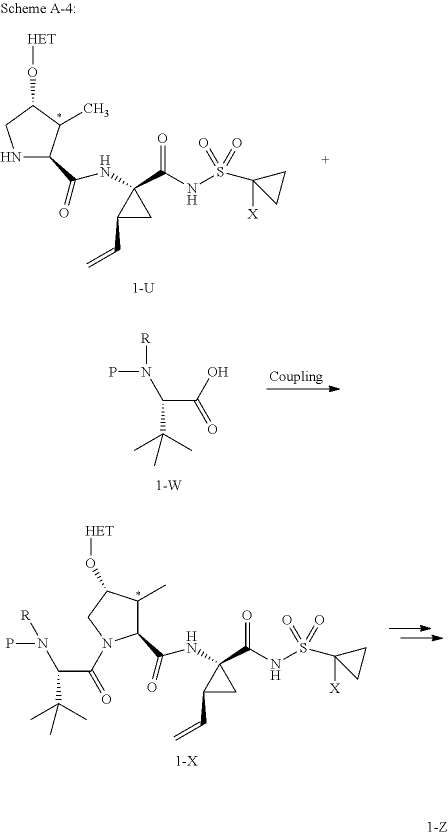

Scheme A-4 illustrates an alternative embodiment, wherein the Compound 1-U is coupled with a suitably protected amino acid, 1-W, to afford Compound 1-X. In Scheme 1H, P is any suitable protecting group, R is hydrogen or a suitable protecting group, and HET and X are as previously described. When P and R are protecting groups, they may be the same or different. To afford a compound of Compound 1-Z, R may be deprotected while P is a BOC group. Optionally, P may be deprotected while R is a BOC group to afford a compound of Compound 1-Z, or a salt thereof. Moreover, both P and R may be deprotected in one or more steps, followed by the reaction of the liberated primary amine with a reagent capable of introducing a BOC group. As a non-limiting example, such a reagent is BOC₂O, or an equivalent thereof.

Final Compound 1-Z may be used to prepare other compounds of Formula IV as described in Scheme A-5.

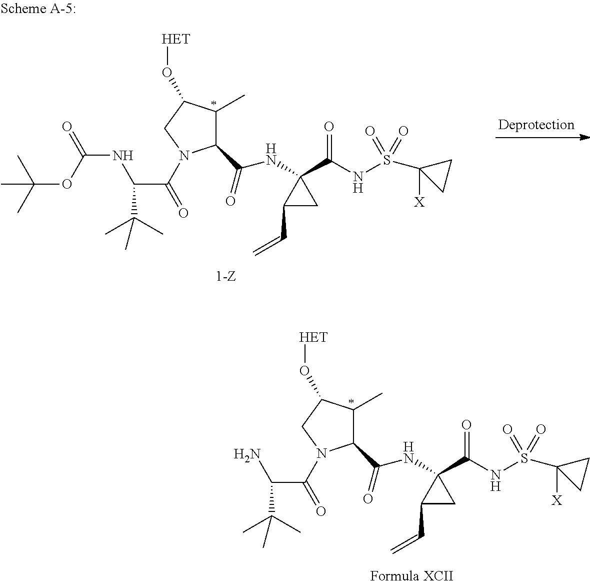

In another embodiment, Scheme A-5 illustrates the use of a compound of Compound 1-Z, or a salt thereof, for the synthesis of another HCV inhibitor, Formula XCII, or a salt thereof. In Scheme A-5, HET and X are as previously described.

The BOC-protected primary amine of Compound 1-Z may be readily deprotected under many different conditions that are known in the art. As a non-limiting example, exposing Compound 1-Z to acid, such as HCl, will readily cleave the BOC protecting group to afford Formula XXIX, or a salt thereof. One will readily appreciate that Formula XCII represents a versatile intermediate for the synthesis of additional compounds that may inhibit HCV. For example, the primary amine of Formula XCII will readily participate in couplings, alkylations, carbamate formations, and a wide variety of other synthetic transformations that are well known in the art. Such reactions represent a versatile method for obtaining new HCV inhibitors.

An alternative peptide coupling method is described in Schemes B-1 to B-5.

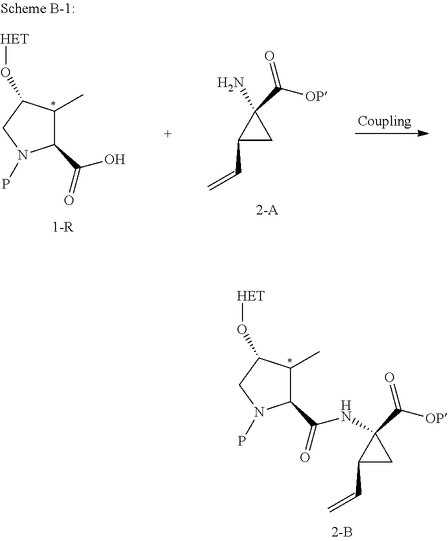

As illustrated in Scheme B-1, coupling Compound 1-R with Compound 2-A will readily afford Compound 2-B. Many reaction conditions are known to effectuate peptide coupling and are described in more detail below. As a non-limiting example, treatment of Compound 1-R with HATU will afford an activated carbonyl containing intermediate, such that the primary amine of Compound 2-A will readily react to form an amide bond. Optionally, a suitable base may be used, such as DIEA.

Regarding the protecting group P′ in Scheme B-1, P′ represents a functional group that is suitable for protecting a carboxylic acid. In one embodiment, P′ is a functional group that protects the carboxylic acid as an ester. As a non-limiting example, a suitable protecting group for P′ is an ethyl group. Regarding the protecting group P in Scheme B-1, P represents a functional group that is suitable for protecting an amine. In one embodiment, P is a functional group that protects the amine as a carbamate. As a non-limiting example, a suitable protecting group for P is a BOC group.

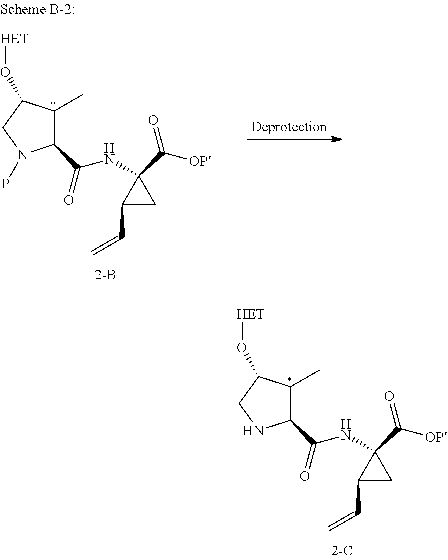

As illustrated in Scheme B-2, deprotecting the protected amino group of Compound 2-B will readily afford Compound 2-C. Depending upon the protecting group for the amine, the deprotection may readily occur under any of several conditions. As a non-limiting example, when P is a carbamate protecting group, such as BOC, deprotection may readily proceed upon exposure to an acid source, such as hydrochloric acid, in a suitable solvent, such as dioxane.

Regarding the protecting group P′ in Scheme B-2, P′ represents a functional group that is suitable for protecting a carboxylic acid. In one embodiment, P′ is a functional group that protects the carboxylic acid as an ester. As a non-limiting example, a suitable protecting group for P′ is an ethyl group. Regarding the protecting group P in Scheme B-2, P represents a functional group that is suitable for protecting an amine. In one embodiment, P is a functional group that protects the amine as a carbamate. As a non-limiting example, a suitable protecting group for P is a BOC group.

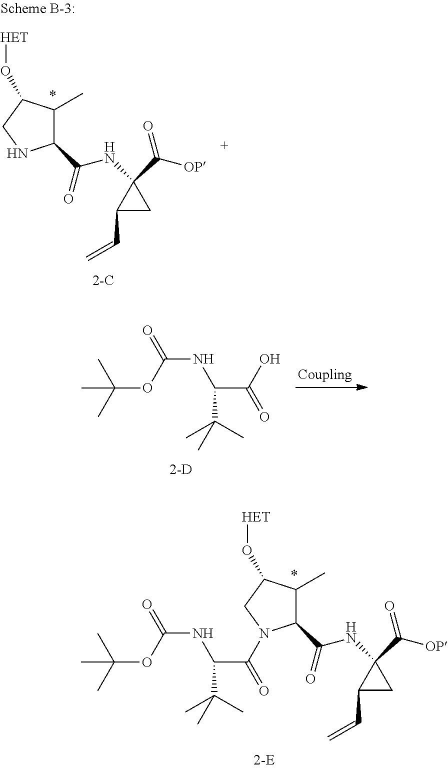

As illustrated in Scheme B-3, coupling Compound 2-C with Compound 2-D will readily afford Compound 2-E. Many reaction conditions are known to effectuate peptide coupling and are described in more detail below. As a non-limiting example, treatment of Compound 2-D with HATU will afford an activated carbonyl containing intermediate, such that the primary amine of Compound 2-C will readily react to form an amide bond. Optionally, a suitable base may be used, such as DIEA.

Regarding the protecting group P′ in Scheme B-3, P′ represents a functional group that is suitable for protecting a carboxylic acid. In one embodiment, P′ is a functional group that protects the carboxylic acid as an ester. As a non-limiting example, a suitable protecting group for P′ is an ethyl group. Regarding the protecting group P in Scheme B-3, P represents a functional group that is suitable for protecting an amine. In one embodiment, P is a functional group that protects the amine as a carbamate. As a non-limiting example, a suitable protecting group for P is a BOC group.

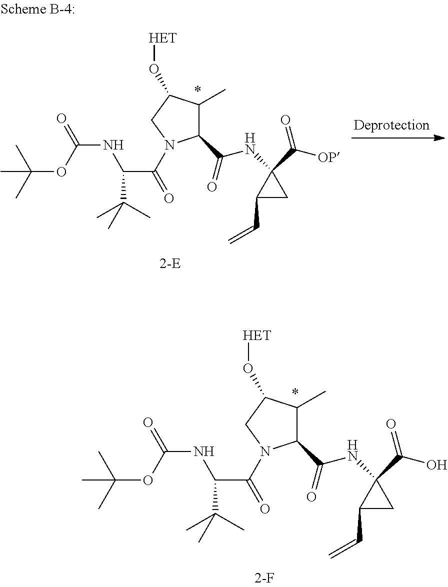

As illustrated in Scheme B-4, deprotecting the protected carboxylic acid of Compound 2-E will readily afford Compound 2-F. Depending upon the protecting group for the carboxylic acid, the deprotection may readily occur under any of several conditions. As a non-limiting example, when the carboxylic acid is protected as an ester, such that P′ is an ethyl group, deprotection may readily proceed via saponification of the ester protecting group in the presence of a base under aqueous conditions, such as in the presence of lithium hydroxide and water. Optionally, ethanol may be present as a co-solvent.

Regarding the protecting group P′ in Scheme B-4, P′ represents a functional group that is suitable for protecting a carboxylic acid. In one embodiment, P′ is a functional group that protects the carboxylic acid as an ester. As a non-limiting example, a suitable protecting group for P′ is an ethyl group. Regarding the protecting group P in Scheme B-4, P represents a functional group that is suitable for protecting an amine. In one embodiment, P is a functional group that protects the amine as a carbamate. As a non-limiting example, a suitable protecting group for P is a BOC group.

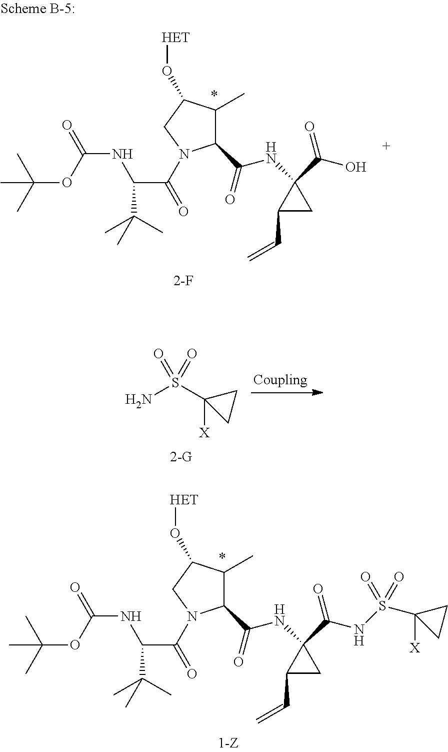

As illustrated in Scheme B-5, coupling Compound 2-F with Compound 2-G will readily afford a compound of Compound 1-Z, or a salt thereof. In Scheme B-5, HET and X are as previously described.

Many reaction conditions are known to effectuate peptide coupling and are described in more detail below. As a non-limiting example, treatment of Compound 2-F with a suitable reagent will afford an activated carbonyl containing intermediate such that the primary amine of Compound 2-G will readily react to form an amide bond. Suitable activating agents are described in detail below, and include carbonyldiimidazole (CDI). Optionally, a suitable base may be used, such as DBU.

Still another alternative peptide coupling method is described in Scheme B-6.

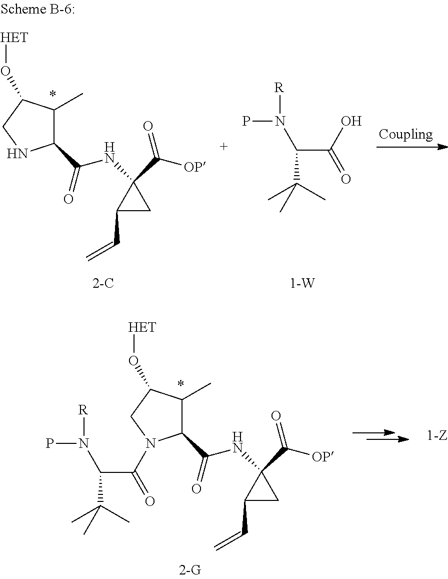

Scheme B-6 illustrates an alternative route to a compound of Compound 1-Z, or a salt thereof. The alternative route in Scheme B-6 employs the coupling of Compound 2-C with Compound 1-W to afford Compound 2-G. In Compound 1-W, P is any suitable protecting group and R is hydrogen or a suitable protecting groups. As illustrated in Schemes B-3 through B-5, P may be a BOC group and R may be hydrogen, in which case the coupling reaction described in Scheme B-3 is obtained. In one embodiment, however, R is hydrogen and P is a protecting group other than BOC. For such an embodiment, a compound of Compound 1-Z is prepared by deprotecting P to afford an unprotected primary amine and then later introducing a BOC group.

In one embodiment P and R are both protecting groups. In another embodiment, P and R are the same protecting group. Moreover, P and R may be different protecting groups. To afford a compound of Compound 1-Z, or a salt thereof, R may be deprotected while P is a BOC group. Optionally, P may be deprotected while R is a BOC group to afford a compound of Compound 1-Z. Moreover, both P and R may be deprotected in one or more steps, followed by the reaction of the liberated primary amine with a reagent capable of introducing a BOC group. As a non-limiting example, such a reagent is BOC₂O, or an equivalent thereof.

An alternative approach to Scheme B-5 for the preparation of HET-functionalized proline moieties is described in Scheme C-1.

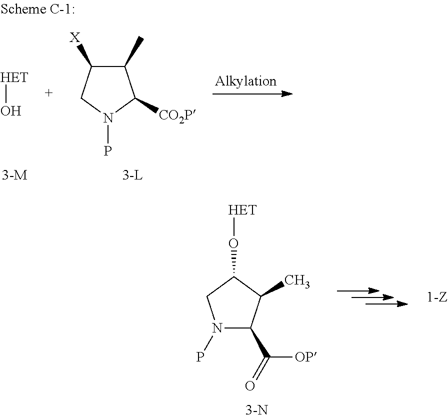

As indicated in Scheme C-1, Compound 3-L will readily react with Compound 3-M to afford Compound 3-N. In Compound 3-L, P′ represents a functional group that is suitable for protecting a carboxylic acid. In Compound 3-M, HET is as previously described. In one embodiment, P′ is a functional group that protects the carboxylic acid as an ester. As a non-limiting example, a suitable protecting group for P′ is a methyl group. Regarding the protecting group P in Scheme C-1, P represents a functional group that is suitable for protecting an amine. In one embodiment, P is a functional group that protects the amine as a carbamate. As a non-limiting example, a suitable protecting group for P is a BOC group.

In Compound 3-L, X is any suitable leaving group. The selection of an appropriate leaving group will depend upon the reaction conditions utilized for the alkylation. As a non-limiting example, X may be a hydroxyl group and the alkylation may occur under Mitsunobu conditions. For example, the hydroxyl group, X, in Compound 3-L may be converted into a suitable leaving group by treatment with DIAD and triphenylphosphine. Nucleophilic displacement of the leaving group by Compound 3-M would then afford Compound 3-N.

Compound 3-N is a suitable intermediate for the ultimate production of a compound of Compound 1-Z, or a salt thereof. For example, deprotection of the carboxylic acid protecting group, P′, will readily afford Compound 1-R. As discussed above, Compound 1-R represents a suitable intermediate for the preparation of a compound of Compound 1-Z.

A second approach for preparing a compound of Formula IV-1 may involve introduction of a HET moiety via an alkylation step. The alkylation step may occur in two principle manners. First, such an alkylation step may proceed using a HET derivative such as Formula XI, or a salt thereof, as a nucleophile for alkylating the proline core of any of compounds of Formulas XII to XVIII, or salts thereof, wherein LG is any suitable leaving group, P and P′ are any orthogonal protecting groups, and R″ and HET are as described above.

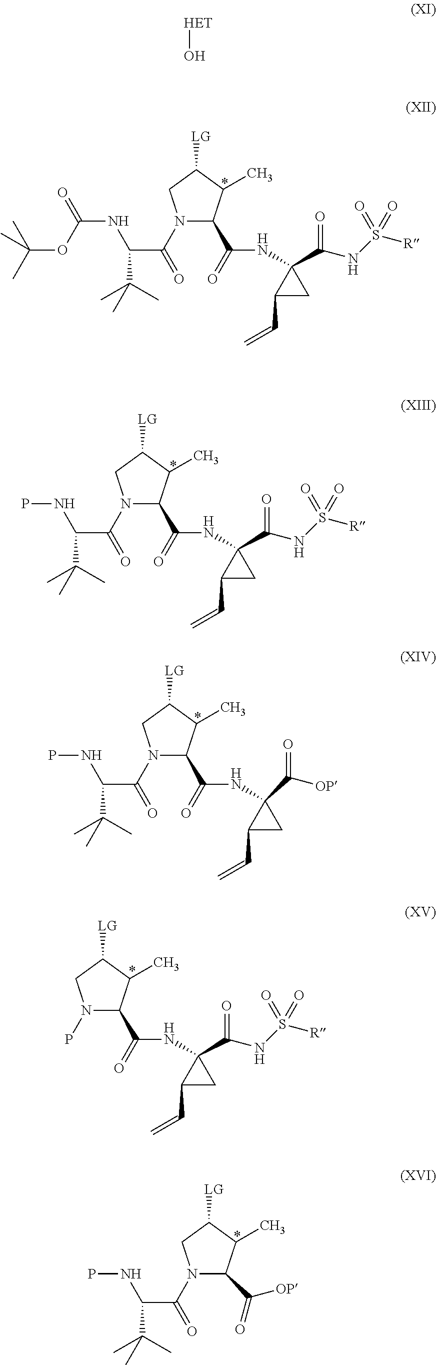

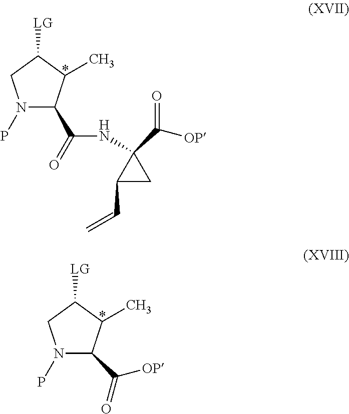

Conversely, such an alkylation step may proceed via alkylation of a HET derivative such as Formula IXX, or a salt thereof, using a suitably functionalized proline core such as Formulas XX to XXVI, or a salt thereof, as the nucleophile. In Formulas IXX to XXVI, LG is any suitable leaving group, P and P′ are any orthogonal protecting groups, R′ includes, but is not limited to, hydrogen or a carbamate, and R″ and HET are as described above.

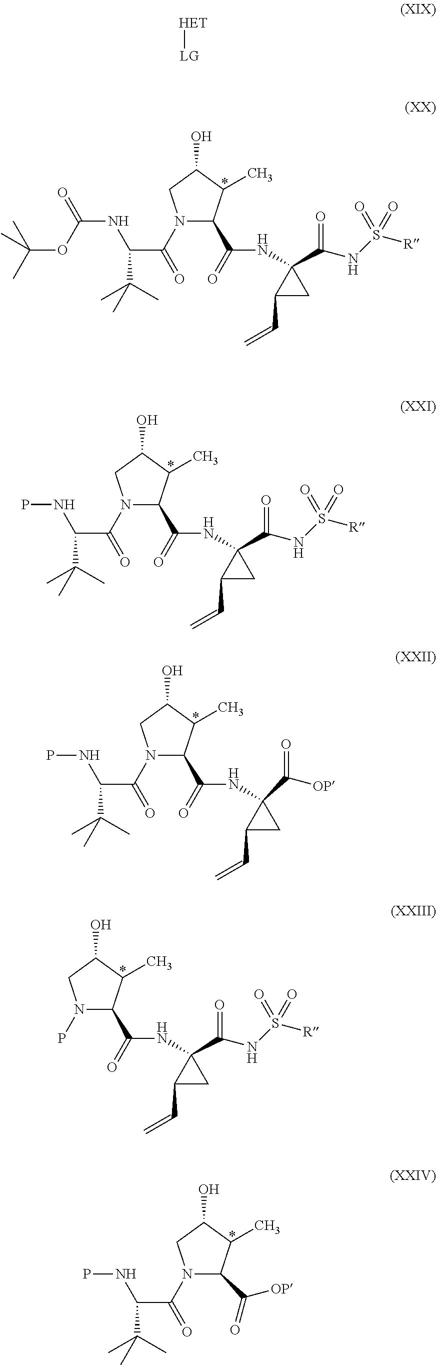

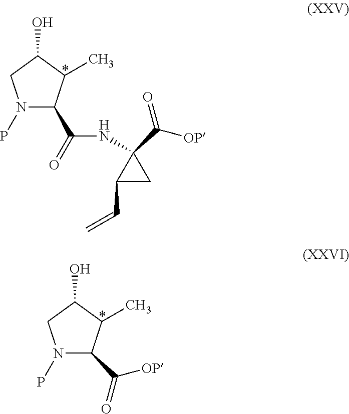

One will readily appreciate that upon alkylation of compounds of Formulas XII to XVIII, or salts thereof, with Formula XI, or a salt thereof, compounds of Formulas A to H will be obtained. Likewise, one will readily appreciate that upon alkylation of a compound of Formula IXX, or a salt thereof, with compounds of Formulas XX to XXVI, or salts thereof, compounds of Formulas A to H will be obtained. Consequently, combinations of the above described synthetic approaches may be utilized to prepare a compound of Formula IV-1 or precursors thereof that are suitable for preparing a compound of Formula IV-1. Non-limiting examples of compounds of Formula IV-1 that are readily prepared by the above described general approach include, but are not limited to the following:

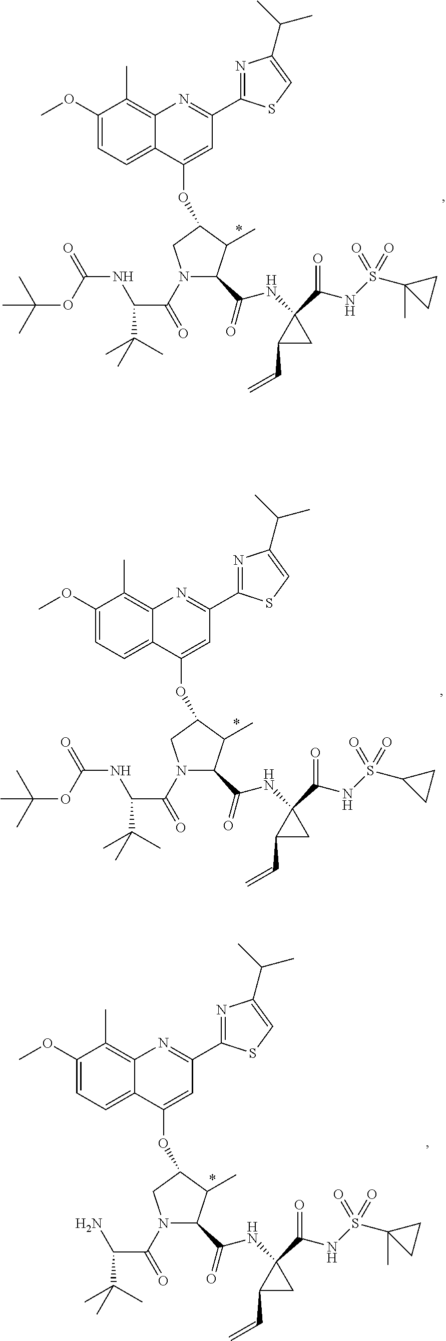

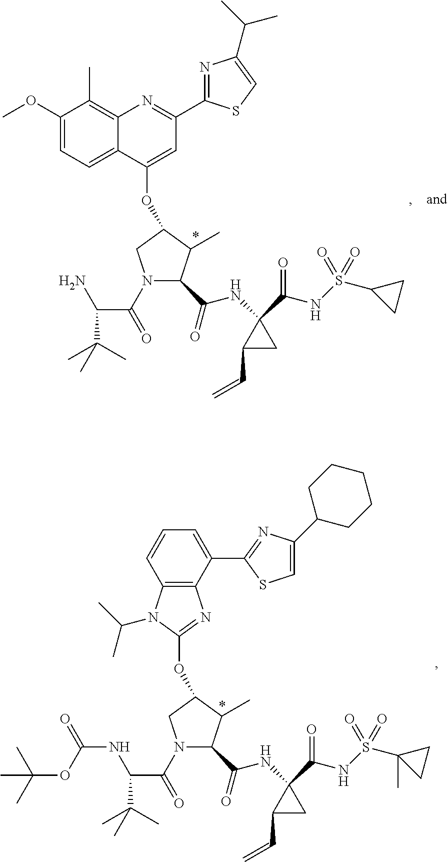

wherein the “*” indicates that the carbon atom may optionally be of an R or S configuration.

Macrocyclic compounds according to Formula I may be prepared in a similar manner to that described above. As a non-limiting illustration, Formula I-1 depicted below is a typical subgenus of compounds to Formula I and identifies the variety of attachment points or coupling steps that may be utilized to produce the final compound.

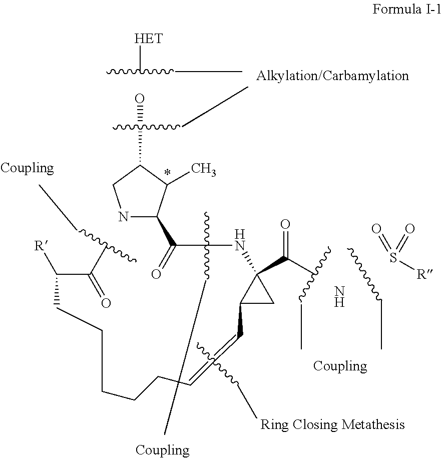

A compound of Formula I-1 or a salt thereof, wherein R′, R″, “*”, and HET are as described above, may be prepared using several synthetic approaches. One such approach may utilize a proline core derivatized with a HET moiety, such as compounds of Formulas XXVII to XXXIV, or salts thereof, and rely on the introduction of peptide fragments and/or peptide bonds via coupling steps.

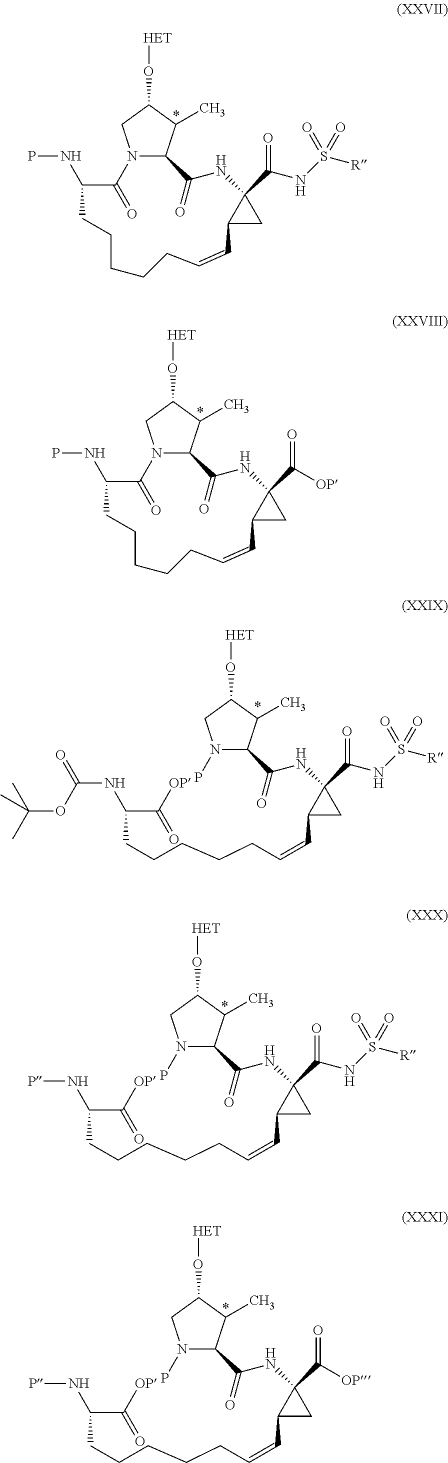

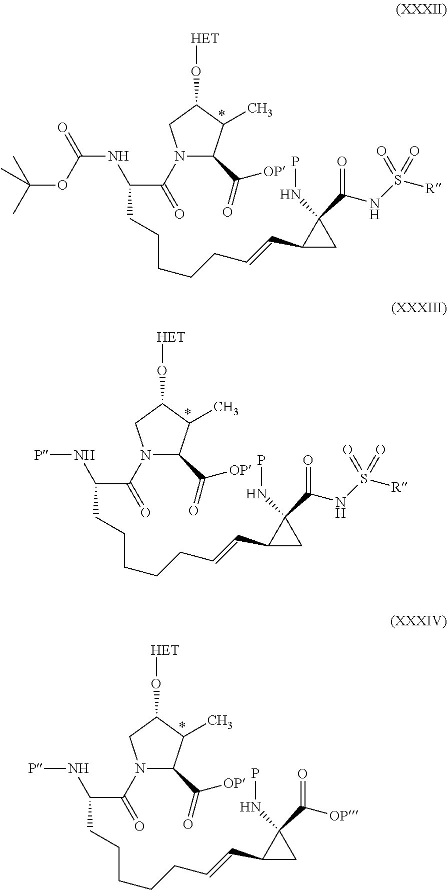

In Formulas XXVII to XXXIV, P, P′, P″, and P′″ are any orthogonal protecting groups and R″ and HET are as described above. It is readily envisioned that protection and/or deprotection of the respective carboxylic acid and amino moieties may be necessary to accomplish the desired coupling. Furthermore, it is readily envisioned that the requisite peptide fragments may be introduced in any desired stepwise manner.

Alternatively, a compound of Formula I-1, or a precursor thereof, may be prepared using ring closing metathesis of compounds of Formulas XXXV to XXXVIII, or salts thereof, and rely on the introduction of peptide fragments via coupling steps.

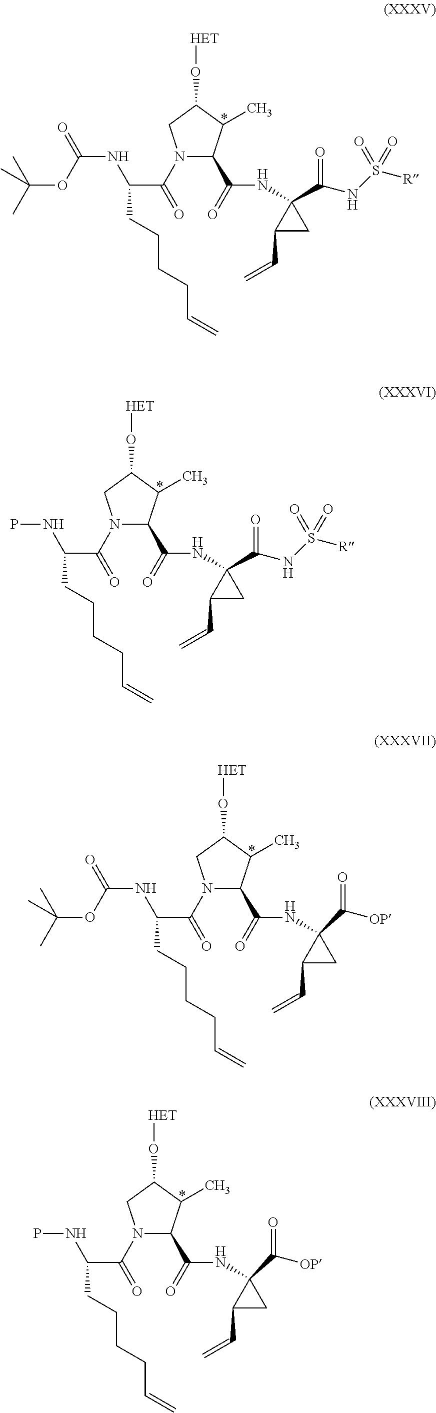

One non-limiting example of peptide coupling to a HET-derivatized proline core followed by ring closing metathesis (RCM) is described in Scheme D-1.

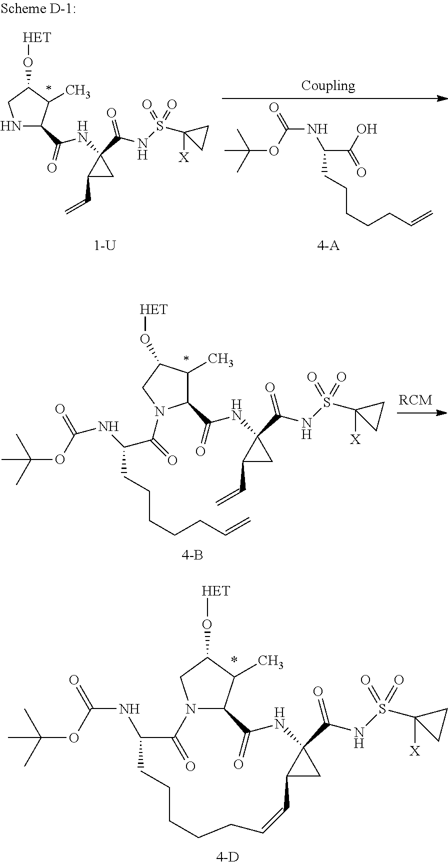

Scheme D-1 illustrates one route for the preparation of Compound 4-D. In Scheme D-1, HET and X are as previously described.

Coupling Compound 1-U with Compound 4-A will readily afford Compound 4-B. Many reaction conditions are known to effectuate peptide coupling and are described in more detail below. As a non-limiting example, treatment of Compound 1-U with HATU will afford an activated carbonyl containing intermediate, such that the primary amine of Compound 1-U will readily react to form an amide bond. Optionally, a suitable base may be used, such as DIEA.

Ring closing metathesis (“RCM”) of Compound 4-B will readily afford a compound of Compound 4-D. Many reaction conditions are known to effectuate RCM. Catalysts for such reactions include, but are not limited to, ruthenium (II) carbine complexes and/or first generation Grubbs' Catalyst, second generation Grubbs' Catalyst, Hoveyda-Grubbs Catalysts, Schrock Catalysts, and Zhan Catalysts.

One will readily appreciate that compounds other than 4-A may be employed for coupling with Compound 1-U to afford additional analogues. As a non-limiting example, the use of Compound 4-C in Scheme D-1 will readily afford Compound 4-E:

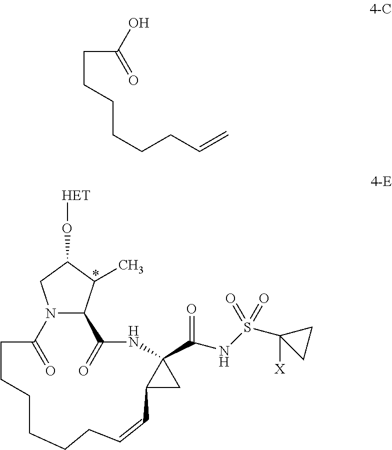

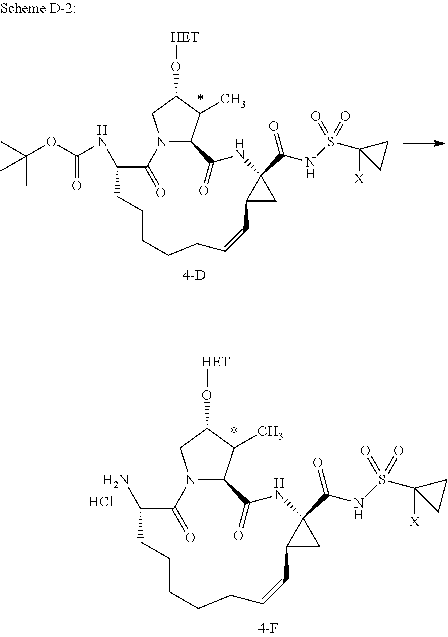

In another embodiment, Scheme D-2 illustrates the use of Compound 4-D for the synthesis of another HCV inhibitor, Compound 4-F. In Scheme D-2, HET and X are as previously described.

The BOC-protected primary amine of Compound 4-D may be readily deprotected under many different conditions that are known in the art. As a non-limiting example, exposing Compound 4-D to acid, such as HCl, will readily cleave the BOC protecting group to afford Compound 4-F. One will readily appreciate that Compound 4-F represents a versatile intermediate for the synthesis of additional compounds that may inhibit HCV. For example, the primary amine of Compound 4-F will readily participate in couplings, alkylations, carbamate formations, and a wide variety of other synthetic transformations that are well known in the art. Such reactions represent a versatile method for obtaining new HCV inhibitors.

Another example of peptide coupling to a HET-derivatized proline core followed by ring closing metathesis (RCM) is described in Schemes E-1 through E-4.

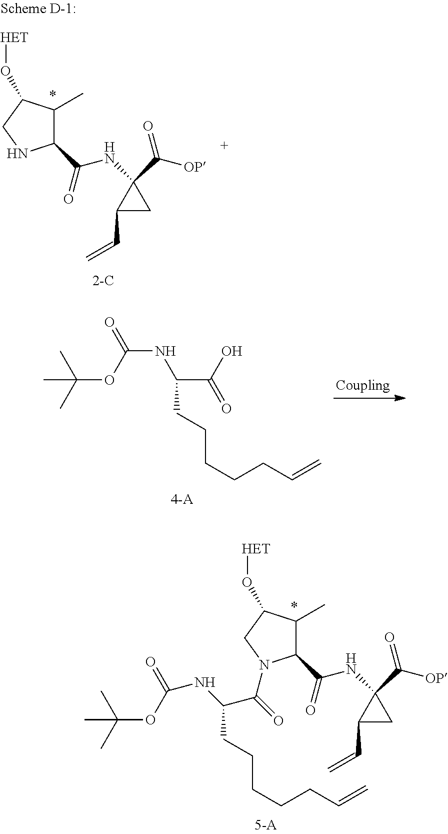

Schemes E-1 through E-4 illustrate an alternative synthetic approach for the ultimate preparation of Compound 4D, or a salt thereof. As illustrated in Scheme E-1, coupling Compound 2-C with Compound 4-A will readily afford Compound 5-A. Many reaction conditions are known to effectuate peptide coupling and are described in more detail below. As a non-limiting example, treatment of Compound 4-A with HATU will afford an activated carbonyl containing intermediate, such that the secondary amine of Compound 2-C will readily react to form an amide bond. Optionally, a suitable base may be used, such as DIEA.

Regarding the protecting group P′ in Scheme E-1, P′ represents a functional group that is suitable for protecting a carboxylic acid. In one embodiment, P′ is a functional group that protects the carboxylic acid as an ester. As a non-limiting example, a suitable protecting group for P′ is an ethyl group. Regarding the protecting group P in Scheme E-1, P represents a functional group that is suitable for protecting an amine. In one embodiment, P is a functional group that protects the amine as a carbamate. As a non-limiting example, a suitable protecting group for P is a BOC group.

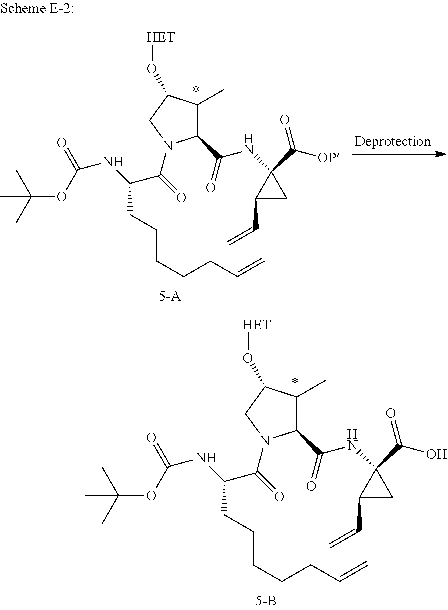

As illustrated in Scheme E-2, deprotecting the protected carboxylic acid of Compound 5-A will readily afford Compound 5-B. Depending upon the protecting group for the carboxylic acid, the deprotection may readily occur under any of several conditions. As a non-limiting example, when the carboxylic acid is protected as an ester, such that P′ is an ethyl group, deprotection may readily proceed via saponification of the ester protecting group in the presence of a base under aqueous conditions, such as in the presence of lithium hydroxide and water. Optionally, ethanol may be present as a co-solvent.

Regarding the protecting group P′ in Scheme E-2, P′ represents a functional group that is suitable for protecting a carboxylic acid. In one embodiment, P′ is a functional group that protects the carboxylic acid as an ester. As a non-limiting example, a suitable protecting group for P′ is an ethyl group. Regarding the protecting group P in Scheme E-2, P represents a functional group that is suitable for protecting an amine. In one embodiment, P is a functional group that protects the amine as a carbamate. As a non-limiting example, a suitable protecting group for P is a BOC group.

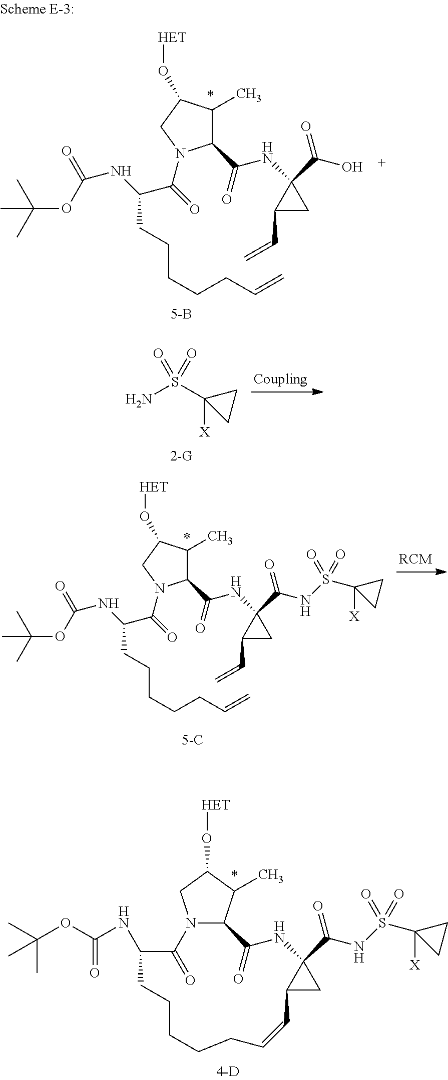

As illustrated in Scheme E-3, coupling Compound 5-B with Compound 2-G will readily afford a Compound 5-C, or a salt thereof. In Scheme E-3, HET and X are as previously described.

Many reaction conditions are known to effectuate peptide coupling and are described in more detail below. As a non-limiting example, treatment of Compound 5-B with a suitable reagent will afford an activated carbonyl containing intermediate such that the primary amine of Compound 2-G will readily react to form an amide bond. Suitable activating agents are described in detail below, and include carbonyldiimidazole (CDI). Optionally, a suitable base may be used, such as DBU.

Subjecting Compound 5-C to conditions known to effectuate ring closing metathesis will readily afford Compound 4-D. Alternatively, Compounds 5-A and 5-B could be subjected to ring closing metathesis prior to coupling with Compound 2-G. Such a modification of Scheme E-3 is illustrated in Scheme E-4.

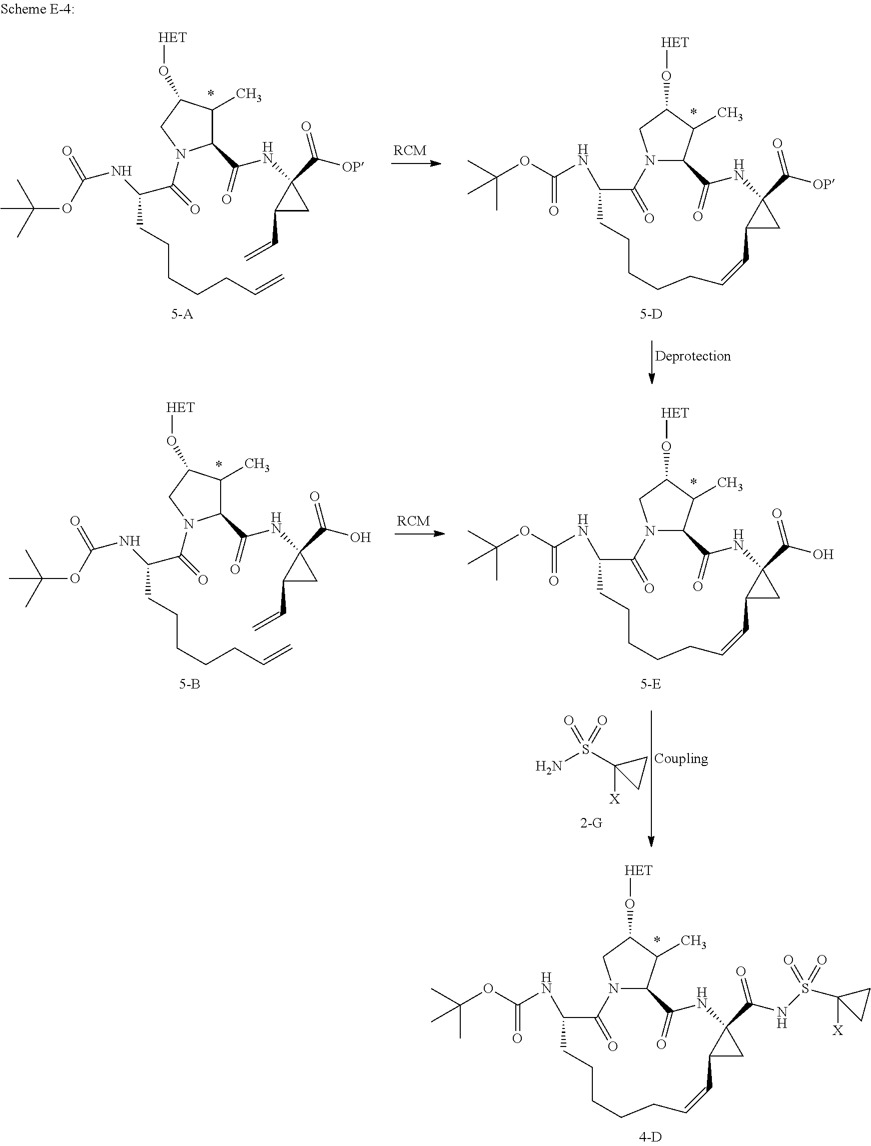

As illustrated in Scheme E-4, ring closing metathesis of Compounds 5-A and 5-B will readily afford Compounds 5-D and 5-E, respectively. Deprotecting the protected carboxylic acid of Compound 5-D will result in the formation of Compound 5-E. Coupling Compound 5-E with Compound 2-G will readily afford Compound 4-D, or a salt thereof.

A second approach for preparing a compound of Formula I-1, or a precursor thereof, may involve introduction of a HET moiety via an alkylation step. The alkylation step may occur in two principle manners. First, such an alkylation step may proceed using a HET derivative such as Formula XI, or a salt thereof, as a nucleophile for alkylating the proline core of any of compounds of Formulas XXXIX to L, or salts thereof, wherein LG is any suitable leaving group, P, P′, P″, and P′″ are any orthogonal protecting groups, and R″ and HET are as described above. Ring closing metathesis may be utilized, as necessary, to provide the desired target compound or precursor.

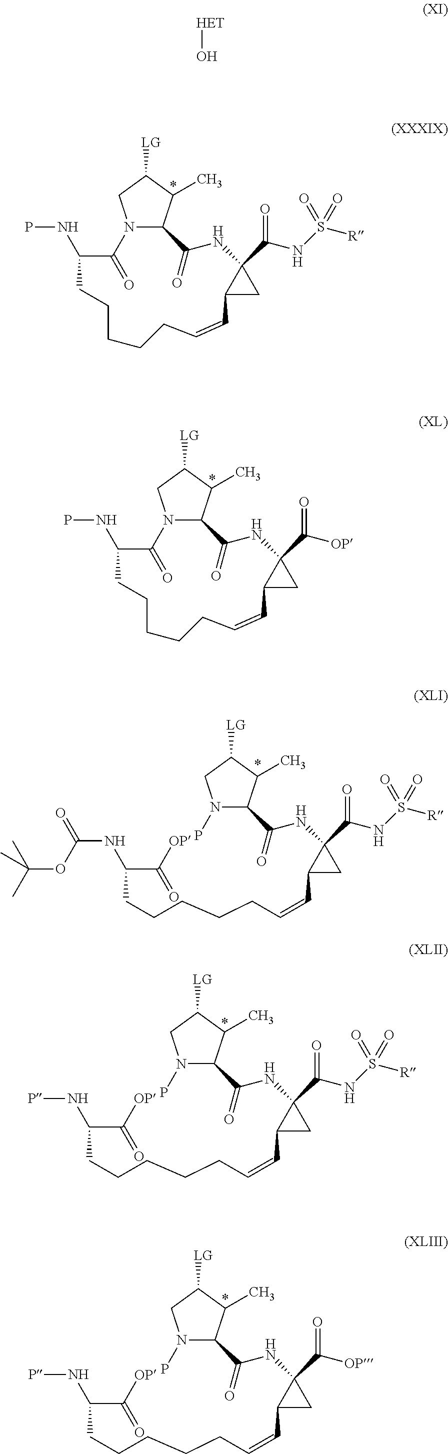

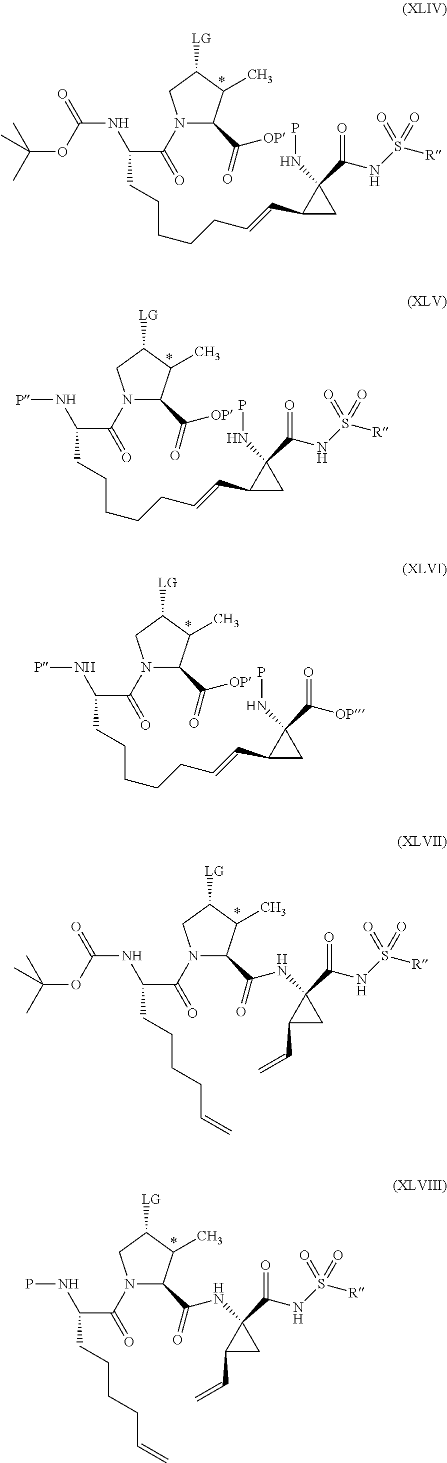

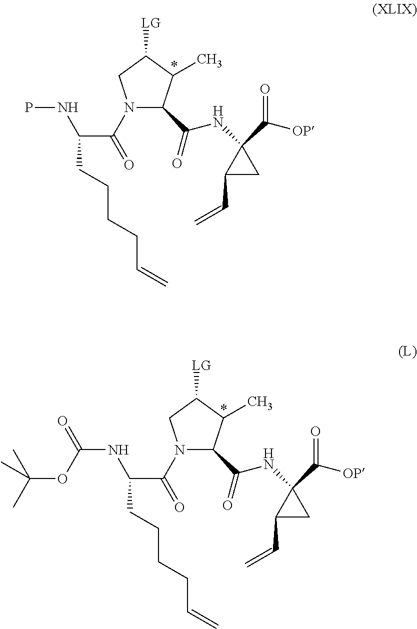

Conversely, the alkylation step may proceed via alkylation of a HET derivative such as Formula XIX, or a salt thereof, using a suitably functionalized proline core such as Formulas LI to LXII, or a salt thereof, as the nucleophile. In Formula XIX, LG is any suitable leaving group. In Formulas LI to LXII, P, P′, P″, and P′″ are any orthogonal protecting groups. R″ and HET are as described above.

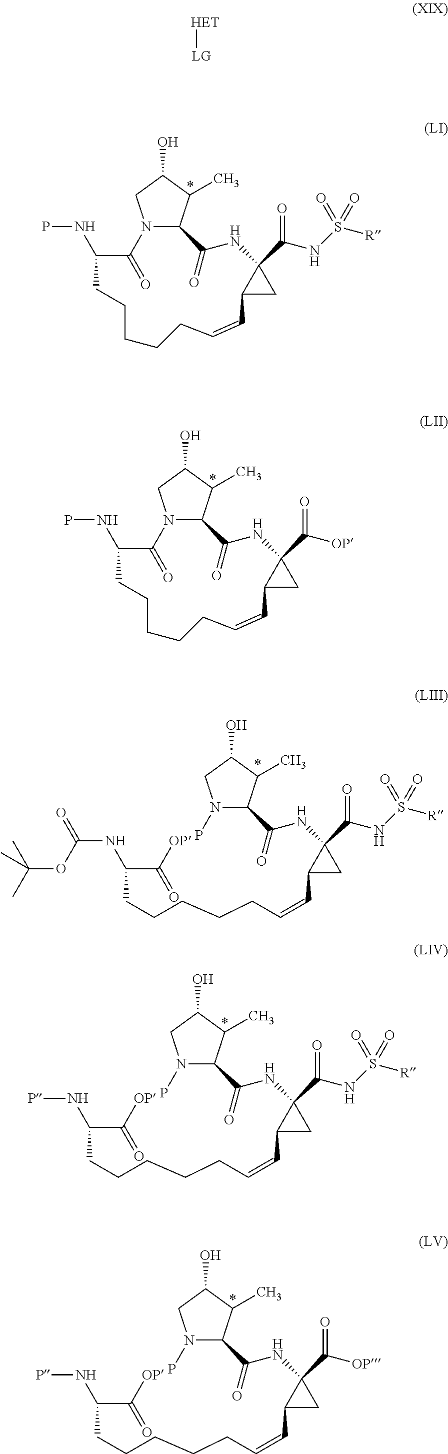

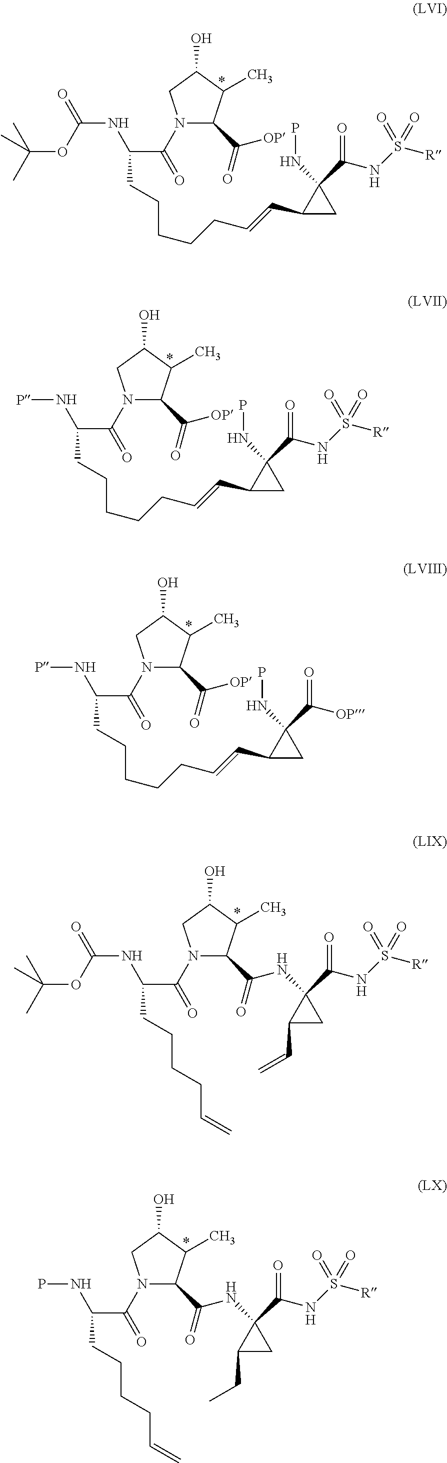

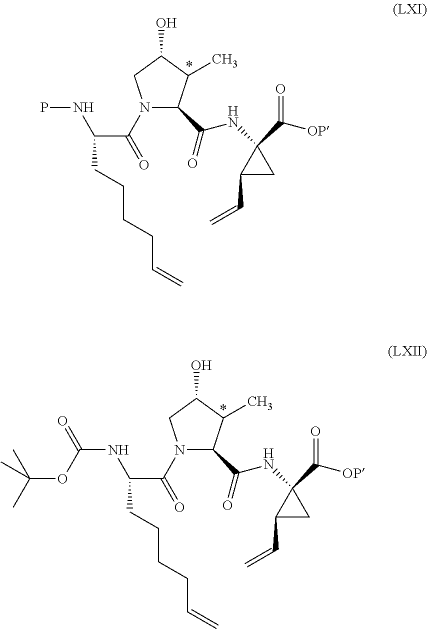

One will readily appreciate that upon alkylation of compounds of Formulas XXXIX to L, or salts thereof, with Formula XI, or a salt thereof, compounds of Formulas XXVII to XXXVIII will be obtained. Likewise, one will readily appreciate that upon alkylation of a compound of Formula XIX, or a salt thereof, with compounds of Formulas LI to LXII, or salts thereof, compounds of Formulas XXXIX to L will be obtained. Consequently, combinations of the above described synthetic approaches may be utilized to prepare a compound of Formula I-1 or precursors thereof that are suitable for preparing a compound of Formula I-1. Non-limiting examples of Compounds of Formula I-1 that are readily prepared by the above described general approach include, but are not limited to the following:

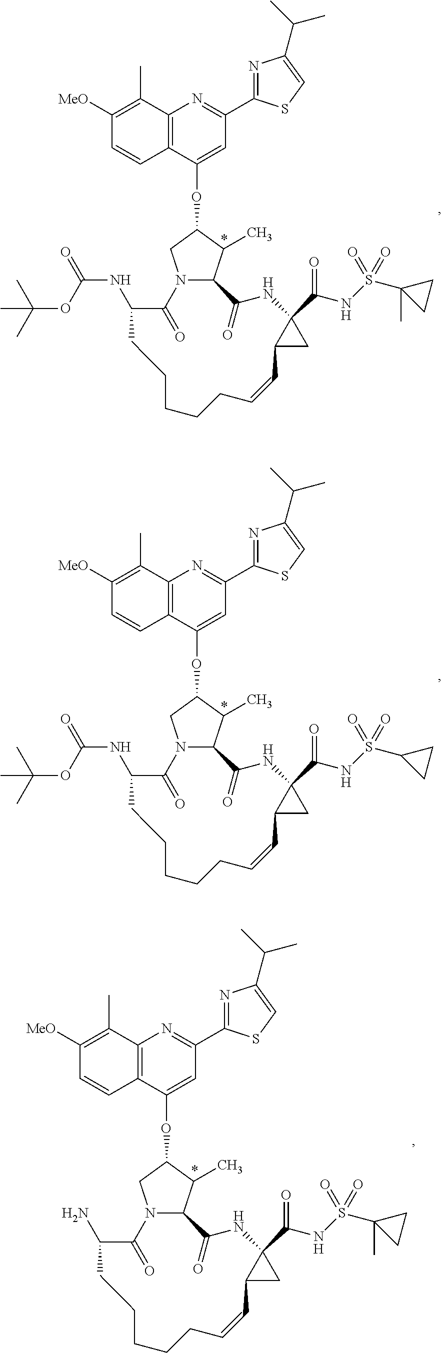

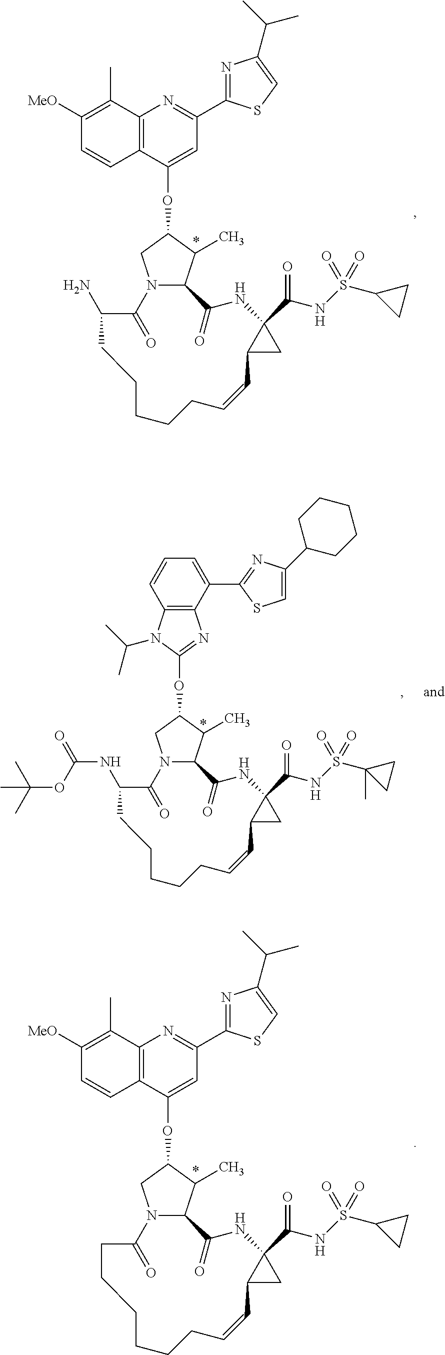

The above various methods described above may be readily generalized to make the full scope of compounds of Formulas I and IV. Accordingly, in some embodiments, compounds of Formula IV, or salts thereof,

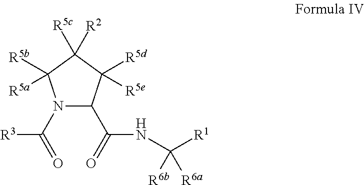

are readily synthesized by adding the R¹ containing moiety as a last step, i.e., by coupling a compound of Formula LXIV, or a salt thereof, with a compound of Formula LXV, or a salt thereof,

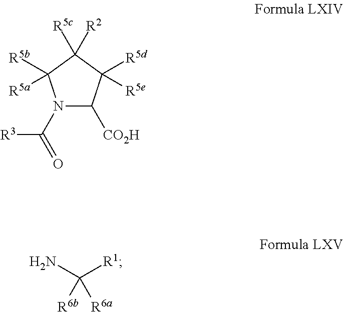

wherein the variable definitions are the same as described above for Formula IV. Couple

In some embodiments, Compound LXIV is obtained by deprotecting a compound having the structure:

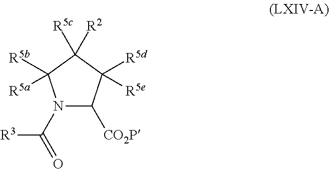

to afford the compound of Formula LXIV, where P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction.

In some embodiments, Compound LXIV-A is obtained by coupling a compound of Formula LXIV-B with a compound of Formula LXVII to afford the compound of Formula LXIV-A (e.g., a peptide coupling to add the R³ containing moiety):

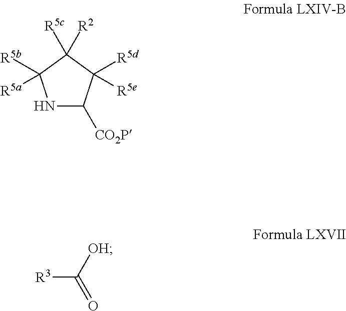

In some embodiments, the coupling is conducted in the presence of an activating reagent and a base. In some embodiments, the activating reagent is HATU and the base is an organic base.

In some embodiments, compound LXIV-B is obtained by deprotecting a compound having the structure

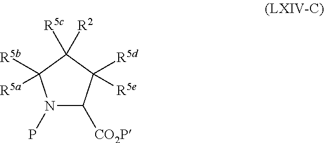

to afford the compound of Formula LXIV-B, where P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In an alternative embodiment, compounds of Formula IV are readily synthesized by adding the R³ containing moiety as a last step, i.e., by coupling a compound of Formula LXVI, or a salt thereof, with a compound of Formula LXVII, or a salt thereof, to afford a compound of Formula IV, or a salt thereof,

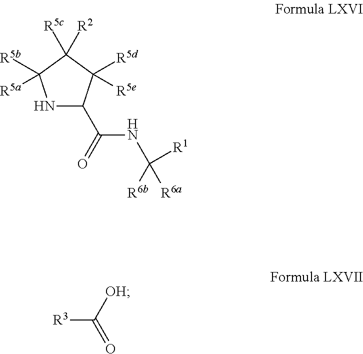

wherein the variable definitions are the same as described above for Formula IV. In some embodiments, the coupling is conducted in the presence of an activating reagent and a base. In some embodiments, the activating reagent is HATU and the base is an organic base.

In some embodiments, Compound LXIV is obtained by deprotecting a compound having the structure

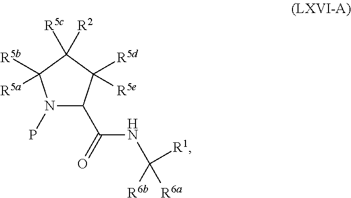

where P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In some embodiments, Compound LXVI-A is obtained by coupling a compound having the structure

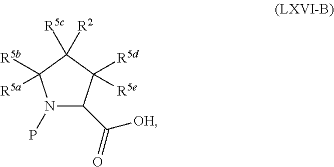

or a salt thereof, with a compound having the structure

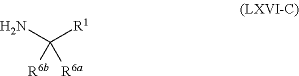

to afford the compound of Formula LXVI-A (e.g., a peptide coupling to add the R¹ containing moiety). In some embodiments, the coupling is conducted in the presence of an activating reagent and a base. In some embodiments, the activating reagent is HATU and the base is an organic base.

In some embodiments, Compound LXVI-B is obtained by deprotecting a compound having the structure

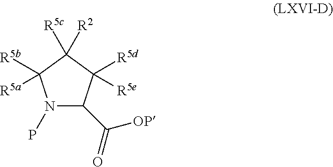

to afford the compound of Formula LXVI-B, where P′ is a protecting group for a carboxylic acid. P′

In some embodiments, Compounds LXIV-C and LXVI-D are obtained by reacting a compound having the structure

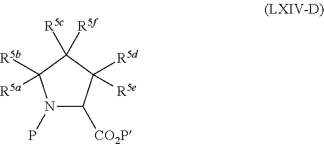

with a compound having the structure selected from X-LG and

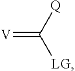

where R^{5f} is OH, NH₂, or SH; LG is a leaving group; and the remaining variables are as above for Formula IV resulting in attachment of the R² moiety to the proline core. In some embodiments, the reaction is conducted in the presence of an alkoxide salt. In some embodiments, the alkoxide salt is potassium t-butoxide. In some embodiments, the leaving group is selected from a halogen, a sulfonate ester, and a diazonium compound. In some embodiments, the leaving group is chloride.

One will readily appreciate that the respective coupling of compounds of Formulas LXIV with LXV, and LXVI with LXVII may be conducted in the presence of a wide variety of suitable activating reagents and/or bases. Alternatively, compounds of Formulas LXIV, LXV, LXVI, and LXVII may be activated in the presence of one another, or optionally they may be activated in a separate step prior to being introduced to their respective coupling partners.

In an alternative embodiment, compounds of Formula IV are readily synthesized by adding the R² moiety as a last step, i.e., by coupling a compound of Formula LXVIII, or a salt thereof, with a compound of Formula LXIX-1, or a salt thereof, a compound of Formula LXIX-2, or a salt thereof, or a compound of Formula LXIX-3, or a salt thereof:

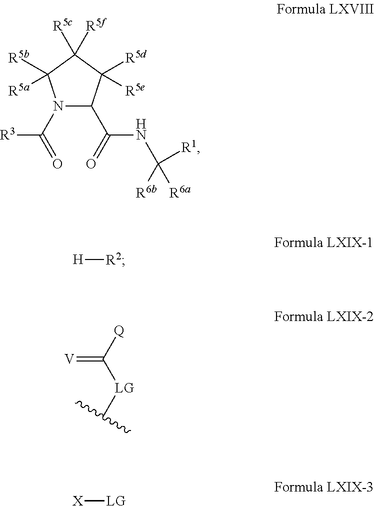

where:
R^{5f} is a leaving group when reacting the compound of Formula LXVIII, or a salt thereof, with a compound of Formula LXIX-1, or a salt thereof, or R^{5f} is —OH, —SH, or NH₂ when reacting the compound of Formula LXVIII, or a salt thereof, with a compound of Formula LXIX-2, or a salt thereof, or a compound of Formula LXIX-3, or a salt thereof;
LG is a leaving group; and
the remaining variables are as defined above for Formula IV.

While the coupling of Compounds LXVIII and LXIX-1, or salts thereof, may occur under numerous conditions, in some embodiments, the coupling of compounds of Formulas LXVIII and LXIX-1, or salts thereof, occurs under Mitsunobu conditions.

In some embodiments, the coupling of compounds of Formula LXVIII, or a salt thereof, with a compound of Formula LXIX-2, or a salt thereof, or a compound of Formula LXIX-3, or a salt thereof, is conducted in the presence of a suitable base. Described in further detail below, there are many suitable bases for use in the described coupling. As a non-limiting example, a suitable base includes an alkoxide salt, such as potassium t-butoxide.

In some embodiments, the leaving group is selected from a halogen, a sulfonate ester, and a diazonium compound. In some embodiments, the leaving group is chloride.

In some embodiments, Compound LXVIII is obtained by coupling a compound having the structure

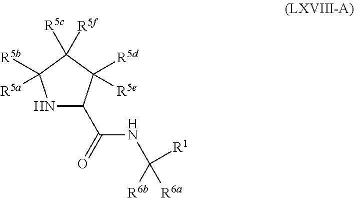

with a compound having the structure

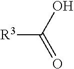

to afford the compound of Formula LXVIII (e.g., a peptide coupling to add the R³ containing moiety). In some embodiments, the coupling is conducted in the presence of an activating reagent and a base. In some embodiments, the activating reagent is HATU and the base is an organic base.

In some embodiments, Compound LXVIII-A is obtained by deprotecting a compound having the structure

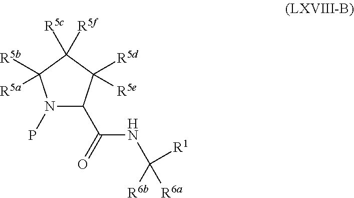

to afford the compound of Formula LXVIII-A, wherein P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In some embodiments, Compound LXVIII-B is obtained by coupling a compound having the structure

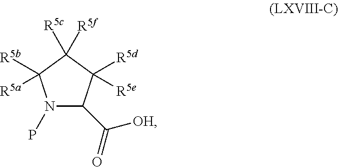

or a salt thereof, with a compound having the structure

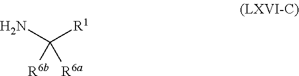

to afford the compound of Formula LXVIII-B (e.g., a peptide coupling to add the R¹ containing moiety). In some embodiments, the coupling is conducted in the presence of an activating reagent and a base. In some embodiments, the activating reagent is HATU and the base is an organic base.

In some embodiments, Compound LXVIII-C is obtained by deprotecting a compound having the structure

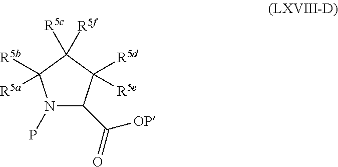

to afford the compound of Formula (LXVIII-C), where P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction.

In some instances, a compound of Formula IV is desired that has certain relative stereochemistries. The general synthetic approaches described above and in more detail below will readily accommodate any desired stereochemistry. In some embodiments, the compound of Formula IV, or a salt thereof, has the structure:

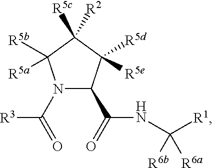

wherein the variable definitions are the same as described above for Formula IV.

In one embodiment, some non-limiting examples of compounds of Formula IV are synthesized according to Scheme 2A-NM:

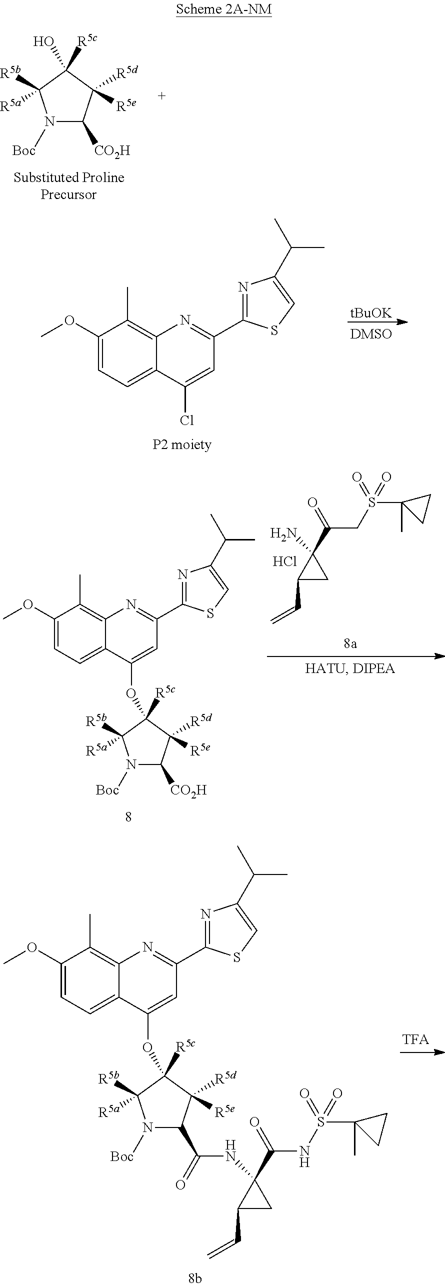

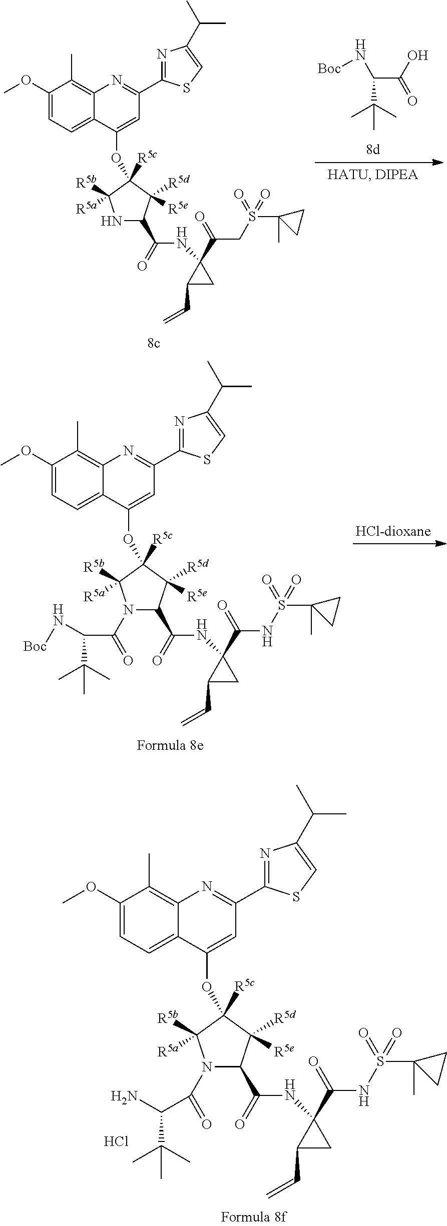

Compounds of formulae 8e and 8f, can be synthesized as shown in Scheme 2A-NM. 4-Hydroxy proline precursor can be treated with 4-chloro-2-(4-isopropylthiazol-2-yl)-7-methoxy-8-methylquinoline under basic conditions, for example potassium tert-butoxide in DMSO, to afford carboxylic acid 8. Carboxylic acid 8 can be coupled with amine 8a using standard coupling conditions, for example HATU in the presence of DIPEA, to afford compound 8b. The Boc protecting group of compound 8b can be removed by treatment with an acid, such as TFA, to afford amine 8c. Amine 8c can be coupled with carboxylic acid 8d using standard coupling conditions, for example HATU in the presence of DIPEA, to afford a compound of formula 8e. The Boc protecting group of the compound of formula 8e can be removed under acidic condition, such as 4M HCl in dioxane, to provide a compound of formula 8f.

A variety of substitution patterns on the proline core of compounds of Formula IV may be obtained by using different substituted proline precursors. To illustrate this point, non-limiting examples of certain substituted proline precursors and exemplary resulting structures are shown in Schemes 2B-NM-1 through 2B-NM-8. In the final compounds depicted in Schemes 2B-NM-1 through 2B-NM-8, non-limiting examples of R include HET as described above. A non-limiting example of P₄ is Boc and a non-limiting example of P₁′ is cycloalkyl.

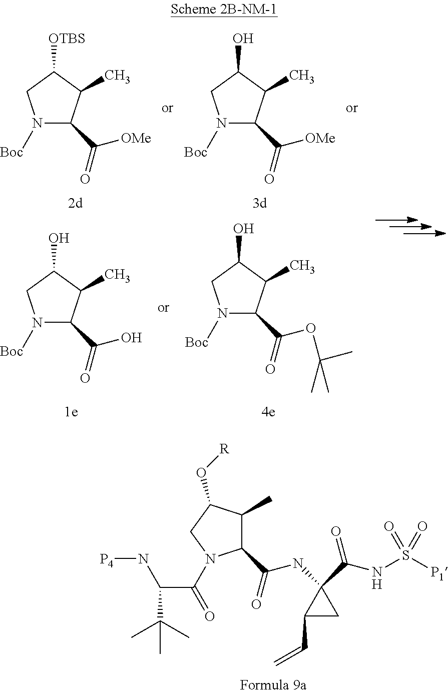

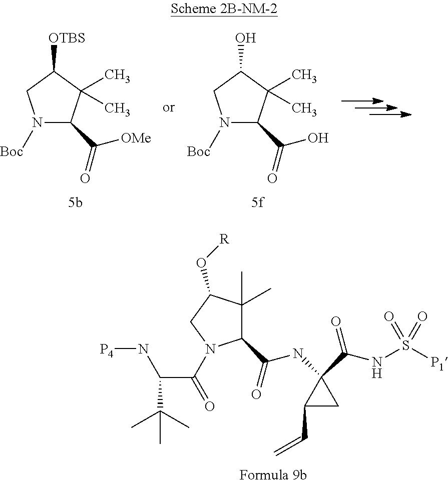

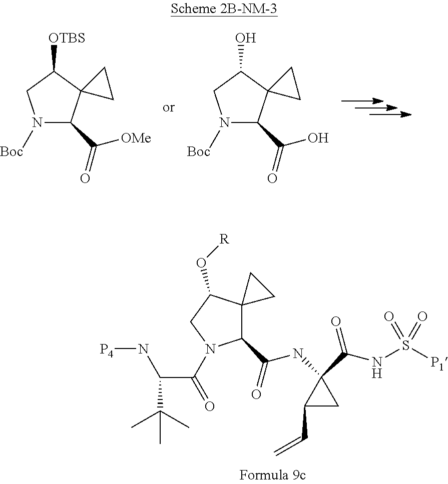

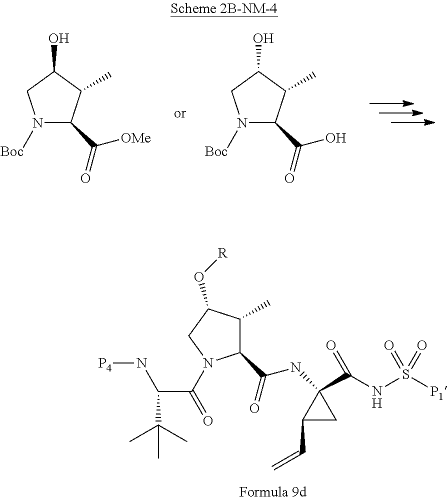

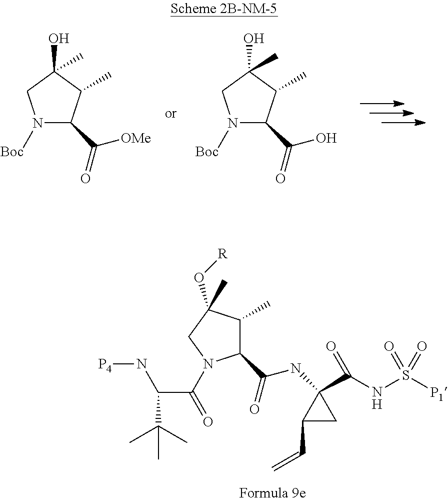

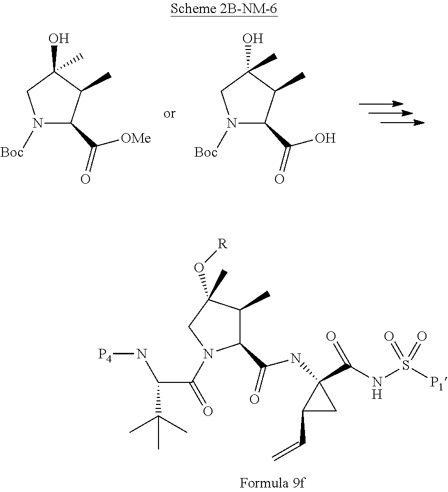

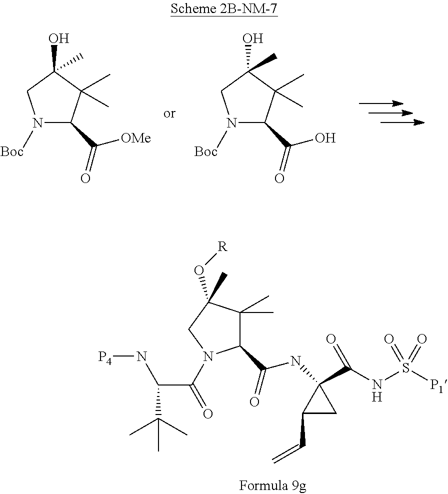

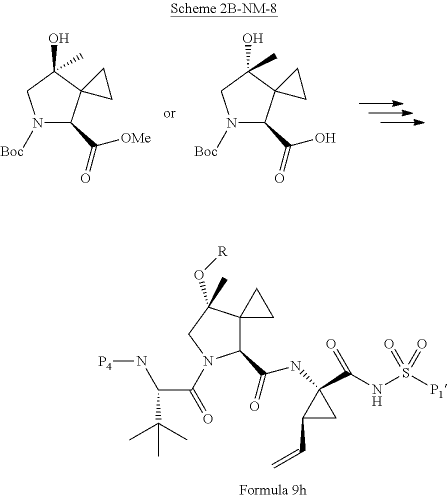

In some embodiments, macrocyclic compounds of Formula I, or salts thereof,

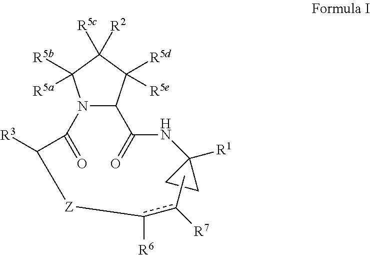

are readily synthesized using ring closing metathesis of a compound of Formula LXXIII as a final step,

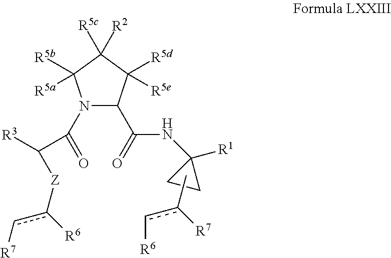

where”
R⁶ and R⁷ are each independently hydrogen, halo, or together with the carbon atoms to which they are attached to form an optionally substituted cycloalkyl; the dashed line represents an optional double bond; and
and all other variable definitions are the same as described above for Formula I.

In some embodiments, Compound LXXIII is obtained by coupling a compound of Formula LXXIII-A with a compound of Formula LXXIII-B, or salt thereof

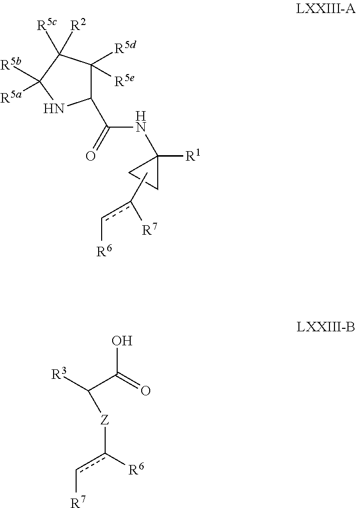

to afford the compound of Formula LXXIII (e.g., a peptide coupling to add the R³ containing moiety as a final step prior to ring closing). In some embodiments, the coupling is conducted in the presence of an activating reagent and a base. In some embodiments, the activating reagent is HATU and the base is an organic base.

In some embodiments, Compound LXXIII-A is obtained by deprotecting a compound having the structure

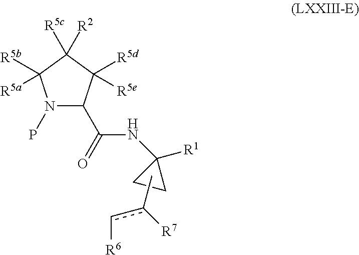

to afford the compound of Formula LXXIII-A, where P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In some embodiments, Compound LXXIII-E is obtained by coupling a compound of Formula LXIII-F with a compound of Formula LXXIII-D

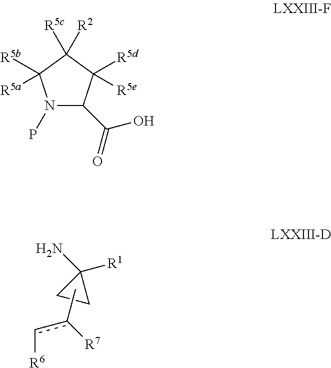

to afford the compound of Formula LXXIII-E (e.g., a peptide coupling to add the R¹ containing moiety). In some embodiments, the coupling is conducted in the presence of an activating reagent and a base and P is a Boc or CBz protecting group. In some embodiments, the activating reagent is HATU and the base is an organic base.

In some embodiments, Compound LXXIII-F is obtained by deprotecting a compound having the structure

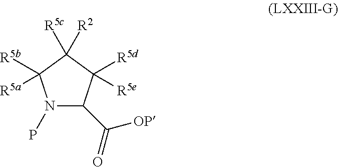

to afford the compound of Formula LXXIII-F, where P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction.

Alternatively, Compound LXXIII is obtained by coupling a compound of Formula LXXIII-C, or salt thereof, with a compound of Formula LXXIII-D

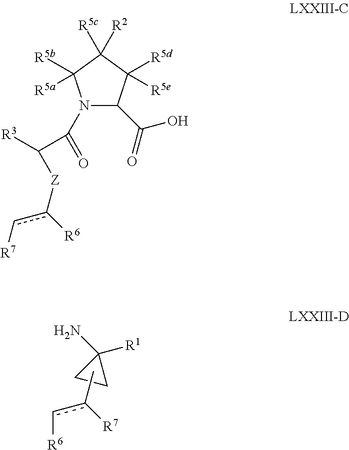

to afford the compound of Formula LXXIII or salt thereof (e.g., a peptide coupling to add the R¹ containing moiety as a final step prior to ring closing).

In some embodiments, Compound LXXIII-C is obtained by deprotecting a compound having the structure

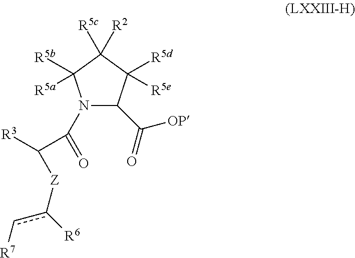

to afford the compound of Formula LXXIII-C, where P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction.

In some embodiments, Compound LXXIII-His obtained by coupling a compound having the structure

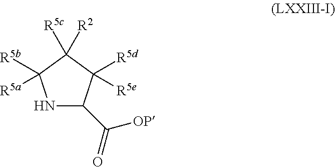

with a compound having the structure

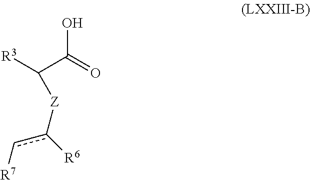

to afford the compound of Formula LXXIII-H (e.g., a peptide coupling to add the R³ containing moiety). In some embodiments, the coupling is conducted in the presence of an activating reagent and a base and P is a Boc or CBz protecting group. In some embodiments, the activating reagent is HATU and the base is an organic base.

In some embodiments, Compound LXXIII-I is obtained by deprotecting a compound having the structure

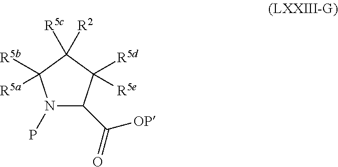

to afford the compound of Formula LXXIII-F; wherein P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In some embodiments, Compound LXXIII-G is obtained by reacting a compound having the structure

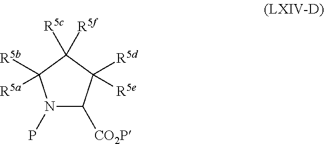

with a compound having the structure selected from X-LG or

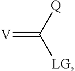

resulting in attachment of the R² moiety to the proline core. In some embodiments, R^{5f} is OH, NH₂, or SH; LG is a leaving group; and the remaining variables are as described above for Formula I. In some embodiments, the coupling step is conducted in the presence of an alkoxide salt. In some embodiments, the alkoxide salt is potassium t-butoxide. In some embodiments, the leaving group is selected from a halogen, a sulfonate ester, and a diazonium compound. In some embodiments, the leaving group is chloride.

In alternative embodiments, macrocyclization may occur through methods other than ring closing metathesis. Thus, for example, in some embodiments, compounds of Formula I may be made by intramolecular cyclization of a compound Formula XC, or a salt thereof as the final step,

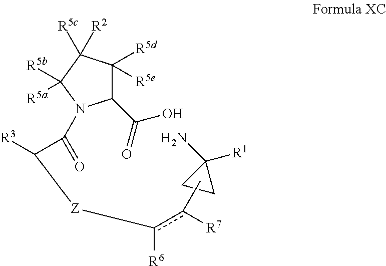

where the variables are as described above for Formula I. In some embodiments, the coupling is conducted in the presence of an activating reagent and a base. In some embodiments, the activating reagent is selected from HATU or CDI and the base is an organic base.

In some embodiments, Compound XC is obtained by deprotecting a compound having the structure

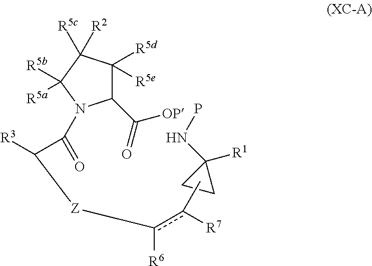

to afford the compound of Formula XC, or a salt thereof; where P is a protecting group for an amine and P′ is a protecting group for a carboxylic acid. In some embodiments, P′ is an alkyl group, cycloalkylalkyl group, or arylalkyl group. In some embodiments, P is a carbamate protecting group selected from Boc, Fmoc, or CBz. In some embodiments, deprotection of P and P′ independently occurs under conditions selected from acidic, basic, or reducing conditions. In some embodiments, deprotection of P and P′ occurs under basic conditions. In other embodiments, deprotection of P and P′ occurs under acidic conditions. In some embodiments, deprotection of P and P′ occurs by reduction.

The various substituent groups may be added using methods analous to those described above.

Alternatively, compounds of Formula I may be made by intramolecular cyclization a Formula XCI, or a salt thereof as a final step:

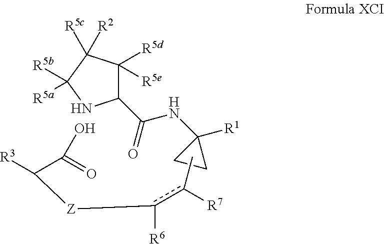

where the variables are as described above for Formula I. In some embodiments, the coupling is conducted in the presence of an activating reagent and a base. In some embodiments, the activating reagent is selected from HATU or CDI and the base is an organic base.

In some embodiments, Compound XCI is obtained by deprotecting a compound having the structure

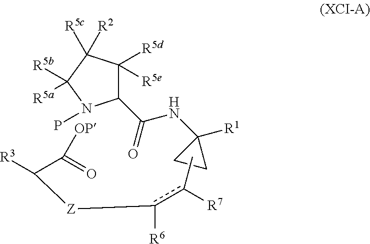

to afford the compound of Formula XCI, or a salt thereof, where P is a protecting group for an amine and P′ is a protecting group for a carboxylic acid. In some embodiments, P′ is an alkyl group, cycloalkylalkyl group, or arylalkyl group. In some embodiments, P is a carbamate protecting group selected from Boc, Fmoc, or CBz. In some embodiments, deprotection of P and P′ independently occurs under conditions selected from acidic, basic, or reducing conditions. In some embodiments, deprotection of P and P′ occurs under basic conditions. In other embodiments, deprotection of P and P′ occurs under acidic conditions. In some embodiments, deprotection of P and P′ occurs by reduction.

In alternative embodiments for making compounds of Formula I, macrocyclic formation may occur prior to the coupling of various substituent groups. Thus, for example, in one embodiment, compounds of Formula I may be obtained by reacting a compound of Formula LXXIX, or a salt thereof, with a compound of Formula LXIX-1, or a salt thereof, a compound of Formula LXIX-2, or a salt thereof, or a compound of Formula LXIX-3, or a salt thereof, resulting in attachment of the R² moiety to the proline core as a final step:

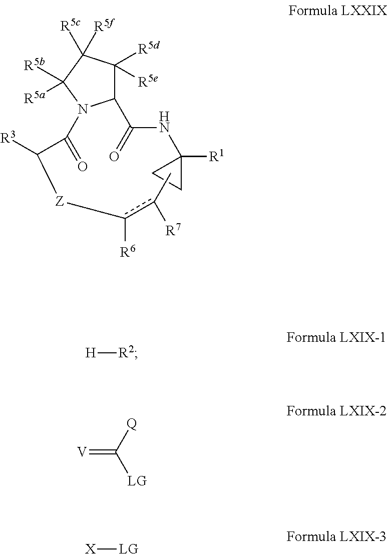

where R^{5f} is OH, NH₂, or SH; LG is a leaving group; and the remaining variables are as described above for Formula I. In some embodiments, when reacting the compound of Formula LXXIX, or a salt thereof, with a compound of Formula LXIX-1, the reaction is conducted under Mitsunobu conditions. In some embodiments, when reacting the compound of Formula LXXIX, or a salt thereof, with a compound of Formula LXIX-2, or a salt thereof, or a compound of Formula LXIX-3, or a salt thereof, the reaction is conducted in the presence of an alkoxide salt. In one embodiments, the alkoxide salt is potassium t-butoxide. In some embodiments, the leaving group is selected from a halogen, a sulfonate ester, and a diazonium compound. In one embodiment, the leaving group is chloride. In some embodiments, the leaving group is selected from a halogen, a sulfonate ester, and a diazonium compound. In some embodiments, the leaving group is chloride.

In some embodiments, Compound LXXIX is obtained by performing a ring closing metathesis reaction on a compound having the structure:

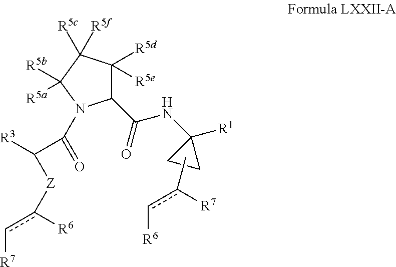

to afford the compound of Formula LXXIX. In some embodiments, ring closing metathesis is conducted in the presence of a Zhan catalyst.

In some embodiments, Compound LXXII-A is obtained by coupling a compound of Formula LXXIII-A with a compound of Formula LXXIII-B, or salt thereof

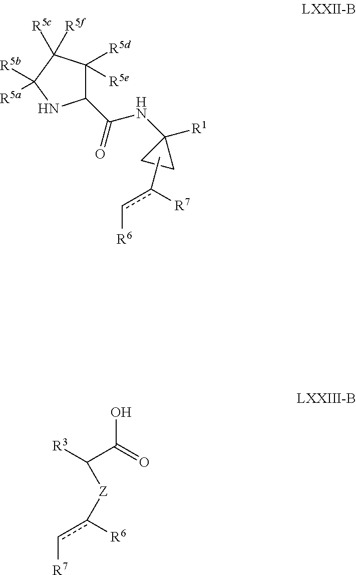

to afford the compound of Formula LXXII-A (e.g., a peptide coupling to add the R³ containing moiety as the step prior to ring closing metathesis). In some embodiments, the coupling is conducted in the presence of an activating reagent and a base. In some embodiments, the activating reagent is HATU and the base is an organic base.

In some embodiments, Compound LXXII-B is obtained by deprotecting a compound having the structure

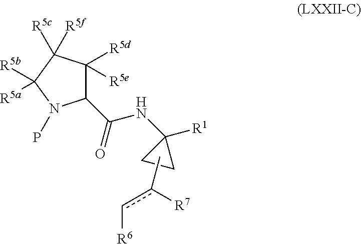

to afford the compound of Formula LXXII-B, where P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In some embodiments, Compound LXXII-B is obtained by coupling a compound of Formula LXXII-D with a compound of Formula LXXIII-D

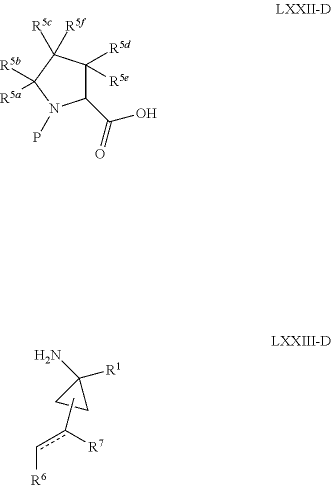

to afford the compound of Formula LXXII-C (e.g., a peptide coupling to add the R¹ containing moiety). In some embodiments, the coupling is conducted in the presence of an activating reagent and a base and P is a Boc or CBz protecting group. In some embodiments, the activating reagent is HATU and the base is an organic base.

In some embodiments, Compound LXXII-D is obtained by deprotecting a compound having the structure

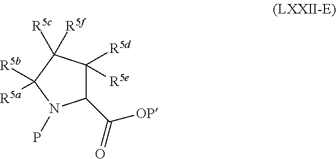

to afford the compound of Formula LXXII-D, where P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction.

Alternatively, Compound LXXII-A is be obtained by coupling a compound of Formula LXXII-F, or salt thereof, with a compound of Formula LXXIII-D

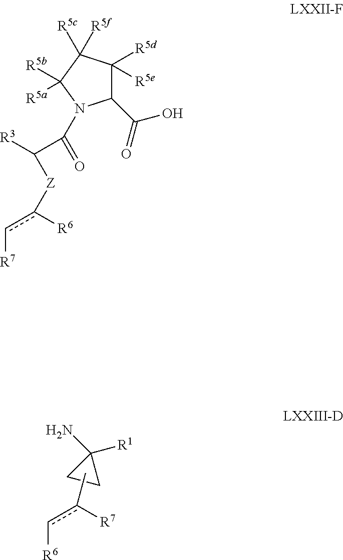

to afford the compound of Formula LXXII-A, or salt thereof (e.g., a peptide coupling to add the R¹ containing moiety as the step prior to ring closing metathesis).

In some embodiments, Compound LXXII-F is obtained by deprotecting a compound having the structure

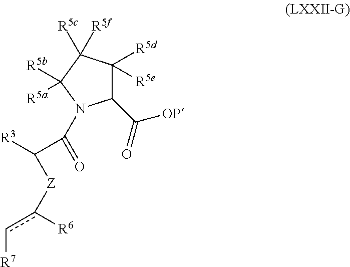

to afford the compound of Formula LXXII-F, where P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction.

In some embodiments, Compound LXXII-G is obtained by coupling a compound having the structure

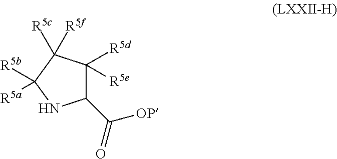

with a compound having the structure

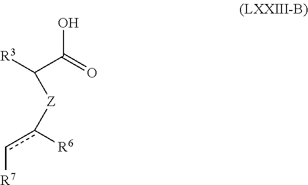

to afford the compound of Formula LXXII-G (e.g., a peptide coupling to add the R³ containing moiety). In some embodiments, the coupling is conducted in the presence of an activating reagent and a base and P is a Boc or CBz protecting group. In some embodiments, the activating reagent is HATU and the base is an organic base.

In some embodiments, Compound LXXII-His obtained by deprotecting a compound having the structure

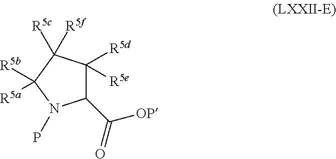

to afford the compound of Formula LXXII-H, where P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In some alternative embodiments, Compound LXXIX is obtained by deprotecting a compound having the structure

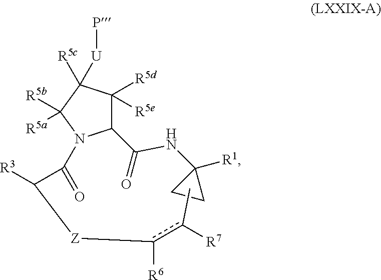

or a salt thereof, to afford the compound of Formula LXXIX, or salt thereof, where, U is —O—, —S—, or —NH—; and P′″ is a protecting group for an alcohol, a thiol or an amine. In some embodiments, U is —O— and P′″ is an silicon-containing protecting group. In some embodiments, the deprotecting is conducted in the presence of fluoride.

In some embodiments, Compound LXXIX-A is obtained by ring-closing metathesis of a compound having the structure:

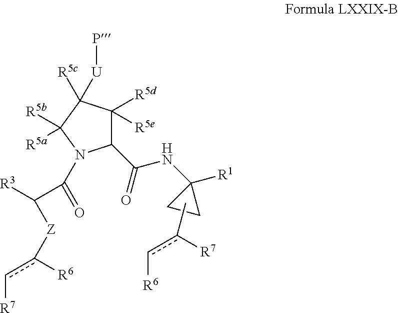

to afford the compound of Formula LXXIX-A. In some embodiments, the ring closing metathesis is conducted in the presence of a Zhan catalyst.

In some embodiments, Compound LXXIX-B is obtained by coupling a compound of Formula LXXIX-C with a compound of Formula LXXIII-B, or salt thereof

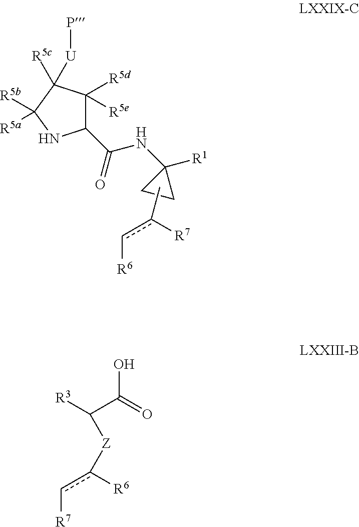

to afford the compound of Formula LXXIX-B (e.g., a peptide coupling to add the R³ containing moiety as the step prior to ring closing metathesis). In some embodiments, the coupling is conducted in the presence of an activating reagent and a base. In some embodiments, the activating reagent is HATU and the base is an organic base.

In some embodiments, Compound LXXIX-C is obtained by deprotecting a compound having the structure

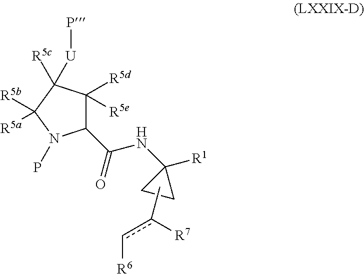

to afford the compound of Formula LXXIX-C; wherein P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In some embodiments, Compound LXXIX-D is obtained by coupling a compound of Formula LXXIX-E with a compound of Formula LXXIII-D

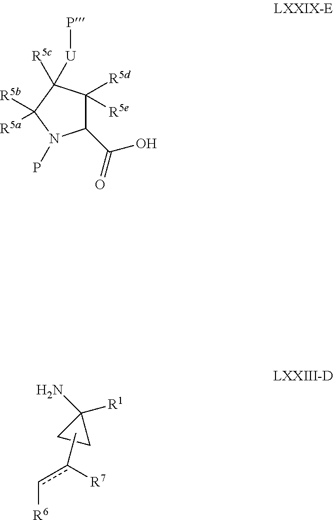

to afford the compound of Formula LXXIX-D (e.g., a peptide coupling to add the R³¹ containing moiety). In some embodiments, the coupling is conducted in the presence of an activating reagent and a base and P is a Boc or CBz protecting group.

In some embodiments, Compound LXXIX-E is obtained by deprotecting a compound having the structure

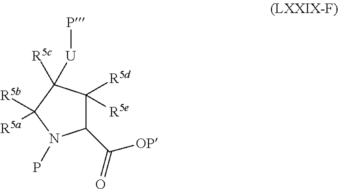

to afford the compound of Formula LXXIX-E; wherein P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction.

Alternatively, Compound LXXIX-B is obtained by coupling a compound of Formula LXXIX-G, or salt thereof, with a compound of Formula LXXIII-D:

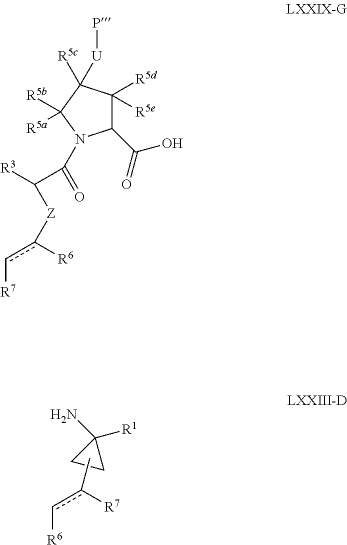

to afford the compound of Formula LXXIX-B, or salt thereof (e.g., a peptide coupling to add the R¹ containing moiety as the step prior to ring closing metathesis). In some embodiments, the coupling is conducted in the presence of an activating reagent and a base. In some embodiments, the activating reagent is selected from HATU or CDI and the base is an organic base.

In some embodiments, Compound LXXIX-G is obtained by deprotecting a compound having the structure

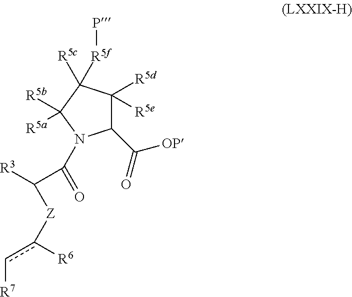

to afford the compound of Formula LXXIX-G, where P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction.

In some embodiments, Compound LXXIX-G is obtained by coupling a compound having the structure

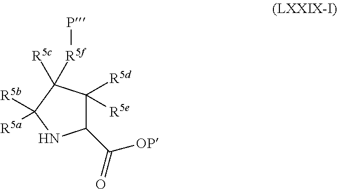

with a compound having the structure

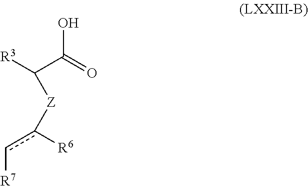

to afford the compound of Formula LXXIX-H (e.g., a peptide coupling to add the R³ containing moiety. In some embodiments, the coupling is conducted in the presence of an activating reagent and a base. In some embodiments, the activating reagent is selected from HATU or CDI and the base is an organic base.

In some embodiments, Compound LXXIX-I is obtained by deprotecting a compound having the structure

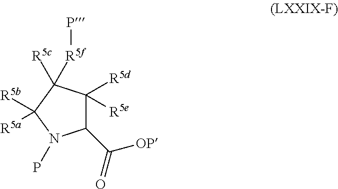

to afford the compound of Formula LXXIX-I, where P is a protecting group for an amine. In some embodiments, P is a carbamate protecting group. In some embodiments, P is a Boc, Fmoc, or CBz protecting group. In some embodiments, the deprotection occurs under acidic conditions. In other embodiments, the deprotection occurs under basic conditions. In some embodiments, the deprotection occurs under reductive conditions.

In other embodiments where macrocyclic formation occurs prior to the coupling of some substituent groups, the R¹ containing moiety may be added to the proline core as a final step. Thus, for example, in one embodiment, compounds of Formula I may be obtained by reacting a compound of Formula LXXXIII, or a salt thereof, with a compound of Formula LXXXIX, or a salt thereof,

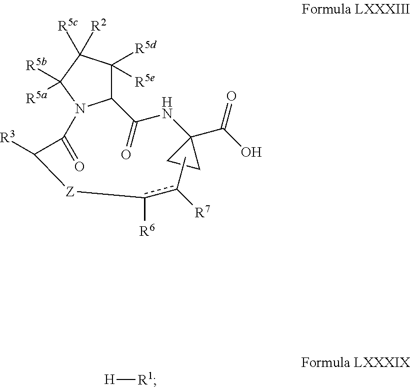

where the variables are as defined above for Formula I. In some embodiments, the coupling is conducted in the presence of an activating reagent and a base. In some embodiments, the activating reagent is selected from HATU or CDI and the base is an organic base.

In some embodiments, Compound LXXXIII is obtained by deprotecting a compound having the structure

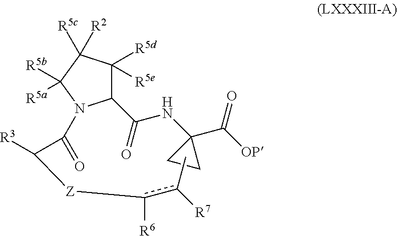

to afford the compound of Formula LXXXIII, where P′ is a protecting group for a carboxylic acid. In some embodiments, P′ is an alkyl group, cycloalkylalkyl group, or arylalkyl group. In some embodiments, the deprotection occurs by saponification. In some embodiments, the deprotection occurs under acidic conditions. In some embodiments, the deprotection occurs by reduction. Earlier steps in the synthese can mirror those described above for adding the R³ and R² containing moieties and ring closing metathesis.

In some instances, a compound of Formula I is desired that has certain relative stereochemistries. The general synthetic approaches described above and in more detail below will readily accommodate any desired stereochemistry. In some embodiments, the compound of Formula I, or a salt thereof, has the following structure:

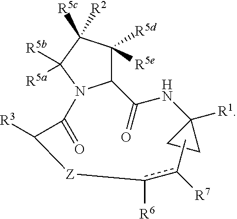

In one embodiment, some non-limiting examples of compounds of Formula I are synthesized according to Scheme 2A-M:

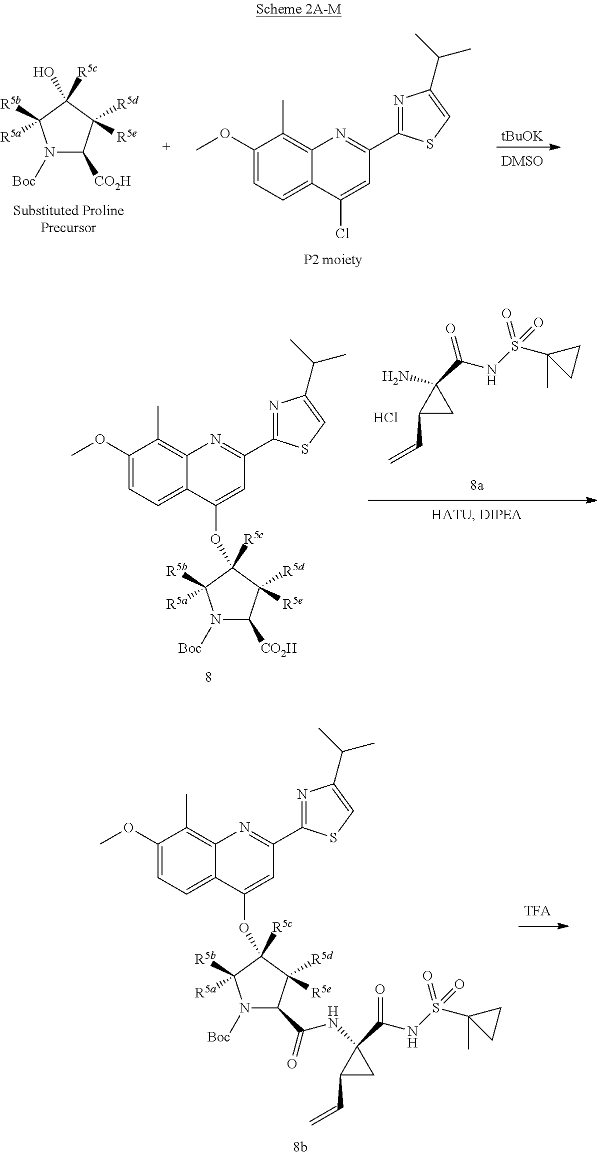

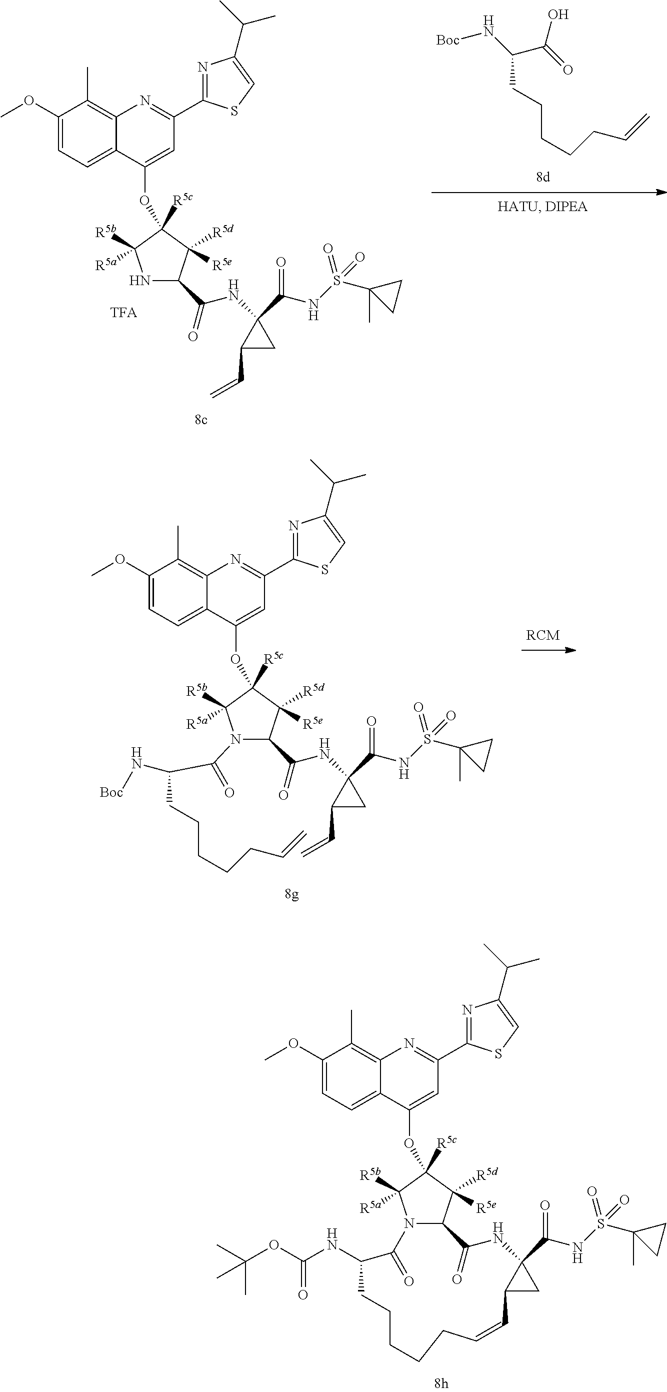

Macrocycles, such as compound 8h, can be synthesized as shown in Scheme 2A-M. 4-Hydroxy proline precursor can be treated with 4-chloro-2-(4-isopropylthiazol-2-yl)-7-methoxy-8-methylquinoline under basic conditions, for example potassium tert-butoxide in DMSO, to afford carboxylic acid 8. Carboxylic acid 8 can be coupled with amine 8a using standard coupling conditions, for example HATU in the presence of DIPEA, to afford compound 8b. The Boc protecting group of compound 8b can be removed by treatment with an acid, such as TFA, to afford amine 8c. Amine 8c can be coupled with carboxylic acid 8d using standard coupling conditions, for example HATU in the presence of DIPEA, to afford compound 8g. Compound 8g can be cyclized in the presence of a catalyst, such as a Zhan catalyst, to provide macrocycles, such as compound 8h.

As above, a variety of substitution patterns on the proline core of compounds of Formula I may be obtained by using different substituted proline precursors. To illustrate this point, non-limiting examples of certain substituted proline precursors and exemplary resulting structures are shown in Schemes 2B-M-1 through 2B-M-8. In the final compounds depicted in Schemes 2B-M-1 through 2B-M-8, non-limiting examples of R include HET as described above. A non-limiting example of P₄ is Boc and a non-limiting example of P₁′ is cycloalkyl.

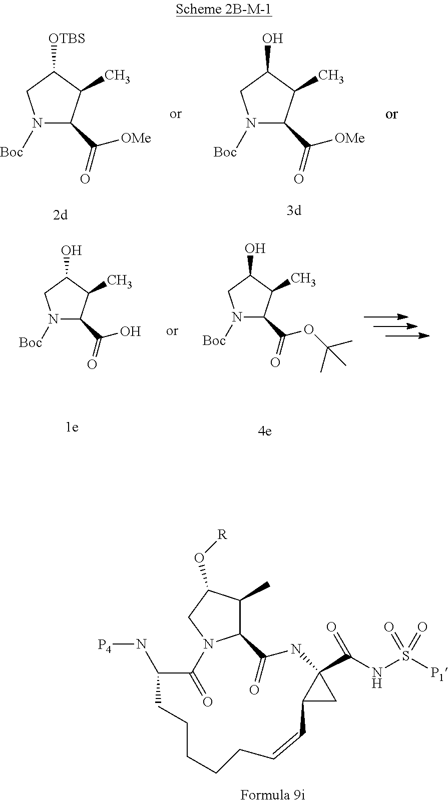

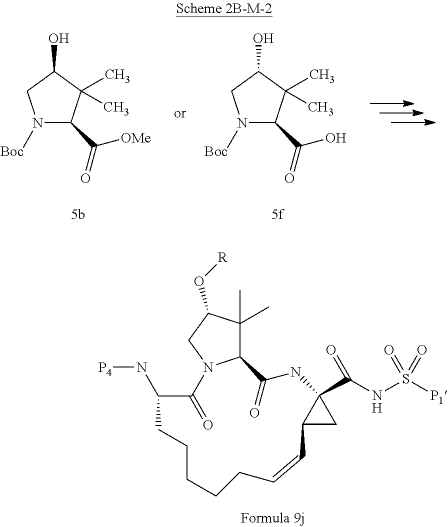

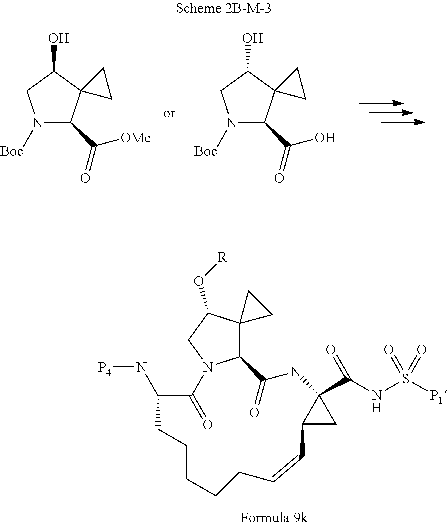

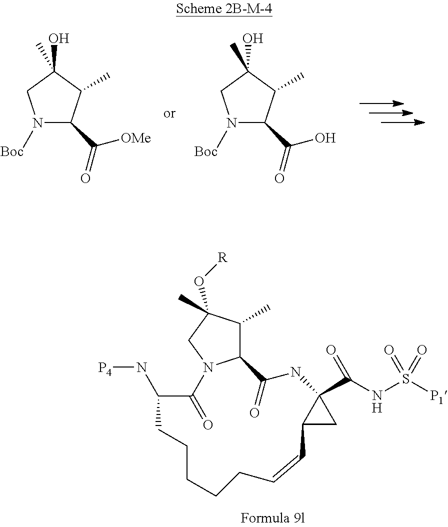

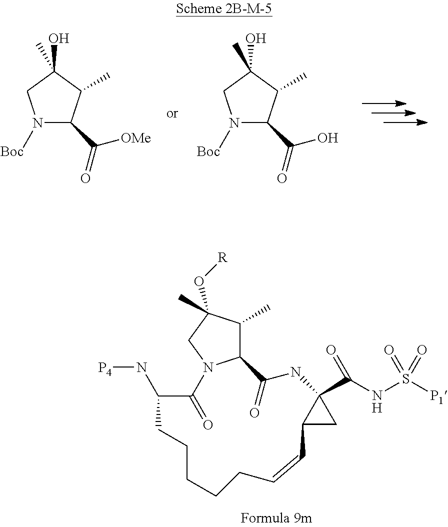

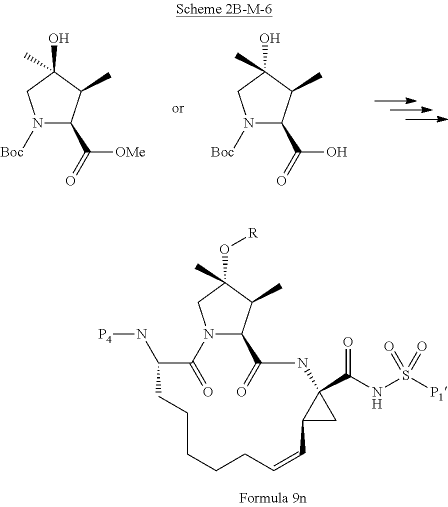

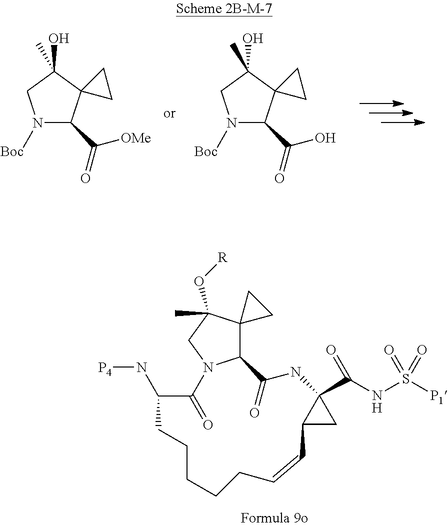

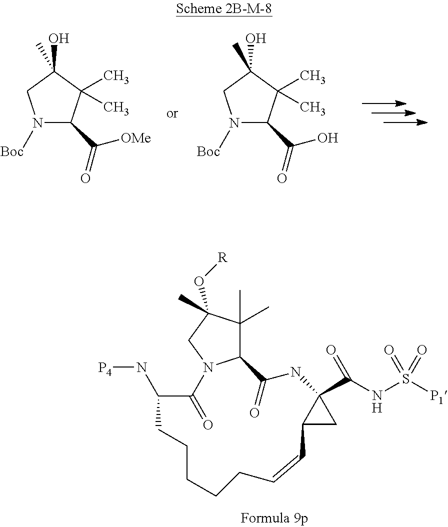

Different P₂ moieties (Scheme 2A-NM and 2A-M) or R moieties (Schemes 2B-NM-1 through 2B-NM-8 and Schemes 2B-M-1 through 2B-NM-8) can be used to prepare substituted proline analogs. Non-limiting examples of P₂ moieties and R moieties include the following:

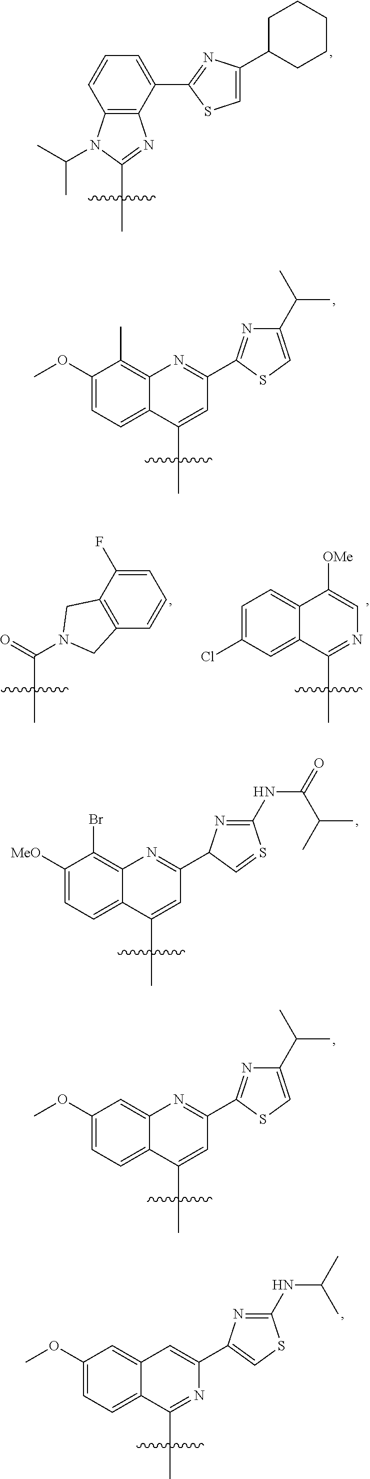

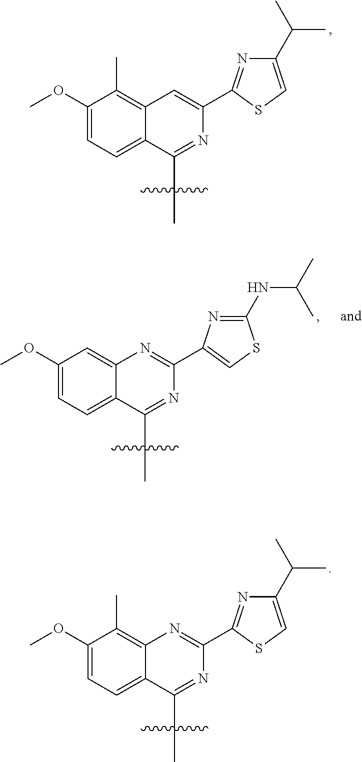

Different P₄ moieties can be used to prepare substituted proline analogs according to Schemes 2A-NM and 2A-M. Non-limiting examples of P₄ moieties include alkyl carbamates, cycloalkyl carbamates, and substituted versions thereof such as the following:

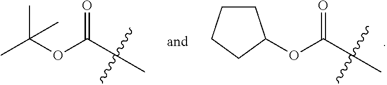

Different P_{1′} moieties can be used to prepare substituted proline analogs according to Schemes 2A-NM and 2A-M. One will readily appreciate that these different P_{1′} moieties are readily introduced by varying compound 8a from Schemes 2A-NM and 2A-M. Non-limiting examples of P_{1′} moieties include alkyl, cycloalkyl, and substituted versions thereof such as the following:

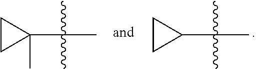

The syntheses described above are achieved using a substituted proline precursor. These precursors can be prepared by a variety of methods described below. For illustrative purposes only and without being limited to such compounds, the description of precursor synthesis is limited to methyl substitution on the proline. Those of skill in the art will recognize that similar procedures may be followed to provide substitution with other moieties at the respective positions described below.

One method for obtaining 4-methyl substitution on the proline precursor is described in Scheme 1:

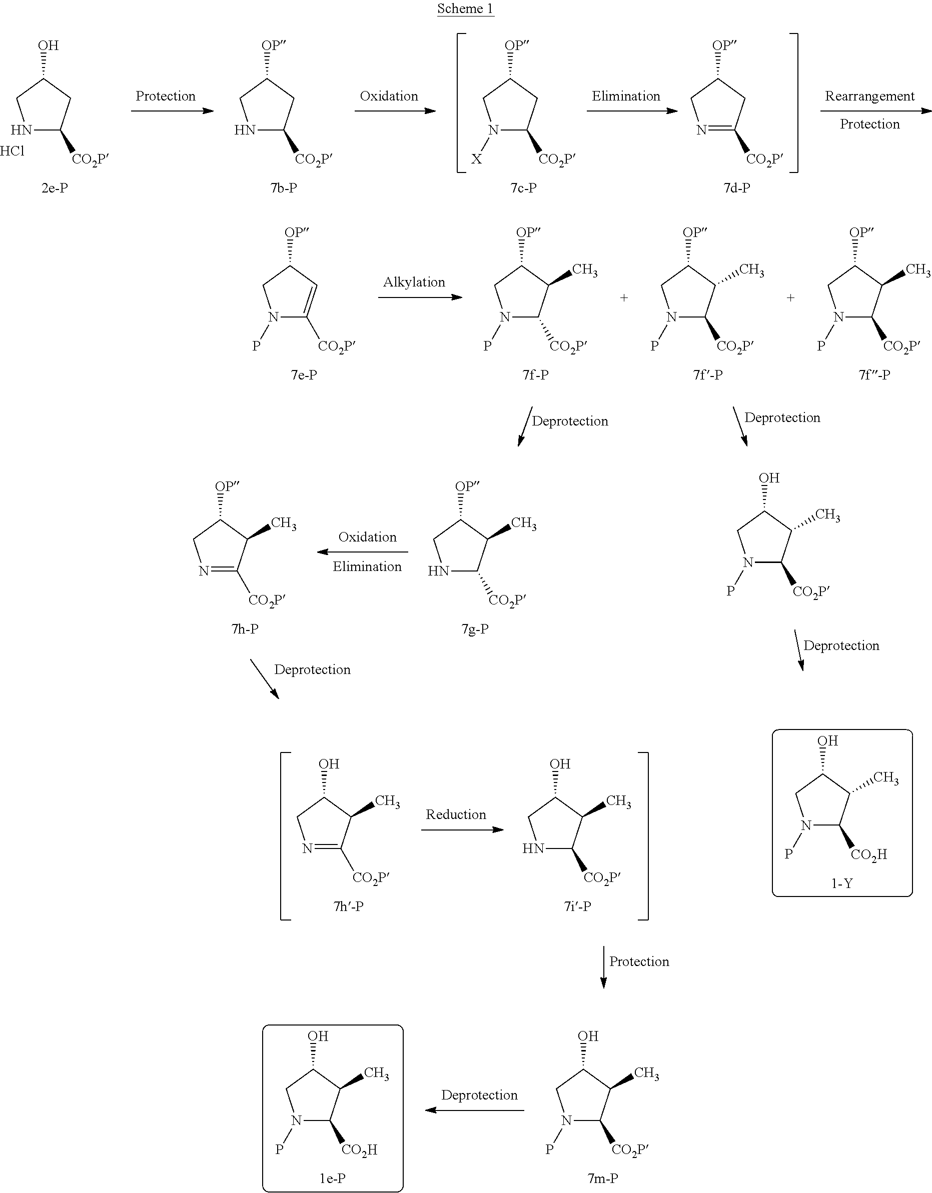

In one embodiment, Compound 2e-P, or a salt thereof, is a suitable starting material for the preparation of Intermediate 7e-P, or a salt thereof, and ultimate preparation of substituted-proline precursors. In the compounds above, P represents a functional group that is suitable protecting group for an amine, P′ represents a functional group that is suitable for protecting a carboxylic acid (e.g., as an ester) and P″ represents a functional group that is suitable for protecting an alcohol. As a non-limiting example, P is a carbamate protecting group, such as a CBz group, P′ is an alkyl group, such as methyl, and P″ is a silyl ether, such as a t-butyldiphenylsilyl ether. As illustrated in Scheme 1, Compound 7b-P may be prepared from Compound 2e-P by protecting the alcohol functional group of Compound 2e-P with a suitable alcohol protecting group. Such a protecting group is readily introduced by the treatment of Compound 2d-P with t-butyldiphenylsilyl chloride in the presence of a base, such as imidazole.

Oxidation of Compound 7b-P will readily afford Compound 7c-P. In Compound 7c-P, X is any suitable leaving group. As a non-limiting example, X may be a halide such as chlorine. A chlorine atom may be readily introduced by treatment of Compound 7b-P with a chlorine-containing oxidant, such as t-butylhypochlorite, in a suitable solvent, such as diethylether. Elimination of hydrogen chloride from Compound 7c-P will readily afford Compound 7d-P. Such an elimination may proceed via the treatment of compound 7c-P with a suitable base. As a non-limiting example, a suitable base includes DBU.

Rearrangement of Compound 7d-P is readily affected by base. Protection of the amino functional group present affords Compound 7e-P. In Compound 7e-P, P is any suitable protecting group for an amine. As a non-limiting example, P may be a carbamate protecting group, such as CBz. Such a protecting group is readily introduced by treatment with CBz-Cl, or an equivalent thereof, in the presence of a suitable base, such as 2,6-lutidine, and a suitable solvent, such as dichloromethane. Optionally, Compound 7e-P may be prepared from Compound 7b-P without isolation of intermediate compounds.

As illustrated in Scheme 1, reacting Compound 7e-P such that methane is added across the double bond will readily afford diastereomers 7f-P, 7-f′-P, and 7-f″P. Such a reaction can be accomplished via a “1,4”- or “Michael”-addition with an organometallic reagent. Many organometallic reagents and reaction conditions suitable for adding methane across a double bond are known in the art. See Huy et al. Organic Letters, 2011, 13, 216-219; Ezquerra, J. et al., Tetrahedron: Assymmetry 1996, 7, 2613-2626; and Toyooka, N. et al. Synlett 2003, 55-58, all of which are herein incorporated by reference in their entirety.

As a non-limiting example, a suitable reagent for accomplishing such a reaction is a dimethyl metal salt, such as lithium dimethylcuprate. In one embodiment, the desired diastereomer is Compound 7f-P, which may be isolated by crystallizing in a suitable non-polar solvent, such as hexane.

As one will readily appreciate, the stereochemical outcome of the alkylation of Compound 7e-P may be favorably influence by sterics. Thus, a bulky protecting group for P″, such as a t-butyldiphenylsilyl ether, will favor alkylation on the face of the heterocycle opposite the protecting group in order to avoid unfavorable steric interactions between the methyl group and the silyl ether. As such, the diasteromers 7f-P and 7f″-P depicted in Scheme 1 will be favored over the diasteromer 7f′P. Moreover, diasteromer 7f-P will be favored over the diasteromer 7f″-P because of unfavorable steric interactions arising between the syn methyl group and ester group present in diasteromer 7f″-P. Thus, P′ may also affect the stereochemical outcome of the alkylation reaction by contributing to the bulkiness of the ester group and unfavorable steric interactions arising between the ester group and the methyl group. When P′ is methyl and P″ is a t-butyldiphenylsilyl ether, Compounds 7f-P, 7f′-P, and 7f″-P are obtained in an approximate ratio of 88:4:8, respectively. Consequently, in one embodiment of the invention, Compound 7f-P is preferentially formed over the other diastereomers. In another embodiment of the invention, Compound 7f-P is formed in diastereomeric excess. In another embodiment, Compound 7f-P is formed in about 50% or more diastereomeric excess. In another embodiment of the invention, Compound 7f-P is formed in about 75% or more diastereomeric excess.

As illustrated in Scheme 1, Compound 7f-P is a suitably functionalized intermediate for the preparation of Compound 7i′-P. Deprotection of the amino protecting group, P, in Compound 7b-P readily affords Compound 7g-P. Depending upon the protecting group for the amine, the deprotection may readily occur under any of several conditions. As a non-limiting example, when P is a CBz group, deprotection may readily proceed upon exposure to hydrogen gas in the presence of palladium on activated carbon.

Compound 7h-P is readily formed under reaction conditions similar to those utilized in the conversion of Compound 7b-P to 7d-P. As a non-limiting example, the amine of Compound 7g-P may be oxidized to introduce a leaving group such as chlorine. The chlorine atom may be readily introduced by treatment of Compound 7g-P with a chlorine-containing oxidant, such as t-butylhypochlorite, in a suitable solvent, such as diethylether. Elimination of hydrogen chloride from Compound 7g-P will readily afford Compound 7h-P. Such an elimination may proceed via the treatment of compound 7g-P with a suitable base. As a non-limiting example, a suitable base includes DBU. One will readily appreciate that because the elimination reaction installs a double bond such that the stereochemistry of the carbon alpha to the carboxyl group is erased. Thus, the second diasteromer in Scheme 1H-B may also be utilized as a starting material for Scheme 1H-C instead of Compound 7f-P.

Deprotection of the alcohol protecting group, P″, in Compound 7h-P readily affords Compound 7h′-P. Depending upon the protecting group for the alcohol, the deprotection may readily occur under any of several conditions. As a non-limiting example, when P″ is a silyl ether, such t-butyldiphenylsilyl ether, deprotection may readily proceed upon exposing Compound 7h-P to TBAF.

Compound 7i′-P and its diastereomer are readily afforded upon reduction of Compound 7h′-P. Because it is the Schiff base, or imine, motif that is reduced in Compound 7h′-P to afford 7i′-P, several known reagents may be utilized and are described below. Surprisingly, the reducing agent and deprotected alcohol had a profound effect upon the stereochemical outcome of the reduction. For instance, reduction of Compound 7h′-P with NaBH₃CN, NaBH(OAc)₃, and hydrogen with Pd/C afforded ratios of Compounds 7i′-P to its diastereomer of 3:2, ˜95:5, and an undetermined ratio. Reduction of Compound 7h′-P with NaBH₃CN, NaBH(OAc)₃, and hydrogen with Pd/C, however, afforded ratios of 1:2, ˜1:9, and less than 5:95, respectively.

As illustrated in Scheme 1, Compound 7i′-P represents a suitable starting material for the preparation of advanced intermediates such as 7m-P and 1e-P that are suitable for ultimately preparing a compound of Formulas I, III, or V. Protection of the amino group present in Compound 7i′-P readily affords Compound 7m-P. Any suitable protecting group for an amine may be utilized. As a non-limiting example, P may be a carbamate protecting group, such as BOC. Such a protecting group is readily introduced by treatment of Compound 7i′-P with BOC₂O, or an equivalent thereof, in the presence of a suitable base, such as sodium bicarbonate.

Deprotection of the carboxylic acid protecting group of Compound 7m-P will afford Compound 1e-P. Depending upon the protecting group for the carboxylic acid, the deprotection may readily occur under any of several conditions. As a non-limiting example, when P′ is a protecting group, such as a methyl group, deprotection may readily proceed via saponification of the ester protecting group in the presence of a base under aqueous conditions, such as with lithium hydroxide in the presence of water.

As illustrated in Scheme 1, diasteromer 7f′-P also represents a suitable starting material for the preparation of an advanced intermediate such as 1-Y that is suitable for ultimately preparing the compounds described herein. P′ and P″ are readily cleaved to afford the hydroxyl group and carboxylic acid group present in Compound 1-Y. Under certain reaction conditions, cleavage of P′ and P″ may occur in a “one pot” reaction. Under other reaction conditions, cleavage of P′ and P″ may occur over multiple steps. Additionally, it may be necessary to alter the nature of P such that the secondary amine is suitably protected for subsequent reactions. Optionally, such alterations in the protecting group may be performed before or after cleavage of P′ or P″.

Substituted proline precursors 1e-P and 1-Y are particularly suited for the preparation of the compounds described herein.

As alternatives to Scheme 1, several other methods may be used to prepare substituted proline precursors. For example, several methods for obtaining 2-methyl or 4-methyl substitution on the proline precursor are described in Scheme 1A:

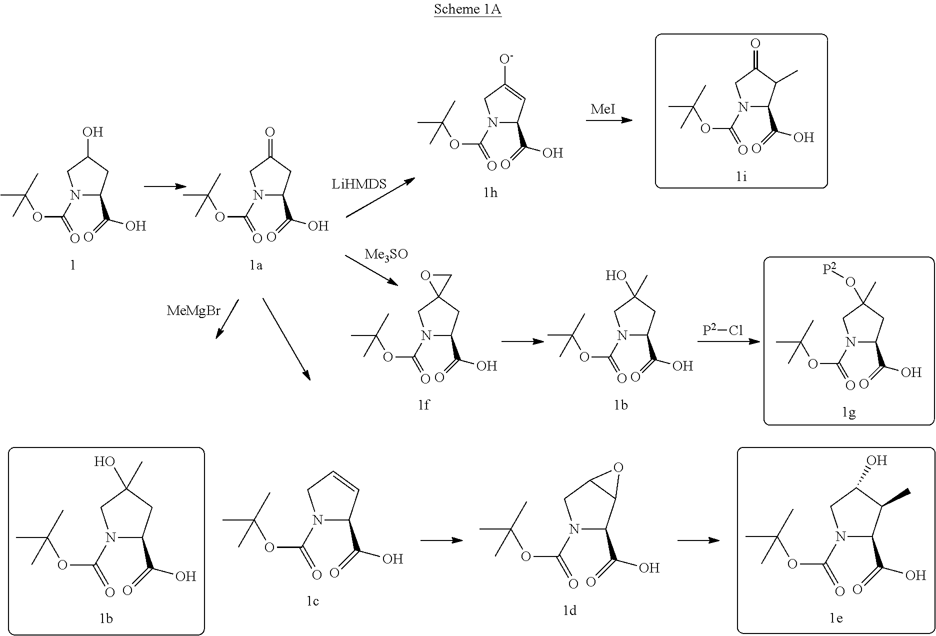

N-Boc-4-hydroxy-L-proline (1) can be oxidized to afford Compound 1a. Such a compound represents a versatile intermediate for the synthesis of proline precursors 1b, 1e, and 1i. For example, 1a can generate an enolate (1 h) upon treatment with a base, such as lithium hexamethyldisylazide. Compound 1h can be alkylated with methyl iodide to afford Compound 1i. Alternatively, Compound 1a could be converted to epoxide 1f, which could subsequently be opened under standard conditions to afford Compound 1b. Moreover, Compound 1a could be alkylated to afford 1b. Furthermore, Compound 1a's ketone moiety could be removed to afford Compound 1c, which could then be epoxidized to afford Compound 1d which in turn can be alkylated to afford Compound 1e.

Another method for obtaining 4-methyl substitution on the proline precursor is described in Scheme 1B:

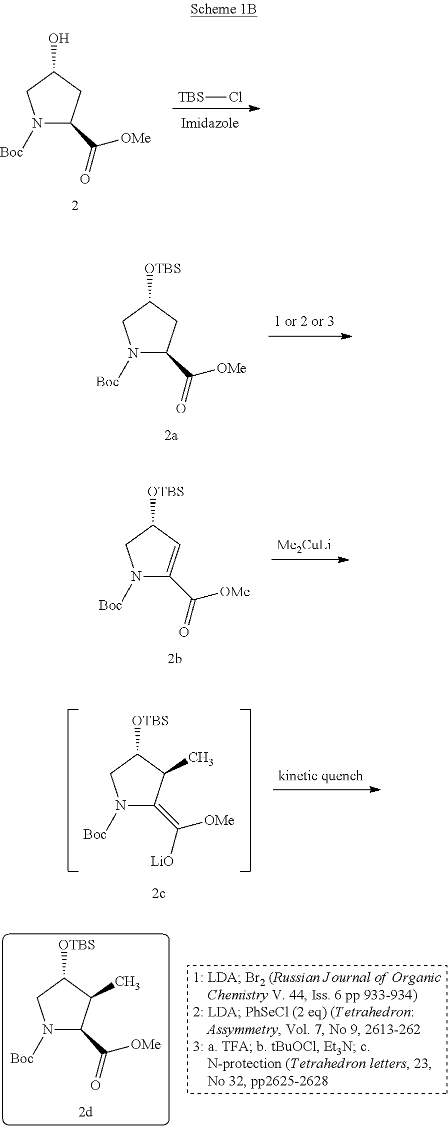

N-Boc-trans-4-hydroxy-L-proline methyl ester (1) can be protected, as a TBS silyl ether, for example using tert-butyldimethylsilyl chloride, and imidazole in an organic solvent such as DMF or methylene chloride to afford TBS silyl ether 2a. TBS silyl ether 2a can be converted to α,β-unsaturated ester 2b by methods known to those of skill in the art. For example, (1) treatment of TBS silyl ether 2a with LDA or lithium hexamethyldisilylazide followed by α-bromination of the resultant enolate at low temperature (e.g. −90° C.) using bromine, followed by dehydrohalogenation can provide α,β-unsaturated ester 2b (Kublitskii et al., “A New Method of Synthesis of Methyl N-Boc-2,3-dehydropyrrolidine- and piperidine-2-carboxylates,” Russian Journal of Organic Chemistry, 2008, 44(6): 933-934, incorporated herein in its entirety); (2) treatment of TBS silyl ether 2a with LDA or lithium hexamethyldisilylazide followed by α-selenation of the resultant enolate at 0° C. using phenylselenium chloride, followed by stirring at room temperature can provide α,β-unsaturated ester 2b (Ezquerra, et al., “4-Benzyl-2,3-didehydroprolinate as a Homochiral Template for Michael Additions. Synthesis of Enantiopure α-Allokainoids, β-Kainoids, 2,3-Methanoprolines and other 3,4-Disubstituted Prolines,” Tetrahedron Asymmetry, 1996, 7(9): 2613-2626, incorporated herein in its entirety); or (3) the Boc group of TBS silyl ether 2a can be removed under acidic conditions, for example by treatment with TFA, to afford a TBS silyl ether amino ester intermediate. The TBS silyl ether amino ester intermediate can then be exposed to N-chlorination with a chlorination agent, such as tert-butyl hypochlorite, followed by dehydrochlorination with a base, such as triethylamine in an one pot operation to afford an imine intermediate. The imine intermediate can be re-protected with a Boc protecting group and isomerized to provide α,β-unsaturated ester 2b or the imine can be isomerized to provide an α,β-unsaturated ester and then re-protected with a Boc protecting group providing α,β-unsaturated ester 2b (Hashimoto, et al., “Synthesis of New Acromelic Acid Congeners: Novel Neuroexcitatory Amino Acids Acting on Glutamate Receptor,” Tetrahedmt L.etters, 1991, 32(23): 2625-2628, incorporated herein in its entirety). Lithium dimethyl cuprate conjugate addition to α,β-unsaturated ester 2b followed by kinetic quench of resultant intermediate 2c can afford compound 2d.

Still other methods for obtaining 4-methyl substitution on the proline precursor is described in Scheme 1C:

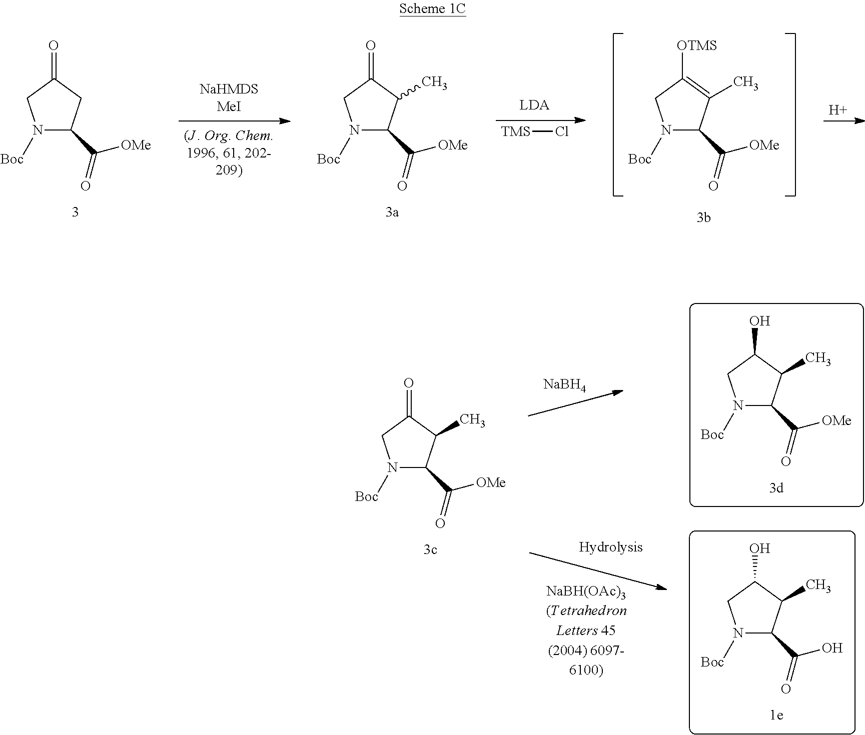

N-Boc-4-oxo-L-proline methyl ester (3) can be treated with a base, such as sodium hexamethyldisilylazide, followed by alkylation with a methylating agent, such as methyl iodide, to afford the α-methyl ketone 3a (Sharma, et al., “Regioselective Enolization and Alkylation of 4-Oxo-N-(9-phenylfluoren-9-yl)proline: Synthesis of Enantiopure Proline-Valine and Hydroxyproline-Valine Chimeras,” J. Org. Chem., 1996, 61(1): 202-209, incorporated herein in its entirety). The α-methyl ketone 3a can be treated with a base, such as LDA, and trimethyl silyl chloride to afford the silyl enol ether intermediate 3b. The silyl enol ether intermediate 3b can be treated with acid to provide the α-methyl β-keto ester 3c. The α-methyl β-keto ester 3c can be either reduced with a reducing agent, such as sodium borohydride, to afford compound 3d, or the ester of the α-methyl β-keto ester 3c can be hydrolyzed, for example, under basic conditions such as 1 N lithium hydroxide in dioxane, to afford a carboxylic acid intermediate. The carboxylic acid intermediate can be reduced with a reducing agent, such as sodium triacetoxyborohydride, to afford compound 1e (Liu, et al., “Carboxy mediated stereoselective reduction of ketones with sodium triacetoxyborohydride: synthesis of novel 3,4-fused tetrahydropyran and tetrahydrofuran prolines,” Tetrahedron Letters, 2004, 45: 6097-6100, incorporated herein in its entirety).

Further methods for obtaining 4-methyl substitution on the proline precursor is described in Scheme 1D:

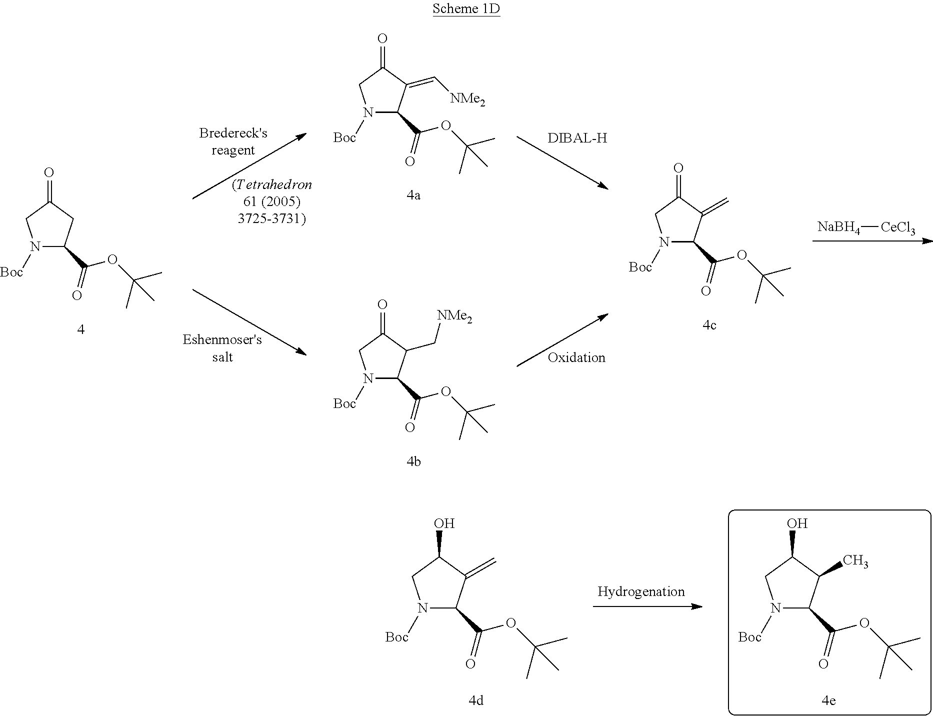

N-Boc-4-oxo-L-proline tert-butyl ester (4) can be treated with Bredereck's reagent (i.e. tert-butoxy bis(dimethylamino)methane) under appropriate conditions to afford the keto eneamine 4a (Chabaud, et al., “Stereoselective synthesis of (3S,4S)-tert-butyl-N-Boc-3-ethyl-4-hydroxy-L-prolinate and (3S,4R)-tert-butyl-N-Boc-3-ethyl-4-hydroxy-L-prolinate,” Tetrahedron, 2005, 61: 3725-3731, incorporated herein in its entirety). The keto eneamine 4a can be treated with a reducing agent, such as diisobutylaluminum hydride to afford the α,β-unsaturated ketone 4c. Alternatively, N-Boc-4-oxo-L-proline tert-butyl ester (4) can be treated with Eschenmoser's salt (i.e. N,N-Dimethylmethyleneiminium chloride) to afford the β-aminoketone 4b. The β-aminoketone 4b can be treated under oxidative conditions to afford α,β-unsaturated ketone 4c. The α,β-unsaturated ketone 4c can be reduced with a reducing agent, such as sodium borohydride in the presence of cerium chloride (e.g. Luche conditions), diisobutylaluminum hydride, lithium triethyl borohydride or lithium triethyl borohydride in the presence of cerium chloride to afford alcohol 4d. The alkene of alcohol 4d can be reduced, for example by hydrogenation, to afford compound 4e.

One method for obtaining a 4,4-dimethyl substituted proline precursor is described in Scheme 1E:

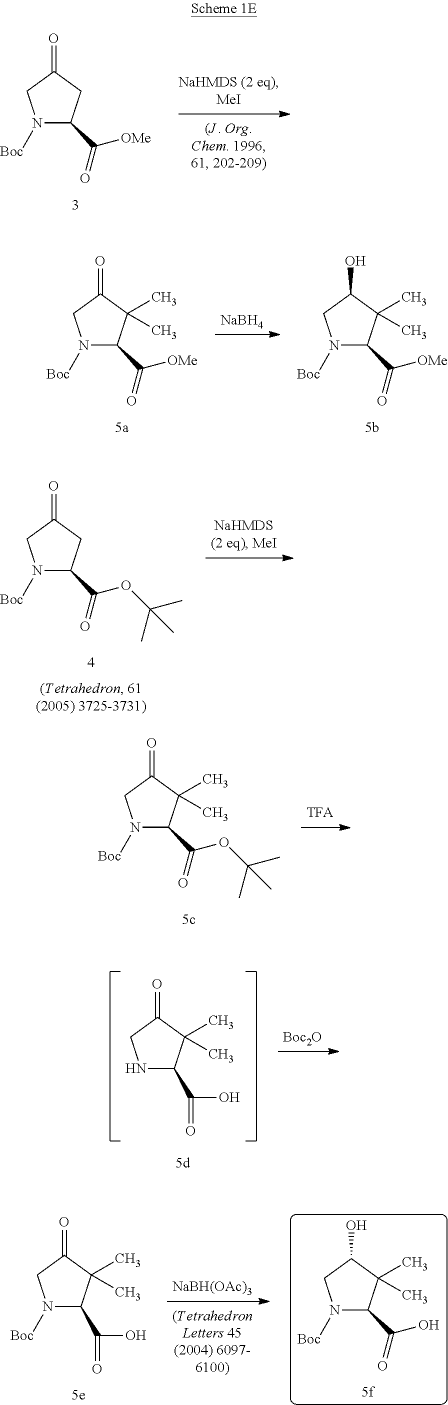

N-Boc-4-oxo-L-proline methyl ester (3) can be treated with a base, such as sodium hexamethyldisilylazide or potassium hexamethyldisilylazide, followed by alkylation with a methylating agent, such as methyl iodide, to afford N-Boc-3,3-dimethyl-4-oxo-L-proline methyl ester (5a) (Sharma, et al., “Regioselective Enolization and Alkylation of 4-Oxo-N-(9-phenylfluoren-9-yl)proline: Synthesis of Enantiopure Proline-Valine and Hydroxyproline-Valine Chimeras,” J. Org. Chem., 1996, 61(1): 202-209, incorporated herein in its entirety). N-Boc-3,3-dimethyl-4-oxo-L-proline methyl ester (5a) can be reduced with a reducing agent, such as sodium borohydride, to afford (4S)—N-Boc-3,3-dimethyl-4-hydroxy-L-proline methyl ester 5b. The epimeric alcohol can be accessed by treating N-Boc-4-oxo-L-proline tert-butyl ester (4) with a base, such as sodium hexamethyldisilylazide or potassium hexamethyldisilylazide, followed by alkylation with a methylating agent, such as methyl iodide, to afford the N-Boc gem dimethyl ketone tert-butyl ester 5c. Ester hydrolysis and Boc removal can be accomplished by treatment of N-Boc gem dimethyl ketone tert-butyl ester 5c with an acid, such as TFA to afford intermediate amino acid 5d. After removal of TFA, the amino acid 5d can be treated with Boc2O to afford N-Boc keto amino acid 5e. N-Boc keto amino acid 5e can be reduced with a reducing agent, such as sodium triacetoxyborohydride, to afford N-Boc amino acid alcohol 5f (Liu, et al., “Carboxy mediated stereoselective reduction of ketones with sodium triacetoxyborohydride: synthesis of novel 3,4-fused tetrahydropyran and tetrahydrofuran prolines,” Tetrahedron Letters, 2004, 45: 6097-6100, incorporated herein in its entirety).

Another method for obtaining 4-methyl substitution on the proline precursor is described in Scheme 1F:

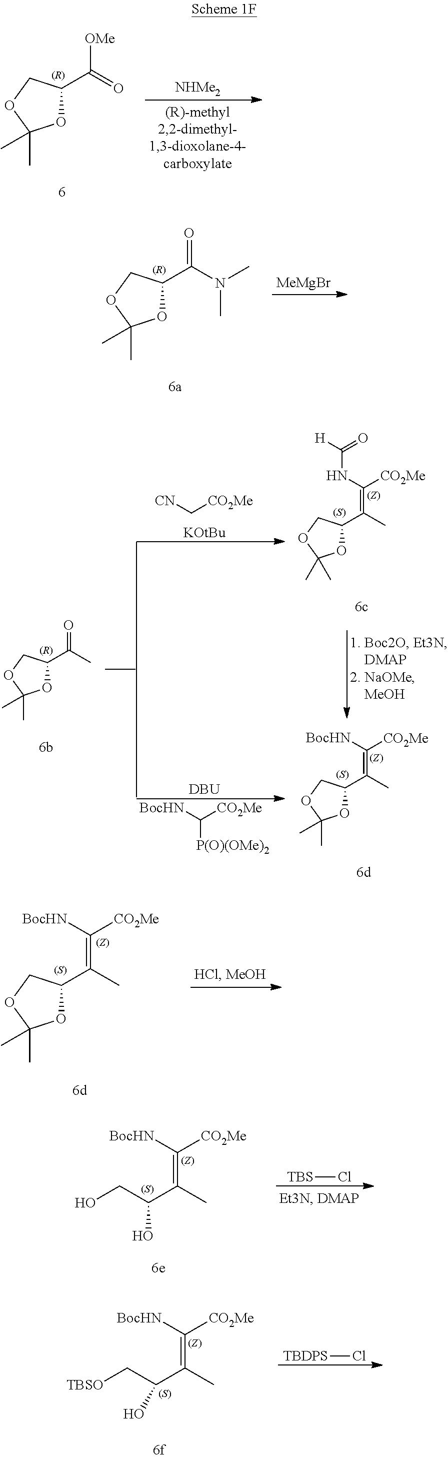

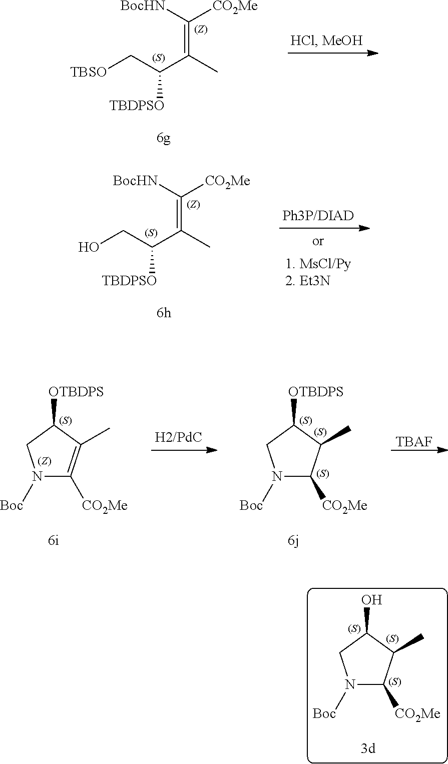

Methyl (R)-(+)-2,2-dimethyl-1,3-dioxolane-4-carboxylate (6) can be treated with dimethylamine to provide dimethylamide 6a. A solution of dimethylamide 6a in a solvent, such as THF, can be treated with methyl magnesium chloride at a temperature, such as 0° C. and then the reaction can be quenched, for example by pouring it rapidly into a vigorously-stirred saturated ammonium chloride solution to afford ketone 6a (U.S. Pat. No. 5,110,957, incorporated herein in its entirety; Example 6). Ketone 6a can be treated with methyl isocyanoacetate in the presence of a base, such as potassium tert-butoxide, to afford amide 6c. Amide 6c can be treated with Boc₂O and then reacted under basic conditions, for example sodium methoxide and methanol, to afford Boc-carbamate 6d. Alternatively, ketone 6a can be treated with Wittig-Horner reagent (±)-Boc-α-phosphonoglycine trimethyl ester in the presence of a base, such as DBU, to afford Boc-carbamate 6d. Boc-carbamate 6d can be reacted under acid conditions, for example HCl in methanol, to afford diol 6e. The primary alcohol of diol 6e can be protected as a silyl ether, for example by using tert-butyldimethylsilyl chloride in an organic solvent, such as DMF or methylene chloride, in the presence of a base, such as imidazole, triethyl amine, DMAP and combinations thereof, to afford TBS silyl ether 6f. The secondary alcohol of TBS silyl ether 6f can be protected as a silyl ether, for example by using tert-butyldiphenylsilyl chloride in an organic solvent, such as DMF or methylene chloride, in the presence of a base, such as imidazole, triethyl amine, DMAP and combinations thereof, to afford Bis silyl ether 6g. Removal of the TBS silyl ether of 6g can be accomplished under acid conditions, for example HCl in methanol, to provide TBDPS silyl ether 6h. TBDPS silyl ether 6h can be cyclized, for example using triphenylphosphine and diisopropyl azodicarboxylate (i.e. DIAD), diphenyl phosphoryl azide, TsCl in the presence of triethyl amine and pyridine, or MsCl in the presence of triethyl amine and pyridine, to afford compound 61. The alkene of compound 61 can be hydrogenated, for example by using formic acid, hydrazine, or H₂ over a catalyst, such as 10% Pd/C, in a solvent, such as methanol, to provide compound 6j. Removal of the TBDPS silyl ether of 6g can be accomplished under appropriate conditions, for example TBAF in THF or HF in pyridine, to afford methyl (2S,3S,4S)-1-Boc-4-hydroxy-3-methylprolinate (3d).

Still other methods for obtaining 4-methyl substitution on the proline precursor is described in Schemes 1G, 1G-1, 1G-2, 1G-3, and 1G-4:

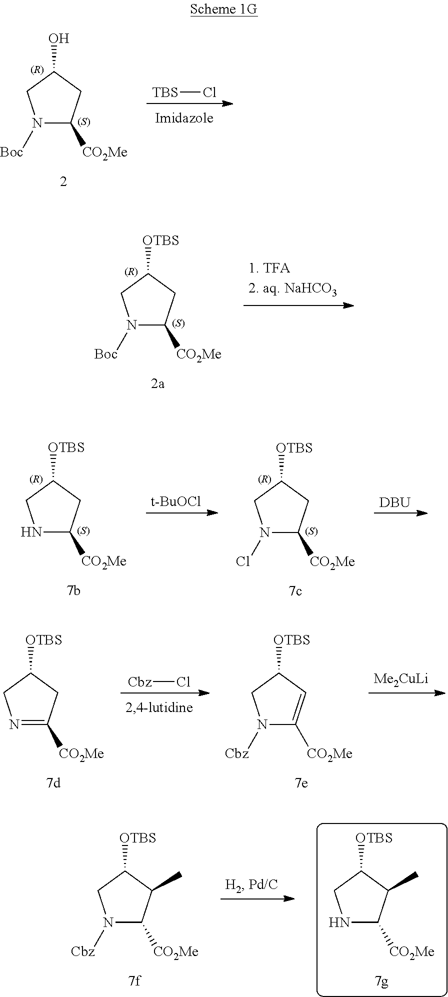

N-Boc-trans-4-hydroxy-L-proline methyl ester (1) hydroxy group can be protected as a silyl ether, for example by using tert-butyldimethylsilyl chloride, and imidazole in an organic solvent such as DMF or methylene chloride, to afford TBS silyl ether 2a. Removal of the Boc of 2a can be accomplished under acidic conditions, for example by using an acid, such as TFA, to afford amino ester 7b. Amino ester 7b can be treated with a chlorination agent, such as tert-butyl hypochlorite, to afford N-chloroamino ester 7c. N-Chloroamino ester 7c can be treated with a base, such as triethylamine or DBU, to afford imine 7d. Imine 7d can be protected as a Cbz carbamate, for example by using Cbz-Cl in the presence of 2,4-lutidine, to afford α,β-unsaturated ester 7e. Lithium dimethyl cuprate conjugate addition to α,β-unsaturated ester 7e can afford compound 7f. Removal of the Cbz group of compound 7f by hydrogenolysis, for example using formic acid, hydrazine, or H₂ over a catalyst, such as 10% Pd/C, in a solvent, such as methanol, can afford methyl (2R,3S,4R)-4-{[tert-butyl(dimethyl)silyl]oxy}-3-methylprolinate (7g).

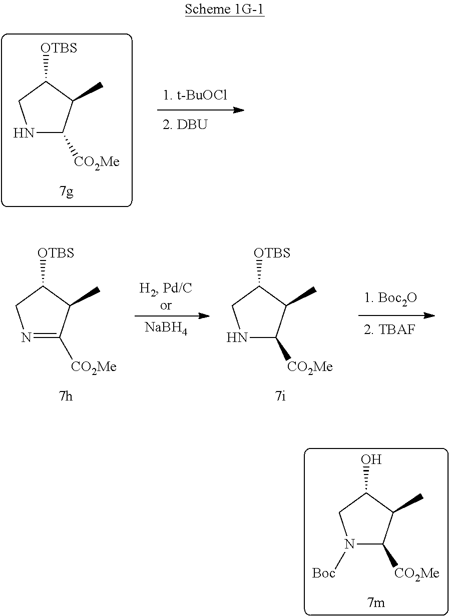

Methyl (2R,3S,4R)-4-{[tert-butyl(dimethyl)silyl]oxy}-3-methylprolinate (7g) can be treated with a chlorination agent, such as ten-butyl hypochlorite, followed by dehydrochlorination with a base, such as triethylamine or DBU, to afford imine 7h. Imine 7h can be reduced with a reducing agent, such as formic acid, hydrazine, or H₂ over a catalyst, such as 10% Pd/C, or using sodium borohydride, to afford amino ester 7i. Amino ester 7i can be protected as a Boc carbamate, for example by using Boc₂O, to afford N-Boc amino ester 7j. Removal of the TBS silyl ether of 7i can be accomplished under appropriate conditions, for example TBAF in TRF or HF in pyridine, to afford methyl (2S,3S,4R)-1-Boc-4-hydroxy-3-methylprolinate (7m).

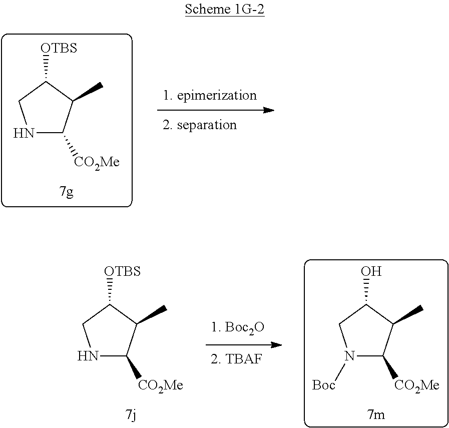

Methyl (2R,3S,4R)-4-{[tert-butyl(dimethyl)silyl]oxy}-3-methylprolinate (7g) can be epimerized at the 2-position, for example under acidic or basic conditions, to afford (after separation) methyl (2S,3S,4R)-4-{[tert-butyl(dimethyl)silyl]oxy}-3-methylprolinate (7j). Methyl (2S,3S,4R)-4-{[tert-butyl(dimethyl)silyl]oxy}-3-methylprolinate (7j) can be treated with Boc₂O to afford a Boc protected intermediate. The Boc protected intermediate can be treated under silyl deprotection conditions, such as TBAF in TRF or HF in pyridine to afford methyl (2S,3S,4R)-1-Boc-4-hydroxy-3-methylprolinate (7m).

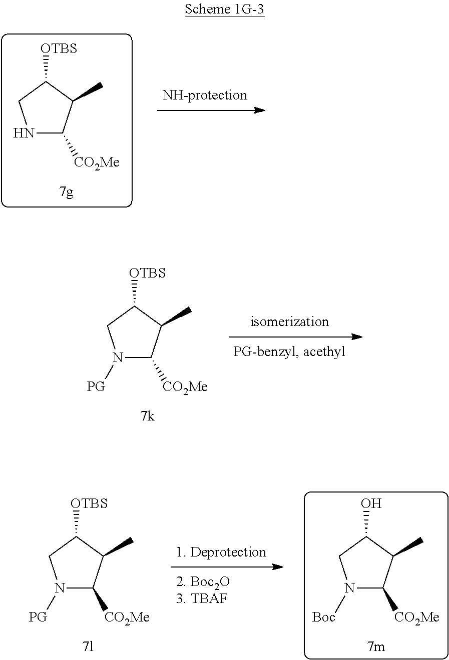

Methyl (2R,3S,4R)-4-{[tert-butyl(dimethyl)silyl]oxy}-3-methylprolinate (7g) can be treated with an acylating or benzylating agent, using methods known to those of skill in the art (Greene and Wuts, Protective Groups in Organic Synthesis; John Wiley and Sons: New York, 1999, incorporated herein in its entirety), to afford compound 7k. Compound 7k can be epimerized at the 2-position, for example, under acidic or basic conditions, to afford compound 7l. Compound 7l can be deprotected, using methods known to those of skill in the art (Greene and Wuts, Protective Groups in Organic Synthesis; John Wiley and Sons: New York, 1999, incorporated herein in its entirety) to afford methyl (2S,3S,4R)-4-{[tert-butyl(dimethyl)silyl]oxy}-3-methylprolinate. Methyl (2S,3S,4R)-4-{[tert-butyl(dimethyl)silyl]oxy}-3-methylprolinate can be protected as a Boc carbamate, for example by using Boc₂O, to afford methyl (2S,3S,4R)-1-Boc-4-{[tert-butyl(dimethyl)silyl]oxy}-3-methylprolinate. Methyl (2S,3S,4R)-1-Boc-4-{[tert-butyl(dimethyl)silyl]oxy}-3-methylprolinate can be treated with a silyl deprotection agent, such as TBAF or HF pyridine, to afford methyl (2S,3S,4R)-1-Boc-4-hydroxy-3-methylprolinate (7m).

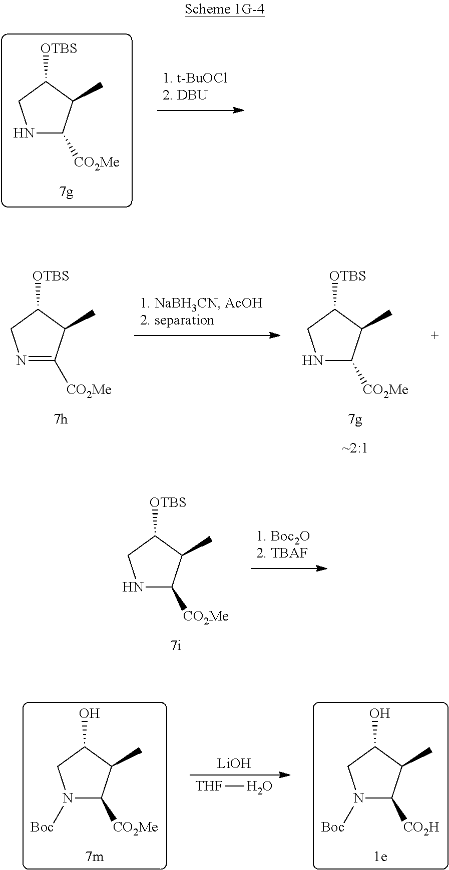

Methyl (2R,3S,4R)-4-{[tert-butyl(dimethyl) silyl]oxy}-3-methylprolinate (7g) can be treated with a chlorination agent, such as tert-butyl hypochlorite, followed by dehydrochlorination with a base, such as triethylamine or DBU, to afford imine 7h. Imine 7h can be reduced with a reducing agent, such as sodium cyanoborohydride in the presence of acetic acid, to afford a mixture of methyl (2R,3S,4R)-4-{[tert-butyl(dimethyl)silyl]oxy}-3-methylprolinate (7g) and methyl (2S,3S,4R)-4-{[tert-butyl(dimethyl)silyl]oxy}-3-methylprolinate (7i) which after separation affords an ˜2:1 ratio of compounds. Methyl (2S,3S,4R)-4-{[tert-butyl(dimethyl)silyl]oxy}-3-methylprolinate (7j) can be protected as a Boc carbamate, for example by using Boc₂O, to afford methyl (2S,3S,4R)-1-Boc-4-{[tert-butyl(dimethyl)silyl]oxy}-3-methylprolinate. Methyl (2S,3S,4R)-1-Boc-4-{[tert-butyl(dimethyl)silyl]oxy}-3-methylprolinate can be treated under silyl deprotection conditions, such as TBAF in THF or HF in pyridine, to afford methyl (2S,3S,4R)-1-Boc-4-hydroxy-3-methylprolinate (7m). Methyl (2S,3S,4R)-1-Boc-4-hydroxy-3-methylprolinate (7m) can be hydrolyzed, for example, under basic conditions, for example 1N lithium hydroxide in THF/water, to afford (2S,3S,4R)-1-Boc-4-hydroxy-3-methylproline (1e).

Still another method for obtaining 4-methyl substitution on the proline precursor is described in Scheme 3A.

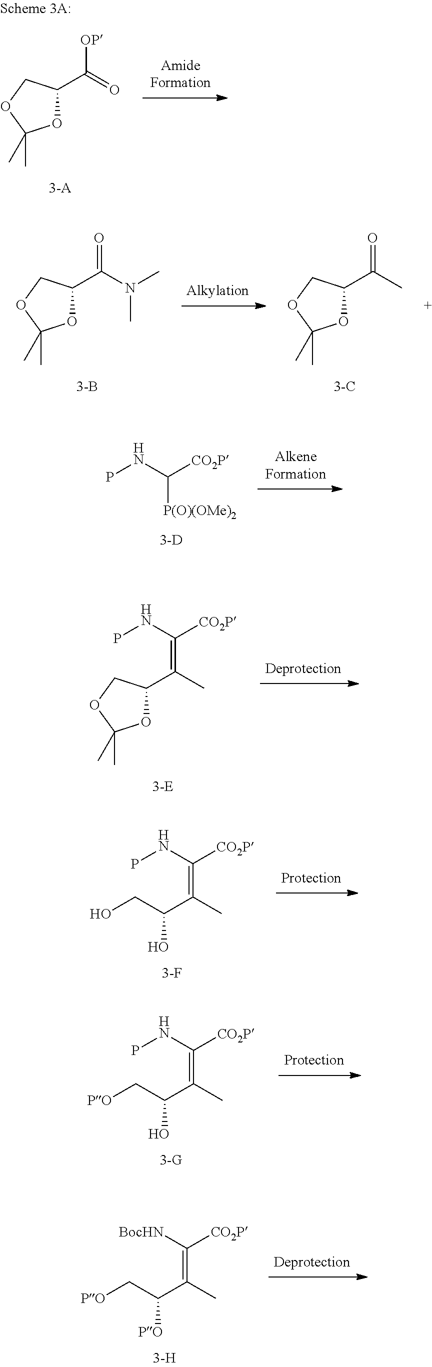

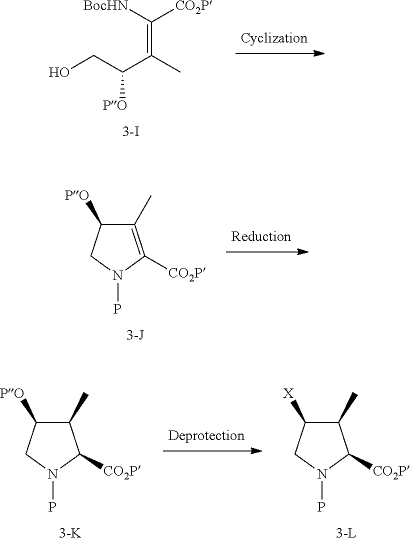

As indicated in Scheme 3A, advanced intermediate 3-L is readily afforded according to the generalized route. For example, treatment of protected Compound 3-A with dimethylamine will readily afford Compound 3-B. The amide carbonyl of 3-B is then readily alkylated to afford ketone 3-C. Suitable alkylating reagents for accomplishing such a transformation are well known in the art. As a non-limiting example, a suitable alkylating reagent is a methyl Grignard reagent. Treatment of Compound 3-C with Compound 3-D in the presence of a suitable base will readily afford alkene 3-E. While Scheme 3A illustrates the use of phosphonate 3-D under Horner-Wadsworth-Emmons reactions conditions, suitable variations of phosphonate 3-D are envisioned. Furthermore, Wittig-type reaction conditions for forming the alkene bond are also envisioned.

Deprotection of the ketal group in Compound 3-E will readily afford Compound 3-F. Such deprotection readily occurs under acidic conditions. As a non-limiting example, HCl may be used as the acid. However, many conditions are known to promote cleavage of ketal protecting groups and any suitable condition for effecting cleavage is contemplated.

Selective protection of the primary alcohol in Compound 3-F will readily afford Compound 3-G. Conditions for achieving selective protections are well known in the art. As a non-limiting example, treatment of Compound 3-F with t-butyldimethylsilyl chloride in the presence of a suitable base, such as triethylamine and DMAP will readily afford such a transformation. Protection of the secondary alcohol present in Compound 3-G will readily afford Compound 3-H. It is readily envisioned that any protecting group suitable for protecting an alcohol may be used. As a non-limiting example, a t-butyldiphenylsilyl protecting group may be used.

Selective deprotection of the primary alcohol in Compound 3-H will readily afford Compound 3-I. Conditions for achieving selective deprotection of a primary alcohol are well known in the art. As a non-limiting example, treatment of Compound 3-H with a suitable acid, such as HCl, will readily affect the desired transformation. Furthermore, it is contemplated that the desired compound, 3-L, may be prepared according to a modified Scheme 3A which omits the steps of protecting and deprotecting the primary and secondary alcohols present in Compounds 3-F through 3-K.

Cyclization of Compound 3-I to afford Compound 3-J can readily occur under any of several reaction conditions. For example, the primary alcohol in Compound 3-I can be converted into a suitable leaving group such that nucleophilic displacement by the protected amine in Compound 3-I will afford Compound 3-J. Alternatively, Mitsunobu conditions may be utilized to accomplish the desired transformation. As a non-limiting example, treatment of 3-I with DIAD in the presence of triphenylphosphine will readily afford Compound 3-J.

Compound 3-K is readily prepared upon the reduction of the double bond present in Compound 3-J. Conditions for reducing double bonds are well known in the art. As a non-limiting example, hydrogen in the presence of Pd/C will readily convert Compound 3-J into Compound 3-K. Deprotection of the alcohol in Compound 3-K will readily afford compound 3-L. Depending upon the protecting group, deprotecting may occur under many conditions. As a non-limiting example, when P″ is a t-butyldiphenylsilyl ether, deprotection may be accomplished upon exposing Compound 3-K to a fluoride source. In some embodiments, the fluoride source is TBAF. In Compound 3-L, X may be any suitable leaving group. As a non-limiting example, X may be a hydroxyl group. However, conditions for converting a hydroxyl group into other suitable leaving groups are well known in the art. In some embodiments, the hydroxyl group is converted into other suitable leaving groups.

Regarding the protecting group P′ in Scheme 3A, P′ represents a functional group that is suitable for protecting a carboxylic acid. In one embodiment, P′ is a functional group that protects the carboxylic acid as an ester. As a non-limiting example, a suitable protecting group for P′ is a methyl group. Regarding the protecting group P in Scheme 3A, P represents a functional group that is suitable for protecting an amine. In one embodiment, P is a functional group that protects the amine as a carbamate. As a non-limiting example, a suitable protecting group for P is a BOC group. Regarding the protecting group P″ in Scheme 3A, P″ represents a functional group that is suitable for protecting an alcohol. In one embodiment P″ is a functional group that protects the alcohol as an ether. In another embodiment, P″ is a silicon-containing ether protecting group. Moreover, in some embodiments, P″ is a t-butyldimethylsilyl protecting group or a t-butyldiphenylsilyl protecting group.

### Protecting Groups

In some circumstances, a chemical reaction may need to be performed selectively at one reactive site in a multifunctional compound. One such method that is useful for accomplishing such selectivity is to temporarily block one or more reactive sites in the multifunctional compound with a protective group. Such a method is often referred to as “protecting” the functional group. Many protecting groups are known in the art. See, e.g., Greene et al., Protective Groups in Organic Synthesis, Third Ed. (John Wiley & Sons, Inc. 1999), herein incorporated by reference in its entirety; Wutz et al., Greene's Protective Groups in Organic Synthesis, Fourth Ed. (John Wiley & Sons, Inc. 2007), herein incorporated by reference in its entirety. When more than one reactive site in a multifunctional compound requires protecting, or when a compound is prepared that will possess more than one protected functional group, it is important to use orthogonal protecting groups. Protecting groups are orthogonal if they are susceptible to selective removal.

In some embodiments, it may be necessary to protect one or more functional groups so as to prevent their interference in the desired reaction. For example, it may be necessary to protect one or more functional groups such as amines, carboxylic acids, and/or hydroxyl groups.

Suitable protecting groups for protecting amines include: carbamates such as alkyl carbamates including methyl, ethyl, propyl, isopropyl, butyl, tert-butyl, sec-butyl, pentyl, neopentyl, hexyl, heptyl, nonnyl, decanyl, and configurational isomers thereof; 9-flurenylmethyl; 9-(2-sulfo)flurenylmethyl; 9-(2,7-dibromo)fluorenylmethyl; 17-tetrabenzo[a,c,g,i]flurenylmethyl; 2-chloro-3-indenylmethyl; benz[f]inden-3-ylmethyl; 2,7-di-t-butyl[9-(10,10-dioxo-10,10,10,10-tetrahydrothioxanthyl)]methyl; 1,1-dioxobenzo[b]thiophene-2-ylmethyl; substituted ethyl carbamates such as 2,2,2-trichloroethyl; 2-trimethylsilylethyl; 2-phenylethyl; 1-(1-adamantyl)-1-methylethyl; 2-chloro ethyl; 1,1-dimethyl-2-halo ethyl; 1,1-dimethyl,2,2-dibromo ethyl; 1,1-dimethyl-2,2,2-trichloro ethyl; 1-methyl-1-(4-biphenylyl)ethyl; 1-(3,5-di-t-butylphenyl)-1-methylethyl; 2-(2′- and 4′-prydyl)ethyl; N-(2-pivaloylamino)-1,1-dimethylethyl; 2-[(2-nitrophenyl)dithio]-1-phenylethyl; 2-(N,N,-dicyclohexylcarboxamido)ethyl; t-butyl; 1-adamantyl; 2-adamantyl; vinyl; allyl; 1-isopropylallyl; cinnamyl; 4-nitro cinnamyl; 3-(3′-pyridyol)prop-2-enyl; 8-quinolyl; N-hydroxypiperidinyl; alkydithio; benzyl; p-methoxybenzyl; p-nitrobenzykl; p-bromobenzyl; p-chlorobenzyl; 2,4-dichlorobenzyl; 4-methylsulfinylbenzyl; 9-anthrylmethyl; diphenylmethyl; 2-methylthio ethyl; 2-methyl sulfonylethyl; 2-(p-toluene sulfonyl)ethyl; [2-(1,3-dithianyl)]methyl; 4-methylthiophenyl; 2,4-dimethylthiophenyl; 2-phosphonioethyl; 1-methyl-1-(triphenylphosphonio)ethyl; 1,1-dimethyl-2-cyanoethyl; 2-dansylethyl; 2-(4-nitrophenyl)ethyl; 4-phenylacetoxybenzyl; 4-azidobenzyl; 4-azidomethoxybenzyl; m-chloro-p-acyloxybenzyl; p-(dihydroxyboryl)benzyl; 5-benzisoxazolylmethyl; 2-(trifluoromethyl)-6-chromonylmethyl; m-nitrophenyl; 3,5-dimethoxybenzyl; 1-methyl-1-(3,5-dimethoxyphenyl)ethyl; α-methylnitropiperonyl; o-nitrobenzyl; 3,4-dimethoxy-6-nitrobenzyl; phenyl(o-nitrophenyl)methyl; 2-(2-nitrophenyl)ethyl; 6-nitroveratryl; 4-methoxyphenacyl; 3′,5′-dimethoxybenzoin; phenothiazinyl-(10)-carbonyl derivatives; N′-p-toluenesulfonylaminocarbonyl; N′-phenylaminothiocarbonyl; t-amyl; S-benzyl thiocarbamate; butynyl; p-cyanobenzyl; cyclobutyl; cyclohexyl; cyclopentyl; cyclopropylmethyl; p-dicyloxybenzyl; diisopropylmethyl; 2,2-dimethoxycarbonylvinyl; o-(N′,N′-dimethylcarboxamido)benzyl; 1,1-dimethyl-3-(N′,N′-dimethylcarboxamido)propyl; 1,1-dimethylpropynyl; di(2-pyridyl)methyl; 2-furanylmethyl; 2-iodoethyl; isobornyl; isobutyl; isonicotinyl; p-(p′-methoxyphenylazo)benzyl; 1-methylcyclobutyl; 1-methylcyclohexyl; 1-methyl-1-cyclopropylmethyl; 1-methyl-1-(p-phenylazophenyl)ethyl; 1-methyl-1-phenylethyl; 1-methyl-1-(4′-pyridyl)ethyl; phenyl; p-(phenylazo)benzyl; 2,4,6-tri-t-butylphenyl; 4-(trimethylammonium)benzyl; 2,4,6-trimethylbenzyl; and other similar carbamates; amides, including, but not limited to, formyl, acetyl, chloroacetyl, trichloroacetyl, trifluoroacetyl, phenylacetyl, propionyl, 3-phenylpropionyl, 4-pentenoyl, picolinoyl, 3-pyridylcarboxamide, benzoylphenylalanyl, benzoyl, p-phenylbenzoyl, amides whose cleavage is induced by nitro group reduction, such as o-nitrophenylacetyl, o-nitrophenoxyacetyl, 3-(o-nitrophenyl)propionyl, 2-methyl-2-(o-nitrophenoxy)propionyl, 3-methyl-3-nitrobutyryl, o-nitrocinnamoyl, o-nitrobenzoyl, and 3-(4-t-butyl-2,6-dinitrophenyl)-2,2-dimethylpropionyl; amides whose cleavage is induced by release of an alcohol, such as o-(benzoyloxymethyol)benzoyl, (2-acetoxymethyl)benzoyl, 2-[(t-butyldiphenylsiloxy)methyl]benzoyl, 3-(3′,6′-dioxo-2′,4′,5′-trimethylcyclohexa-1′,4′-diene-3,3-dimethylpropionyl, and o-hydroxy-trans-cinnamoyl; amides whose cleavage is induced by other chemical reactions, such as 2-methyl-2-(o-phenylazophenoxy)propionyl, 4-chlorobutyryl, acetoacetyl, 3-(p-hydroxyphenyl)propionyl, (N′-dithiobenzyloxycarbonylamino)acetyl, N-acetylmethionine, and 4,5-diphenyl-3-oxazolin-2-one; cyclic imide derivatives such as N-phthaloyl, N-tetrachlorophthaloyl, N-4-nitrophthaloyl, N-dithiasuccinoyl, N-2,3-diphenylmaleoyl, N-2,5-dimethylpyrrolyl, N-2,5-bis(triisopropylsiloxy)pyrrolyl, N-1,1,4,4,-tetramethyldisilylazacyclopentane adduct, N-1,1,3,3,-tetramethyl-1,3-disilaisoindolyl, 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridonyl, and 1,3,5-dioxazinyl; N-alkyl and N-aryl derivatives, such as N-methyl, N-t-butyl, N-allyl, N-[2-(trimethylsilyl)ethoxy]methyl, N-3-acetoxypropyl, N-cyanomethyl, N-(1-isopropyl-4-nitro-2-oxo-3-pyrrolin-3-yl), N-2,4-dimethoxybenzyl, N-2-azanorbornenyl, N-2,4-dinitrophenyl, quaternary ammonium salts, N-benzyl, N-4-methoxybenzyl, N-2,4-dimethoxybenzyl, N-2-hydroxybenzyl, N-diphenylmethyl, N-bis(4-methoxyphenyl)methyl, N-5-dibenzosuberyl, N-triphenylmethyl, N-(4-methoxyphenyl)duiphenylmethyl, N-9-phenylfluorenyl, N-ferrocenylmethyl, and N-2-picolylamine N′-oxide; imine derivatives, such as N-1,1-dimethylthiomethylene, N-benzylidene, N-p-methoxybenzylidine, N-diphenylmethylene, N-[(2-pyridyl)mesityl]methylene, N—(N′,N′-dimethylaminomethylene), N—(N′,N′-dibenzylaminomethylene), N—(N′-t-butylaminomethylene), N,N′-isopropylidene, N-p-nitrobenzylidene, N-salicylidene, N-5-chloro salicylidene, N-(5-chloro-2-hydroxyphenyl)phenylmethylene, N-cyclohexylidene, and N-t-butylidene; enamine derivatives, such as N-(5,5-dimethyl-3-oxo-1-cyclohexenyl), N-2,7-dichloro-9-fluorenylmethylene, N-2-(4,4-dimethyl-2,6-dioxocyclohexylidene)ethyl, N-4,4,4-trifluoro-3-oxo-1-butenyl, and N-1-isopropyl-4-nitro-2-oxo-3-pyrrolin-3-yl; and N-heteroatom derivatives such as N-metal, N-borane, N-diphenylborinic acid, N-diethylborinic acid, N-difloroborinic acid, N,N′-3,5-bis(trifluoromethyl)phenylboronic acid, N-[phenyl(pentacarbonylchromium-or-tungsten)]carbonyl, N-copper chelates, N-zinc chelates, and 18-crown-6 derivatives, N—N derivatives such as N-nitro, N-nitroso, N-oxide, and triazene derivatives, N—P derivatives such as N-diphenylphosphinyl, N-dimethylthiophosphinyl, N-diphenylthiophosphinyl, N-dialkylphosphoryl, N-dibenzylphosphoryl, N-diphenylphosphoryl, and iminotriphenylphosphorane derivatives, N—Si derivatives, N-sulfenyl derivatives such as N-benzenesulfonyl, N-o-nitrobenzenesulfenyl, N-2,4-dinitrobenzenesulfenyl, N-pentachlorobenzenesulfenyl, N-2-nitro-4-methoxybenzenesulfenyl, N-triphenylmethylsulfenyl, N-1-(2,2,2-trifluoro-1,1-diphenyl)ethylsulfenyl, and N-3-nitro-2-pyridine sulfenyl, and/or N-sulfonyl derivatives such as N-p-toluenesulfonyl, N-benzenesulfonyl, N-2,3,6-trimethyl-4-methoxybenzenesulfonyl, N-2,4,6-trimethoxybenzene sulfonyl, N-2,6-dimethyl-4-methoxybenzene sulfonyl, N-pentamethylbenzenesulfonyl, N-2,3,5,6-tetramethyl-4-methoxybenzenesulfonyl, N-4-methoxybenzenesulfonyl, N-2,4,6-trimethylbenzenesulfonyl, N-2,6-dimethoxy-4-methylbenzene sulfonyl, N-3-methoxy-4-t-butylbenzene sulfonyl, N-2,2,5,7,8-pentamethylchroman-6-sulfonyl, N-2-nitrobenzenesulfonyl, N-4-nitrobenzenesulfonyl, N-2,4-dinitrobenzene sulfonyl, N-benzothiazole-2-sulfonyl, N-methanesulfonyl, N-2-(trimethylsilyl)ethanesulfonyl, N-9-anthracenesulfonyl, N-4-(4′,8′-dimethoxynaphthylmethyl)benzenesulfonyl, N-benzylsulfonyl, N-trifluoromethylsulfonyl, N-phenacylsulfonyl, and N-t-butylsulfonyl.

Suitable protecting groups for carboxylic acids include: esters such as enzymatically cleavable esters including heptyl, 2-N-(morpholino)ethyl, choline, (methoxyethoxy)ethyl, methoxyethyl; alkyl esters such as methyl, ethyl, propyl, isopropyl, butyl, tert-butyl, sec-butyl, pentyl, neopentyl, hexyl, heptyl, nonnyl, decanyl, and configurational isomers thereof; substituted methyl esters such as 9-fluoroenylmethyl, methoxymethyl, methylthiomethyl, tetrahydropyranyl, teatrahydrofuranyl, methoxyethoxymethyl, 2-(trimethylsilyl)ethoxymethyl, benzyloxymethyl, pivaloyloxymethyl, phenylacetoxymethyl, triisopropylsilylmethyl, cyanomethyl, acetol, phencacyl, p-bromophenacyl, α-methylphenacyl, p-methoxyphenacyl, desyl, carboamidomethyl, p-azobenzenecarboxamidomethyl, N-phthalidimdomethyl; 2-substituted ethyl esters such as 2,2,2-trichloroethyl, 2-haloethyl, ω-chloroalkyl, 2-(trimethylsilyl)ethyl, 2-methylthioethyl, 1,3-dithianyl-2-methyl, 2-(p-nitrophenylsulfenyl)ethyl, 2-(p-toluenesulfonyl)ethyl, 2-(2′-pyridyl)ethyl, 2-(p-methoxyphenyl)ethyl, 2-(diphenylphosphino)ethyl, 1-methyl-1-phenylethyl, 2-(4-acetyl-2-nitrophenyl)ethyl, 2-cyanoethyl, 3-methyl-3-pentyl, dicyclopropylmethyl, 2,4-dimethyl-3-pentyl, cyclopentyl, cyclohexyl, allyl, methallyl, 2-methylbut-e-en-2-yl, 3-methylbut-2-(prenyl), 3-buten-1-yl, 4-(trimethylsilyl)-2-buten-1-yl, cinnamyl, α-methylcinnamyl, prop-2-ynyl, phenyl; 2,6-dialkylphenyl esters such as 2,6-dimethylphenyl, 2,6-diisopropylphenyl, 2,6-di-t-butyl-4-methylphenyl, 2,6-di-t-butyl-4-methoxyphenyl, p-(methylthio)phenyl, pentafluorophenyl, benzyl; substituted benzyl esters such as triphenylmethyl, diphenylmethyl, bis(o-mitrophenyl)methyl, 9-anthrylmethyl, 2-(9,10-dioxo)anthrylmethyl, 5-dibenzosuberyl, 1-pyreneylmethyl, 2-(trifluoromethyl)-6-chromonylmethyl, 2,4,6-trimethylbenzyl, p-bromobenzyl, o-nitrobenzyl, p-nitrobenzyl, p-methoxybenzyl, 2,6-dimethoxybenzyl, 4-(methylsulfinyl)benzyl, 4-sulfobenzyl, 4-azidomethoxybenzyl, 4-{N-[1-(4,4-dimethyl-2,6-dioxocyclohexylidene)-3-methylbutyl]amino}benzyl, piperonyl, 4-picolyl, polymer supported p-benzyl; silyl esters such as trimethylsilyl, triethylsilyl, t-butyldimethylsilyl, i-propyldimethylsilyl, phenyldimethylsilyl, di-t-butylmethylsilyl, triisopropylsilyl; activated esters such as thiol esters; oxazoles; 2-alkyl-1,3-axazoline; 4-alkyl-5-oxo-1,3-oxazolidine; 2,2-bistrifluoromethyl-4-alkyl-5-oxo-1,3-oxazolidine; 5-alkyl-4-oxo-1,3-dioxolane; dioxanones; ortho esters; pentaminocobalt(III) complexes; and stannyl esters such as triethylstannyl and tri-n-butylstannyl; amides such as N,N-dimethyl, pyrrolidinyl, piperidinyl, 5,6-dihydrophenanthridinyl, o-nitroanilide, N-7-nitroindolyl, N-8-nitro-1,2,3,4-tetrahydroquinolyl, 2-(2-aminophenyl)acetaldehyde dimethyl acetal amide, and polymer supported p-benzenesulfonamide; hydrazides such as N-phenyl, N,N′diisopropyl; and tetraalkylammonium salts such as methyl, ethyl, propyl, isopropyl, butyl, tert-butyl, sec-butyl, pentyl, neopentyl, hexyl, heptyl, nonnyl, decanyl, and configurational isomers thereof.

Suitable protecting groups for hydroxyl groups include: silyl ethers such as trimethylsilyl, triethylsilyl, triisopropylsilyl, dimethylisopropylsilyl, diethylisopropylsilyl, dimethylthexylsilyl, 2-norbornyldimethylsilyl, t-butyl-dimethylsilyl, t-butyldiphenylsilyl, tribenzylsilyl, tri-p-xylylsilyl, triphenylsilyl, diphenylmethylsilyl, di-t-butylmethylsilyl, bis(t-butyl)-1-pyrenylmethoxysilyl, tris(trimethylsilyl)silyl:sisyl; (2-hydroxystyryl)dimethylsilyl; (2-hydroxystyryl)diisopropylsilyl, t-butylmethoxyphenylsilyl, t-butoxydiphenylsilyl, 1,1,3,3-tetraisopropyl-3-[2-(triphenylmethoxy)ethoxy]disiloxane-1-yl, fluorous silyl; C₁₋₁₀alkyl ethers such as methyl, ethyl, propyl, isopropyl, butyl, tert-butyl, sec-butyl, pentyl, neopentyl, hexyl, heptyl, nonnyl, decanyl, and configurational isomers thereof; substituted methyl ethers such as methoxymethyl, methylthiomethyl, (phenyldimethylsilyl)methoxymethyl, benzyloxymethyl, p-methoxybenzyloxymethyl, [(3,4-dimethoxybenzypoxy]methyl, p-nitrobenzyloxymethyl, o-nitrobenzyloxymethyl, [(R)-1-(2-nitrophenyl)ethoxy]methyl, (4-methoxyphenoxy)methyl, guaiacolmethyl, t-butoxymethyl, 4-pentenyloxymethyl, siloxymethyl, 2-methoxyethoxymethyl, 2-cyanoethoxymethyl, 2,2,2-trichloroethoxymethyl, bis(2-chloroethoxy)methyl, 2-(trimethylsilyl)ethoxymethyl, methoxymethyl, O-Bis(2-acetoxyethoxy)methyl, tetrahydropyranyl, fluorous tetrahydropyranyl, 3-bromotetrahydropyranyl, tetrahydrothiopyranyl, 1-methoxycyclohexyl, 4-methoxytetrahydropyranyl, 4-methoxytetrahydrothiopyranyl, 4-methoxytetrahydrothiopyranyl S,S-dioxide, 1-[(2-chloro-4-methyl)phenyl]-4-methoxypiperidin-4-yl, 1-(2-fluorophenyl)-4-methoxypiperidin-4-yl, 1-(4-chlorophenyl)-4-methoxypiperidin-4-yl, 1,4-dioxan-2-yl, tetrahydrofuranyl, tetrahydrothiofuranyl, and 2,3,2a,4,5,6,7,7a-octahydro-7,8,8-trimethyl-4,7-methanobenzofuran-2-yl; substituted ethyl ethers such as 1-ethoxyethyl, 1-(2-chloroethoxy)ethyl, 2-hydroxyethyl, 2-bromoethyl, 1-[2-(trimethylsilyl)ethoxy]ethyl, 1-methyl-1-methoxyethyl, 1-methyl-1benzyloxyethyl, 1-methyl-1-benzyloxy-2-fluoro ethyl, 1-methyl-1-phenoxyethyl, 2,2,2-trichloro ethyl, 1,1-dianisyl-2,2,2-trichloro ethyl, 1,1,1,3,3,3-hexafluoro-2-phenylisopropyl, 1-(2-cyanoethoxy)ethyl, 2-trimethylsilylethyl, 2-(benzylthio)ethyl, 2-(phenylselenyl)ethyl, t-butyl, allyl, prennyl, cinnamyl, 2-phenallyl, propargy, p-chlorophenyl, p-methoxyphenyl, p-nitrophenyl, 2,4-dinitrophenyl, 2,3,5,6-tetrafluoro-4-(trifluoromethyl)phenyl; benzyl; substituted benzyl ethers such as p-methoxybenzyl, 3,4-dimethoxybenzyl, o-nitrobenzyl, p-nitrobenzyl, pentadienylnitrobenzyl, pentadienylnitropiperonyl, halobenzyl, 2,6-dichlorobenzyl, 2,4-dichlorobenzyl, p-cyanobenzyl, p-phenylbenzyl, 2,6-difluorobenzyl, fluorous benzyl, 4-fluorousalkoxybenzyl, trimethylsilylxylyl, 2-phenyl-2-propyl (Cumyl), p-acylaminobenzyl, p-azidobenzyl, 4-azido-3-chlorobenzyl, 2-trifluoromethylbenzyl, 4-trifluoromethylbenzyl, p-(methylsulfinyl)benzyl, p-siletanylbenzyl, 4-acetoxybenzyl, 4-(2-trimethylsilyl)ethoxymethoxybenzyl, 2-napthylmethyl, 2-picolyl, 4-picolyl, 3-methyl-2-picolyl N-oxido, 2-quinolinylmethyl, 6-methoxy-2-(4-methylphenyl)-4-quinolinemethyl, 1-pyrenylmethyl, diphenylmethyl, 4-methoxydiphenylmethyl, 4-phenyldiphenylmethyl, p,p′-dinitrobenzhydryl, 5-dibenzosuberyl, triphenylmethyl, α-napthyldiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-dimethoxy-3″-[N-imidazolylmethyl)]trityl, 4,4′-dimethoxy-3″-[N-imidazolylethyl)carbamoyl]trityl, 1,1-bis(4-methoxyphenyl)-1′-pyrenylmethyl, 4-(17-tetrabenzo[a,c,g,i]fluorenylmethyl)-4,4″-dimethoxytrityl, 9-anthryl, 9-(9-phenyl)xanthenyl, 9-phenylthioxanthyl, 9-(9-phenyl-10-oxo)anthryl, 1,3-benzodithiolan-2-yl, 4,5-bis(ethoxycarbonyl)-[1,3]-dioxolan-2-yl, benzisothiazolyl S,S-dioxido; C₁₋₁₀alkyl esters such as formyl, acetyl, propionyl, isopropionyl, butyryl, tert-butyryl, sec-butyryl, pentanoyl, neopentanoyl, hexanoyl, heptanoyl, nonanoyl, decanoyl, and configurational isomers thereof, esters such as benzoylformate, chloroacetate, dichloroacetate, trichloroacetate, trifluoroacetate, methoxyacetate, triphenylmethoxyacetate, phenoxyacetate, p-chlorophenoxyacetate, phenylacetate, polymer supported p-phenylacetate, diphenylacetate, bisfluorous chain type propanoyl, nicotinate, 3-phenylpropionate, 4-pentenoate, 4-oxopentanoate, 4,4-(ethylenedithio)pentanoate, 5-[3-bis(4-methoxyphenyl)hydroxymethylphenoxy]levulinate, pivaloate, 1-adamantoate, crotonate, 4-methoxycrotonate, benzoate, p-phenylbenzoate, 2,4,6-trimethylbenzoate, picolinate, nicotinate, 4-bromobenzoate, 2,5-difluorobenzoate, p-nitrobenzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, 2-methyl-2-butenoate, (E)-2-methyl-2-butenoate, (Z)-2-methyl-2-butenoate, o-(methoxycarbonyl)benzoate, polymer supported p-benzoate, α-naphthoate, nitrate, alkyl N,N,N′,N′-tetramethylphosphorodiamidate, 2-chlorobenzoate, 3′,5′-dimethoxybenzoin, N-phenylcarbamate, borate, dimethylphosphinothioyl, 2,4-dinitrophenylsulfenate, and photolabile esters; carbonates, including methyl, methoxymethyl, 9-fluorenylmethyl, ethyl, 2,2,2-trichloroethyl, 1,1-dimethyl-2,2,2-trichloro ethyl, 2-(trimethylsilyl)ethyl, 2-(triphenylsulfonyl)ethyl, 2-(triphenylphosphonia)ethyl, isobutyl, vinyl, allyl, p-nitrophenyl, benzyl, p-methoxybenzyl, 3,3-dimethoxybenzyl, o-nitrobenzyl, p-nitrobenzyl, and silyl esters; carbonates cleaved by β-elimination such as 2-dansylethyl, 2-(4-nitrophenyl)ethyl, 2-(2,4-dinitrophenyl)ethyl, 2-cyano-1-phenylethyl, S-benzyl thiocarbonate, 4-ethoxy-1-mapthyl, and methyl dithiocarbonate, carbonates cleaved with assisted cleavage such as 2-iodobenzoate, 4-azidobutyrate, 4-nitro-4-methylpentanoate, o-(dibromomethyl)benzoate, 2-formylbenzenesulfonate, 2-(methylthiomethyoxy)ethyl, 4-(methylthiomethoxymethyl)butyrate, 2-(methylthiomethoxymethyl)benzoate, 2-(chloroacetoxymethyl)benzoate, 2-[(2-chloroacetoxy)ethyl]benzoate, 2-[2-(benzyloxy)ethyl]benzoate, 2-[2-(4-methoxybenzyloxy)ethyl]benzoate; and sulfonates such as sulfate, allylsulfate, C₁₋₁₀alkyl sulfonates such as methyl, ethyl, propyl, isopropyl, butyl, tert-butyl, sec-butyl, pentyl, neopentyl, hexyl, heptyl, nonnyl, decanyl, and configurational isomers thereof, benzylsulfonate, tosylate, and 2-[(4-nitrophenyl)ethyl]sulfonate.

### Protection and Deprotection Reactions:

Reagents, solvents, and reaction conditions useful for protecting amines, carboxylic acids, and alcohols are well-known in the art. Likewise, reagents, solvents, and reaction conditions useful for deprotecting amines, carboxylic acids, and alcohols are well known in the art. See, e.g., Greene et al., Protective Groups in Organic Synthesis, Third Ed. (John Wiley & Sons, Inc. 1999), herein incorporated by reference in its entirety; Wutz et al., Greene's Protective Groups in Organic Synthesis, Fourth Ed. (John Wiley & Sons, Inc. 2007), herein incorporated by reference in its entirety. While references have been made to specific reagents, solvents, and reaction conditions in the schemes described above, it is readily envisioned that equivalent reagents, solvents, and reaction conditions may be utilized to protect and deprotect amines, carboxylic acids, and alcohols.

### Coupling Reactions

In some circumstances, embodiments of the invention may require coupling reactions, such as the formation of a single bond between a nitrogen atom and a carbonyl's carbon atom. Such single bond formation may result in the preparation of an amide, or alternatively, a carbamate. Coupling reactions generally require the activation of a carbonyl group's carbon atom. Activation may take one of many forms that are known in the art. For example, a carbonyl group may be activated by forming symmetrical anhydrides. Alternatively, a carbonyl group may be activated by forming unsymmetrical anhydrides. In some circumstances, a carbonyl group may be activated as the corresponding carbonyl-halide, wherein the halide is fluorine, chlorine, bromine, or iodine.

Several types of “activating reagents” useful for activating the carbonyl carbon of a carboxylic acid are known in the art. Any of which may be considered an equivalent of any other reagent. One such type of reagent is carbodiimides, including but not limited to dicyclohexylcarbodiimide, diisopropylcarbodiimide, and 1-ethyl-3-(3-dimethylaminopropyl) carbodiimide. Other useful types of reagents include triazoles, including but not limited to HOBt, HOAt, and ethyl 2-cyano-2-(hydroxyimino)acetate, uranium salts such as TBTU, HATU, HBTU, HCTU, TOTU, COMU, phosphonium salts such as PyBOP, PyBrOP, BOP-Cl, and benzotriazole-1-yl-oxy-tris-(dimethylamino)-phosphonium hexafluorophosphate, and pentafluorophenyl esters such as FDPP and PFPOH. Moreover, coupling agents such as chloroformates (e.g. isopropyl chloroformate), acid chlorides (e.g. Pivaloyl chloride), phosphene equivalents (e.g. CDI), and N-hydroxysuccinimide may be used. In some embodiments, combinations of the above mentioned reagents may be utilized. While references have been made to specific reagents, it is readily envisioned that equivalent reagents may be utilized to promote coupling reactions.

Coupling reactions may be performed with all reagents in solution, such as for “liquid-phase” syntheses. Alternatively, coupling reactions may be performed with one or more reagents immobilized on a solid support, such as for “solid-phase” syntheses. Moreover, in some embodiments, combinations of liquid-phase and solid-phase synthesis may be utilized. When necessary, embodiments may also be microwave- and/or sonication-assisted.

### Mitsunobu Conditions

Mitsunobu conditions are reaction conditions that convert a hydroxyl into a variety of functional groups, such as a heteroaryl ether, with inversion of stereochemistry. Such reaction conditions are well known in the art and several reaction conditions collectively represent Mitsunobu conditions. Mitsunobu conditions typically utilize triphenylphosphine and DEAD to activate the hydroxyl group for nucleophilic displacement. However, other reagents are useful as well. See Kumara Swamy, K. C.; Bhuvan Kumar, N. N.; Balaraman, E.; Pavan Kumar, K. V. P., “Mitsunobu and Related Reactions: Advances and Applications” Chem. Rev. 2009, 109, 2551-2651, and references therein which are herein incorporated by reference. These reagents include polymer supported triphenylphosphine, phosphorane ylides such as (cyanomethylene)trimethylphosphorane and (cyanomethylene)tributylphosphorane, DIAD, di-t-butylazodicarboxylate, Di-p-chlorobenzyl azodicarboxylate (DCAD), and 1,1′-(azodicarbonyl)dipiperidine (ADDP)

### Oxidation and Elimination Reactions:

In the schemes above, N—H containing compounds may be oxidized to N—X containing compounds, where X is a leaving group. Such oxidations are well known in the art. When X is a halogen, such reactions are often referred to as N-halogenation or N-halo-de-hydrogenation. It is well known that treatment with hypohalites, including sodium hypochlorite and hypobromite, converts primary amines into N-haloamines. Secondary amines can be converted to N-halo secondary amines. N-halogenation is also accomplished with other reagents, e.g. sodium bromite, benzyltrimethylammonium tribromide, NCS, and NBS. Moreover, N-halogenation readily occurs in the presence of hypohalites. Hypohalites may be fluorine, chlorine, bromine, or iodine containing. Hypohalites may also be metal salts, such as alkali and/or alkaline earth metal hypohalites such as sodium hypochlorite. Moreover, hypohalites may be organic-containing, including but not limited to C₁₋₁₀alkylhypohalites like t-butylhypochlorite. N-fluorination can also be accomplished by direct treatment of amines with F₂. Treatment of an N—X containing compound with a suitable base will lead to the elimination of HX from a molecule and result in the formation of a Schiff base or imine.

In other embodiments, it is envisioned that X may be a hydroxyl group and/or a hydroxygroup converted into a more active leaving group, such as an ether, ester, and/or sulfonate ester. Treatment with a suitable base will lead to the elimination of HX from a molecule and result in the formation of a Schiff base or imine.

Oxidation of amines to produce N-oxides or N-hydroxides is known in the art. Such oxidations readily occur upon exposure of amines to peroxides and peroxyacides. Suitable oxidants include, but are not limited to, hydrogen peroxide, alkyl peroxides, cycloalkyl peroxides, alkyl peracids, cycloalkyl peracids, and aryl peracids like mCPBA.

### Reduction Reactions:

In the schemes above, Schiff bases or imine motifs may need to be reduced. Such reductions require the equivalent of the addition of hydrogen gas across the double bond of the Schiff base. Several known reagents may be utilized to accomplish the desired transformation with varying degrees of success. These reagents include, but are not limited to lithium aluminum hydride, diisobutylaluminum hydride, sodium borohydride, NaBH₃CN, NaBH(OAc)₃, Na-EtOH, hydrogen and a catalyst, Bu₂SnClH in HMPA, and other reducing agents as described in Harada, K. in Patai The Chemistry of the Carbon-Nitrogen Double Bond, Ref 40, p. 276; and Rylander, P. N. Catalytic Hydrogenation over Platinum Metals, Ref 165, p. 123, both of which are incorporated herein by reference

### Suitable Acids and Bases:

Suitable acids include Lewis acids, such as any species with a vacant orbital. Suitable acids also include Bronstead acids, which are proton donors. Acids may be used in anhydrous or aqueous solutions. Acids may be organic acids, such as those acids which contain a —COOH group, such as C₁₋₁₀alkyl carboxylic acids optionally substituted with halogens, aryl carboxylic acids optionally substituted with halogens, and sulfonic acids, such as C₁₋₁₀alkyl sulfonic acids and aryl sulfonic acids. Acids may also include mineral acids, such as sulfuric and sulfurous acids, including fuming variants, halogen containing acids such as hydrofluoric, hydrochloric, hydrobromic, and hydroiodic acids, phosphoric and phosphorous acids, including polyphosphoric acid, and nitric and nitrous acids, including fuming variants. Combinations of any of the above described acids may optionally be used.

Suitable bases include Lewis bases. Suitable bases also include Bronstead bases. Bases may be used in anhydrous or aqueous solutions. Bases may consist of carbonates such as metal carbonates and C₀₋₁₀alkylammonium carbonates. Bases may also consist of bicarbonates such as metal bicarbonates and C₀₋₁₀alkylammonium bicarbonates. Bases may also consist of hydroxides, such as metal hydroxides and/or C₀₋₁₀alkylammonium hydroxides.

Moreover, bases may be organic, such as amines and nitrogen-containing heterocyclic compounds. Suitable organic bases include primary, secondary, and tertiary amines and nitrogen-containing heterocycles. Examples of organic bases include, but are not limited to, alkylamines, dialkylamines, and trialkylamines, metal salts of hexamethyldisilazides, such as NaHMDS, KHMDS, and LiHMDS, and metal salts of alkylamines, dialkylamines, and trialkylamines such as LDA. Organic bases may also be nitrogen containing heterocycles. Examples of nitrogen-containing heterocycles include, but are not limited to, aziridine, azirine, diazirine, azetidine, azete, diazetidine, azolidine, pyrrole, imidazolidine, pyrazolidine, imidazole, imidazoline, pyrazole, pyrazoline, oxazolidine, isoxazolidine, oxazole, oxazoline, isoxazole, isoxazoline, thiazolidine, isothiazolidine, thiazole, thiazoline, isothiazole, isothiazoline, triazole, dithiazole, furazan, oxadiazole, thiadiazole, tetrazole, pyridine, DMAP, piperazine, diazine, morpholine, oxazine, thiazine, triazine, tetrazine, azepane, azepine, diazepine, azocane, azocine, and bicyclic heterocycles such as DBU.

While references have been made to specific acids and bases in the schemes described above, it is readily envisioned that equivalent reagents, solvents, and reaction conditions may be utilized to facilitate the desired reaction.

### Leaving Groups

A leaving group is a molecular fragment that departs with a pair of electrons in heterolytic bond cleavage. Many leaving groups are known in the art, and they can be anions or neutral molecules. Common anionic leaving groups are halides anions such as iodide, bromide, chloride, and fluoride; sulfonate esters such as C₁₋₁₀alkyl sulfonates such as methyl, ethyl, propyl, isopropyl, butyl, tert-butyl, sec-butyl, pentyl, neopentyl, hexyl, heptyl, nonnyl, decanyl, and configurational isomers thereof, benzylsulfonate, arylsulfonates such as phenylsulfonate or optionally substituted arylsulfonates such as tosylate. Common neutral molecule leaving groups are water, ammonia, triC₁₋₁₀alkyl substituted ammonia such as trimethyl, triethyl, tripropyl, triisopropyl, tributyl, tri-t-butyl, tri-sec-butyl, tripentyl, trineopentyl, trihexyl, triheptyl, trinonnyl, tridecanyl, and configurational isomers thereof, and alcohols such as methyl, ethyl, propyl, isopropyl, butyl, tert-butyl, sec-butyl, pentyl, neopentyl, hexyl, heptyl, nonnyl, decanyl, and configurational isomers thereof, aryl alcohols such as phenol, optionally substituted aryl alcohols, benzyl alcohol, and optionally substituted benzyl alcohols. Additional leaving groups known in the art include diazonium salts, oxonium ions, nonaflates, triflates, fluorosulfonates, nitrates, phosphates, thionium ions, esters, acid anhydrides, and phenoxides.

It is also known that alcohols may be readily converted into the above mentioned leaving groups. Many known methods will retain any stereochemistry of the molecule, while other known methods will invert any stereochemistry at the reactive site carbon. Furthermore, alcohols may activated using triakylphosphines, such as triphenylphosphine, to generate an oxyphosphonium intermediate. Oxyphosphonium intermediates are generally regarded as good leaving groups. One method for generating such a leaving group is to utilize a trialkylphosphine, such as triphenylphospine, in combination with a dialkyl azodicarboxylate, such as DEAD. Alternatively, an oxyphosphonium leaving group may be generated utilizing a trialkylphosphine, such as triphenylphospine, in combination with a tetrahalomethane, such as carbon tetrachloride, carbon tetrabromide, and/or carbon tetraiodide.

### Intermediates

As described above, once obtained, substituted proline precursors can readily be used to make a wide variety of macrocyclic and non-macrocyclic HCV inhibitors. Accordingly, some embodiments include the substituted proline precursors or intermediates used in preparing such precursors. Some embodiments of such compounds include Compound C-A:

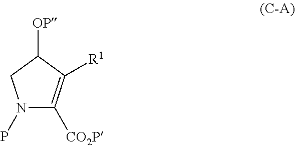

or a salt there of where P is hydrogen or a protecting group for an amine; P′ is hydrogen or a protecting group for a carboxylic acid; P″ is hydrogen or a protecting group for an alcohol; and R¹ is hydrogen or an alkyl group. It will be readily apparent that Compound C-A encompasses intermediate 7e-P described above in Scheme 1. Compound C-A also encompasses intermediate 2b described above in Scheme 1B. Compound C-A also encompasses intermediate 61 described above in Scheme 1-F. Compound C-A also encompasses intermediate 7e described above in Scheme 1G.

In various embodiments, C-A is selected from:

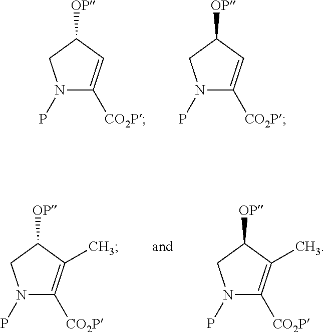

Some embodiments include Compound CI-A:

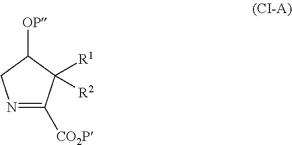

or a salt thereof, where P′ is hydrogen or a protecting group for a carboxylic acid; P″ is hydrogen or a protecting group for an alcohol; and R¹ and R² are independently hydrogen or an alkyl group, or R¹ and R² are taken together to form an optionally substituted cycloalkyl group. It will be readily apparent that Compound CI-A encompasses intermediates 7d-P, 7h-P, and 7h′-P described above in Scheme 1. Compound CI-A also encompasses intermediate 7d described above in Scheme 1G. Compound CI-A also encompasses intermediate 7h described above in Scheme 1G-1.

In various embodiments, CI-1 is selected from:

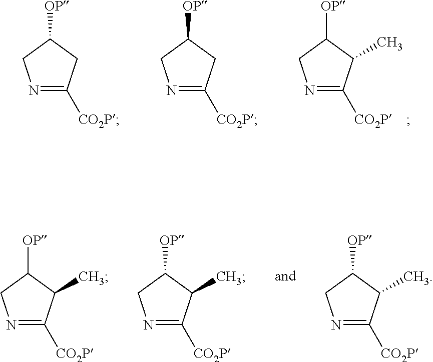

Some embodiments include Compound CII-A:

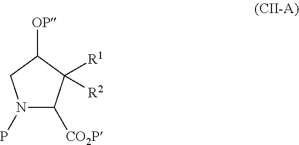

or salts thereof, where P is a hydrogen or protecting group for an amine; P′ is hydrogen or a protecting group for a carboxylic acid; P″ is hydrogen or a protecting group for an alcohol; and R¹ and R² are independently hydrogen or an alkyl group, or R¹ and R² are taken together to form a cycloalkyl group. It will be readily apparent that Compound CII-A encompasses intermediates 7b-P, 7f-P, 7f′-P, 7f″-P, 7g-P, 7i′-P, 7m-P, 1e-P, and 1-Y described above in Scheme 1. Compound CII-A also encompasses intermediates 2a and 2d described above in Scheme 1B. Compound CII-A also encompasses intermediates 3d described above in Schemes 1C and 1F. Compound CII-A also encompasses intermediate 4e described above in Scheme 1D. Compound CII-A also encompasses intermediates 5b and 5f described above in Scheme 1E. Compound CII-A also encompasses intermediate 6j described above in Scheme 1F. Compound CII-A also encompasses intermediates 2, 2a, 7b, 7f, and 7g described above in Scheme 1G. Compound CII-A also encompasses intermediates 7i and 7m described above in Scheme 1G-1. Compound CII-A also encompasses intermediate 7j described above in Scheme 1G-2. Compound CII-A also encompasses intermediates 7k and 7l described above in Scheme 1G-3. Compound CII-A also encompasses intermediate 3-K described above in Scheme 3A. In some embodiments of Compound CII-A, if P is hydrogen and R¹ and R² are hydrogen, then P″ is not hydrogen and P′ is not methyl.

In various embodiments, CII-A is selected from:

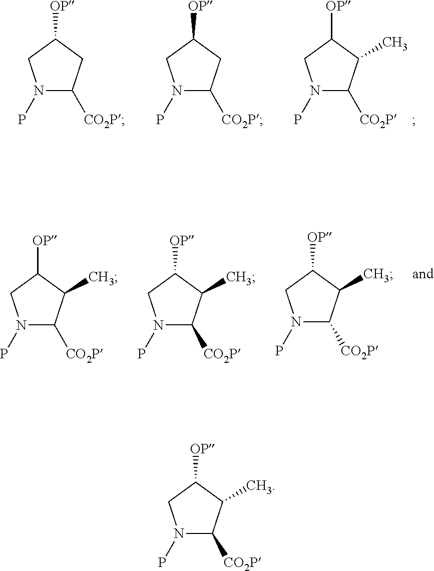

In some embodiments of Compounds C-A, CI-A, and CII-A, R¹ is C₁₋₄ alkyl or a C₁₋₈ alkyl (e.g., methyl). In some embodiments, R¹ is hydrogen. In some embodiments, R¹ and R² are taken together to form an optionally substituted C₁₋₆ cycloalkyl group (e.g., optionally substituted cyclopropyl). In some embodiments, P is a carbamate protecting group. In some embodiments, P′ is an ester protecting group. In some embodiments, P″ is an ether protecting group. In some embodiments, P is an optionally substituted CBz, BOC, or Fmoc protecting group. In some embodiments, P′ is an optionally substituted C₁₋₈ alkyl, C₁₋₁₀ cycloalkylalkyl, or C₁₋₁₀ aryl-alkyl group. In some embodiments, P″ is a silyl ether.

### EXAMPLES

### Example 1

### Synthesis of 4,4-Dimethyl Proline Precursor

The 4,4-dimethyl proline precursor 5f was synthesized according to Scheme 1E described above.

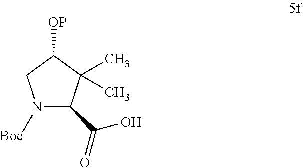

Synthesis of (S)-di-tert-butyl 3,3-dimethyl-4-oxopyrrolidine-1,2-dicarboxylate (5c): A flame-dried flask was purged with argon and then charged with anhydrous THF (10 ml), DMPU (8 ml) and 1M NaHMDS THF-solution (5 ml, 5 mmol). After being cooled to −78° C. the solution was treated with a solution of (S)-di-tert-butyl 4-oxopyrrolidine-1,2-dicarboxylate 4 (570 mg, 2 mmol) in THF (10 ml). After stirring for 5 min methyl iodide was added and the reaction was stirred for 2 hours at −78° C. The reaction was quenched with saturated aqueous ammonium chloride (30 ml) and extracted with hexane. Organic layer was washed with brine, dried over magnesium sulfate and concentrated under reduced pressure to dryness. The target di-methyl proline was isolated by column chromatography in 10-20% ethyl acetate-hexane as a white solid. Yield: 225 mg (36%). ¹H-NMR (chloroform-d), δ: 4.24 and 4.16 (two s., 1H), 4.06-3.86 (m, 2H), 1.48 (s, 9H), 1.46 and 1.44 (two s., 9H), 1.27 and 1.26 (two s., 3H), 1.13 and 1.12 (two s., 3H).

Preparation of (S)-1-(tert-butoxycarbonyl)-3,3-dimethyl-4-oxopyrrolidine-2-carboxylic acid (5e): To a solution of the tert-butyl ester 5c (220 mg, 0.7 mmol) in DCM (1 ml) was added TFA and the reaction was allowed to proceed for 2 h at room temperature followed by 2 h at 35° C. The reaction mixture was diluted with toluene and the solvent was removed under reduced pressure. The residue was taken into dioxane (2 ml) and water (2 ml). Sodium bicarbonate (294 mg, 3.5 mmol) and di-tret-butyl-dicarbonate (305 mg, 1.4 mmol) were added and the reaction was left stirred at room temperature. After one hour it was acidified to pH 2-3 and extracted with DCM. Organic layer was dried over sodium sulfate and concentrated under reduced pressure to give keto-acid intermediate 5e as a white solid of which was used without any further purification.

Preparation of (2S,4R)-1-(tert-butoxycarbonyl)-4-hydroxy-3,3-dimethylpyrrolidine-2-carboxylic acid (5f): To a solution of the intermediate 5e in DCM (5 ml) was added sodium cyanoborohydride (445 mg, 2.1 mmol) at 0° C. followed by acetic acid (0.28 ml) and the reaction was stirred overnight at room temperature. The reaction was diluted with brine, acidified to pH 2 and extracted with several portions of ethyl acetate. Organic phase was dried over magnesium sulfate and concentrated under reduced pressure. The intermediate 5f was isolated as white solid after crystallization from ether. Yield: 145 mg (80%). ¹H-NMR (DMSO-d⁶), δ: 5.14 (br. s, 1H), 3.75 (m, 2H), 3.53 (m, 1H), 3.07 (dd, 1H), 1.38 and 1.32 (two s, 9H), 1.02 an 0.99 (two s, 3H), 0.91 and 0.88 (two s, 3H).

### Example 2

### Synthesis of 4-Methyl Proline Precursors

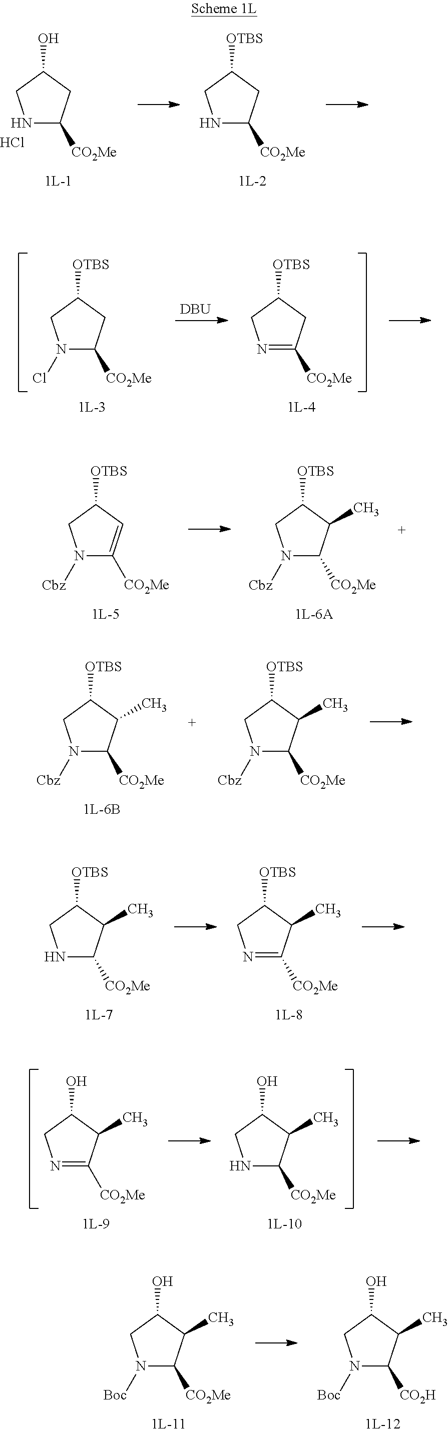

### Compound 1L-2

To a solution of L-4-hydroxyproline methyl ester hydrochloride (1L-1, 9.62 g, 53 mmol) in DCM (100 ml) were added imidazole (10.2 g, 102 mmol) and TBS-Cl (8.73 g, 58 mmol). The mixture was stirred overnight at room temperature and the solids were filtered off. The filtrate was concentrated under reduced pressure and the residue was partitioned between ethyl acetate and water. The organic phase was separated and the aqueous layer was back extracted with ethyl acetate. Combined organic solution was washed with brine, dried over magnesium sulfate end concentrated under reduced pressure. The residue was dissolved in hexane (˜100 ml) and solids were filtered off. The filtrate was concentrated under vacuum to give Compound 1L-2 (13.93 g, 98%). ¹H-NMR (chloroform-d), δ: 4.38 (m, 1H), 4.00 (dd, 1H), 3.73 (s, 3H), 3.11 (dd, 1H), 2.84 (dd, 1H), 2.20 (br. S, 1H), 2.06 (m, 1H), 1.94 (m, 1H), 0.88 (s, 9H), 0.06 (s, 3H), 0.05 (s, 3H).

### Compound 1L-4

To a stirred at 0° C. solution of Compound 1L-2 (13.93 g, 53.7 mmol) in ether (100 ml) was added tert-butyl hypochlorite (5.83 g, 53.7 mmol). The reaction mixture was stirred for 30 min at 0° C. when DBU (8.4 ml, 56.4 mmol) was added dropwise under vigorous stirring. The reaction mixture was stirred for 10 min at 0° C. and 20 min at room temperature. The solids were filtered off and washed with hexane. The filtrate was concentrated under reduced pressure to provide Compound 1L-4 as a yellow oil.

### Compound 1L-5

Compound 1L-4 was dissolved in anhydrous DCM (60 ml) and the solution was cooled to −10° C. To this solution was added 2,6-lutidine (15.6 ml, 134 mmol) followed by addition of benzyl chloroformate (9.8 ml, 69.8 mmol). The reaction was stirred for 15 min at −10° C., then 2 h at room temperature and overnight at 30° C. More benzyl chloroformate (2.3 ml, 16 mmol) and lutidine (3.2 ml, 27 mmol) was added and the stirring was continued at 40° C. for another 3 hours. The reaction was quenched by addition of ethylenediamine, stirred for ˜15 min at room temperature and concentrated under vacuum. The residue was taken into hexane and 1 M citric acid (250 ml), organic layer was washed successively with water and aqueous sodium bicarbonate, dried over sodium sulfate and concentrated under vacuum. Compound 1L-5 was isolated by column chromatography in 5-25% ethyl acetate-hexane. Yield 14.8 g (70.5%). ¹H-NMR (chloroform-d), δ: 7.32-7.37 (m, 5H), 5.66 (d, 1H), 5.15 (s, 2H), 4.92-4.96 (m, 1H), 3.98 (dd, 1H), 3.78 (dd, 1H), 3.67 (s, 3H), 0.87 (s, 9H), 0.07 (s, 6H).

### Compound 1L-6A and 1L-6B

Copper (I) bromide-dimethyl sulfide complex (9.85 g, 47.9 mmol) was suspended in anhydrous ether (100 ml) under argon atmosphere and the reaction mixture was cooled to −30° C. Methyl lithium (1.6 M in ether, 60 ml, 95.8 mmol) was added dropwise maintaining the temperature below −25° C. The resulted colorless solution of lithium dimethylcuprate was stirred for 45 min at −25° C. to −30° C. and cooled to −50° C. A solution of Compound 1L-5 (14.43 g, 36.9 mmol) in ether (˜40 ml) was added dropwise via cannula while maintaining the temperature below −40° C. The reaction mixture was stirred 45 min at −50° C. to −40° C. and then slowly transferred via cannula to a vigorously stirred saturated aqueous ammonium chloride (250 ml). The mixture was stirred open to the air until most solid dissolved (˜30 min). Organic layer was separated and washed with saturated ammonium chloride. Aqueous phases were back-extracted with hexane. The combined organic solution was dried over sodium sulfate and concentrated under reduced pressure. Compound 1L-6A was isolated as a white solid by crystallization from hexane. Yield 10.31 g (68.7%). ¹H-NMR (chloroform-d), δ (two rotamers): 7.29-7.38 (m, 5H), 5.01-5.20 (m, 2H), 3.99 and 3.93 (d and d, 1H), 3.81-3.86 (m, 1.5H), 3.74 (s and m, 1.8H), 3.57 (s, 1.6H), 3.29-3.33 (m, 1H), 2.26-2.35 (m, 1H), 1.09-1.12 (d and d, 3H), 0.86 (s, 9H), 0.05 (s, 3H), 0.04 (s, 3H).

The mother liquor after the first crystallization was concentrated under reduced pressure to give an oily residue. Approximately 5 g of this oil was purified by column chromatography in 5-15% ethyl acetate-hexane to provide Compound 1L-6B. Yield 600 mg (approximately 6%). ¹H-NMR (chloroform-d), δ: (two rotamers) 7.28-7.34 (m, 5H), 5.01-5.22 (m, 2H), 4.15-4.18 (m, 1H), 4.03 and 3.97 (two d, !H), 3.77 (s, 1.4H), 3.56-3.55 (m, 2 H), 3.53 (s, 1.6H), 2.20-2.26 (m, 1H), 1.11 and 1.08 (two d, 3H), 0.87 and 0.86 (two s, 9H), 0.06 (s, 6H).

### Compound 1L-7

Compound 1L-6A (10.3 g, 25.3 mmol) was dissolved in methanol (100 ml), 10% Pd/C (340 mg) was added and the mixture was hydrogenated at ˜40 psi hydrogen pressure overnight. The reaction mixture was filtered through celite and concentrated to give Compound 1L-7 which was used in subsequent reactions without any further purification. Yield 6.96 g (100%). ¹H-NMR (chloroform-d), δ: 3.79 (ddd, 1H), 3.68 (s, 3H), 2.97 (dd, 1H), 2.86 (dd, 1H), 2.65 (br. s. 1H), 2.06-2.15 (m, 1H), 1.08 (d, 3H), 0.81 (s, 9H), 0.04 (s, 3H), 0.03 (s, 3H).

### Compound 1L-8

To a solution of Compound 1L-7 (6.46 g, 23.5 mmol) in anhydrous ether (80 ml) was added tert-butyl hypochlorite (2.68 g, 24.7 mmol) at 0° C. After 20 min DBU (3.68 ml, 24.7 mmol) was added under stirring at 0° C. and the reaction mixture was stirred for 30 min at room temperature. The reaction mixture was filtered through celite and the solids were washed with hexane. The filtrate was concentrated under vacuum and the residue was separated by column chromatography in 10-50% ethyl acetate-hexane to furnish Compound 1L-8. Yield 6.35 g (99.5%). ¹H-NMR (chloroform-d), δ: 4.12 (dd, 1H), 4.02 (ddd, 1H), 3.93 (dd, 1H), 3.88 (s, 3H), 3.00-3.06 (m, 1H), 1.15 (d, 3H), 0.87 (s, 9H), 0.07 (s, 3H), 0.06 (s, 3H).

### Compound 1L-11

To a solution of Compound 1L-10 (6.35 g, 23.4 mmol) in THF (100 ml) was added TBAF (1M in THF, 25 ml, 25 mmol) and the reaction was kept at 0° C. After 25 min acetic acid (4.2 ml, 70.5 mmol) was added followed by addition of NaBH(OAc)₃. The reaction was stirred at 0° C. for 1 h and then left stirred overnight. Saturated aqueous sodium bicarbonate (250 ml) was added to the reaction mixture under vigorous stirring and 15 min later Boc₂O (7.7 g, 35.2 mmol) was added. The reaction was allowed to stir at room temperature for 1 hour, when organic phase was separated. Aqueous phase was additionally extracted with ethyl acetate, combined organic solution was washed with 1 M citric acid and sodium bicarbonate. Organic solution was dried over magnesium sulfate and concentrated in vacuo. The residue was purified by column chromatography in 30-70% ethyl acetate-hexane and Compound 1L-11 was isolated by crystallization from hexane-ethyl acetate (4:1). Yield 4.2 g (69%). ¹H-NMR (chloroform-d), δ (two rotamers): 4.40 (d and d, 1H), 4.07-4.13 (m, 1H), 3.84-3.92 (dd and dd, 1H), 3.73 and 3.72 (s and s, 3H), 3.25 and 3.19 (dd and dd, 1H), 2.30-2.42 (m, 1H), 1.76 (d and d, ex, 1H), 1.45 and 1.40 (s and s, 9H), 1.01 and 0.99 (d and d, 3H).

### Compound 1L-12

To a solution of Compound 7m (78 mg, 0.3 mmol) in ethanol (2 ml) was added 2 M aqueous lithium hydroxide (1.5 ml, 3 mmol) and the reaction was stirred for 2 hours at 40° C. The reaction was neutralized by 2 N hydrochloric acid (1.5 ml) and concentrated under reduced pressure. The residue was partitioned between ethyl acetate and brine, water phase was separated and back extracted with ethyl acetate. Combined organic solution was dried over magnesium sulfate, filtered and concentrated under vacuum to give Compound 1L-12. Yield 73 mg (100%). ¹H-NMR (chloroform-d, 60° C.) δ: 6.15 (br. s, ex, 1H), 4.38 (d, 1H), 4.08 (m, 1H), 3.82 (m, 1H), 3.26 (m, 1H), 2.41 (m, 1H), 1.43 (s, 9H), 1.07 (d, 3H).

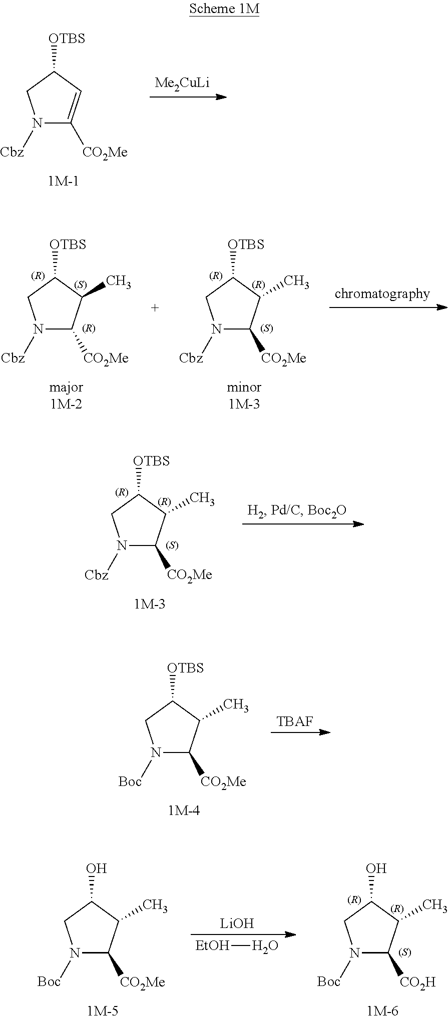

### Compounds 1M-2 and 1M-3

Cupper (I) bromide-dimethyl sulfide complex (9.85 g, 47.9 mmol) was suspended in anhydrous ether (100 ml) under argon atmosphere and the reaction mixture was cooled to −30° C. Methyl lithium (1.6 M in ether, 60 ml, 95.8 mmol) was added dropwise maintaining the temperature below −25° C. The resulted colorless solution of lithium dimethylcuprate was stirred for 45 min at −25° C. to −30° C. and cooled to −50° C. A solution of Compound 1M-1 (14.43 g, 36.9 mmol) in ether (˜40 ml) was added dropwise via cannula while maintaining the temperature below −40° C. The reaction mixture was stirred 45 min at −50° C. to −40° C. and then slowly transferred via cannula to a vigorously stirred saturated aqueous ammonium chloride (250 ml). The mixture was stirred open to the air until most solid dissolved (˜30 min). Organic layer was separated and washed with saturated ammonium chloride. Aqueous phases were back-extracted with hexane. The combined organic solution was dried over sodium sulfate and concentrated under reduced pressure. Compound 1M-2 was isolated as a white solid by crystallization from hexane. Yield 10.31 g (68.7%). ¹H-NMR (chloroform-d), δ (two rotamers): 7.29-7.38 (m, 5H), 5.01-5.20 (m, 2H), 3.99 and 3.93 (d and d, 1H), 3.81-3.86 (m, 1.5H), 3.74 (s and m, 1.8H), 3.57 (s, 1.6H), 3.29-3.33 (m, 1H), 2.26-2.35 (m, 1H), 1.09-1.12 (d and d, 3H), 0.86 (s, 9H), 0.05 (s, 3H), 0.04 (s, 3H).

The mother liquor after the first crystallization was concentrated under reduced pressure to give an oily residue. Approximately 5 g of this oil was purified by column chromatography in 5-15% ethyl acetate-hexane to provide Compound 1M-3. Yield 600 mg (approximately 6%). ¹H-NMR (chloroform-d), δ: (two rotamers) 7.28-7.34 (m, 5H), 5.01-5.22 (m, 2H), 4.15-4.18 (m, 1H), 4.03 and 3.97 (two d, !H), 3.77 (s, 1.4H), 3.56-3.55 (m, 2 H), 3.53 (s, 1.6H), 2.20-2.26 (m, 1H), 1.11 and 1.08 (two d, 3H), 0.87 and 0.86 (two s, 9H), 0.06 (s, 6H).

### Compound 1M-4

A solution of Compound 1M-3 (408 mg, 1 mmol) and Boc₂O (327 mg, 1.5 mmol) in THF (20 ml) was hydrogenated overnight in the presence of 10% Pd/C (50 mg). The catalyst was filtered off and the solvent was removed under reduced pressure. The residue was purified by column chromatography in 10-30% ethyl acetate-hexane to afford Compound 1M-4. Yield 250 mg (67%). ¹H-NMR (chloroform-d), δ: (two rotamers) 4.14 (m, 1H), 3.97 and 3.88 (d and d, 1H), 3.76 and 3.74 (s and s, 3H), 3.42-3.54 (m, 2H), 2.18-2.25 (m, 1H), 1.45 and 1.41 (s and s, 9H), 1.10 and 1.08 (d and d, 3H), 0.88 (s, 9H), 0.06 (s, 6H).

### Compound 1M-5

To a solution of Compound 1M-4 (250 mg, 0.67 mmol) in THF (3 ml) was added 1 M solution of TBAF in THF (0.87 ml, 0.87 mmol). After 1 hour the reaction was quenched by addition of saturated aqueous sodium bicarbonate and then extracted with ethyl acetate. The solvent was removed under reduced pressure and the residue was purified by column chromatography in 30-70% ethyl acetate-hexane to afford Compound 1M-5. Yield 177 mg (100%). ¹H-NMR (chloroform-d), δ: (two rotamers) 4.17 (m, 1H), 3.92 and 3.88 (d and d, 1H), 3.72 and 3.71 (s and s, 3H), 3.48-3.61 (m, 2H), 2.50-2.56 (br. s, 1H), 2.23 (m, 1H), 1.41 and 1.36 (s and s, 9H), 1.13 (d, 3H).

### Compound 1M-6

To a solution of Compound 1M-5 (177 mg, 0.67 mmol) in ethanol (3 ml) was added 2 N aqueous lithium hydroxide (3.4 ml, 6.8 mmol). The reaction mixture was stirred for one hour at 40° C. when it was acidified to pH ˜2 with 2 N aqueous hydrochloric acid and extracted with ethyl acetate. The organic extract was dried over magnesium sulfate and the solvent was removed under reduced pressure to afford Compound 1M-6 which was used in additional steps without any further purification. Yield 160 mg (97%).

### Example 3

### Alternative Synthesis of 4-Methyl Proline Precursors

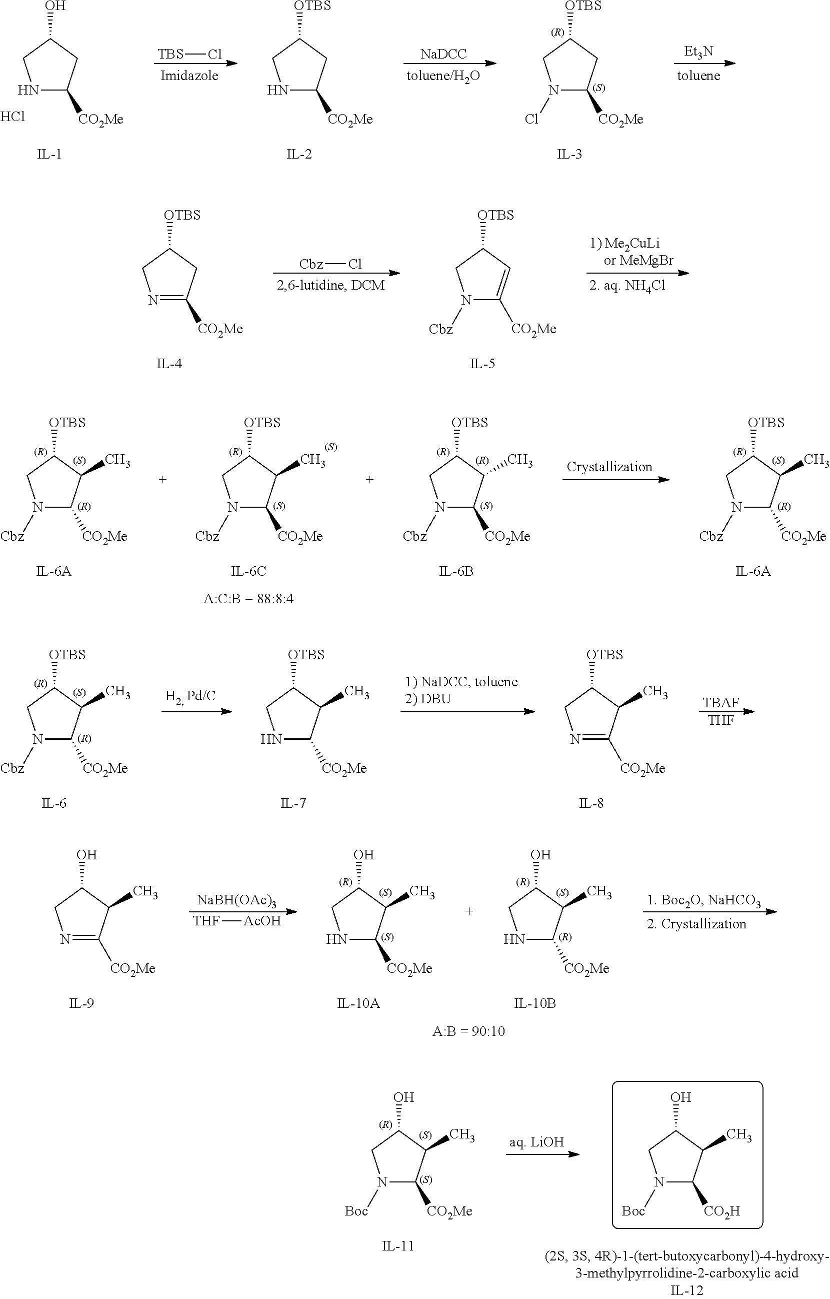

### Compound IL-4

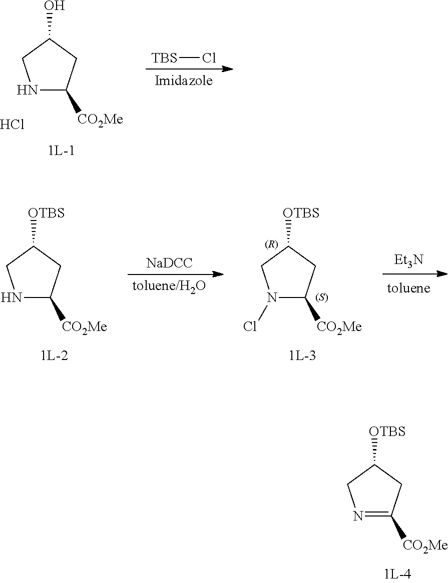

To a stirred at 0° C. mixture of L-4-hydroxyproline methyl ester hydrochloride (200 g, 1.1 mol) and DCM (1500 ml) were added imidazole (172.2 g, 2.5 mol, 2.3 eq) and TBS-Cl (180.6 g, 1.2 mol, 1.1 eq). The mixture was left stirred overnight. The resulting mixture was cooled to 0° C., and to the stirred reaction mixture was added the solution of 10% aq. solution of sodium carbonate (1500 ml). The organic solution was separated, and the water phase was extracted with DCM (1000 mL), the combined organic solution was washed with water, concentrated to 600˜700 mL, and residual DCM was substituted with toluene (1500 mL) under reduced pressure. The toluene solution was concentrated under reduced pressure to a volume ˜1000 ml and washed with water. The toluene phase was cooled to 0° C., to this solution was added water (500 ml) followed by NaDCC (sodium dichloroisocyanurate, 133 g, 0.6 mol, 0.55 eq). After 30 min TLC indicated that the formation of N-chloro intermediate was completed. The mixture was filtered through celite pad, washed with ˜500 mL toluene, water phase was separated. Organic phase was washed once with water, cooled to 0° C. and then triethylamine (133 g, 1.3 mol, 1.2 eq) was added. The reaction was stirred 1 h at 0° C. followed by stirred overnight at room temperature, when TLC indicated complete conversion of N—Cl intermediate to the imine. Organic solution was washed twice with water and solvent was removed under reduced pressure to afford crude imine as colorless oil, which was used directly without further purification.

### Compound IL-5

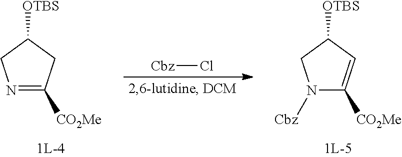

The previous step oil was dissolved into DCM (2000 mL) and solution was cooled to −10° C. To this solution was added 2,6-lutidine (235 g, 2.2 mol, 2 eq), followed by Cbz-Cl (206 g, 1.2 mol, 1.1 eq), the reaction was stirred 2 h at −10° C., then stirred overnight at room temperature. To the resulting solution was added ethylenediamine (˜10 mL) to quench the excess of Cbz-Cl, the reaction mixture was stirred for ˜15 min at room temperature and washed with a mixture of ˜1200 mL 1 M citric acid (266 g, 1.26 mol, 1.15 eq) and 2N HCl (˜470 mL, 0.85 eq), then organic layer was washed with water, aqueous sodium bicarbonate, and then washed with water, the organic layer dried over sodium sulfate, filtrated and concentrated under reduce pressure, then the residue was dissolved ˜300 mL DCM, poured into silicon gel column, quickly eluted with 5% ethyl-petroleum ether to 10% ethyl-petroleum ether, to afford compound IL-5 as a pale yellow oil (250 g, 60.3% based on the starting amino ester hydrochloride).

¹HNMR (CDCl₃), δ: 7.26-7.30 (m, 5H), 5.59 (d, J=2.8 Hz, 1H), 5.08 (d, J=1.6 Hz, 2H), 4.85-4.89 (m, 1H), 3.88 (dd, 1H), 3.75 (dd, 1H), 3.60 (s, 3H), 0.80 (s, 9H), 0.00 (s, 6H).

### Compound IL-6A

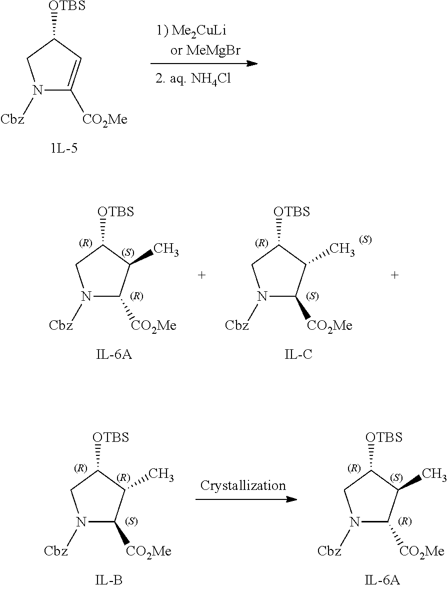

A 3-L 3-neck flask was charged with cupper (I) bromide-dimethyl sulfide complex (136.4 g, 0.66 mol, 1.29 eq) and anhydrous ether (1000 ml) under N₂ atmosphere and the suspension was cooled to −30° C. Methyl lithium (1.6M in ether, 840 mL, 1.34 mol) was added dropwise maintaining the temperature below −25° C. The resulted colorless solution of lithium dimethylcuprate was stirred for 45 min at −25-−30° C. and then cooled to −60° C. A solution of the compound IL-5 (200 g, 0.51 mol) in ether (˜500 ml) was added dropwise over 15 min while maintaining the temperature below −50° C. The reaction mixture was stirred 45 min at −50-−40° C. and then slowly transferred via canula to a vigorously stirred mixture of saturated aqueous ammonium chloride (2000 ml) and ice (500 g). The mixture was stirred over 30 min, then open to air. The organic layer was separated; the water solution was then extracted with Petroleum ether (1000 m L×3). Combined organic solution was filtrated, and then the filtration was washed with NH₄Cl solution, then dried over sodium sulfate and concentrated under reduced pressure. Then residue was dissolved into DCM and then filtrated through silicon gel pad, the filtration was then concentrated under reduce pressure, to the residue was added hexane (1200 mL), then crystal seed was added, the solution was left overnight. The crystal was filtrated, and the filtrated cake was washed with hexane, and the filtrated cake solid afford 100 g of pure desired compound IL-6A, the mother liquid was purified by silicon gel column by 5-15% ethyl acetate-Petroleum ether, and then crystallized with hexane, added crystal seed overnight, the solid was filtrated to afford another 30 g pure compound IL-6A. Combined yield: 130 g (yield, 62.4%).

¹H-NMR (CDCl₃), δ (two rotamers): 7.29-7.38 (m, 5H), 4.97-5.16 (m, 2H), 3.96 and 3.90 (d and d, 1H), 3.76-3.80 (m, 1.6H), 3.68 (s and m, 1.8H), 3.53 (s, 1.6H), 3.23-3.28 (m, 1H), 2.23-2.28 (m, 1H), 1.06-1.08 (d and d, 3H), 0.81 (s, 9H), 0.02 (s, 3H), 0.00 (s, 3H).

### Compound IL-8

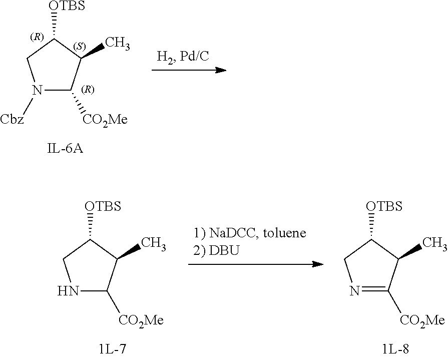

Compound IL-6A (100 g×2, 0.245 mol×2) was dissolved in methanol (600 ml×2), 5% Pd/C (5 g×2) was added and the mixture was hydrogenated at ˜40 psi hydrogen pressure for 2 hour. The two batches were combined. The catalyst was filtered off and the filtrate was concentrated to ˜1/2 volume under reduced pressure. Residual methanol was chased with toluene (˜1500 ml). The solution was concentrated to ˜800 ml and cooled to 0° C. Water (400 ml) and sodium dichloroisocyanurate (59.3 g, 0.27 mol) were added and the reaction was stirred vigorously for 1 hour at 0° C. The reaction mixture was filtered through a celite pad; organic phase was separated and washed with water (˜500 ml). Organic layer was cooled to 0° C. and treated with DBU (85.8 g, 0.56 mol) under stirring. After 15 min at 0° C. the reaction mixture was left stirred at room temperature 2 hours, when it was washed with water until water phase remains neutral. Combined aqueous phases were back-extracted with toluene. Combined organic solution was dried over sodium sulfate and concentrated under reduced pressure to furnish the title compound IL-8 as a pale-yellow oil (168 g, yield ˜100%, contains 1:1 of residual toluene) which was used on the next step without any further purification.

Compound IL-7: ¹HNMR (CDCl₃), δ: 3.79 (ddd, 1H), 3.70 (s, 3H), 3.29 (dd, J=6.4 Hz, 1H), 2.97 (dd, 1H), 2.88 (dd, 1H), 2.06-2.15 (m, 1H), 1.10 (d, J=6.4 Hz, 3H), 0.83 (s, 9H), 0.01 (s, 3H), 0.00 (s, 3H)

Compound IL-8: (contains 1:1 of residual toluene). ¹HNMR (CDCl₃), δ: 4.12 (dd, 1H), 4.02 (ddd, 1H), 3.93 and 3.89 (dd, 1H), 3.81 (s, 3H), 3.00-3.06 (m, 1H), 1.09 (d, 3H), 0.79 (s, 9H), 0.00 (s, 6H)

### Compound IL-11

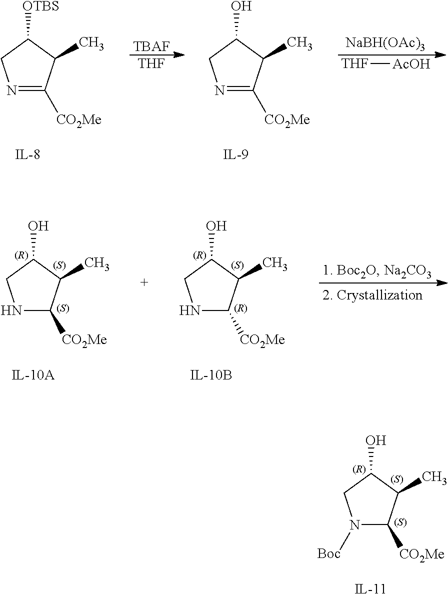

To a stirred at 0° C. solution of the crude compound IL-8 obtained on the previous step (168 g, ˜0.49 mol) in THF (750 ml) was added TBAF (1M in THF, 500 ml, 0.5 mol) and the reaction was allowed to proceed for 2 hour at 0° C. The reaction mixture was next cooled to −40° C. and acetic acid (150 g, 2.5 mol) was added dropwise over 10-15 min followed by addition of NaBH(OAc)₃ (208 g, 0.98 mol) in a single portion. The reaction was slowly (over 3-5 hours) warmed to room temperature and left stirred overnight. To the reaction mixture was added slowly 2M aqueous sodium carbonate solution (˜1000 mL) and after 15 min Boc₂O (139.5 g, 0.64 mol) was added. The reaction was stirred at room temperature for 1 hour, when organic phase was separated; aqueous phase was additionally extracted two times with ethyl acetate. The combined organic solution was concentrated under reduced pressure; the residue was partitioned between ethyl acetate (˜1000 mL) and water (1000 mL). The mixture was acidified with 2N aqueous hydrochloric acid to pH ˜2 (˜260 mL), organic layer was washed successively with water and 5% aqueous sodium bicarbonate. Aqueous phases were back-extracted three times with ethyl acetate. Combined organic solution was dried over magnesium sulfate and concentrated in vacuo. The residue was crystallized from hexane (500 mL) to afford the title hydroxyprolinate as off-white solid. 55 g, and the mother liquid was purified by column give a crude oil, the oil was then recrystallized from hexane to give another 5 g, combined 60 g pure desired compound IL-11 (yield, 47.3%)

¹H-NMR (CDCl₃), δ (two rotamers): 4.40 (d and d, J=8.4 Hz, 1H), 4.07-4.10 (m, 1H), 3.87-3.90 (dd and dd, 1H), 3.73 and 3.72 (s and s, 3H), 3.25 and 3.19 (dd and dd, 1H), 2.30-2.42 (m, 1H), 1.76 (d and d, ex, 1H), 1.45 and 1.40 (s and s, 9H), 1.01 and 0.99 (d and d, 3H).

Optical rotation: [a]=15.0, C=0.01 g/mL, CHCl₃

### Compound IL-12

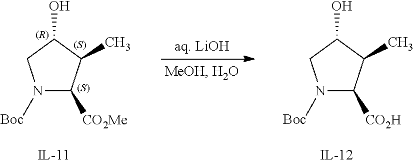

Compound IL-11 (100 g, 0.38 mol) was dissolved in 500 mL methanol and 500 mL water, then to the mixture was added 2 N LiOH solution (33.5 g, 0.76 mol), the reaction mixture was stirred overnight at room temperature. The resulting solution was removed methanol under reduce pressure, then the solution was neutralized by 2N hydrochloric acid (˜380 mL to pH=2), saturated with sodium chloride solid and extracted with ethyl acetate (3×500 mL). Combined organic solution was washed with brine, dried over sodium sulfate, filtered and concentrated under vacuum to give ˜60 g desired compound IL-12. The aqueous solution pH changed to 6-7, added 2 N hydrochloric acid to pH=2 again, then extracted with isopropanol/DCM (300 mL×6), combined organic solution was washed with brine, dried over sodium sulfate, filtered and concentrated under vacuum to give another ˜20 g compound IL-12, the combined oil compound IL-12 was triturated with 5% ethyl acetic ester in hexane to give 80 g of desired compound IL-12 as a white solid. Yield 80 g (yield, 84.5%).

¹HNMR (CDCl₃, 50° C.) δ: 4.39 (d, J=7.6 Hz, 1H), 4.10 (m, 1H), 3.84 (m, 1H), 3.27 (m, 1H), 2.41 (m, 1H), 1.45 (s, 9H), 1.07 (d, J=7.2 Hz, 3H).

Optical rotation: [a]=12.8, C=0.1 g/mL, Methanol

### Example 4

### Synthesis of 2-Methyl Proline Macrocyclic Analog

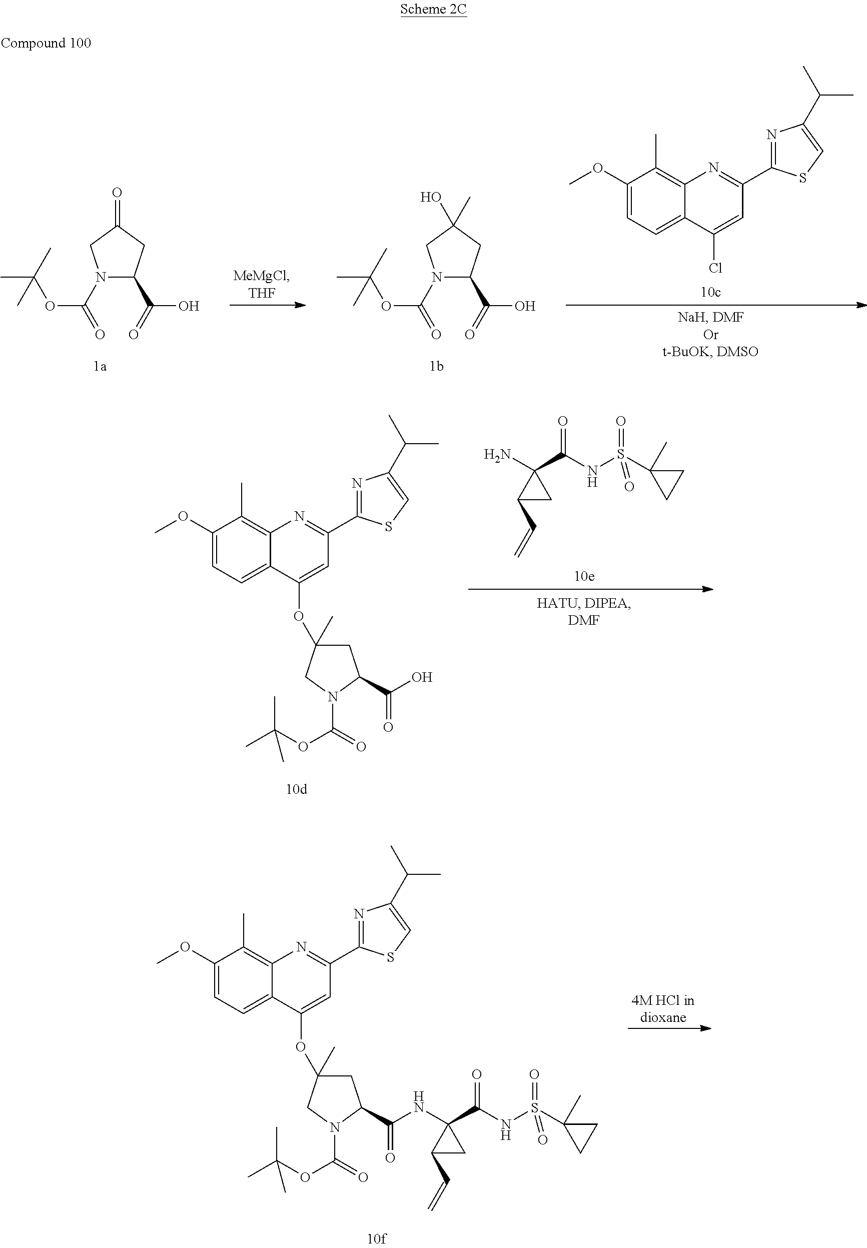

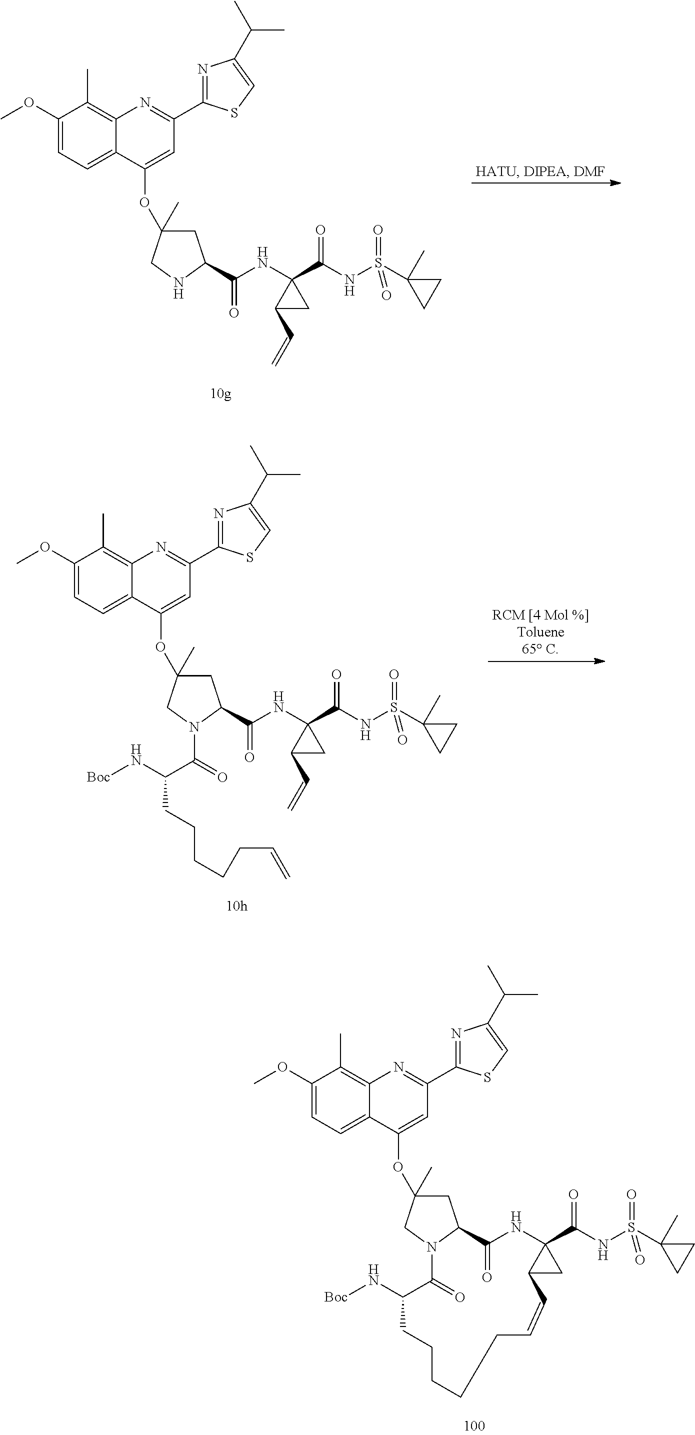

Preparation of compound 100: Macrocycles, such as compound 100, can be synthesized as shown in Scheme 2C. N-Boc-4-oxo-L-proline (1a) can be reacted with an organometallic reagent, for example a Grignard reagent such as methyl magnesium chloride, to afford N-Boc-4-hydroxy-4-methyl-L-proline (1b). N-Boc-4-hydroxy-4-methyl-L-proline (1b) can be treated with 4-chloro-2-(4-isopropylthiazol-2-yl)-7-methoxy-8-methylquinoline under basic conditions, such as sodium hydride in DMF or potassium tert-butoxide in DMSO, to afford carboxylic acid 10d. Carboxylic acid 10d can be coupled with amine 10e using standard coupling conditions, for example HATU in the presence of DIPEA, to afford compound 10f. The Boc protecting group of compound 10f can be removed under acid conditions, such as 4M HCl in dioxane, to afford amine 10g. Amine 10g can be coupled with carboxylic acid 8d using standard coupling conditions, for example HATU in the presence of DIPEA, to afford compound 10h. Compound 10h can be cyclized in the presence of a catalyst, such as a Zhan catalyst, to provide macrocycles, such as compound 100.

### Example 5

### Synthesis of 4,4-Dimethyl Proline Macrocyclic Analog

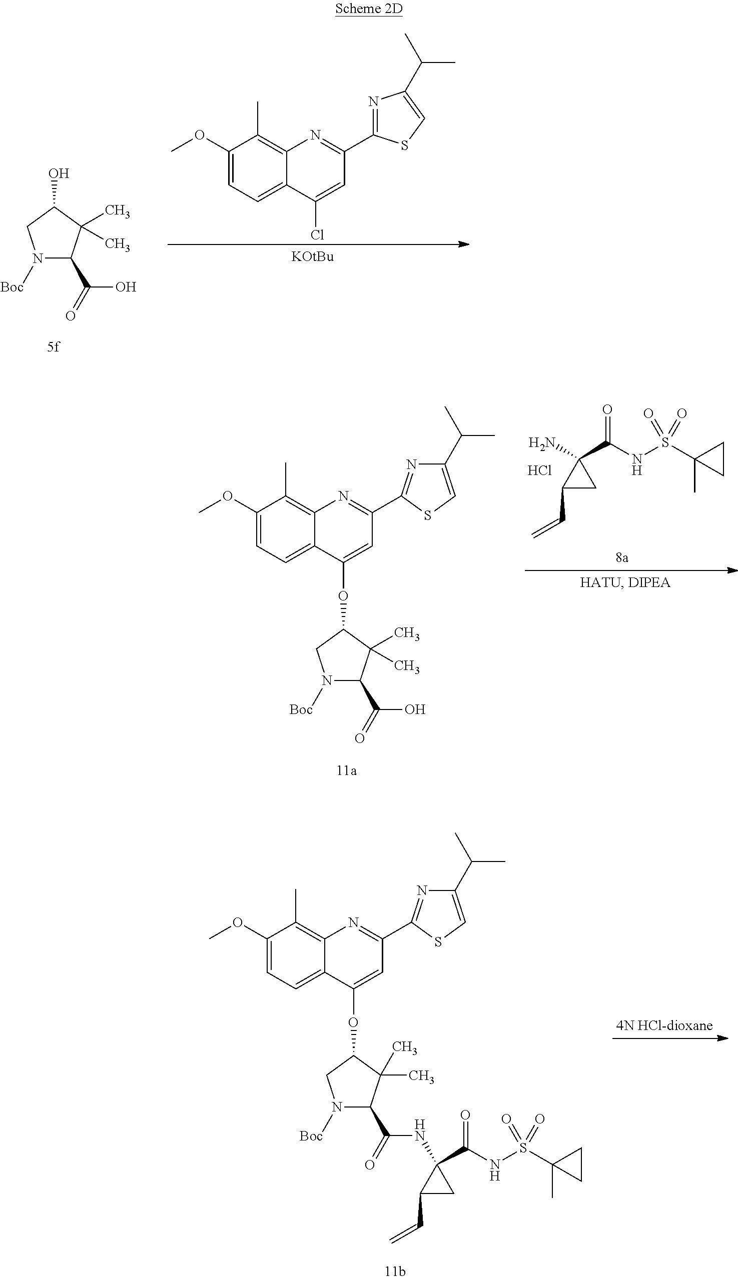

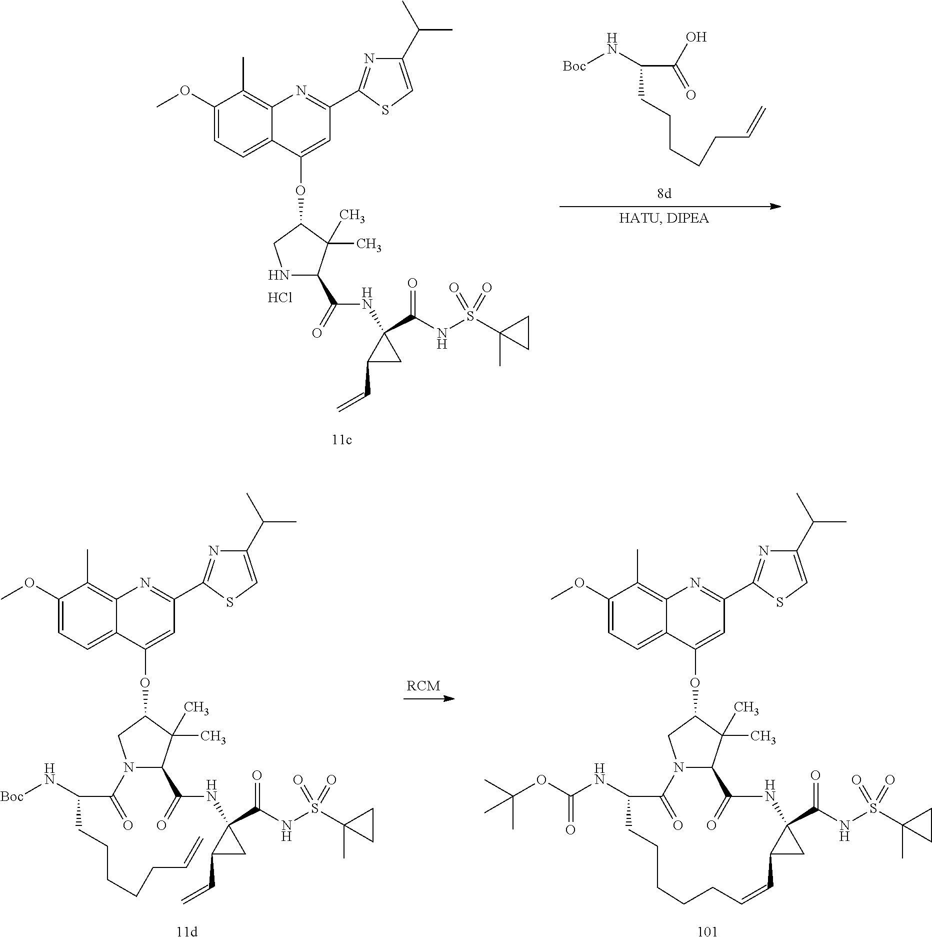

Preparation of compound 101: Macrocycles, such as compound 101, can be synthesized as shown in Scheme 2D. Compound 5f can be treated with 4-chloro-2-(4-isopropylthiazol-2-yl)-7-methoxy-8-methylquinoline under basic conditions, for example potassium tert-butoxide in DMSO, to afford carboxylic acid 11a. Carboxylic acid 11a can be coupled with amine 8a using standard coupling conditions, for example HATU in the presence of DIPEA, to afford compound 11b. The Boc protecting group of compound 8b can be removed under acid conditions, for example 4N HCl in dioxane, to afford amine 11c. Amine 11c can be coupled with carboxylic acid 8d using standard coupling conditions, for example HATU in the presence of DIPEA, to afford compound 11d. Compound 11d can be cyclized in the presence of a catalyst, such as a Zhan catalyst, to provide macrocycles, such as compound 101.

Preparation of P2 intermediate 11a: To a solution of hydroxyl acid 5f (100 mg, 0.386 mmol) in DMSO (3 ml) was added potassium tert-butylate (110 mg, 0.98 mmol). After being stirred for 5 min the reaction was treated with 2-(4-chloro-7-methoxy-8-methylquinolin-2-yl)-4-isopropylthiazole (200 mg, 0.60 mmol) and stirring was continued for 3 h when the reaction mixture was diluted with water, acidified to pH ˜3 and extracted with ethyl acetate. Organic phase was washed with brine, dried over magnesium sulfate and concentrated under vacuum. The intermediate 11a was isolated as a yellow foam by column chromatography in 1-10% methanol-DCM. Yield: 270 mg (87%). ¹H-NMR (chloroform-d, 60° C.), δ: 7.99 (d, 1H), 7.50 (s, 1H), 7.23 (d, 1H), 7.01 (s, 1H), 4.90 (dd, 1H), 4.35 (s, 1H), 4.11 (dd, 1H), 3.98 (s, 3H), 3.69 (m, 1H), 3.21 (m, 1H), 2.70 (s, 3H), 1.48 (s, 3H), 1.42 (s, 9H), 1.39 (d, 6H), 1.26 (s, 3H).

Preparation of P1′-P1-P2 intermediate 11b: To a stirred at 0° C. solution of the acid 11a (250 mg, 0.450 mmol) and P1′-P1 fragment 8a (as HCl salt; 189.5 mg, 0.675 mmol) in DMF (5 ml) were added at DIPEA (0.78 ml, 4.5 mmol) and HATU (256.5 mg, 0.675 mmol). The reaction was allowed to proceed overnight at room temperature when it was quenched with water and acidified to pH ˜3. Resulted mixture was extracted with ethyl acetate and the organic phase was washed with brine and dried over magnesium sulfate. The solvent was removed under reduced pressure and the residue was purified by column chromatography in 30 to 70% ethyl acetate-hexane to provide compound 11b as pale yellow foam. Yield 280 mg (79.5%). ¹H-NMR (chloroform-d, 60° C.), δ: 9.64 (br. s, 1H), 7.94 (d, 1H), 7.48 (s, 1H), 7.24 (d, 1H), 7.02 (s, 1H), 6.53 (br. s, 1H), 5.83 (ddd, 1H), 5.35 (d, 1H), 5.19 (d, 1H), 4.91 (br. dd, 1H), 4.21 (s, 1H), 4.02-3.97 (s and dd, 4H), 3.75 (br. dd, 1H), 3.21 (m, 1H), 2.70 (s, 1H), 2.21 (dd, 1H), 1.95 (dd, 1H), 1.75-1.69 (m, 1H), 1.64-1.60 (m, 1H), 1.55 (s, 3H), 1.47-1.38 (5 s, 18H), 1.19 (s, 3H), 0.83 (m, 2H).

Preparation of P1′-P1-P2 intermediate HCl salt 11c: To a solution of N-Boc intermediate 11b (280 mg, 0.358 mmol) in ethyl acetate (4 ml) was added HCl-dioxane (4N, 0.9 ml, 3.6 mmol) and the reaction was kept overnight. After volatiles were removed under vacuum the residue was crystallized from ether to provide the title hydrochloric salt 11c as a yellow solid. Yield: 244 mg (95%).

Preparation of compound 101: To a solution of hydrochloride 11c (50 mg, 0.07 mmol) in DMF (3 ml) was added (S)-2-((tert-butoxycarbonyl)amino)non-8-enoic acid 8d (28 mg, 0.104 mmol), DIPEA (0.061 ml, 0.35 mmol) and HATU 39.5 mg (0.104 mmol). Reaction was stirred for 1 h at room temperature and quenched with water. After it was acidified to pH ˜3 it was extracted with ethyl acetate. Organic phase was washed with brine, dried over magnesium sulfate and concentrated under vacuum. The residue was purified by column chromatography in 25-70% ethyl acetate-hexane to provide 55 mg (84%) of the target diene intermediate 11d.

Compound 11d (55 mg, 0.059 mmol) was dissolved in toluene (10 ml). After being degassed by bubbling of argon through the solution at 65° C. for 15 min, Zhan catalyst (2 mg, 0.003 mmol) was added to the solution. The reaction was kept for 40 min at 65° C. with continuous stream of argon and after 40 min more Zhan catalyst (1.5 mg, 0.002 mmol) was added. After 40 min the reaction was cooled down and concentrated under reduced pressure. The compound 101 was isolated as off-white foam by column chromatography in 20-40% acetone-hexane. Yield: 30 mg (56%). ¹H-NMR (chloroform-d), δ: 10.20 (br s, 1H), 7.95 (d, 1H), 7.59 (s, 1H), 7.21 (d, 1H), 7.11 (br. s, 1H), 7.05 (s, 1H), 5.74 (dt, 1H), 5.30 (dd, 1H), 5.10 (br. d, 1H), 5.01 (dd, 1H), 4.59 (dd, 1H), 4.37 (dd, 1H), 4.29 (s, 1H), 3.97 (s, 3H), 3.91 (dd, 1H), 3.20 (m, 1H), 2.70 (s, 3H), 2.64-2.52 (m, 2H), 2.32 (dd, 1H), 2.20-1.25 (m, 36H), 0.82 (m, 2H).

### Example 6

### Synthesis of 4-Methyl Proline Macrocyclic Analogs

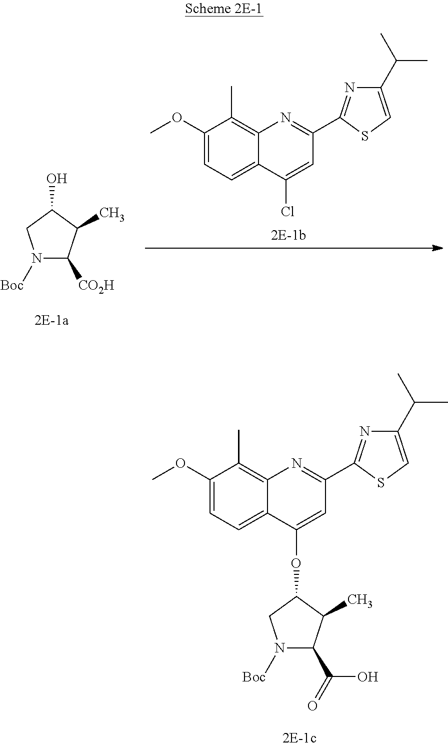

### Compound 2E-1c

To a solution of Compound 2E-1a (73 mg, 0.3 mmol) and Compound 2E-11 (131 mg, 0.39 mmol) in DMSO (2 ml) was added potassium tert-butoxide (74 mg, 0.66 mmol) and the reaction was allowed to stir for 6 h at room temperature. Water (10 ml) was added to the reaction mixture followed by 2 N aqueous HCl to pH ˜3 (0.25 ml). The mixture was extracted with ethyl acetate, organic extract was washed with brine, dried over potassium sulfate and concentrated under vacuum. The residue was purified by column chromatography in 1-10% MeOH-DCM to provide Compound 2E-1c. Yield 141 mg (87%). ¹H-NMR (chloroform-d), 60° C., δ: 7.96 (d, 1H), 7.50 (s, 1H), 7.19 (d, 1H), 6.98 (s, 1H), 4.98 (m, 1H), 4.55 (m, 1H), 4.19 (m, 1H), 3.96 (s, 3H), 3.20 (m, 1H), 2.96 (m, 1H), 2.68 (s, 3H), 1.45 (s, 9H), 1.36 (d, 6H), 1.27 (d, 3H).

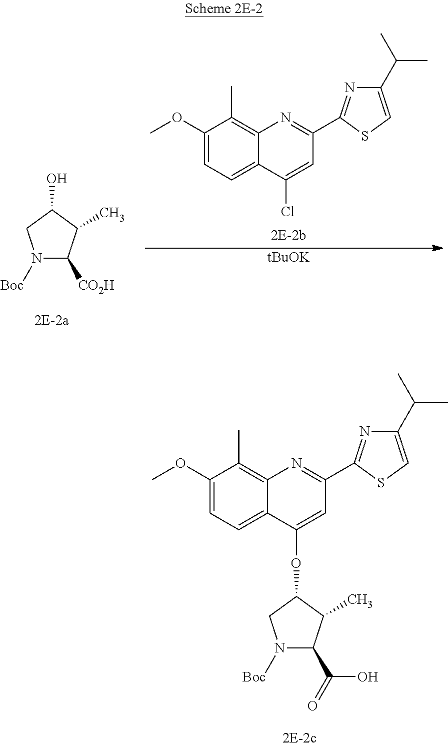

### Compound 2E-2c

To a stirred solution of Compound 2E-2a (100 mg, 0.408 mmol) in DMSO (2 ml) was added Compound 2E-2b (176 mg, 0.53 mmol) followed by addition of potassium tert-butoxide (105 mg, 0.94 mmol). The reaction was allowed to proceed overnight at room temperature, when it was quenched with water and acidified with aqueous hydrochloric acid to pH ˜2. The mixture was extracted with ethyl acetate; organic phase was washed with water, dried over magnesium sulfate and the solvent was removed under reduced pressure. The residue was purified by column chromatography in 0-15% methanol in DCM to afford Compound 2E-2c. Yield: 196 mg (89%). ¹H-NMR (DMSO-d⁶, 70° C.), δ: 12.05-12.80 (br. s, 1H), 8.00 (d, 1H), 7.51 (s, 1H), 7.43 (d, 1H), 7.40 (s, 1H), 5.33 (m, 1H), 4.01 (d, 1H), 3.95 (s, 3H), 3.67-3.80 (m, 2H), 3.15 (m, 1H), 2.62-2.72 (m, 1H), 2.56 (s, 3H), 1.31-1.35 (m, 15H), 1.25 (d, 3H).

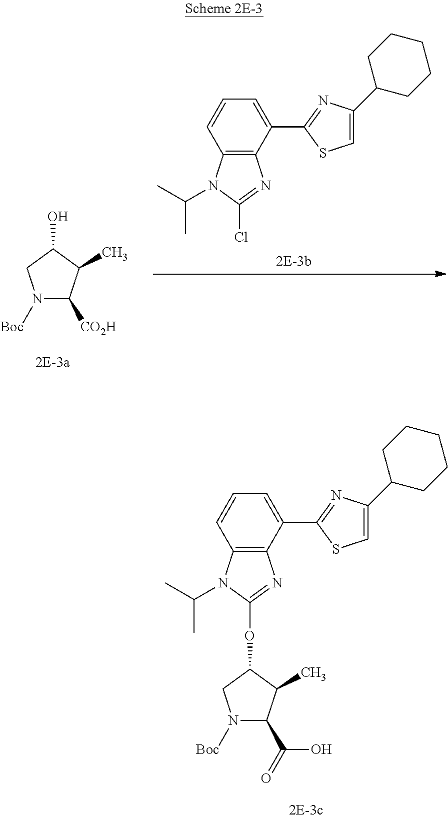

### Compound 2E-3c

To a stirred solution of Compound 2E-3a (140 mg, 0.57 mmol) and 2-(2-chloro-1-isopropyl-1H-benzo[d]imidazol-4-yl)-4-cyclohexylthiazole (2E-31, 206 mg, 0.57 mmol) in DMSO (3 ml) was added potassium tert-butoxide (147 mg, 1.31 mmol) and the reaction was allowed to stir for 2 hours at room temperature followed by stirring at 40° C. for 2 hours. The reaction was partitioned between ethyl acetate-water and acidified with 2 N aqueous hydrochloric acid to pH ˜2. The organic phase was separated, washed with water, dried over magnesium sulfate and concentrated under reduced pressure. The residue was purified by column chromatography in 0-10% methanol-DCM to afford Compound 2E-3c. Yield 264 mg (81%). ¹H-NMR (DMSO-d⁶, 70° C.), δ: 12.52 (br. s, 1H), 7.97 (d, 1H), 7.51 (d, 1H), 7.28 (s, 1H), 7.20 dd, 1H), 5.27 (m, 1H), 4.72 (m, 1H), 4.34 (d, 1H), 4.05-4.27 (m, 1H), 3.58 (dd, 1H), 3.10 (m, 1H), 2.87-2.98 (m, 1H), 2.79 (m, 1H), 2.03-2.10 (m, 2H), 1.78-1.84 (m, 2H), 1.68-1.76 (m, 1H), 1.52 (d, 3H), 1.49 (d, 3H), 1.39 (s, 9H), 1.14 (d, 3H).

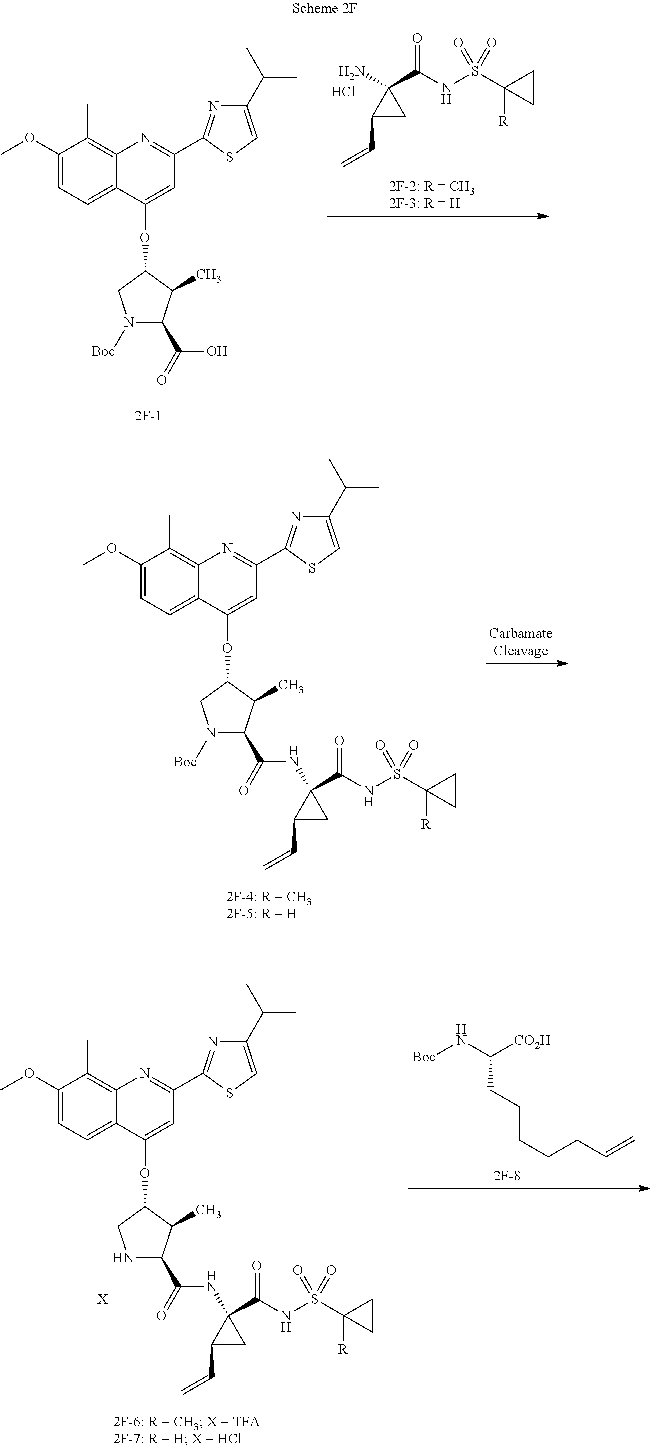

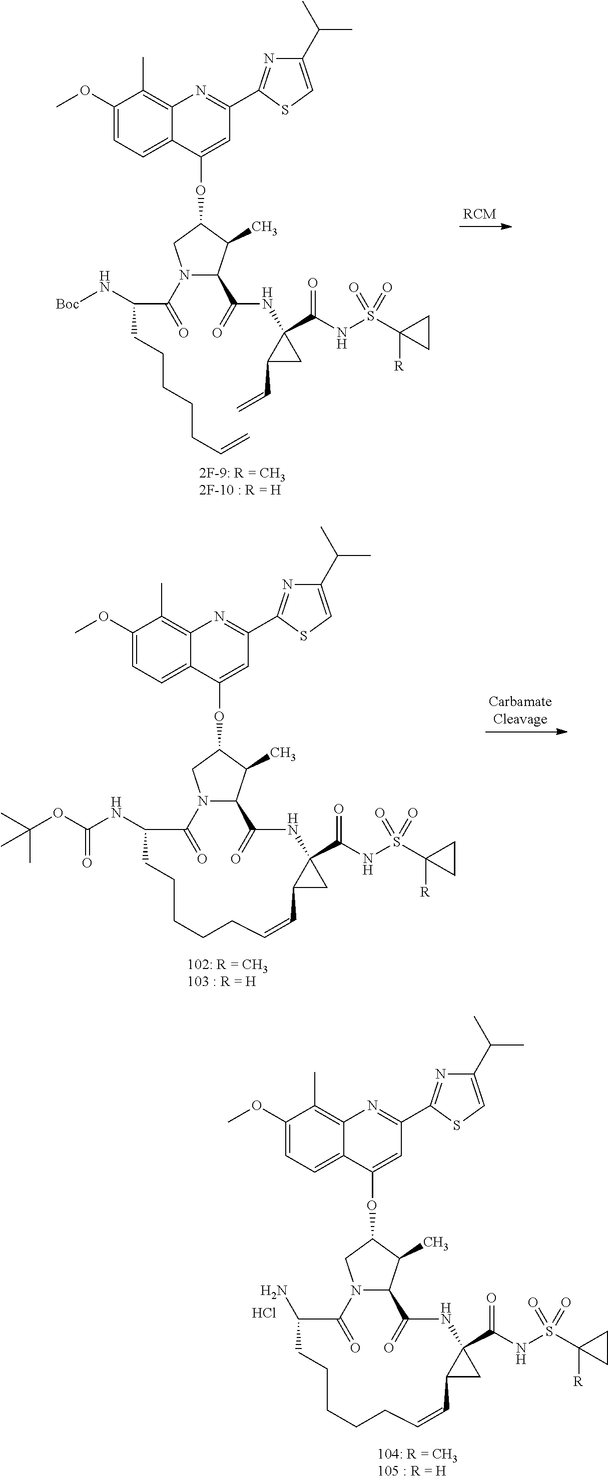

### Compound 2F-4:

To a solution of Compound 2f-1 (140 mg, 0.26 mmol) and (1R,2S)-1-amino-N-((1-methylcyclopropyl)sulfonyl)-2-yinylcyclopropanecarboxamide hydrochloride (2F-2, 109 mg, 0.39 mmol) in DMF (5 ml) was added DIPEA (0.23 ml, 1.3 mmol) followed by HATU (148 mg, 0.39 mmol). The reaction mixture was stirred for 2 hours at room temperature, diluted with water and acidified to pH ˜3 with 2N hydrochloric acid (0.6 ml) and extracted with ethyl acetate. The organic phase was washed with brine, dried over magnesium sulfate and concentrated under reduced pressure. The residue was purified by column chromatography in 20-40% acetone-hexane to afford Compound 2F-4. Yield: 174 mg (87%). ¹H-NMR (chloroform-d, 60° C.) δ: 9.65 (br. s, 1H), 7.93 (d, 1H), 7.52 (s, 1H), 7.20 (d, 1H), 7.01 (s, 1H), 5.76 (m, 1H), 5.31 (d, 1H), 5.17 (d, 1H), 5.05 (m, 1H), 4.43 (d, 1H), 4.14 (dd, 1H), 3.96 (s, 3H), 3.67 (m, 1H), 3.21 (m, 1H), 2.95 (m, 1H), 2.69 (s, 3H), 2.18 (dd, 1H), 1.94 (dd, 1H), 1.65 (m, 2H), 1.53 (s, 3H), 1.47 (s, 9H), 1.39 (d, 6H), 1.20 (d, 3H), 0.82 (m, 2H).

### Compound 2F-6:

To a stirred solution of Compound 2F-4 (174 mg, 0.226 mmol) in DCM (2 ml) was added TFA (0.75 ml) and the reaction was allowed to proceed for 2 hours at room temperature. The reaction mixture was concentrated under reduced pressure and co-evaporated with toluene to give Compound 2F-6 which was used in subsequent reactions without any additional purification. Yield 191 mg (100%).

### Compound 2F-5

Compound 2F-1 (540 mg, 1 mmol) was converted to Compound 2F-5 as described for the synthesis of Compound 2F-4. Yield 670 mg (89%). ¹H-NMR (chloroform-d, 60° C.) δ: 9.77 (s, 1H), 7.92 (d, 1H), 7.51 (s, 1H), 7.21 (d, 1H), 7.01 (s, 1H), 6.66 (br. s, 1H), 5.75-5.85 (m, 1H), 5.31 (d, 1H), 5.17 (d, 1H), 5.06 (m, 1H), 4.40 (d, 1H), 4.09-4.14 (m, 1H), 3.97 (s, 3H), 3.65-3.75 (m, 1H), 3.21 (m, 1H), 2.89-3.00 (m, 2H), 2.70 (s, 3H), 2.18 (dd, 1H), 1.97 (dd, 1H), 1.47 (s, 9H), 1.39 (d, 6H), 1.18 (d, 3H), 0.95-1.15 (m, 2H).

### Compound 2F-7:

Compound 2F-7 was synthesized from Compound 2F-5 (670 mg, 0.89 mmol) as described for the synthesis of Compound 2F-6 using 4 N HCl-dioxane for BOC group cleavage. Yield 570 mg (92.9%). ¹H-NMR (DMSO-d⁶), δ: 11.73 (s, 1H), 10.95 (m, ex, 1H), 9.27 (s, 1H), 9.12 (m, ex., 1H), 8.19 (d, 1H), 7.50 (d, 1H), 7.48 (s, 1H), 7.47 (d, 1H), 5.47-5.56 (m, 1H), 5.43 (d, 1H), 5.26 (dd, 1H), 5.10 (dd, 1H), 4.64 (m, 1H), 3.98 (s, 3H), 3.61-3.65 (m, 1H), 3.11-3.20 (m, 2H), 2.90-3.00 (m, 1H), 2.60 (s, 3H), 2.28 (m, 1H), 1.85 (dd, 1H), 1.35 (d, 6H), 1.23 (dd, 1H), 0.97-1.12 (m, 4H), 0.94 (d, 3H).

### Compound 2F-9

(S)-2-((tert-butoxycarbonyl)amino)non-8-enoic acid (2F-8, 33 mg, 0.123 mmol) was combined with a solution of Compound 2F-6 (64 mg, 0.082 mmol) in DMF (2 ml). DIPEA (0.21 ml, 1.23 mmol) was added to the solution followed by addition of HATU (47 mg, 0.123 mmol). The reaction was allowed to proceed for 3 hours at room temperature when it was diluted with water (10 ml), acidified to pH 2 with 2N hydrochloric acid (0.5 ml) and extracted with ethyl acetate. Organic solution was washed with brine, dried over magnesium sulfate and concentrated under reduced pressure. The residue was purified by column chromatography in 20-30% acetone-hexane to provide Compound 2F-9. Yield 54 mg (71.5%). ¹H-NMR (chloroform-d), δ: 9.70 (s, 1H), 7.94 (d, 1H), 7.50 (s, 1H), 7.41 (br. s, 1H), 7.19 (d, 1H), 7.04 (d, 1H), 5.70-5.86 (m, 2H), 5.27-5.34 (m, 2H), 5.12-5.17 (m, 2H), 4.87-4.97 (m, 2H), 4.62 (d, 1H), 4.34 (dd, 1H), 4.25 (dd, 1H), 4.02 (dd, 1H), 3.97 (s, 3H), 3.20 (m, 1H), 2.98 (m, 1H), 2.69 (m, 1H), 2.19 (dd, 1H), 1.96 (m, 3H), 1.56-1.71 (m, 5H), 1.51 (s, 3H), 1.25-1.43 (m, 29H), 1.18 (d, 3H), 0.80-0.90 (m, 2H).

### Compound 2F-10:

Compound 2F-10 (150 mg, 0.217 mmol) was prepared as described for the synthesis of Compound 2F-9. Yield 177 mg (89.9%). ¹H-NMR (chloroform-d) δ: 9.90 (s, 1H), 7.92 (d, 1H), 7.50 (s, 1H), 7.20 (d, 1H), 7.06 (br. s, 1H), 7.04 (s, 1H), 5.87 (m, 1H), 5.74 (m, 1H), 5.14-5.32 (m, 3H), 4.88-4.97 (m, 2H), 4.57 (d, 1H), 4.31 (m, 1H), 4.23 (m, 1H), 4.02 (dd, 1H), 3.98 (s, 3H), 3.20 (m, 1H), 2.91-3.15 (m, 2H), 2.78 (s, 3H), 2.17 (m, 1H), 1.94-2.01 (m, 3H), 1.50-1.78 (m, 3H), 1.22-1.44 (m, 25H), 1.18 (d, 3H), 1.05 (m, 2H).

### Compound 102:

A solution of Compound 2F-9 (54 mg, 0.059 mmol) in toluene (10 ml) was degassed by bubbling argon for 15 min at 65° C. To this solution was added Zhan catalyst (2.3 mg, 0.003 mmol) as a single portion and the reaction mixture was stirred at 65° C. for 30 minutes with argon gas bubbling. The reaction mixture was left to cool down to ambient temperature and the solvent removed in vacuo. The residue was purified by column chromatography in 20 to 25% acetone-hexane to afford Compound 102. Yield 36 mg (68%). ¹H-NMR (chloroform-d), δ: 10.30 (s, 1H), 7.96 (d, 1H), 7.55 (s, 1H), 7.35 (s, 1H), 7.18 (d, 1H), 7.04 (s, 1H), 5.18 (m, 1H), 5.22-5.28 (m, 2H), 4.99 (m, 1H), 4.60-4.70 (m, 2H), 4.38 (dd, 1H), 3.97 (s, 1H), 3.86 (dd, 1H), 3.19 (m, 1H), 2.96 (m, 1H), 2.70 (s, 3H), 2.56 (m, 1H), 2.29 (m, 1H), 1.70-1.98 (m, 5H), 1.47-1.53 (m, 9H), 1.39 (d, 6H), 1.31-1.35 (m, 13H), 0.82 (m, 2H). LCMS (M+1)⁺: 893.7.

### Compound 103:

Compound 2F-10 (177 mg, 0.195 mmol) was converted to Compound 103 as described for the synthesis of Compound 102. Yield 140 mg (81.7%). ¹H-NMR (chloroform-d) δ: 10.14 (s, 1H), 7.98 (d, 1H), 7.55 (s, 1H), 7.20 (d, 1H), 7.04 (s, 1H), 6.60 (br. s, 1H), 5.74 (dt, 1H), 5.28 (dd, 1H), 5.05 (d, 1H), 5.00 (dd, 1H), 4.57-4.61 (m, 2H), 4.32-4.39 (m, 1H), 3.98 (s, 1H), 3.91-3.98 (m, 1H), 3.19 (m, 1H), 2.84-3.03 (m, 2H), 2.70 (s, 3H), 2.26-2.38 (m, 1H), 1.86-2.00 (m, 3H), 1.42-1.67 (m, 9H), 1.38 (d, 6H), 1.35 (s, 9H), 1.33 (d, 3H), 1.02-1.25 (m, 5H), 0.95-1.00 (m, 2H).

### Compound 104:

To a stirred solution of Compound 102 (50 mg, 0.056 mmol) in DCM (1 ml) was added 4 M HCl-dioxane (0.3 ml, 0.12 mmol). The reaction mixture was allowed to stir for 2 hours and the precipitated material was filtered, washed with ether and dried under reduced pressure to afford Compound 104. Yield 43 mg (92.6%). ¹H-NMR (DMSO-d⁶), δ: 10.63 (br. s, 1H), 9.54 (s, 1H), 8.29 (br. s, 3H), 8.09 (d, 1H), 7.56 (s, 1H), 7.49 (s, 1H), 7.46 (d, 1H), 5.66 (m, 1H), 5.27 (m, 1H), 4.98 (dd, 1H), 4.76 (d, 1H), 4.44 (m, 1H), 4.33 (m, 1H), 4.00 (m, 1H), 3.97 (s, 3H), 3.16 (m, 1H), 3.00 (m, 1H), 2.60 (s, 3H), 2.30-2.40 (m, 1H), 2.11 (dd, 1H), 1.98-2.08 (m, 1H), 1.76-1.85 (m, 1H), 1.68 (dd, 1H), 1.43-1.50 (m, 6H), 1.41 (s, 3H), 1.35 (d, 6H), 1.27-1.32 (m, 3H), 1.24 (d, 3H), 0.91 (m, 2H).

### Compound 105:

Compound 105 was prepared from Compound 103 as described for the synthesis of Compound 104. ¹H-NMR (DMSO-d⁶), δ: 10.58 (s, 1H), 9.39 (s, 1H), 8.29 (s, 3H), 8.09 (d, 1H), 7.55 (s, 1H), 7.49 (s, 1H), 7.46 (d, 1H), 5.65 (dt, 1H), 5.26 (m, 1H), 5.03 (dd, 1H), 4.72 (d, 1H), 4.28 (m, 1H), 4.33 (m, 1H), 3.97 (s, 3H), 3.15 (m, 1H), 2.88-3.01 (m, 2H), 2.59 (s, 3H), 2.30-2.39 (m, 1H), 2.11 (dd, 1H), 1.98-2.08 (m, 1H), 1.76-1.88 (m, 2H), 1.68 (dd, 1H), 1.39-1.53 (m, 5H), 1.35 (d, 6H), 1.25-1.34 (m, 3H), 1.21 (d, 3H), 0.97-1.14 (m, 5H).

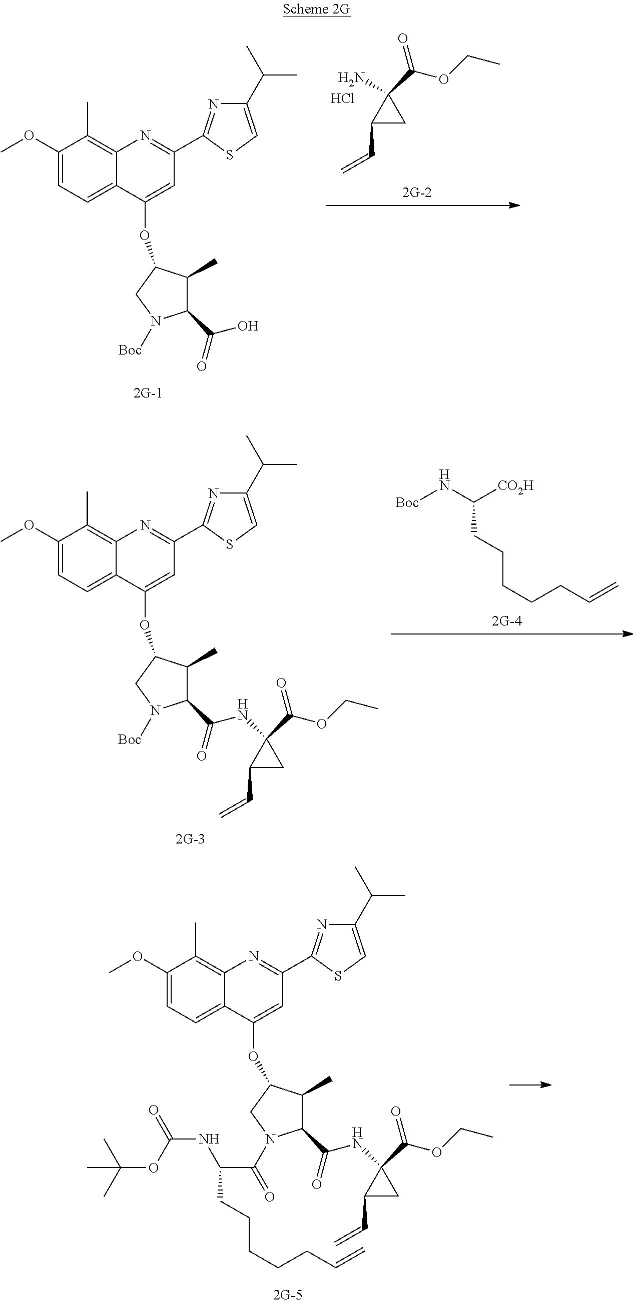

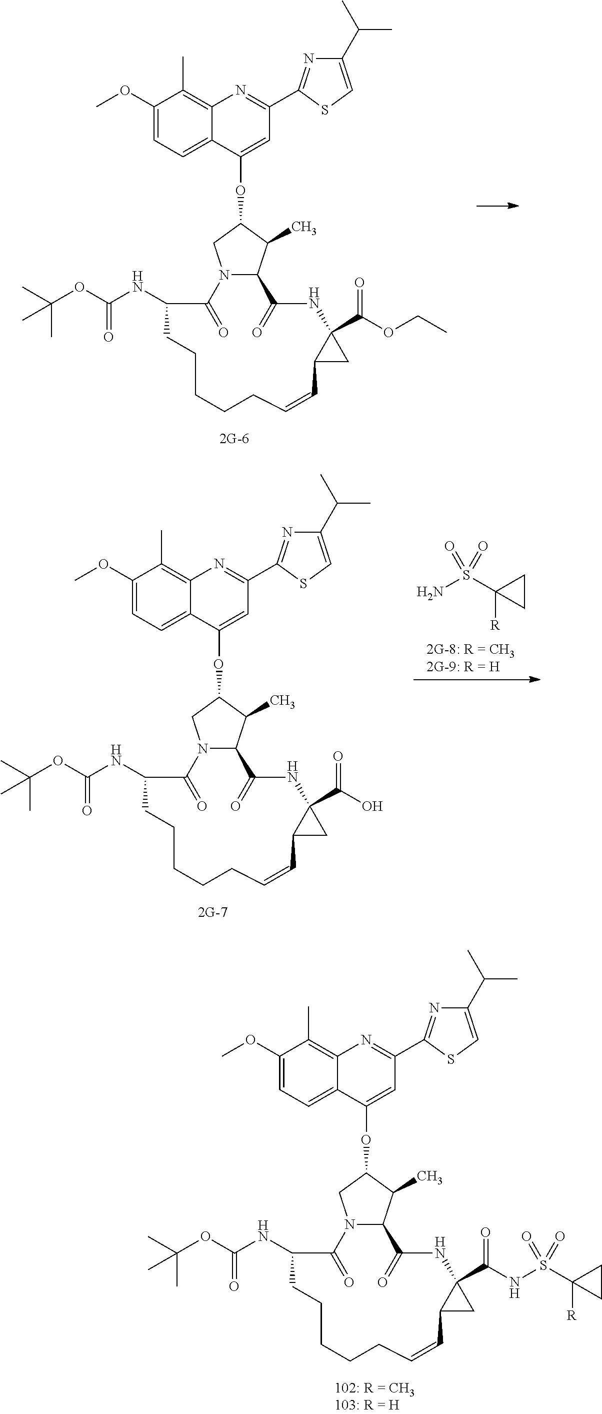

### Compound 2G-3:

To a stirred at 0° C. solution of Compound 2G-1 (30.24 g, 55.8 mmol) and Compound 2G-2 (13 g, 90% pure, 61.4 mmol) in DMF (200 ml) was added DIPEA. After 5 min HATU (24.4 g, 64.2 mmol) was added and the reaction was allowed to proceed for 1 hour at room temperature. The reaction mixture was diluted with water and ethyl acetate and acidified with 2 N hydrochloric acid to pH 2 (85 ml). Organic phase was separated; water phase was back-extracted 3 times with ethyl acetate. Combined organic solution was washed with water, 5% sodium bicarbonate and brine. The resulted organic solution was dried over magnesium sulfate and the solvent was removed under reduced pressure to afford Compound 2G-3 which was used on the next step without any further purification. Yield 42.6 g (100%), ˜90% purity (NMR). ¹H-NMR (chloroform-d), δ: 7.95 (d, 1H), 7.50 (s, 1H), 7.21 (d, 1H), 7.02 (s, 1H), 6.69 (s, 1H), 5.78 (ddd, 1H), 5.32 (dd, 1H), 5.15 (d, 1H), 5.07 (m, 1H), 4.42 (d, 1H), 4.10-4.22 (m, 4H), 3.98 (s, 3H), 3.21 (m, 1H), 2.70 (s, 3H), 2.11 (dd, 1H), 1.92-1.96 (m, 1H), 1.58 (dd, 1H), 1.43 (s, 9H), 1.40 (d, 3H), 1.38 (d, 3H), 1.28 (d, 3H), 1.24 (t, 3H).

### Compound 2G-5

To a stirred at 0° C. solution of Compound 2G-3 (34.35 g, 55.9 mmol) and (S)-2-((tert-butoxycarbonyl)amino)non-8-enoic acid (2G-4, 16.7 g, 61.4 mmol) in DMF (300 ml) was added DIPEA 49 ml (279.5 mmol) followed by HATU (24.4 g, 64.3 mmol). The reaction was allowed to proceed for 1 hour at room temperature. The reaction mixture was taken into ethyl acetate-water and acidified with 2 N aqueous hydrochloric acid to pH ˜2. Aqueous phase was separated and extracted 3 times with ethyl acetate. Combined organic phase was washed with water, 5% aqueous sodium bicarbonate and brine. The organic solution was dried over magnesium sulfate and the solvent was removed under reduced pressure to afford Compound 2G-5 which was used on the next step without any further purification. Yield 48.7 g (94%, 90% purity by NMR). ¹H-NMR (chloroform-d), δ: 7.94 (d, 1H), 7.50 (s, 1H), 7.20 (d, 1H), 7.03 (s, 1H), 6.85 (s, 1H), 5.65-5.83 (m, 2H), 5.29 (dd, 1H), 5.22 (m, 1H), 5.11 (dd, 1H), 5.01 (dd, 1H), 4.88-4.96 (m, 2H), 4.67 (d, 1H), 4.06-4.27 (m, 4H), 3.98 (s, 3H), 3.95 (dd, 1H), 3.20 (m, 1H) 2.88 (m, 1H), 2.69 (s, 3H), 2.21 (dd, 1H), 1.95 (m, 2H), 1.89 (dd, 1H), 1.58-1.69 (m, 2H), 1.51 (dd, 1H), 1.20-1.45 (m, 32H).

### Compound 2G-6

A heated to 70° C. solution of Compound 2G-5 (47.3 g, 56 mmol) in toluene (4.8 L) was deoxygenated by passing a stream of argon for 25 min. To this solution was added Zhan catalyst portionwise over two hours (3.4 g, 5 mmol). The reaction was quenched by addition of imidazole (2 g) and the solvent was removed under reduced pressure. The residue was purified by column chromatography in 20-40% acetone-hexane to afford Compound 2G-6. Yield 33.8 g (75%). ¹H-NMR (chloroform-d), δ: 7.99 (d, 1H), 7.58 (s, 1H), 7.22 (d, 1H), 7.02 (s, 1H), 6.45 (s, 1H), 5.68 (dt, 1H), 5.43 (dd, 1H), 5.22-5.35 (m, 2H), 5.57-4.67 (m, 2H), 4.44 (m, 1H), 4.08-4.25 (m, 3H), 3.99 (s, 3H), 3.82 (dd, 1H), 3.16 (m, 1H), 2.93 (m, 1H), 2.71 (s, 3H), 2.44 (m, 1H), 2.23 (dd, 1H), 2.05-2.12 (m, 1H), 1.80-1.92 (m, 1H), 1.61-1.74 (m, 3H), 1.30-1.52 (m, 23H), 1.22 (t, 3H).

### Compound 2G-7

To a stirred at 45° C. solution of Compound 2G-6 (33.8 g, 42 mmol) in ethanol (120 ml) was added 2 N aqueous sodium hydroxide (105 ml, 210 mmol) dropwise over 2 hours. The reaction was allowed to proceed for another 2 h at 45° C. and then left overnight at room temperature. The reaction mixture was concentrated under reduced pressure; the residue was taken into water-ethyl acetate and the mixture was acidified to pH ˜2 with 2 N aqueous hydrochloric acid. Aqueous phase was separated and extracted with ethyl acetate. The combined organic solution was washed with water, dried over magnesium sulfate and the solvent was removed under vacuum to afford Compound 2G-7 which was used on the next step without any further purification. Yield 32.1 g (98.5%). ¹H-NMR (DMSO-d⁶), δ: 12.29 (s, 1H), 8.81 (s, 1H), 8.08 (d, 1H), 7.52 (s, 1H), 7.48 (s, 1H), 7.43 (d, 1H), 6.89 (d, 1H), 5.53 (dt, 1H), 5.35 (dd, 1H), 5.16 (m, 1H), 4.56 (d, 1H), 4.44 (dd, 1H), 4.16 (dd, 1H), 3.96 (s, 3H), 3.87 (dd, 1H), 3.15 (m, 1H), 2.87 (dd, 1H), 2.59 (s, 3H), 2.43 (m, 1H), 2.06 (dd, 1H), 1.86-1.98 (m, 1H), 1.62-1.73 (m, 1H), 1.39-1.52 (m, 6H), 1.35 (d, 6H), 1.27 (s, 9H), 1.24 (d, 3H).

### Compound 102

To a solution of Compound 2G-7 (16.0 g (20.6 mmol) in DCM (100 ml) was added CDI (4.01 g, 24.7 mmol). The reaction was kept for 3 hours at room temperature when 1-methylcyclopropane-1-sulfonamide (3.62 g, 26.8 mmol) was added followed by addition of DBU (4.0 ml, 26.8 mmol). The reaction was allowed to proceed overnight at 40° C. and then it was concentrated under reduced pressure. The residue was taken into ethyl acetate-water and the mixture was acidified with 2 N aqueous hydrochloric acid to pH ˜2. Organic phase was separated; aqueous layer was additionally extracted with ethyl acetate. The combined organic phase was washed with brine, dried over magnesium sulfate and the solvent was removed under vacuum. The residue was purified by column chromatography in 20-30% acetone-hexane to afford Compound 102. Yield: 13.7 g (74.4%). The analytical data was identical to the data described herein.

### Compound 103

Compound 103 was prepared as described for Compound 102 using cyclopropane-1-sulfonamide. Yield 14.3 g (79.0%). The analytical data was identical to the data described herein.

### Alternative Synthesis of Compounds 102 and 103

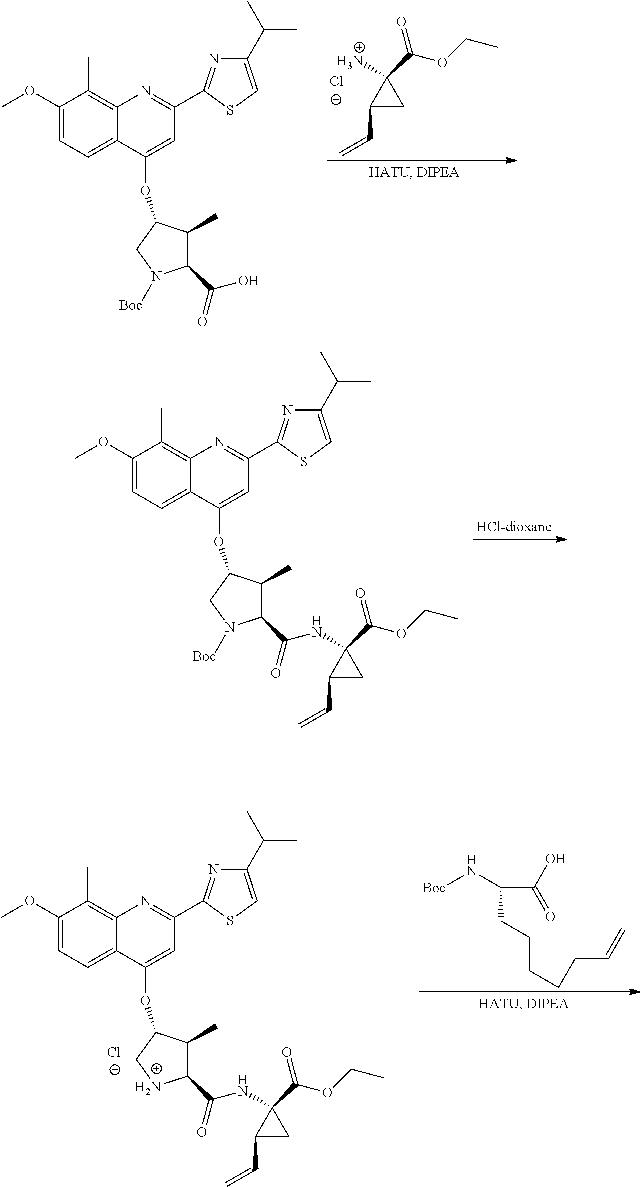

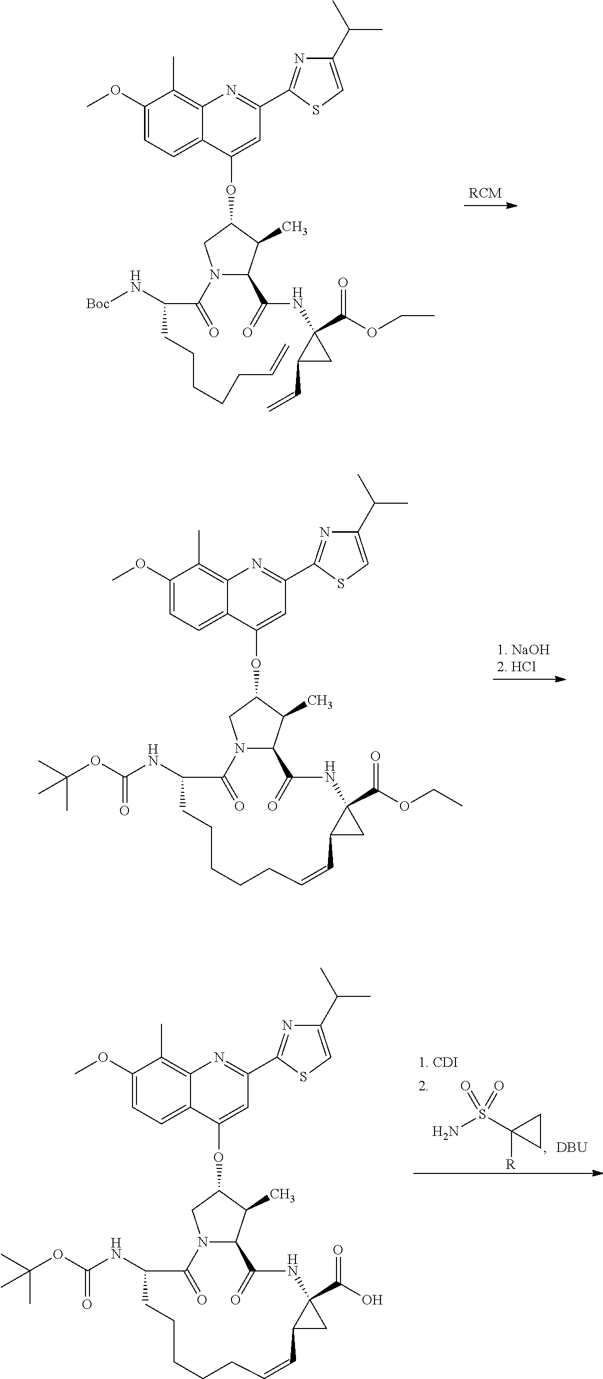

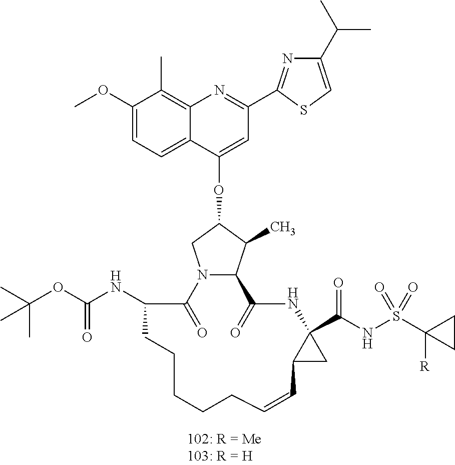

### Compound 2G-3

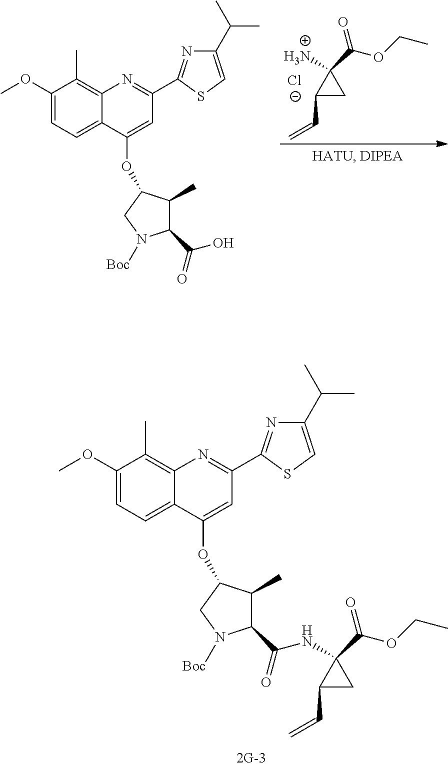

To a stirred at 0° C. solution of the MMQ-methyl proline intermediate (30.24 g, 55.8 mmol) and P1 amino hydrochloride (13 g, 90% pure, 61.4 mmol) in DMF (200 ml) was added DIPEA After 5 min HATU (24.4 g, 64.2 mmol) was added and the reaction was allowed to proceed for 1 hour at room temperature. The reaction mixture was diluted with water and ethyl acetate and acidified with 2N hydrochloric acid to pH 2 (85 ml). Organic phase was separated; water phase was back-extracted 3 times with ethyl acetate. Combined organic solution was washed with water, 5% sodium bicarbonate and brine. The resulted organic solution was dried over magnesium sulfate and the solvent was removed under reduced pressure to afford the title dipeptide as a beige foam which was used on the next step without any further purification.

Yield 42.6 g (100%), ˜90% purity (NMR). ¹H-NMR (chloroform-d), δ: 7.95 (d, 1H), 7.50 (s, 1H), 7.21 (d, 1H), 7.02 (s, 1H), 6.69 (s, 1H), 5.78 (ddd, 1H), 5.32 (dd, 1H), 5.15 (d, 1H), 5.07 (m, 1H), 4.42 (d, 1H), 4.10-4.22 (m, 4H), 3.98 (s, 3H), 3.21 (m, 1H), 2.70 (s, 3H), 2.11 (dd, 1H), 1.92-1.96 (m, 1H), 1.58 (dd, 1H), 1.43 (s, 9H), 1.40 (d, 3H), 1.38 (d, 3H), 1.28 (d, 3H), 1.24 (t, 3H).

### Compound 2G-3A

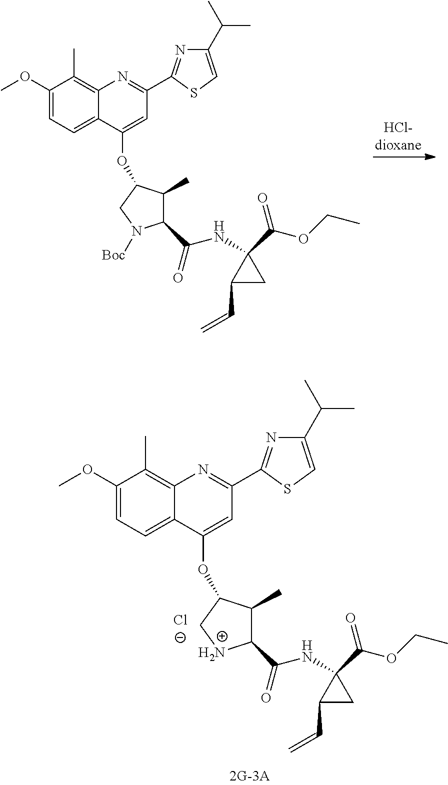

To a solution of the Boc intermediate from the previous step (32.0 g, 47.1 mmol) in DCM (150 ml) was added 4M HCl-dioxane (82 ml, 328 mmol). The reaction was stirred for one hour at room temperature and concentrated under reduced pressure. The residue was diluted with ethyl acetate with stirring and the resulted yellow solid was filtered off, washed with ethyl acetate and dried in vacuo.

Yield: 29.9 g (98%; 95% purity by HPLC). ¹H-NMR (DMSO-d⁶), δ: 10.98 (m, ex., 1H), 9.39 (s, 1H), 9.11 (m, ex., 1H), 8.22 (d, 1H), 7.50 (s, 1H), 7.49 (d, 1H), 7.48 (s, 1H), 5.66 (ddd, 1H), 5.45 (d, 1H), 5.26 (dd, 1H), 5.10 (dd, 1H), 4.48 (ddd, 1H), 4.06-4.13 (m, 2H), 4.00 (s, 3H), 3.92 (m, 1H) 3.62 (m, 1H), 3.15 (m, 1H), 2.60 (s, 3H), 2.19 (dd, 1H), 1.69 (dd, 1H), 1.35 (d, 6H), 1.15-1.19 (m, 3H), 1.05 (d, 3H).

### Compound 2G-5

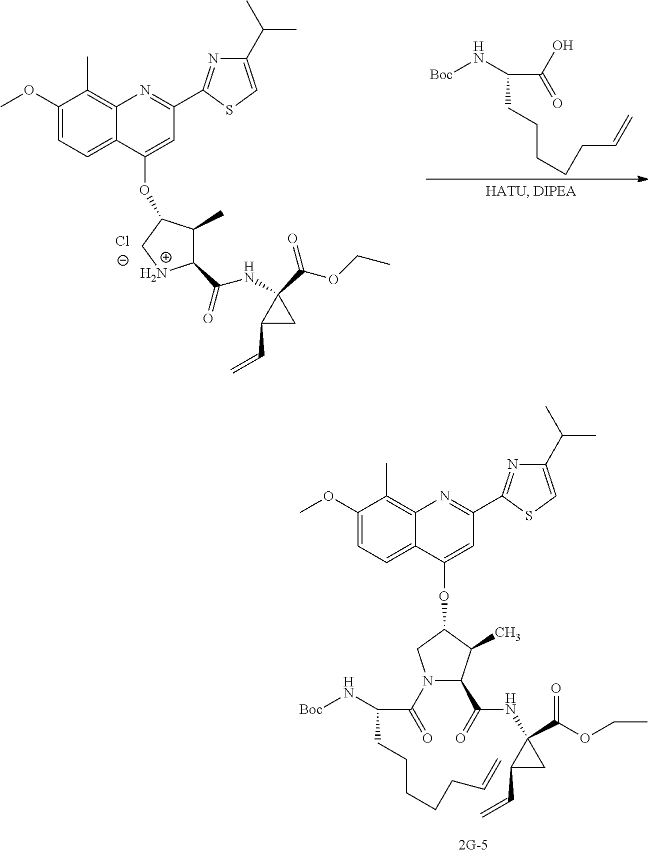

To a stirred at 0° C. solution of the amino hydrochloride from the previous step (34.35 g, 55.9 mmol) and (S)-2-((tert-butoxycarbonyl)amino)non-8-enoic acid (16.7 g, 61.4 mmol) in DMF (300 ml) was added DIPEA 49 ml (279.5 mmol) followed by HATU (24.4 g, 64.3 mmol). The reaction was allowed to proceed for 1 hour at room temperature. The reaction mixture was taken into ethyl acetate-water and acidified with 2N aqueous hydrochloric acid to pH˜2. Aqueous phase was separated and extracted 3 times with ethyl acetate. Combined organic phase was washed with water, 5% aqueous sodium bicarbonate and brine. The organic solution was dried over magnesium sulfate and the solvent was removed under reduced pressure to afford crude diene which was used on the next step without any further purification.

Yield 48.7 g (94%, 90% purity by NMR). ¹H-NMR (chloroform-d), δ: 7.94 (d, 1H), 7.50 (s, 1H), 7.20 (d, 1H), 7.03 (s, 1H), 6.85 (s, 1H), 5.65-5.83 (m, 2H), 5.29 (dd, 1H), 5.22 (m, 1H), 5.11 (dd, 1H), 5.01 (dd, 1H), 4.88-4.96 (m, 2H), 4.67 (d, 1H), 4.06-4.27 (m, 4H), 3.98 (s, 3H), 3.95 (dd, 1H), 3.20 (m, 1H) 2.88 (m, 1H), 2.69 (s, 3H), 2.21 (dd, 1H), 1.95 (m, 2H), 1.89 (dd, 1H), 1.58-1.69 (m, 2H), 1.51 (dd, 1H), 1.20-1.45 (m, 32H).

### Compound 2G-6

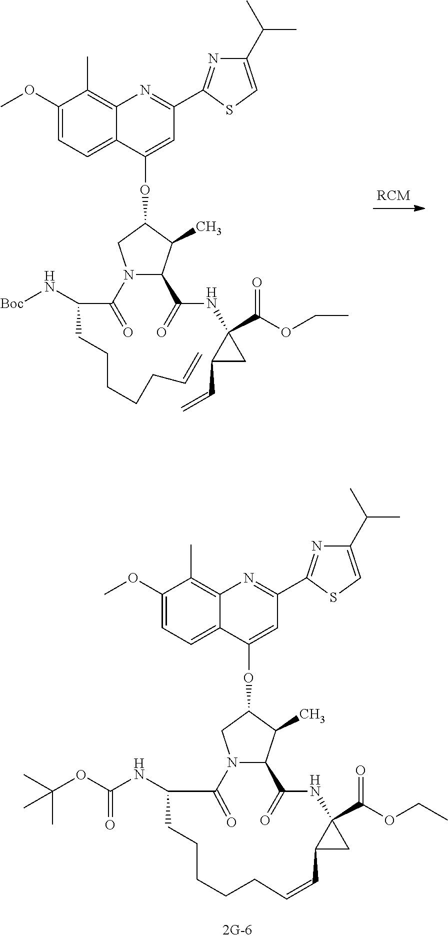

A heated to 70° C. solution of the diene from the previous step (47.3 g, 56 mmol) in toluene (4.8 L) was deoxygenated by passing a stream of argon for 25 min. To this solution was added Zhan catalyst portionwise over two hours (3.4 g, 5 mmol). The reaction was quenched by addition of imidazole (2 g) and the solvent was removed under reduced pressure. The residue was purified by column chromatography in 20-40% acetone-hexane to afford the target macro cycle as a beige foam.

Yield 33.8 g (75%). ¹H-NMR (chloroform-d), δ: 7.99 (d, 1H), 7.58 (s, 1H), 7.22 (d, 1H), 7.02 (s, 1H), 6.45 (s, 1H), 5.68 (dt, 1H), 5.43 (dd, 1H), 5.22-5.35 (m, 2H), 5.57-4.67 (m, 2H), 4.44 (m, 1H), 4.08-4.25 (m, 3H), 3.99 (s, 3H), 3.82 (dd, 1H), 3.16 (m, 1H), 2.93 (m, 1H), 2.71 (s, 3H), 2.44 (m, 1H), 2.23 (dd, 1H), 2.05-2.12 (m, 1H), 1.80-1.92 (m, 1H), 1.61-1.74 (m, 3H), 1.30-1.52 (m, 23H), 1.22 (t, 3H).

### Compound 2G-7

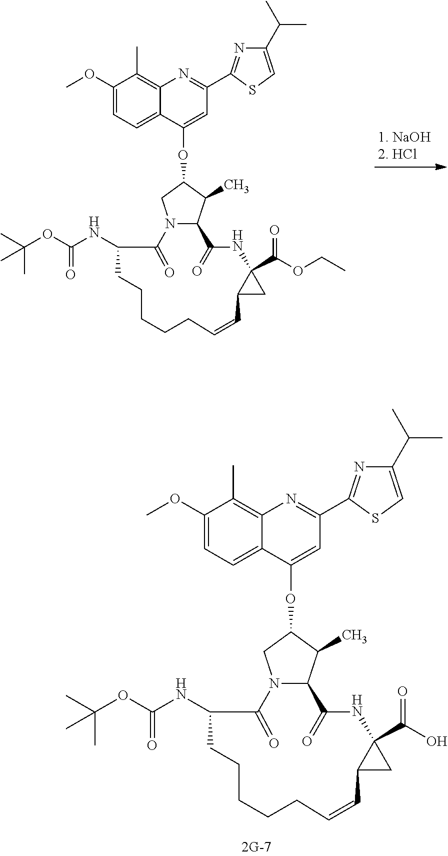

To a stirred at 45° C. solution of the ester from the previous step (33.8 g, 42 mmol) in ethanol (120 ml) was added 2N aqueous sodium hydroxide (105 ml, 210 mmol) dropwise over 2 hours. The reaction was allowed to proceed for another 2 h at 45° C. and then left overnight at room temperature. The reaction mixture was concentrated under reduced pressure; the residue was taken into water-ethyl acetate and the mixture was acidified to pH ˜2 with 2N aqueous hydrochloric acid. Aqueous phase was separated and extracted with ethyl acetate. The combined organic solution was washed with water, dried over magnesium sulfate and the solvent was removed under vacuum to afford the target carboxylic acid as a beige foam which was used on the next step without any further purification.

Yield 32.1 g (98.5%). ¹H-NMR (DMSO-d⁶), δ: 12.29 (s, 1H), 8.81 (s, 1H), 8.08 (d, 1H), 7.52 (s, 1H), 7.48 (s, 1H), 7.43 (d, 1H), 6.89 (d, 1H), 5.53 (dt, 1H), 5.35 (dd, 1H), 5.16 (m, 1H), 4.56 (d, 1H), 4.44 (dd, 1H), 4.16 (dd, 1H), 3.96 (s, 3H), 3.87 (dd, 1H), 3.15 (m, 1H), 2.87 (dd, 1H), 2.59 (s, 3H), 2.43 (m, 1H), 2.06 (dd, 1H), 1.86-1.98 (m, 1H), 1.62-1.73 (m, 1H), 1.39-1.52 (m, 6H), 1.35 (d, 6H), 1.27 (s, 9H), 1.24 (d, 3H).

### Compound 102

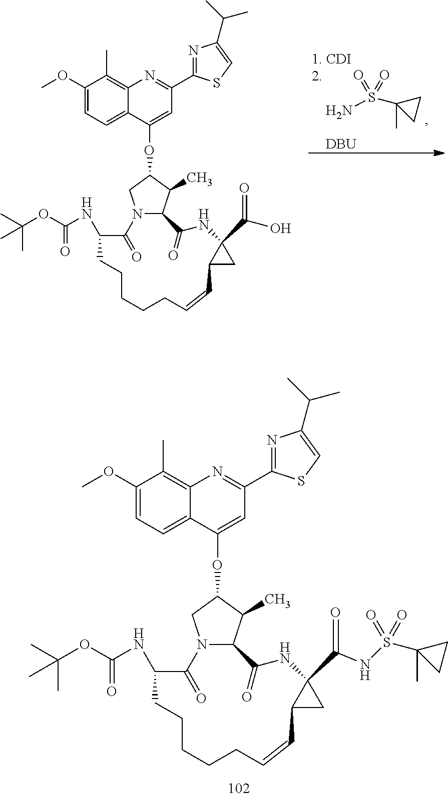

To a solution of the carboxylic acid from the previous step (16.0 g (20.6 mmol) in DCM (100 ml) was added CDI (4.01 g, 24.7 mmol). The reaction was kept for 3 hours at room temperature when 1-methylcyclopropane-1-sulfonamide (3.62 g, 26.8 mmol) was added followed by addition of DBU (4.0 ml, 26.8 mmol). The reaction was allowed to proceed overnight at 40° C. and then it was concentrated under reduced pressure. The residue was taken into ethyl acetate-water and the mixture was acidified with 2N aqueous hydrochloric acid to pH ˜2. Organic phase was separated; aqueous layer was additionally extracted with ethyl acetate. The combined organic phase was washed with brine, dried over magnesium sulfate and the solvent was removed under vacuum. The residue was purified by column chromatography in 20-30% acetone-hexane to afford the target sulfonamide as off-white foam.

Yield: 13.7 g (74.4%). Analytical data is identical to described before in this application.

### Compound 103

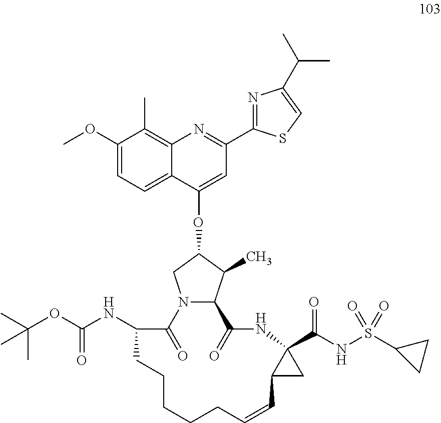

The target sulfonamide was synthesized as described in example 1 step 6 from carboxylic acid intermediate (16.0 g, 20 6 mmol) using cyclopropane-1-sulfonamide.

Yield 14.3 g (79.0%). Off-white foam; analytical data is identical to described before in this application.

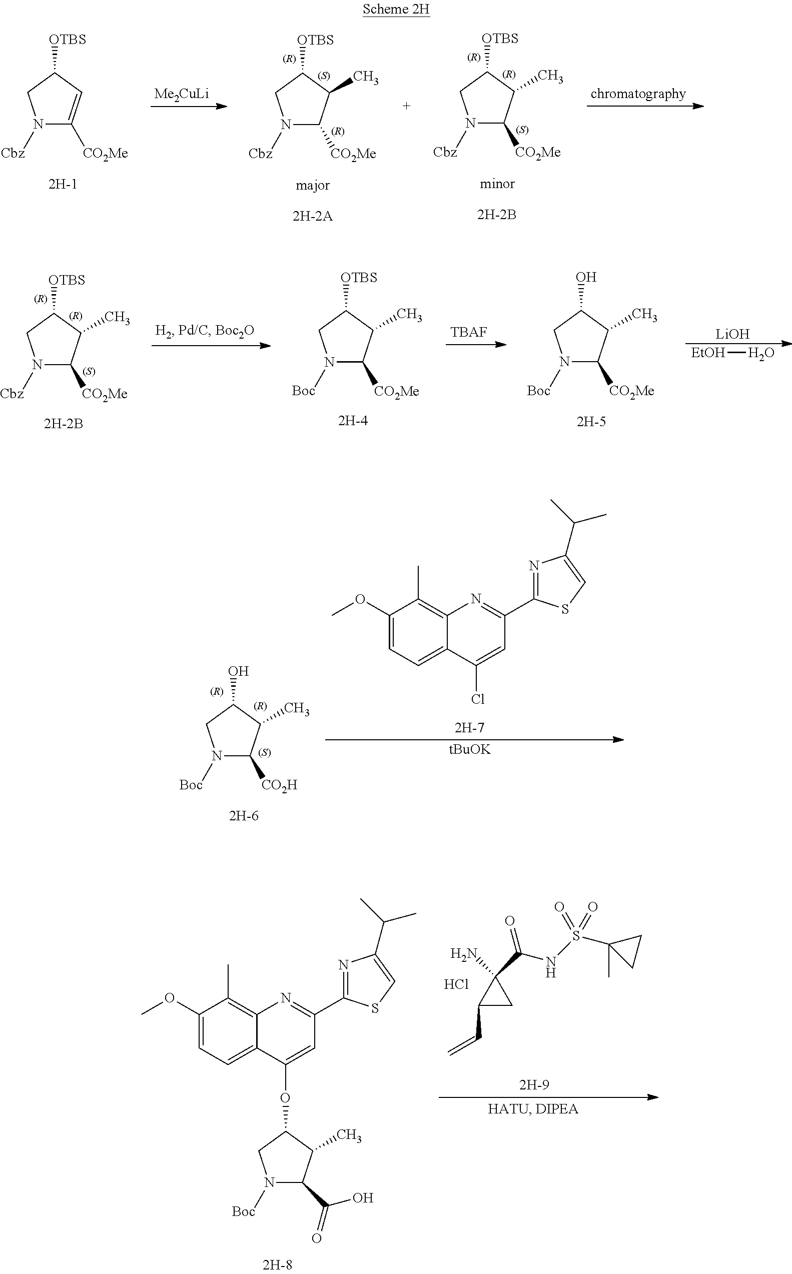

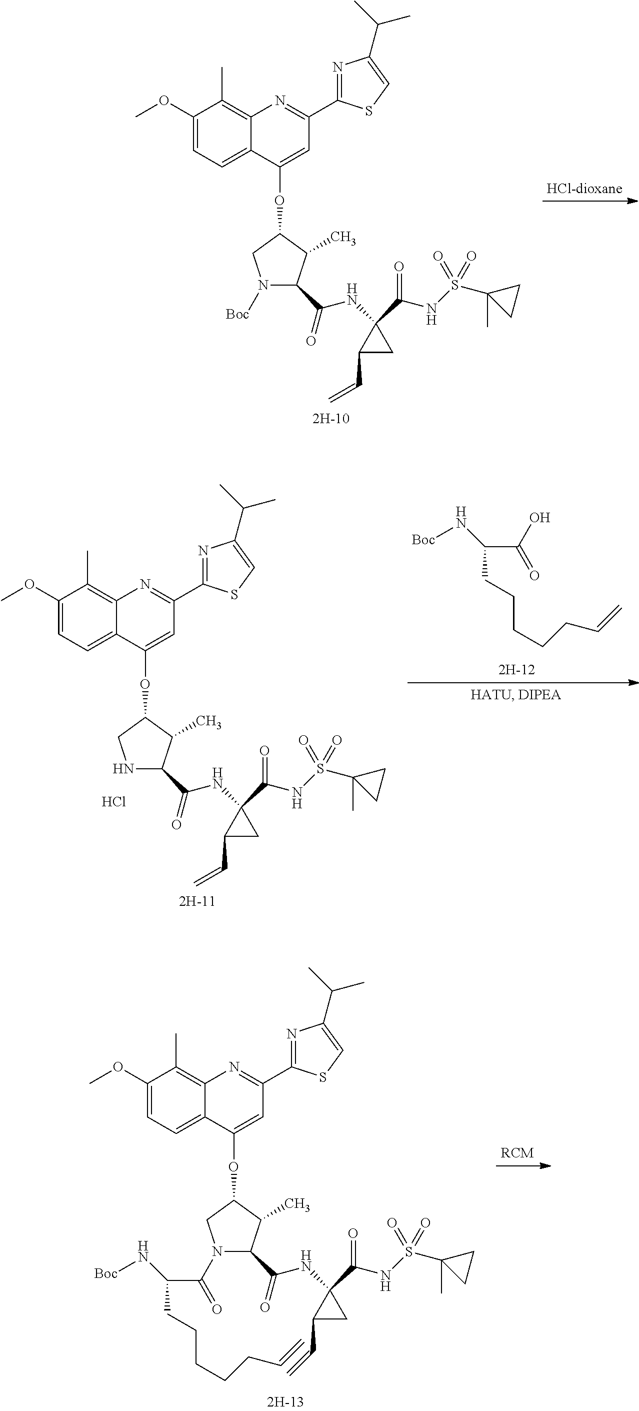

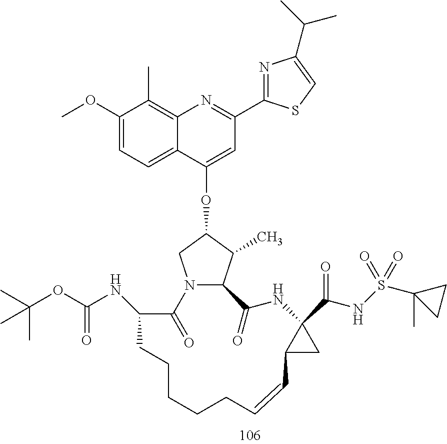

### Compound 2H-2B

The mother liquor after first crystallization of (2R,3S,4R)-diastereoisomer was concentrated under reduced pressure to give ˜36 g of an oily residue. Approximately 5 g of this oil was purified by column chromatography in 5-15% ethyl acetate-hexane to provide the target 2H-B.

Yield 600 mg (approximately 6%). ¹H-NMR (chloroform-d), δ: (two rotamers) 7.28-7.34 (m, 5H), 5.01-5.22 (m, 2H), 4.15-4.18 (m, 1H), 4.03 and 3.97 (two d, !H), 3.77 (s, 1.4H), 3.56-3.55 (m, 2H), 3.53 (s, 1.6H) 2.20-2.26 (m, 1H), 1.11 and 1.08 (two d, 3H), 0.87 and 0.86 (two s, 9H), 0.06 (s, 6H).

### Compound 2H-4

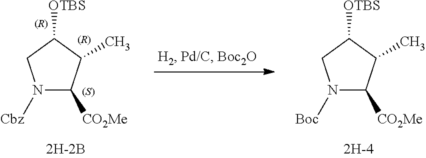

A solution of 2H-2B from the previous step (408 mg, 1 mmol) and Boc₂O (327 mg, 1.5 mmol) in THF (20 ml) was hydrogenated overnight in the presence of 10% Pd/C (50 mg). The catalyst was filtered off and the solvent was removed under reduced pressure. The residue was purified by column chromatography in 10-30% ethyl acetate-hexane to afford 2H-4 as an oil.

Yield 250 mg (67%). ¹H-NMR (chloroform-d), δ: (two rotamers) 4.14 (m, 1H), 3.97 and 3.88 (d and d, 1H), 3.76 and 3.74 (s and s, 3H), 3.42-3.54 (m, 2H), 2.18-2.25 (m, 1H), 1.45 and 1.41 (s and s, 9H), 1.10 and 1.08 (d and d, 3H), 0.88 (s, 9H), 0.06 (s, 6H).

### Compound 2H-5

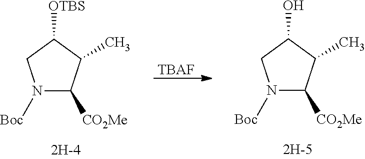

To a solution of 2H-4 from the previous step (250 mg, 0.67 mmol) in THF (3 ml) was added 1M solution of TBAF in THF (0.87 ml, 0.87 mmol). After 1 hour the reaction was quenched by addition of saturated aqueous sodium bicarbonate and then extracted with ethyl acetate. The solvent was removed under reduced pressure and the residue was purified by column chromatography in 30-70% ethyl acetate-hexane) to afford 2H-5 as a colorless oil.

Yield 177 mg (100%). ¹H-NMR (chloroform-d), δ: (two rotamers) 4.17 (m, 1H), 3.92 and 3.88 (d and d, 1H), 3.72 and 3.71 (s and s, 3H), 3.48-3.61 (m, 2H), 2.50-2.56 (br. s, 1H), 2.23 (m, 1H), 1.41 and 1.36 (s and s, 9H), 1.13 (d, 3H),

### Compound 2H-6

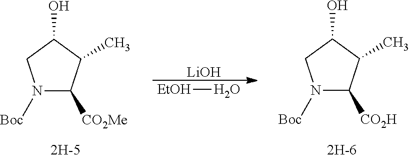

To a solution of the hydroxy acid from the previous example (177 mg, 0.67 mmol) in ethanol (3 ml) was added 2N aqueous lithium hydroxide (3.4 ml, 6.8 mmol). The reaction mixture was stirred for one hour at 40° C. when it was acidified to pH ˜2 with 2N aqueous hydrochloric acid and extracted with ethyl acetate. The organic extract was dried over magnesium sulfate and the solvent was removed under reduced pressure to afford the title hydroxy acid as an oil which was used on the next step without any further purification. Yield 160 mg (97%).

### Compound 2H-8

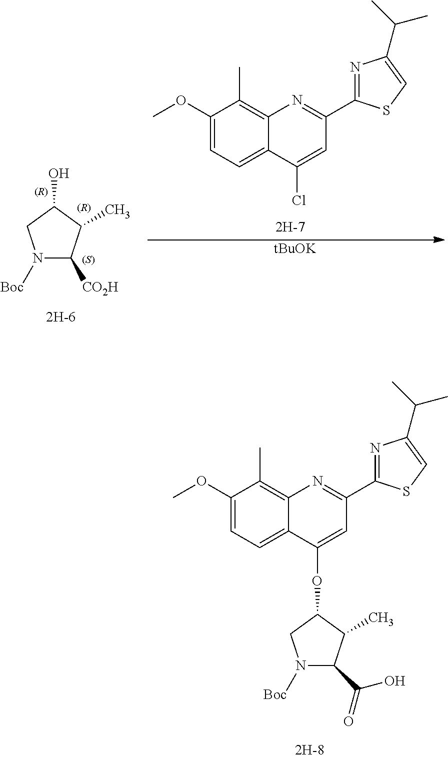

To a stirred solution of 2H-6 from the previous example (100 mg, 0.408 mmol) in DMSO (2 ml) was added 2H-7 (176 mg, 0.53 mmol) followed by addition of potassium tert-butoxide (105 mg, 0.94 mmol). The reaction was allowed to proceed overnight at room temperature, when it was quenched with water and acidified with aqueous hydrochloric acid to pH ˜2. The mixture was extracted with ethyl acetate; organic phase was washed with water, dried over magnesium sulfate and the solvent was removed under reduced pressure. The residue was purified by column chromatography in 0-15% methanol in DCM to afford 2H-8 as a yellow foam.

Yield: 196 mg (89%). ¹H-NMR (DMSO-d⁶, 70° C.), δ: 12.05-12.80 (br. s, 1H), 8.00 (d, 1H), 7.51 (s, 1H), 7.43 (d, 1H), 7.40 (s, 1H), 5.33 (m, 1H), 4.01 (d, 1H), 3.95 (s, 3H), 3.67-3.80 (m, 2H), 3.15 (m, 1H), 2.62-2.72 (m, 1H), 2.56 (s, 3H), 1.31-1.35 (m, 15H), 1.25 (d, 3H).

### Compound 2H-10

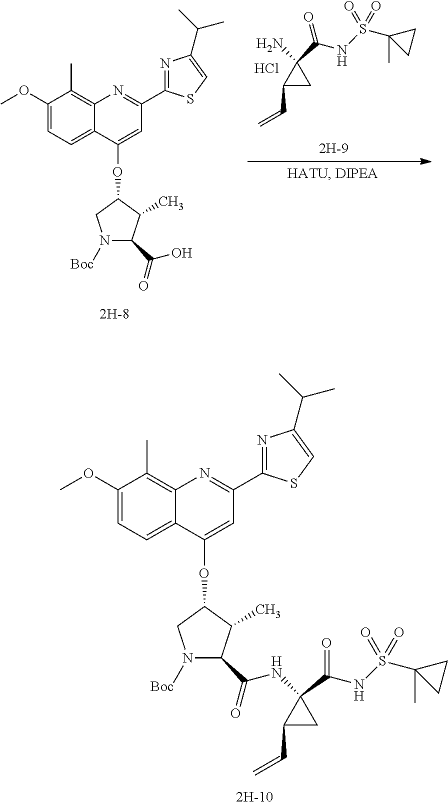

To a solution of 2H-8 from the previous step (196 mg, 0.36 mmol) in DMF (3 ml) was added 2H-9 (132 mg, 0.47 mmol) followed by DIPEA (0.7 ml, 4 mmol) and HATU (179 mg, 0.47 mmol). The reaction was allowed to proceed for overnight. The reaction was quenched with water, acidified with aqueous hydrochloric acid to pH ˜2 and extracted with ethyl acetate. Organic extract was washed with water, dried over magnesium sulfate and the solvent was removed under reduced pressure. The residue was purified by column chromatography in 20-70% acetone-hexane to afford 2H-10 as a pale-yellow foam.

Yield 190 mg (69%). ¹H-NMR (chloroform-d), δ: 9.84 (s, 1H), 7.95 (d, 1H), 7.48 (s, 1H), 7.24 (d, 1H), 7.05 (s, 1H), 6.95 (s, 1H), 5.824 (s, 1H), 5.32 (d, 1H), 5.23 (m, 1H), 5.18 (d, 1H), 4.05 (d, 1H), 4.00 (s, 3H), 3.80-3.88 (m, 2H), 3.20 (m, 1H), 2.87 (m, 1H), 2.71 (s, 3H), 2.19 (dd, 1H), 2.03 (dd, 1H), 1.54-1.78 (m, 3H), 1.53 (s, 3H), 1.36-1.48 (m, 13H), 1.24-1.26 (m, 4H), 0.80-0.92 (m, 2H).

### Compound 2H-11

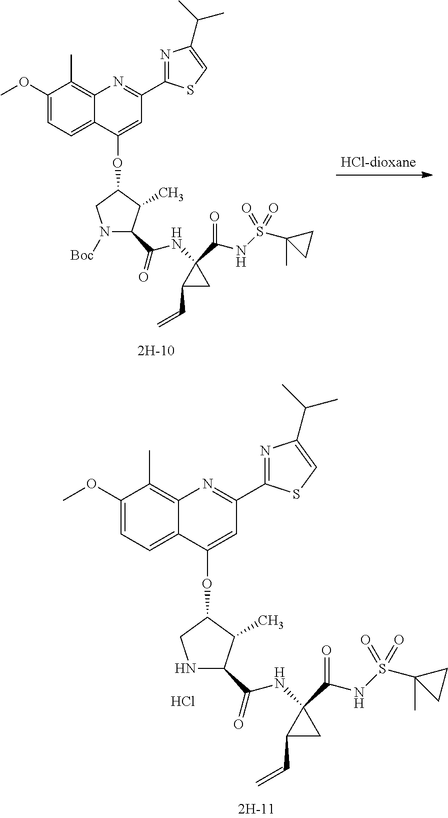

To a solution of 2H-10 from the previous example (190 mg, 0.247 mmol) in DCM (3 ml) was added 4M HCl-dioxane (0.62 ml, 2.5 mmol). The reaction was allowed to proceed for 1.5 h at room temperature and the solvent was removed in vacuo to afford 2H-11 as a yellow foam which was used on the next step without any further purification.

Yield 174 mg (100%). ¹H-NMR (DMSO-d⁶), δ: 11.40 (s, 1H), 10.10 (m, 1H), 9.65 (s, 1H), 9.13 (m, 1H), 8.16 (d, 1H), 7.52 (s, 1H), 7.49 (s, 1H), 7.46 (d, 1H), 5.52-5.62 (m, 2H), 5.36 (d, 1H), 5.16 (d, 1H), 4.28 (m, 1H), 3.99 (s, 3H), 3.69-3.87 (m, 1H), 3.59-3.64 (m, 1H), 3.15 (m, 1H), 2.56-2.65 (s and m, 4H), 2.38 (dd, 1H), 1.86 (dd, 1H), 1.38-1.42 (s and m, 5H), 1.34 (d, 6H), 1.20 (d, 3H), 0.86-0.96 (m, 2H).

### Compound 2H-13

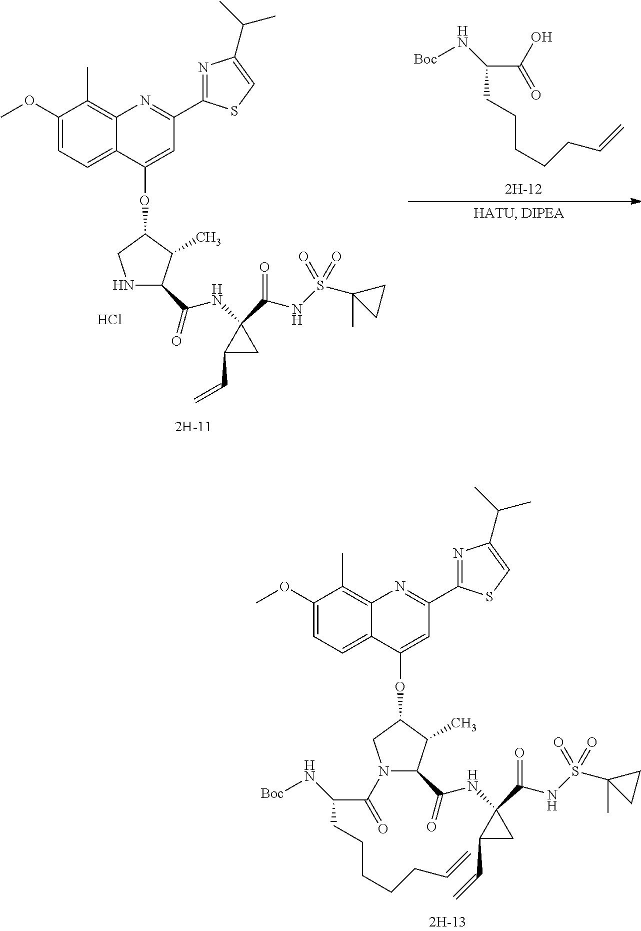

To a solution of 2H-11 from the previous example (70 mg, 0.099 mmol) and 2H-12 (35 mg, 0.13 mmol) in DMF (1 ml) was added HATU (49 mg, 0.13 mmol). The reaction was allowed to proceed for 1 hour at room temperature when it was taken into ethyl acetate-water. The mixture was acidified to pH 2 with 2N aqueous hydrochloric acid. Organic layer was separated, washed with brine, dried over magnesium sulfate and the solvent was removed under reduced pressure. The residue was purified by column chromatography in 20-40% acetone-hexane to furnish 2H-13 as a off-white foam.

Yield: 70 mg (77%). ¹H-NMR (chloroform-d), δ: 10.02 (s, 1H), 7.93 (d, 1H), 7.50 (s, 1H), 7.22 (d, 1H), 7.05 (s, 1H), 5.73-5.92 (m, 2H), 4.90-5.42 (m, 6H), 4.15-4.29 (m, 3H), 3.99 (s, 3H), 3.20 (m, 1H), 2.89 (m, 1H), 2.70 (s, 3H), 1.94-2.14 (m, 5H), 1.20-1.78 (m, 35H), 0.80-0.92 (m, 2H).

### Compound 106

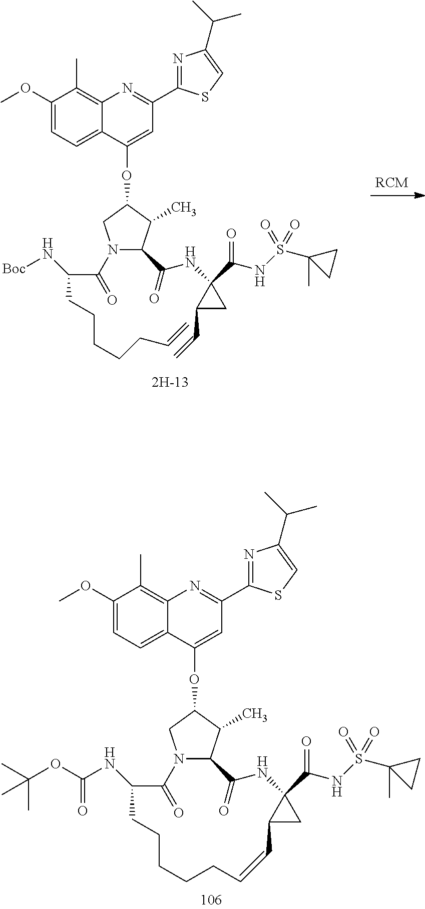

A solution of 2H-13 from the previous step (70 mg, 0.076 mmol) was heated to 70° C. and deoxygenated by bubbling a stream of argon for 15 min. To this solution was added Zhan catalyst (2 mg, 0.003 mmol) and the reaction was allowed to proceed for 30 min at 70° C. with bubbling of argon. The reaction mixture was concentrated under reduced pressure and the residue was purified by chromatography in 20-40% acetone-hexane to provide 106 as a beige foam.

Yield: 40 mg (59%). ¹H-NMR (chloroform-d), δ: 10.19 (s, 1H), 7.94 (d, 1H), 7.51 (s, 1H), 7.16 (d, 1H), 7.08 (s, 1H), 7.02 (s, 1H), 5.73 (dt, 1H), 5.34 (m, 1H), 5.09 (d, 1H), 5.01 (dd, 1H), 4.62 (d, 1H), 4.30 (d, 1H), 4.18 (dd, 1H), 4.05 (dd, 1H), 3.95 (s, 1H), 3.21 (m, 1H), 3.01 (m, 1H), 2.68 (s, 3H), 2.56 (m, 1H), 2.43 (dt, 1H), 1.76-2.05 (m, 5H), 1.25-1.58 (m, 23H), 1.03 (s, 9H), 0.84 (m, 2H).

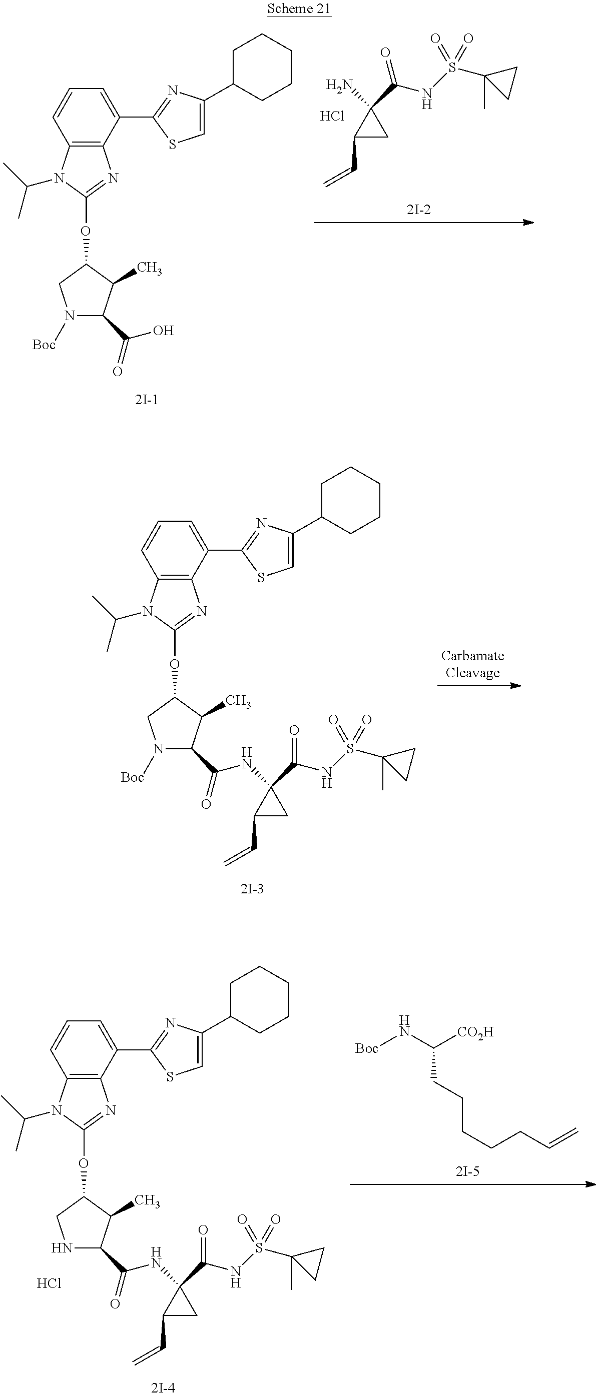

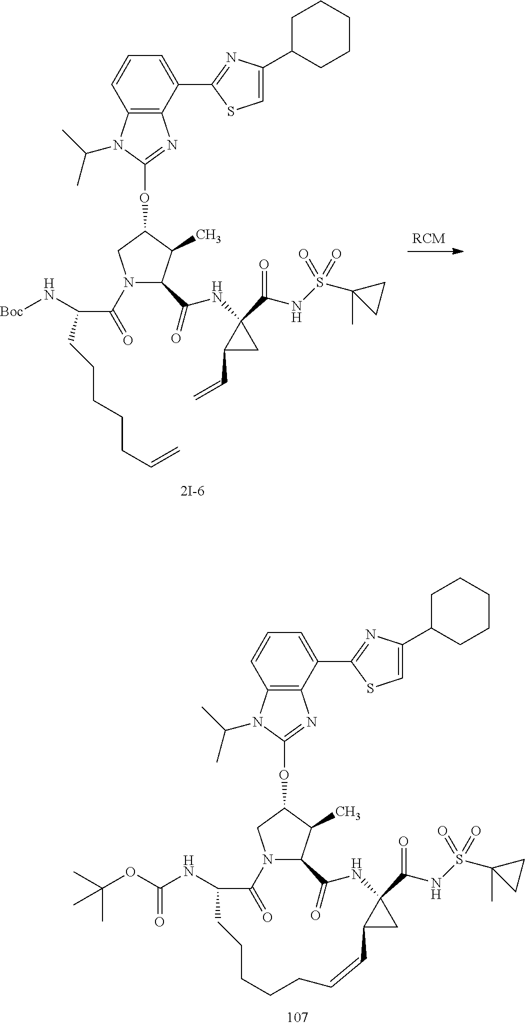

### Compound 2I-3

To a stirred solution of Compound 2I-1 (200 mg, 0.35 mmol) and (1R,2S)-1-amino-N-((1-methylcyclopropyl)sulfonyl)-2-vinylcyclopropanecarboxamide hydrochloride (2I-2, 128 mg, 0.46 mmol) in DMF (3 ml) were added DIPEA (0.61 ml, 3.5 mmol) and HATU (175 mg, 0.46 mmol). The reaction was allowed to stir for 1 h, when it was taken into ethyl acetate-water and acidified to pH ˜3 with 2 N aqueous hydrochloric acid. The organic phase was separated, washed with water, dried over magnesium sulfate and the solvent was removed in vacuo. The residue was purified by column chromatography in 20-40% acetone-hexane to provide Compound 2I-3. Yield 265 mg (95%). ¹H-NMR (chloroform-d, 60° C.) δ: 9.61 (s, 1H), 8.14 (d, 1H), 7.17-7.25 (d, 1H), 7.21 (dd, 1H), 6.95 (s, 1H), 6.57 (s, 1H), 5.78 (ddd, 1H), 5.47 m, 1H), 5.32 (d, 1H), 5.18 (d, 1H), 4.59 (m, 1H), 4.39 (d, 1H), 4.10-4.20 (m, 1H), 3.81-3.85 (m, 1H), 2.98-3.05 (m, 1H), 2.88 (m, 1H), 2.14-2.19 (m, 2H), 1.94 (dd, 1H), 1.82-1.88 (m, 2H), 1.60-1.80 (m, 3H), 1.55 (d, 3H), 1.54 (d, 3H), 1.50 (s, 9H), 1.22-1.42 (m, 4H), 1.20 (d, 3H), 0.82 (m, 2H).

### Compound 2I-4

To a stirred solution of Compound 2I-3 (248 mg, 0.31 mmol) in DCM (2 ml) was added 4 M HCl-dioxane (0.8 ml, 3.2 mmol) and the mixture was stirred for 1 hour a room temperature. The solvent was removed under reduced pressure. Compound 2I-4 was obtained by trituration of the residue with ethyl acetate. Yield 226 mg (100%). ¹H-NMR (DMSO-d⁶), δ: 11.45 (s, 1H), 10.56 (br. m, 1H), 9.26 (s, 1H), 9.12 (br. m, 1H), 7.98 (d, 1H), 7.61 (d, 1H), 7.33 (s, 1H), 7.22 (dd, 1H), 5.42-5.51 (m, 2H), 5.26 (d, 1H), 5.10 (d, 1H), 4.75 (m, 1H), 4.55 (ddd, 1H), 3.94-4.00 (m, 1H), 3.65-3.72 (m, 1H), 3.21 (m, 1H), 2.77 (m, 1H), 2.28 (dd, 1H), 2.03-2.06 (m, 2H), 1.84 (dd, 1H), 1.74-1.83 (m, 2H), 1.66-1.74 (m, 1H), 1.56 (d, 3H), 1.54 (d, 3H), 1.37-1.48 (m, 8H), 1.14-1.28 (m, 3H), 0.96 (d, 3H), 0.89 (m, 2H).

### Compound 2I-6

To a stirred solution Compound 2I-4 (200 mg, 0.29 mmol) and (S)-2-((tert-butoxycarbonyl)amino)non-8-enoic acid (2I-5, 101.4 mg, 0.37 mmol) in DMF (2 ml) was added DIPEA (0.52 ml, 3 mmol) followed by HATU (141 mg, 0.37 mmol). The reaction was allowed to proceed for 1 hour at room temperature, when it was partitioned between ethyl acetate-water, made acidic (pH ˜2) with 2 N aqueous hydrochloric acid and the organic phase was separated and washed with brine. The solution was dried over magnesium sulfate and concentrated under reduced pressure. Column chromatography in 20-40% acetone-hexane afforded Compound 2I-6. Yield 245 mg (89%). ¹H-NMR (chloroform-d,) δ: 9.70 (s, 1H), 8.15 (dd, 1H), 7.28 (dd, 1H), 7.22 (dd, 1H), 6.94 (s, 1H), 5.71-5.90 (m, 2H), 5.54 (m, 1H), 5.14-5.31 (m, 3H), 4.75-5.05 (m, 3H), 4.61 (m, 1H), 4.48 (m, 1H), 4.30-4.40 (m, 1H), 4.04 (dd, 1H), 2.85-3.03 (m, 2H) 2.10-2.22 (m, 2H), 1.90-2.30 (m, 5H), 1.52-1.88 (m, 15H), 1.40-1.51 (m, 14H), 1.15-1.40 (m, 14H), 0.80-0.92 (m, 2H).

### Compound 107

A solution of Compound 2I-6 (245 mg, 0.258 mmol) in toluene (50 ml) was degassed by bubbling argon for 15 min at 65° C. To this solution was added Zhan catalyst (4.6 mg, 0.006 mmol) as a single portion and the reaction mixture was stirred at 65° C. for 30 minutes with argon gas bubbling. After 30 min more catalyst (4.6 mg, 0.006 mmol) was added. After 30 min the reaction mixture was left to cool down to ambient temperature and the solvent removed in vacuo. The residue was purified by column chromatography in 20 to 30% acetone-hexane to afford Compound 107. Yield 128 mg (53.9%). ¹H-NMR (chloroform-d), δ: 1010 (s, 1H), 8.15 (d, 1H), 7.29 (d, 1H), 7.22 (dd, 1H), 5.74 (dt, 1H), 5.63 (dd, 1H), 5.08 (d, 1H), 4.94-5.03 (two dd, 1H), 4.63 (m, 1H), 4.55 (d, 1H), 4.41 (m, 1H), 3.92 (dd, 1H), 2.85-2.91 (m, 2H), 2.50-2.62 (m, 1H), 2.18-2.25 (m, 2H), 1.68-2.0 (m, 8H), 1.41-1.62 (m, 19H), 1.38 (s, 9H), 1.37 (d, 3H), 0.78-0.88 (m, 2H).

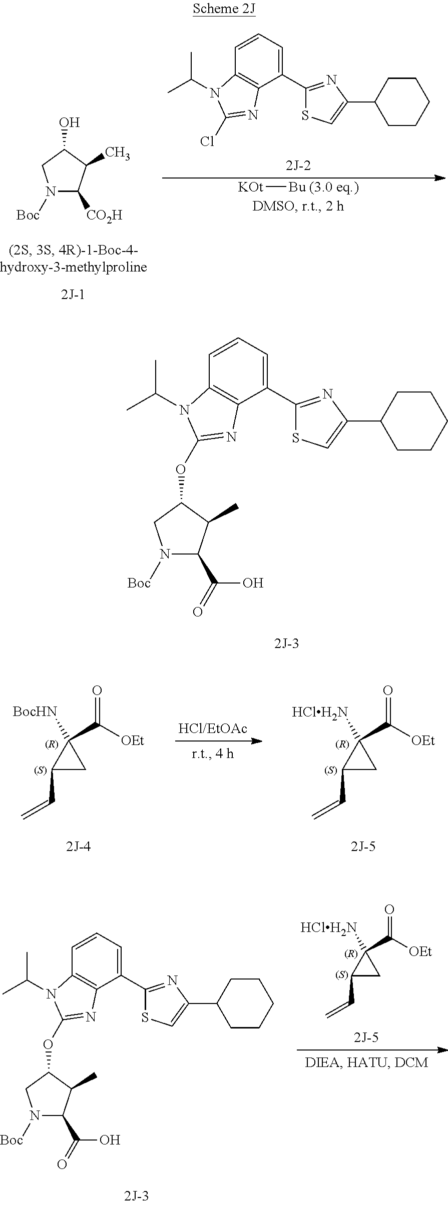

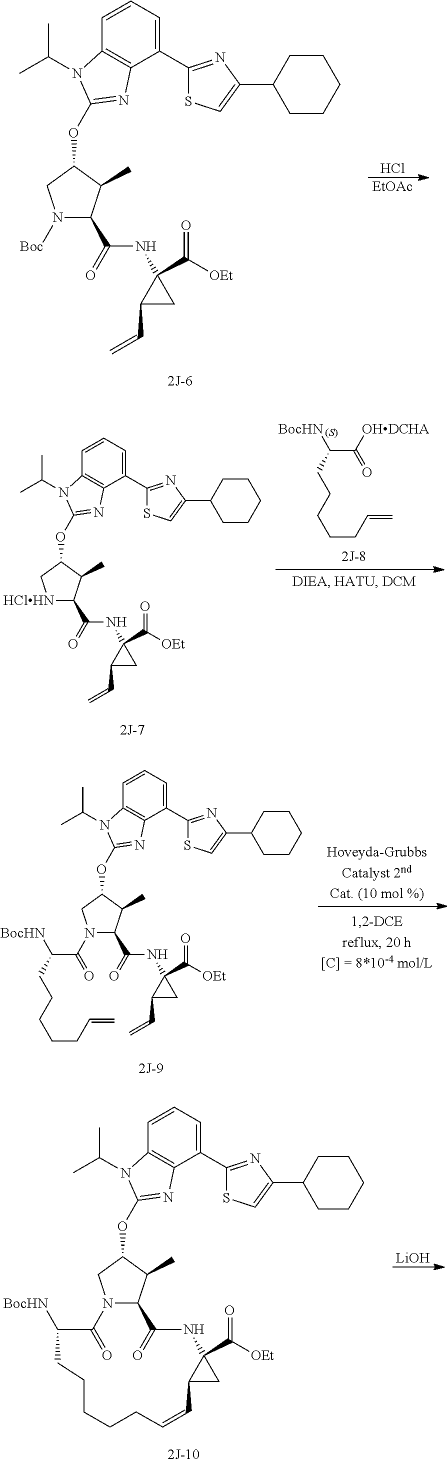

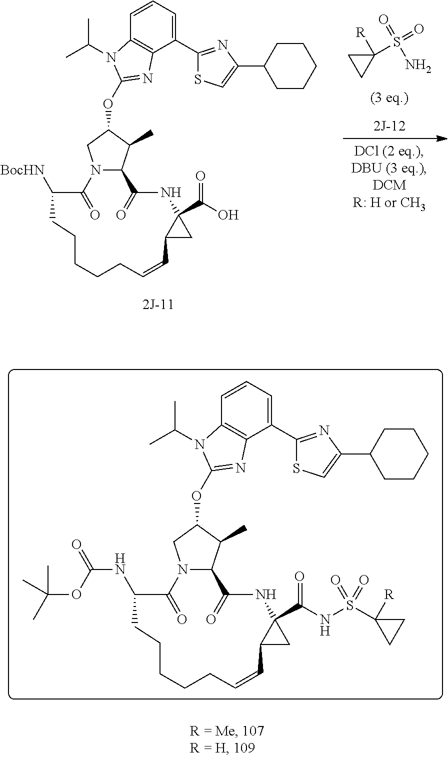

### Compound 2J-3

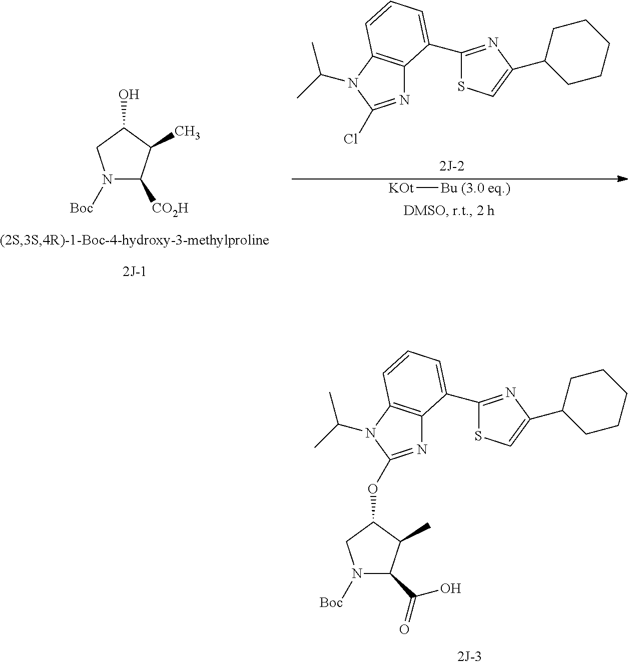

A flask was charged with compound 2J-1 (820 mg, 3.4 mmol, 1.0 eq.) and dimethylsulfoxide (8 mL). Potassium tert-Butoxide (1.39 g, 12.4 mmol, 3.0 eq.) was added portion-wise. The reaction mixture was stirred at ambient temperature for 15 minutes. Compound 2J-2 (1.23 g, 3.4 mmol, 1.0 eq.) was added dropwise to the reaction mixture. Stirring was continued for 2 hours. LCMS indicated the reaction complete. The reaction mixture was quenched with water and acidified to pH 3-4 with citric acid (aq.). The aqueous layer was extracted with ethyl acetate. The organic layers were combined, washed with brine, dried over magnesium sulfate, filtered and the solvent was removed in vacuo. The residue was purified by silica column chromatography (PE:EA=5:1) to provide compound 2J-3 as pale yellow solid (1.7 g, yield 86%). MS (ESI) m/z (M+H)⁺ 569.1.

### Compound 2J-5

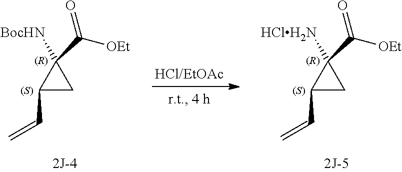

Compound 2J-4 (2.5 g, 9.8 mmol) was taken up with a solution of HCl (g) in EtOAc (4 M, 30 mL). The mixture was stirred at ambient temperature for 12 hrs. After that, the reaction mixture was concentrated under reduced pressure to afford compound 2J-5 (1.9 g, yield 99%) as a brown oil.

### Compound 2J-6

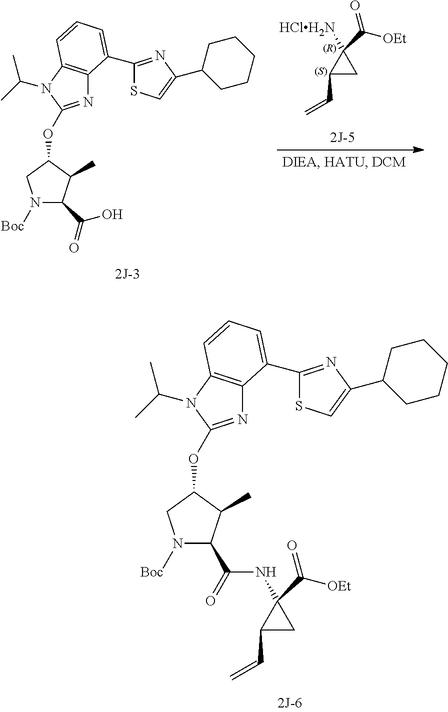

A flask was charged with compound 2J-3 (1.67 g, 2.9 mmol, 1.0 eq.) and DCM (40 mL). HATU (1.68 g, 4.4 mmol, 1.5 eq.) was added as a single portion and the reaction mixture stirred at ambient temperature for 5 minutes, and then diisopropylethylamine (2.6 mL, 17.6 mmol, 6 eq.) was added as a single portion followed by compound 2J-5 (1.1 g, 5.88 mmol, 2.0 eq.). The reaction mixture was allowed to warm up to ambient temperature and stirred for 12 hrs. The reaction mixture was concentrated, diluted with EtOAc and water, the aqueous layer was acidified to pH=3-4 with citric acid (aq.). The combined organic layer was washed with brine, dried over sodium sulfate, filtered and the solvent was removed in vacuo. The residue was purified by silica column chromatography (PE:EA=10:1 to 4:1) to provide compound 2J-6 as a light yellow solid (1.8 g, yield 87%). MS (ESI) m/z (M+H)⁺ 706.0.

### Compound 2J-7

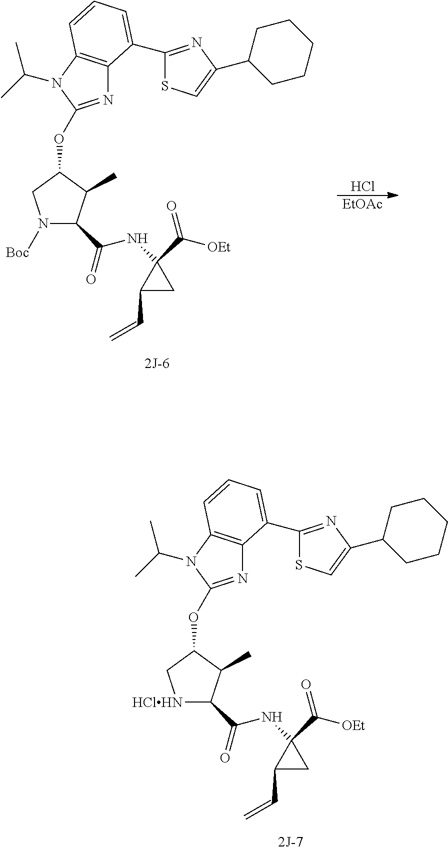

Compound 2J-6 (1.8 g, 2.6 mmol) was dissolved in a solution of HCl (g) in EtOAc (4 M, 20 mL). The mixture was stirred at ambient temperature for 12 hrs. After that, the reaction mixture was concentrated under reduced pressure to afford compound 7 (1.6 g, yield 99%) as a brown oil. MS (ESI) m/z (M+H)⁺ 606.2.

### Compound 2J-9

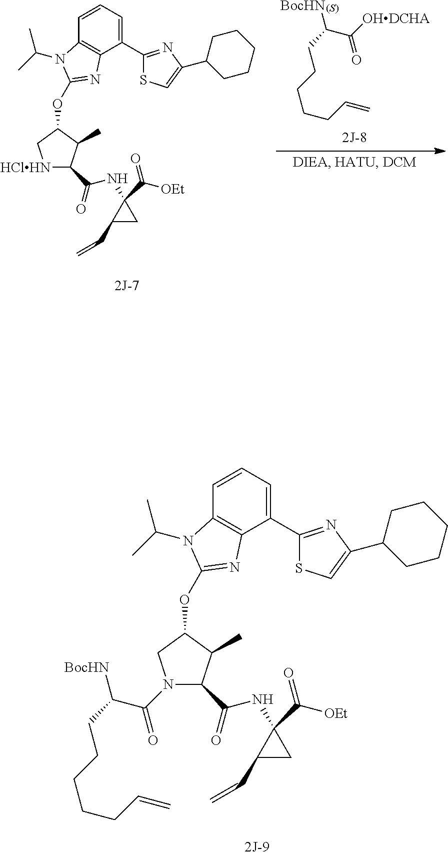

To a solution of compound 2J-7 (1.4 g, 3.1 mmol, 1.1 eq.) in DCM (30 mL) was added HATU (1.8 g, 4.7 mmol, 1.6 eq.) was added as a single portion and the reaction mixture stirred at ambient temperature for 5 minutes, and then diisopropylethylamine (2.8 mL, 18.7 mmol, 6.6 eq.) was added as a single portion followed by compound 2J-8 (1.8 g, 2.8 mmol, 1.0 eq.). The reaction mixture was stirred at ambient temperature for 12 hrs. The reaction mixture was concentrated, diluted with EtOAc and water, the aqueous layer was acidified to pH=3-4 with citric acid (aq.). The combined organic layer was washed with brine, dried over sodium sulfate, filtered and the solvent was removed in vacuo. The residue was purified by silica column chromatography (P:E=10:1 to 4:1) to provide compound 2J-9 as a light yellow solid (1.8 g, yield 87%). MS (ESI) m/z (M+H)⁺ 859.6.

### Compound 2J-10

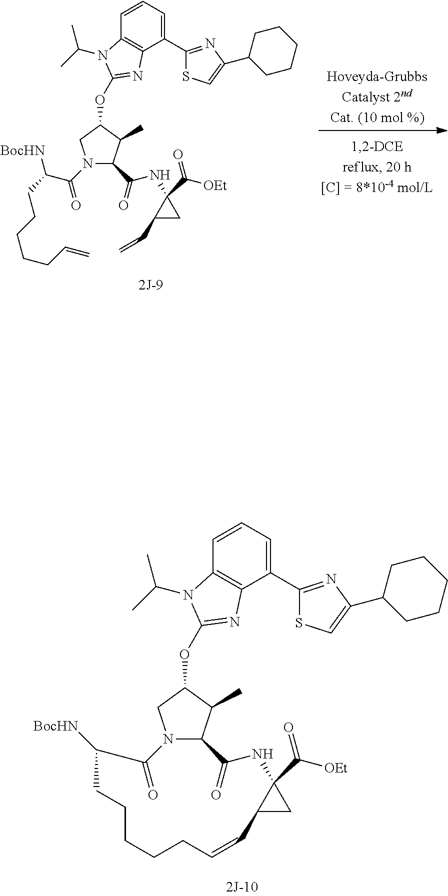

A flask was charged with compound 2J-9 (820 mg, 0.96 mmol, 1.0 eq.), Hoveyda-Grubbs catalyst (56 mg, 0.09 mmol, 0.1 eq.) and anhydrous 1,2-DCE (1 L). The reaction mixture was heated to reflux overnight. And then the reaction mixture was allowed to cool down to ambient temperature. The solvent was removed in vacuo, the resulting residue was purified by prep-TLC (petroleum ether/EtOAc=1/1) to provide 2J-10 (660 mg, yield 83%) as a white solid. MS (ESI) m/z (M+H)⁺ 831.4.

### Compound 2J-11

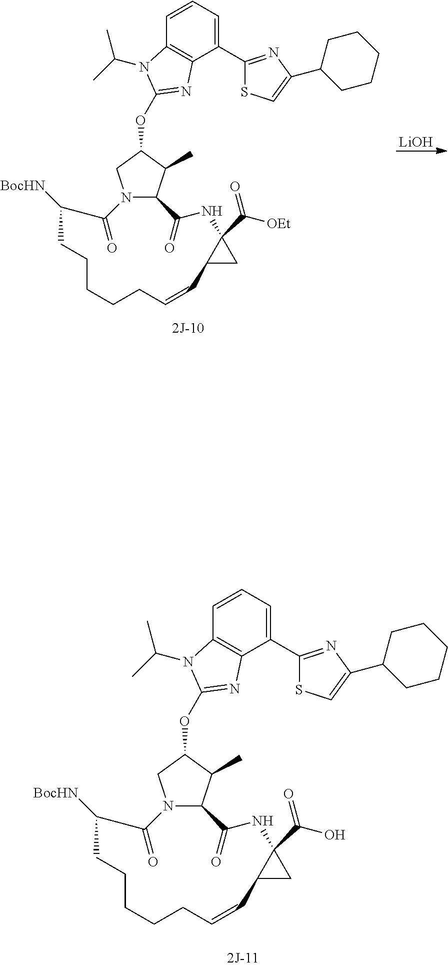

To a solution of compound 2J-10 (660 mg, 0.79 mmol, 1.0 eq.) in dioxane (10 mL) was added Lithium hydroxide monohydrate (332 mg, 7.9 mmol, 10.0 eq.) was added portion-wise and the reaction mixture was stirred at ambient temperature for 12 hours. The reaction mixture was acidified to pH=3-4 and extracted with EtOAc. The combined organic layer was washed with water and brine, dried over sodium sulfate, filtered and concentrated in vacuo to give compound 2J-11 (630 mg, yield 99%) as a white solid, which was used in the next step without further purification. MS (ESI) m/z (M+H)⁺ 803.4.

### Compounds 107 and 109

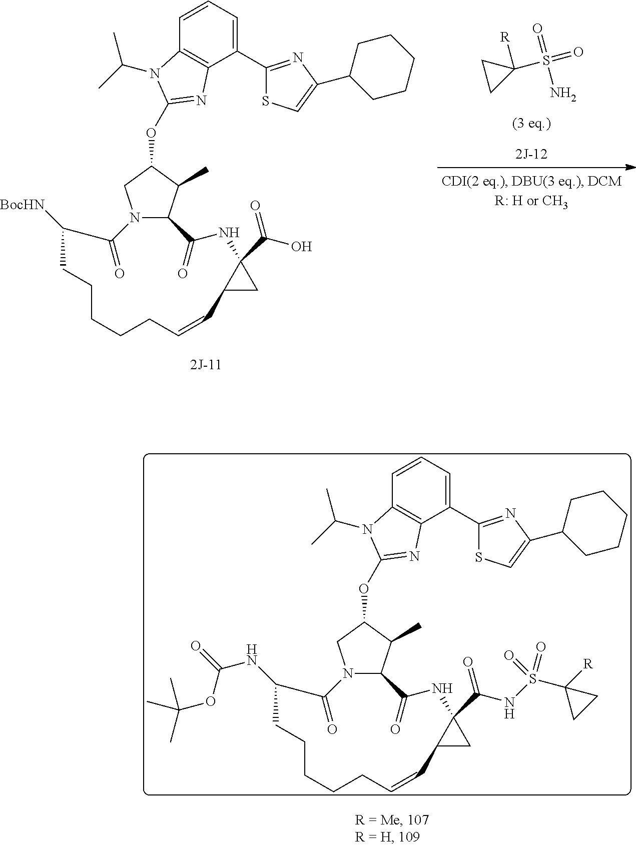

Compound 2J-11 (300 mg, 0.37 mmol, 1.0 eq.) and dichloromethane (6 mL) were charged into a round bottom flask (25 mL). 1,1′Carbonyldiimidazole (121 mg, 0.75 mmol, 2.0 eq.) was added and the reaction mixture stirred at reflux for 4 hrs. The resulting mixture was cooled to r.t., and then compound 2J-12 (3 eq.) and DBU (3 eq.) were added thereto. After that, the reaction mixture was heated to reflux and stirring was continued for 4 hrs. The reaction mixture was concentrated, diluted with water (20 mL) and adjusted to pH=4-5 with citric acid (aq.). The aqueous phase was extracted with EtOAc. The combined organic layer was washed with brine, dried over sodium sulfate, filtered and the solvent removed in vacuo. The residue was purified by prep-TLC (petroleum ether/EtOAc=1/1) to give a light yellow solid. The crude product was washed with methanol to give the desired product as a white solid.

Compound 107: 178 mg, yield 52%. MS (ESI) m/z (M+H)⁺ 920.5.

Compound 109: 260 mg, yield 66%. MS (ESI) m/z (M+H)⁺ 906.5.

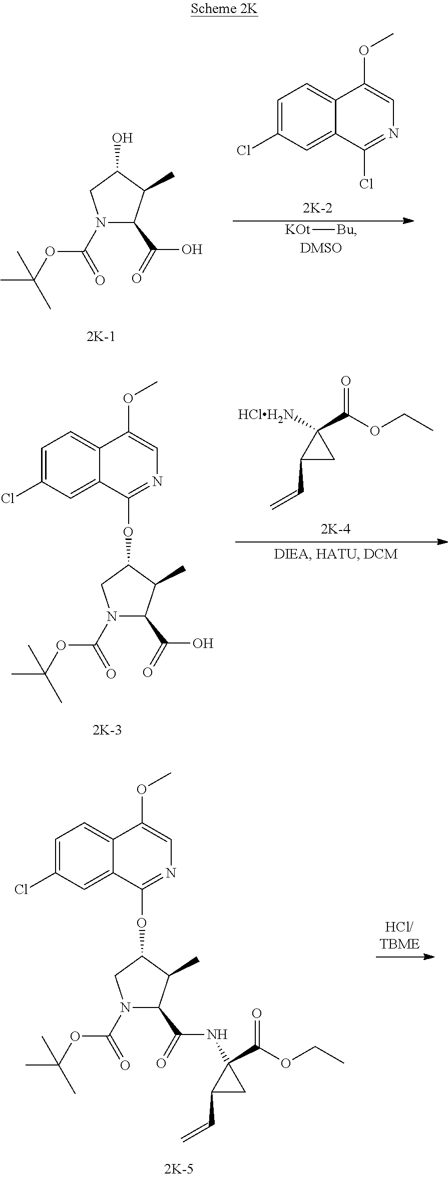

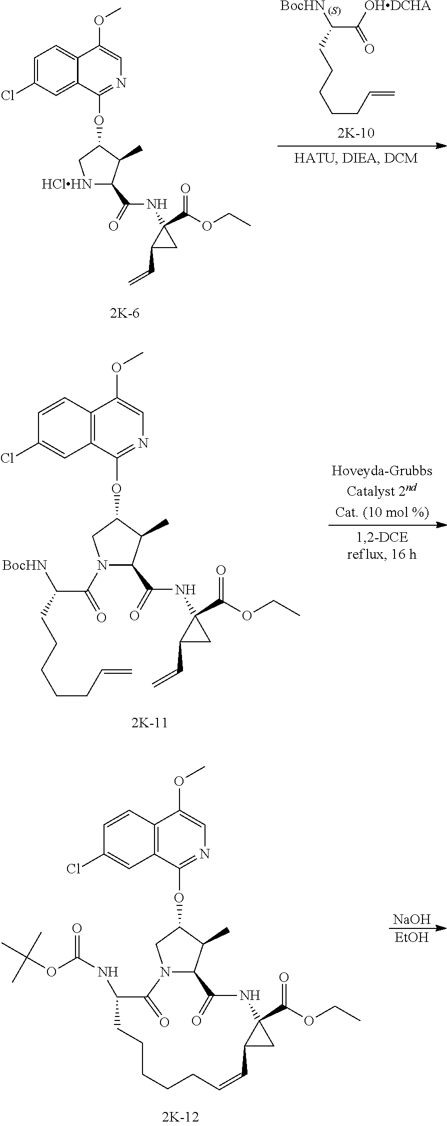

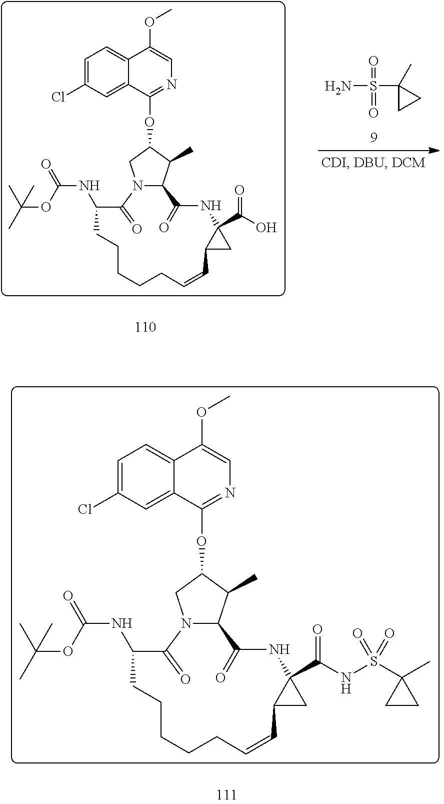

### Compound 2K-3

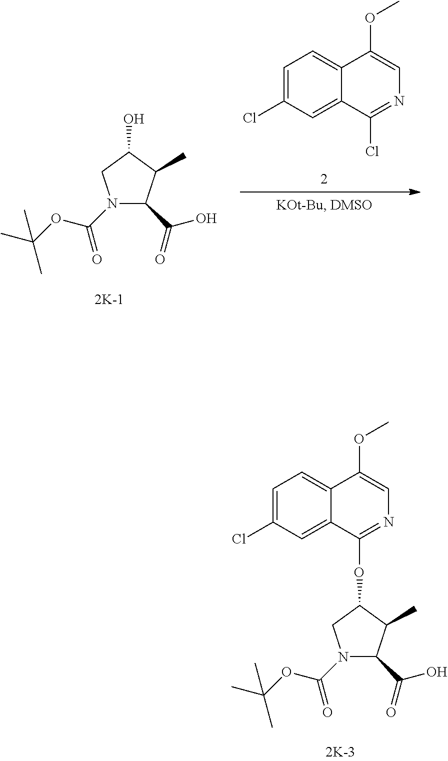

To a solution of compound 2K-1 (500 mg, 2.03 mmol, 1.05 eq) in 10 mL of DMSO was added KOt-Bu (683 mg, 6.09 mmol, 3.15 eq) at 0° C. The suspension was stirred for 15 min at 0° C. After that, compound 2K-2 (439 mg, 1.93 mmol, 1 eq) was added into the flask. The resulting mixture was stirred for another 18 h. TLC analysis showed the reaction completed. The reaction mixture was diluted with water, adjusted to pH=5-6 with aq. HCl (2 N), extracted with EA (50 mL×3). The combined organic layer was washed with brine, dried over anhydrous sodium sulfate, filtered and concentrated under reduced pressure. Purification by prep-TLC (DCM:MeOH=13:1) gave compound 2K-3 (690 mg, yield 82%). MS (ESI) m/z (M+H)⁺ 437.1.

### Compound 2K-5

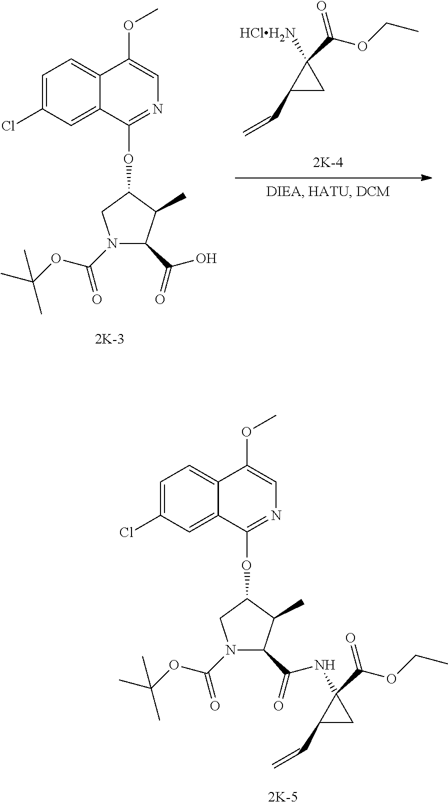

To a solution of compound 2K-3 (690 mg, 1.58 mmol, 1 eq) in 30 mL of DCM was added HATU (902 mg, 2.37 mmol, 1.5 eq), DIEA (815 mg, 6.32 mmol, 4 eq), and compound 2K-4 (604 mg, 3.16 mmol, 2 eq). The mixture was stirred for 18 hrs at room temperature. TLC (PE:EA=2:1) analysis showed the reaction completed. All the volatiles were removed under reduced pressure. The residual was diluted with water, extracted with EA (50 mL×3). The combined organic layer was washed with brine, dried over anhydrous sodium sulfate, filtered and concentrated under reduced pressure. Purification by column chromatography (PE:EA=2:1) gave compound 2K-5 as yellow solid (700 mg, yield 77%). MS (ESI) m/z (M+H)⁺ 574.1.

### Compound 2K-6

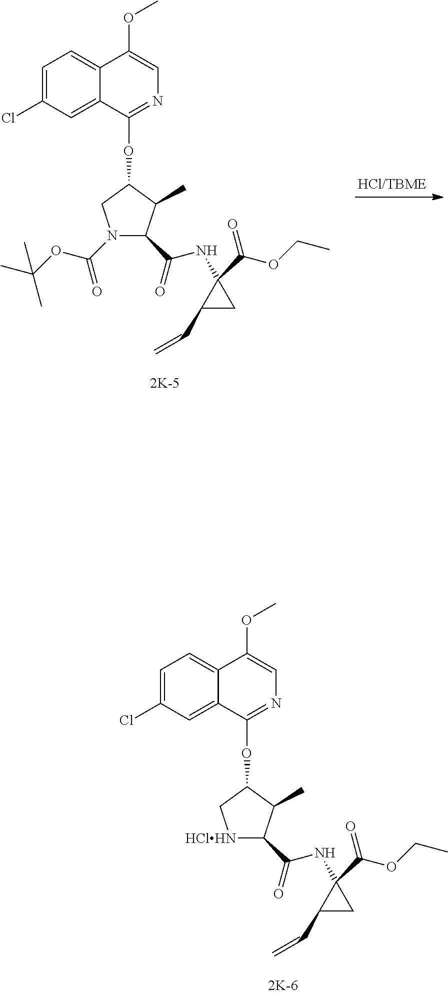

The mixture of compound 2K-5 (465 mg, 0.81 mmol) in 10 mL of solution of HCl (g) in TBME (saturated) was stirred at r.t. for 2 hrs. LCMS analysis showed the reaction completed. The solvent was removed under reduced pressure. The residual was dried and used directly for the next step without purification (400 mg, yield 97%). MS (ESI) m/z (M+H)⁺ 474.0.

### Compound 2K-11

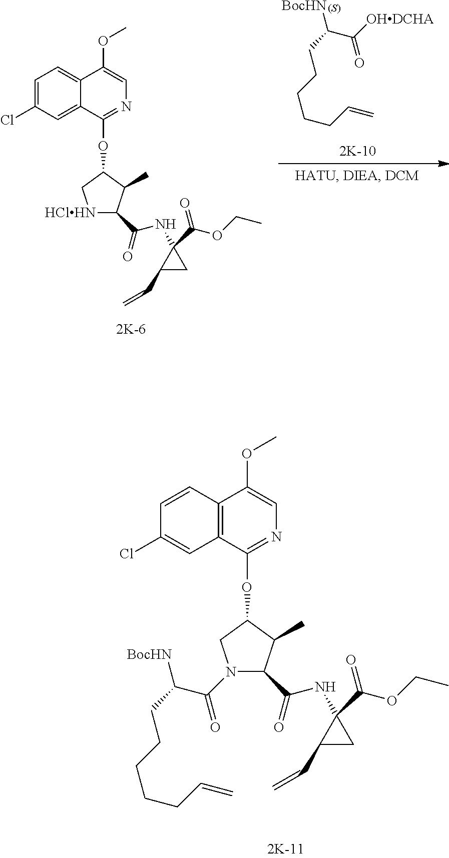

To a solution of compound 2K-6 (250 mg, 0.49 mmol, 1.0 eq.) and HATU (280 mg, 0.74 mmol, 1.5 eq.) in DCM (5 mL) was added diisopropylethylamine (380 mg, 2.94 mmol, 6.0 eq.) and compound 2K-10 (266 mg, 0.59 mmol, 1.2 eq.). The reaction mixture was stirred at ambient temperature for 12 hrs. The reaction mixture was concentrated, diluted with EtOAc and water, the aqueous layer was acidified to pH=6-7 with citric acid (aq.). The combined organic layer was washed with brine, dried over sodium sulfate, filtered and the solvent was removed in vacuo. The residue was purified by pre-TLC (PE:EA=3:1) to provide compound 2K-11 as a light yellow solid (340 mg, yield 96%). MS (ESI) m/z (M+H)⁺ 727.4.

### Compound 2K-12

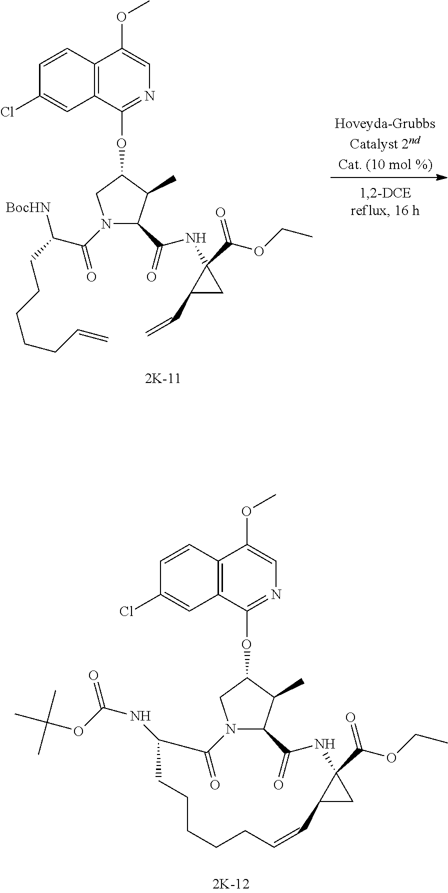

To a solution of compound 2K-11 (340 mg, 0.48 mmol, 1.0 eq.) in anhydrous 1,2-DCE (600 mL) was added Hoveyda-Grubbs catalyst (30 mg, 0.05 mmol, 0.1 eq.). The reaction mixture was stirred at reflux for 12 hrs. The solvent was concentrated in vacuo, the residue was purified by pre-TLC (PE:EA=3:1) to give compound 2K-12 (300 mg, yield 92%) as a light yellow solid. MS (ESI) m/z (M+H)⁺ 699.2.

### Compound 110

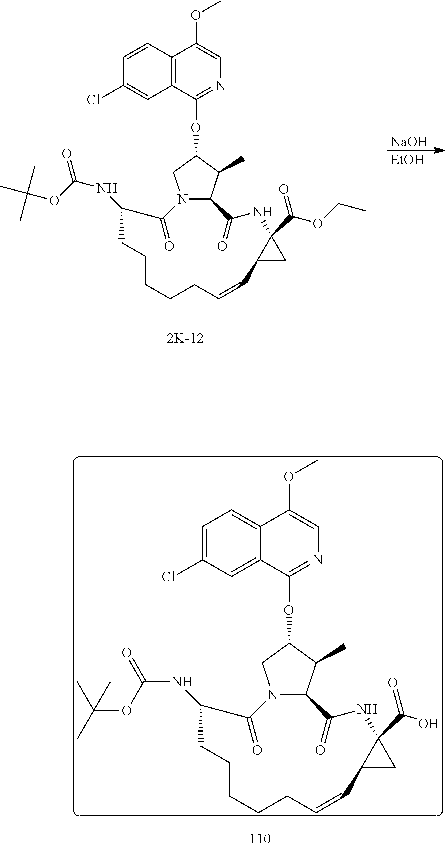

To a solution of compound 2K-12 (300 mg, 0.43 mmol, 1.0 eq.) in ethanol (10 mL) was added sodium hydroxide (1N, 4.3 mmol, 10.0 eq.) portion-wise and the reaction mixture was stirred at ambient temperature for 24 hours. The reaction mixture was acidified to pH 3-4 with citric acid (aq.) and extracted with EtOAc. The combined organic layer was washed with water and brine, dried over sodium sulfate, filtered and concentrated in vacuo to give Compound 110 (287 mg, yield 90%) as a white solid, which was used in the next step without further purification. MS (ESI) m/z (M+H)⁺ 671.3.

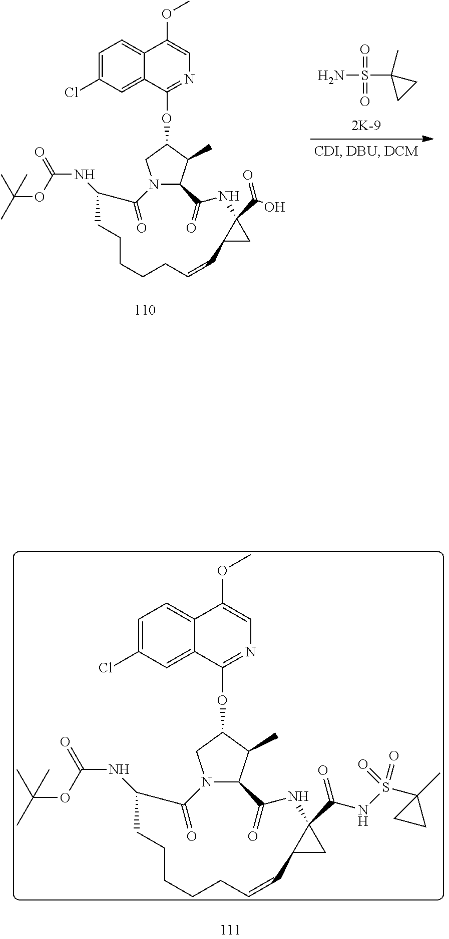

Compound 110 (100 mg, 0.14 mmol, 1.0 eq.) and dichloromethane (2 mL) were charged into a round bottom flask (25 mL). 1,1′Carbonyldiimidazole (46 mg, 0.28 mmol, 2.0 eq.) was added and the reaction mixture stirred at reflux for 4 hrs. The resulting mixture was cooled to r.t., and then compound 2K-9 (57 mg, 0.42 mmol, 3 eq.) and DBU (65 mg, 0.42 mmol, 3 eq.) were added thereto. After that, the reaction mixture was heated to reflux and stirring was continued for 3 hrs. The reaction mixture was concentrated, diluted with water (20 mL) and adjusted to pH=4-5 with citric acid (aq.). The aqueous phase was extracted with EtOAc. The combined organic layer was washed with brine, dried over sodium sulfate, filtered and the solvent removed in vacuo. The residue was purified by prep-TLC (petroleum ether/EtOAc=2/1) to give Compound III (60 mg, yield 60%) as a white solid. MS (ESI) m/z (M+H)⁺ 788.3.

### Example 6

### Synthesis of 2-Methyl Proline Non-Macrocyclic Analogs

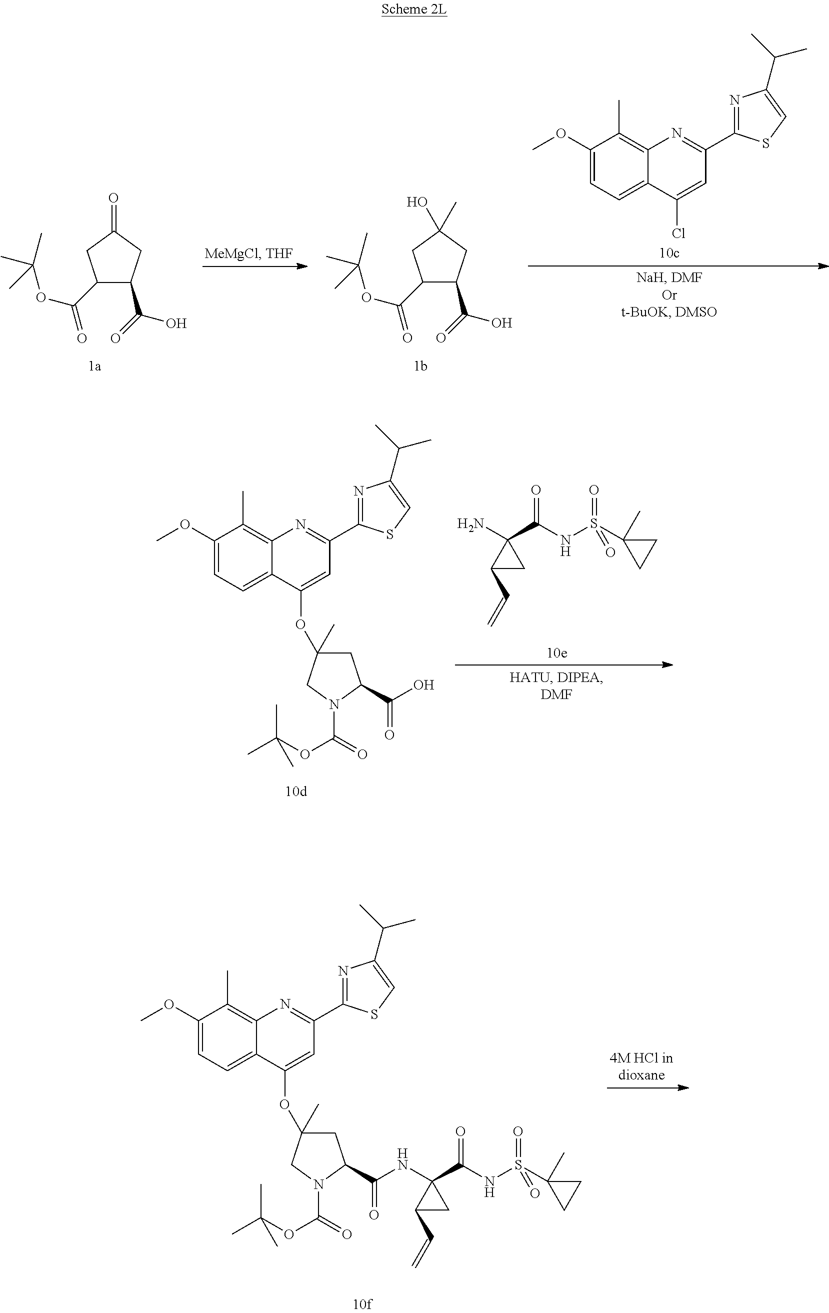

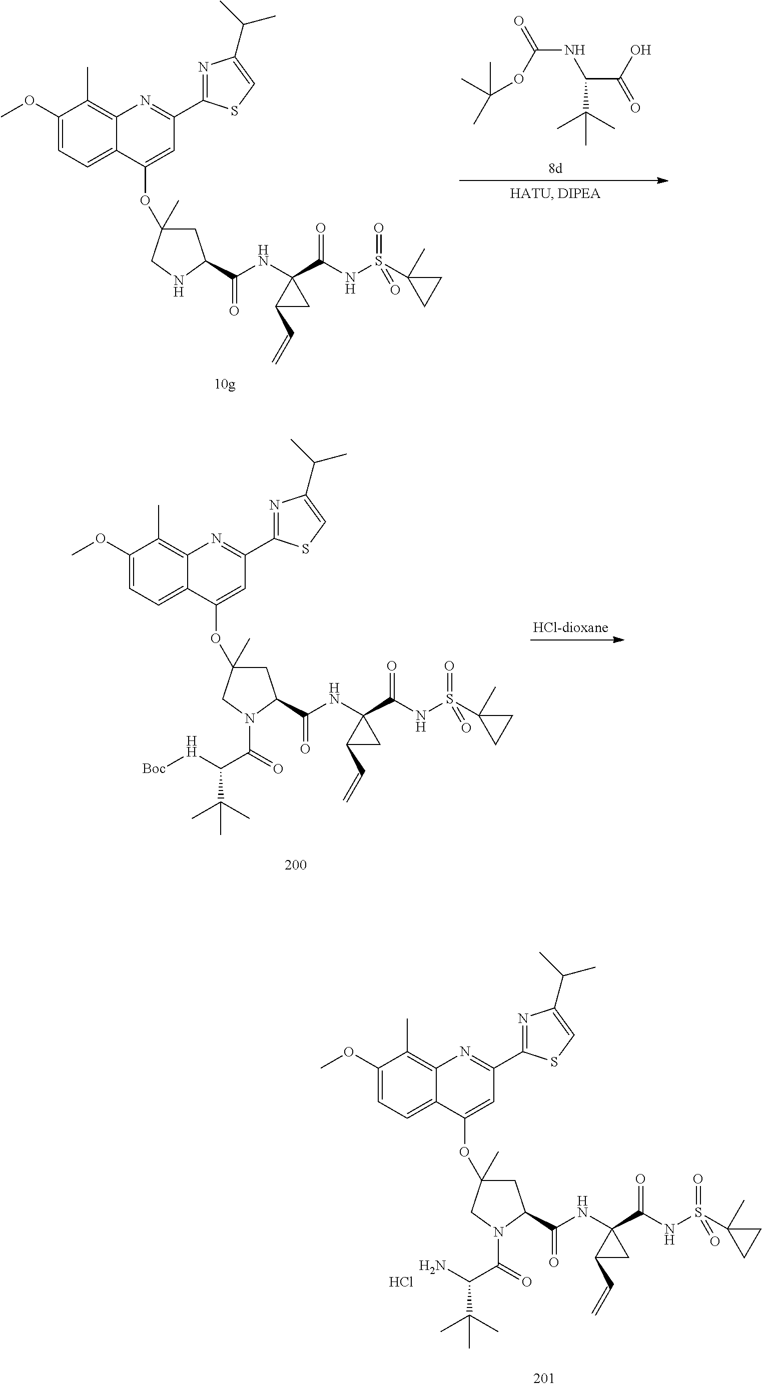

Preparation of compounds 200 and 201: Compounds 200 and 201 can be synthesized as shown in Scheme 2L. N-Boc-4-oxo-L-proline (1a) can be reacted with an organometallic reagent, for example a Grignard reagent such as methyl magnesium chloride, to afford N-Boc-4-hydroxy-4-methyl-L-proline (1b). N-Boc-4-hydroxy-4-methyl-L-proline (1b) can be treated with 4-chloro-2-(4-isopropylthiazol-2-yl)-7-methoxy-8-methylquinoline (10c) under basic conditions, such as sodium hydride in DMF or potassium tert-butoxide in DMSO, to afford carboxylic acid 10d. Carboxylic acid 10d can be coupled with amine 10e using standard coupling conditions, for example HATU in the presence of DIPEA, to afford compound 10f. The Boc protecting group of compound 10f can be removed under acid conditions, such as 4M HCl in dioxane, to afford amine 10g. Amine 10g can be coupled with carboxylic acid 8d using standard coupling conditions, for example HATU in the presence of DIPEA, to afford compound 200. Compound 201 can be synthesized by removing the Boc protecting group of compound 200 under acid conditions, such as 4M HCl in dioxane.

### Example 7

### Synthesis of 4,4-Dimethyl Proline Non-Macrocyclic Analog

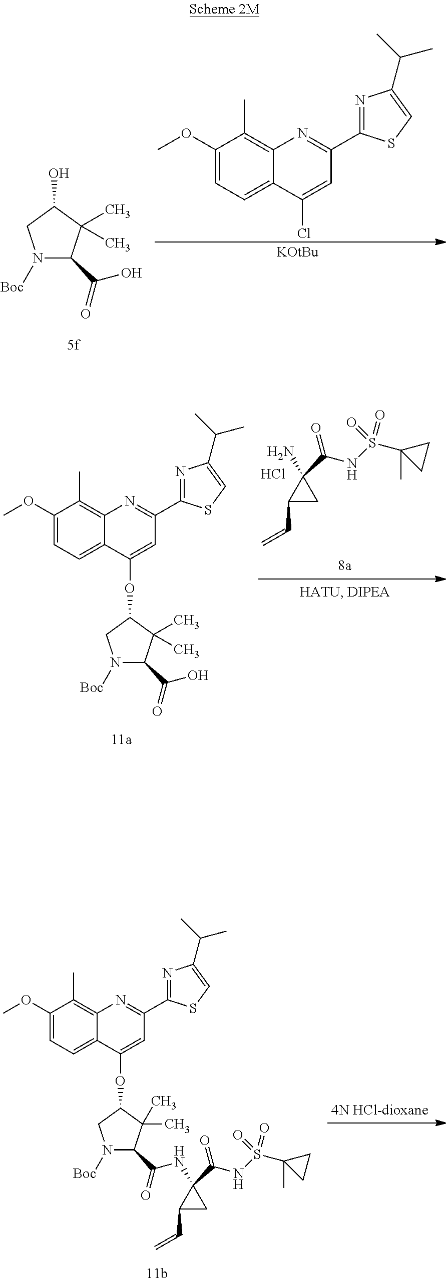

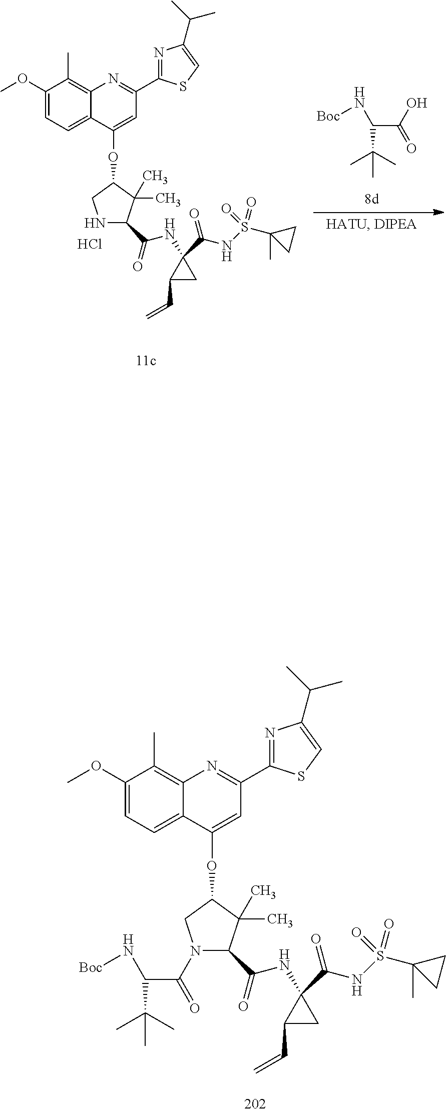

Compound 202, can be synthesized as shown in Scheme 2M. Compound 5f can be treated with 4-chloro-2-(4-isopropylthiazol-2-yl)-7-methoxy-8-methylquinoline under basic conditions, for example potassium tert-butoxide in DMSO, to afford carboxylic acid 11a. Carboxylic acid 11a can be coupled with amine 8a using standard coupling conditions, for example HATU in the presence of DIPEA, to afford compound 11b. The Boc protecting group of compound 8b can be removed under acid conditions, for example 4N HCl in dioxane, to afford amine 11c. Amine 11c can be coupled with carboxylic acid 8d using standard coupling conditions, for example HATU in the presence of DIPEA, to afford compound 202.

### Example 8

### Synthesis of 4-Methyl Proline Non-Macrocyclic Analogs

### Preparation of Compounds 204 and 203:

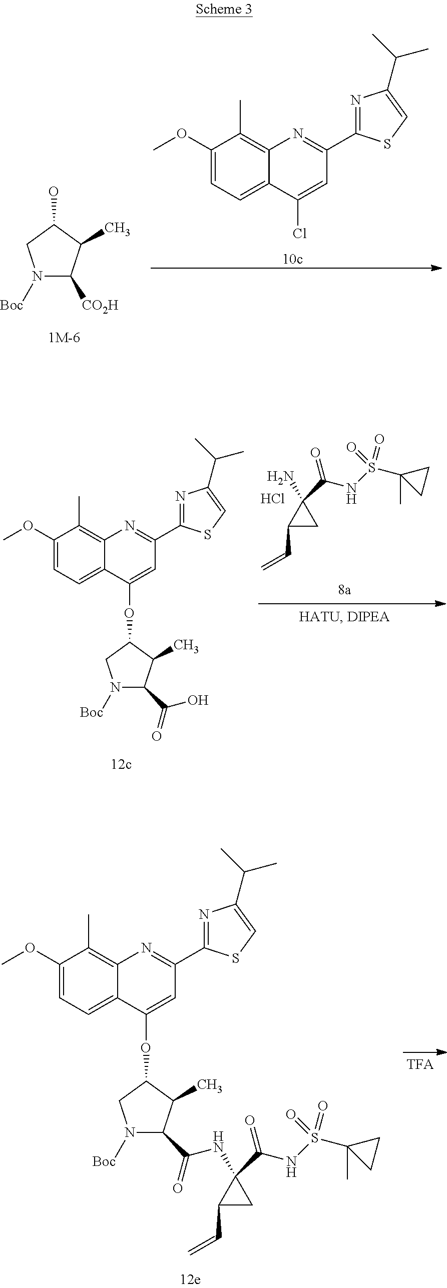

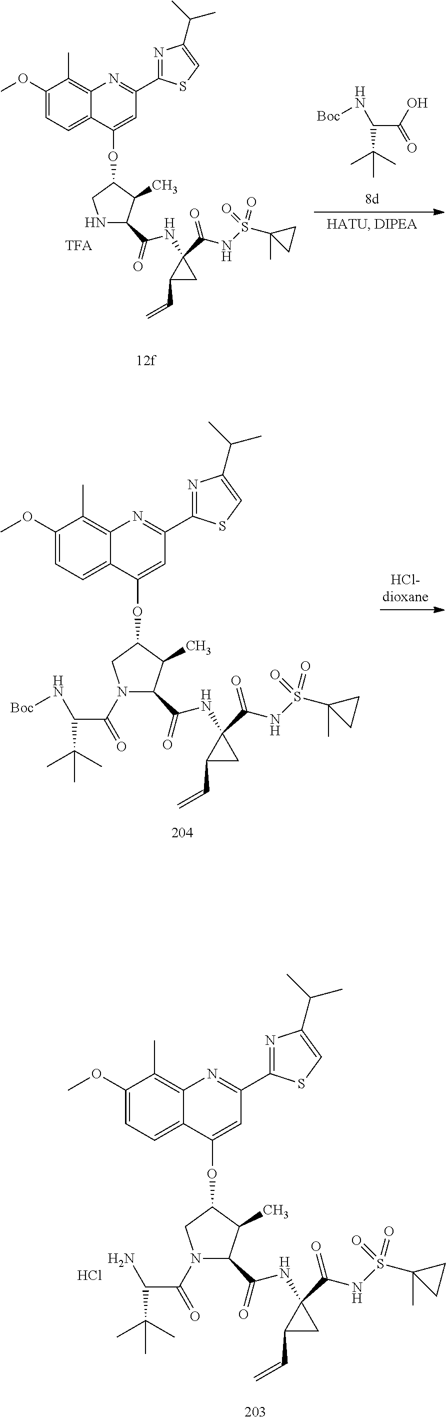

### Synthesis of Compound 204

### Compound 12c:

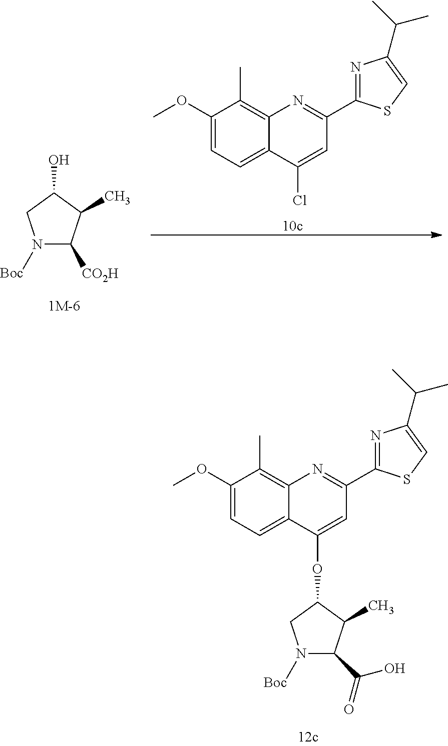

To a solution of Compound 1M-6 (73 mg, 0.3 mmol) and Compound 10c (131 mg, 0.39 mmol) in DMSO (2 ml) was added potassium tert-butoxide (74 mg, 0.66 mmol) and the reaction was stirred 6 h at room temperature. Water (10 ml) was added to the reaction mixture followed by 2 N aqueous HCl to pH ˜3 (0.25 ml). The mixture was extracted with ethyl acetate, organic extract was washed with brine, dried over potassium sulfate and concentrated under vacuum. The residue was purified by column chromatography in 1-10% MeOH-DCM to provide Compound 12c as an yellow oil. Yield 141 mg (87%). ¹H-NMR (chloroform-d), 60° C., δ: 7.96 (d, 1H), 7.50 (s, 1H), 7.19 (d, 1H), 6.98 (s, 1H), 4.98 (m, 1H), 4.55 (m, 1H), 4.19 (m, 1H), 3.96 (s, 3H), 3.20 (m, 1H), 2.96 (m, 1H), 2.68 (s, 3H), 1.45 (s, 9H), 1.36 (d, 6H), 1.27 (d, 3H).

### Compound 12e:

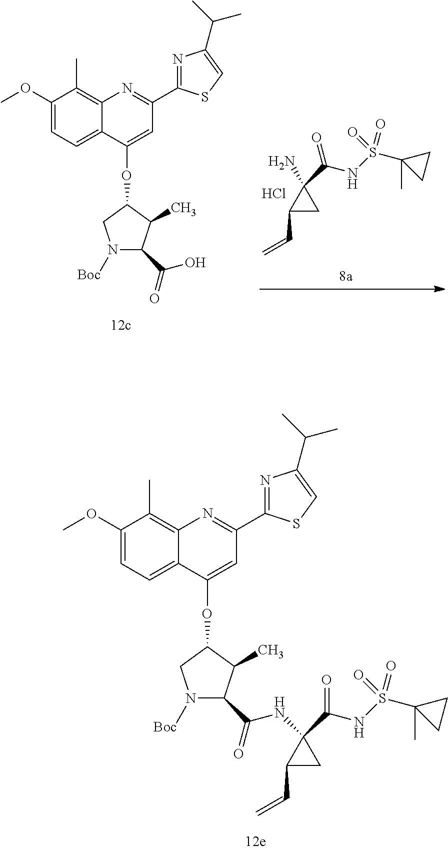

To a solution of Compound 12c (140 mg, 0.26 mmol) and (1R,2S)-1-amino-N-((1-methylcyclopropyl)sulfonyl)-2-vinylcyclopropanecarboxamide hydrochloride (109 mg, 0.39 mmol) in DMF (5 ml) was added DIPEA (0.23 ml, 1.3 mmol) followed by HATU (148 mg, 0.39 mmol). The reaction mixture was stirred for 2 hours at room temperature, diluted with water and acidified to pH ˜3 with 2N hydrochloric acid (0.6 ml) and extracted with ethyl acetate. Organic phase was washed with brine, dried over magnesium sulfate and concentrated under reduced pressure. The residue was purified by column chromatography in 20-40% acetone-hexane to afford Compound 12e. Yield: 174 mg (87%). ¹H-NMR (chloroform-d, 60° C.) δ: 9.65 (br. s, 1H), 7.93 (d, 1H), 7.52 (s, 1H), 7.20 (d, 1H), 7.01 (s, 1H), 5.76 (m, 1H), 5.31 (d, 1H), 5.17 (d, 1H), 5.05 (m, 1H), 4.43 (d, 1H), 4.14 (dd, 1H), 3.96 (s, 3H), 3.67 (m, 1H), 3.21 (m, 1H), 2.95 (m, 1H), 2.69 (s, 3H), 2.18 (dd, 1H), 1.94 (dd, 1H), 1.65 (m, 2H), 1.53 (s, 3H), 1.47 (s, 9H), 1.39 (d, 6H), 1.20 (d, 3H), 0.82 (m, 2H).

### Compound 12f:

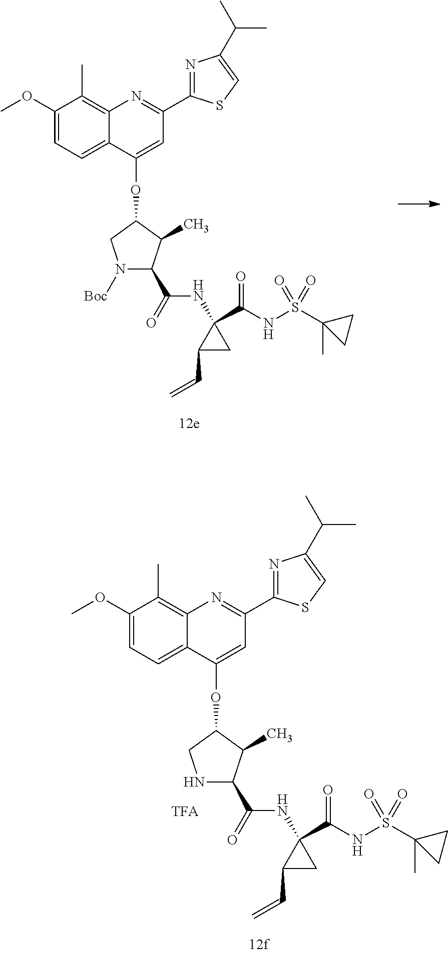

To a stirred solution of Compound 12e (174 mg, 0.226 mmol) in DCM (2 ml) was added TFA (0.75 ml) and the reaction was allowed to proceed for 2 hours at room temperature. The reaction mixture was concentrated under reduced pressure and co-evaporated with toluene to give Compound 12f as yellowish oil which was used in subsequent reactions without any additional purification. Yield 191 mg (100%).

### Compound 204:

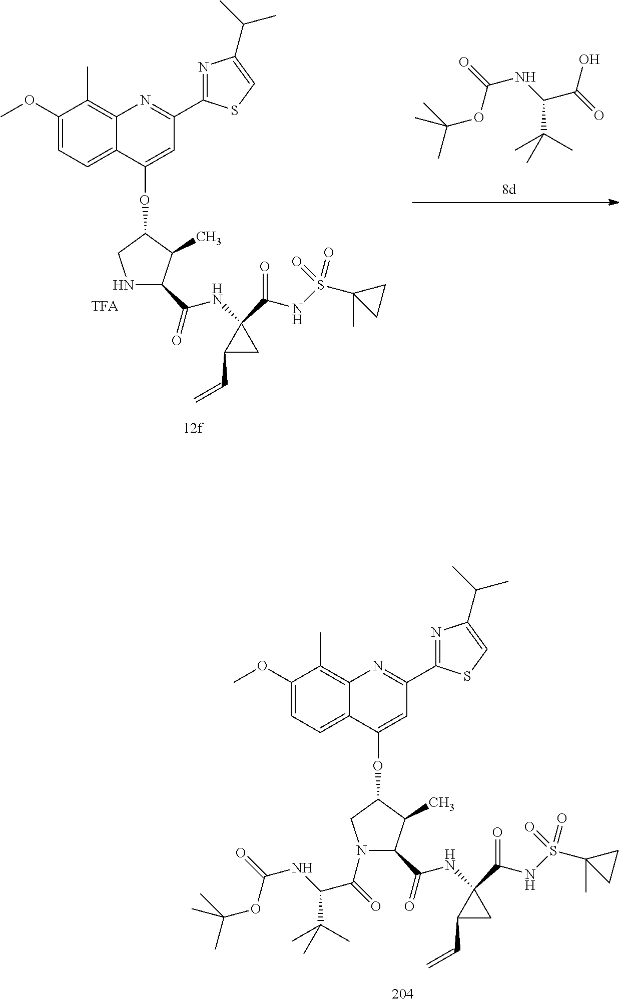

To a stirred solution of Compound 12f (127 mg, 0.15 mmol) and Boc-tert-butyl leucine (52 mg, 0.225 mmol) was added DIPEA (0.26 ml, 1.5 mmol) followed by HATU (86 mg, 0.225 mmol). The reaction was stirred for 3 hours at room temperature when it was quenched with addition of 2 N aqueous hydrochloric acid (0.6 ml) and water (10 ml). The reaction mixture was extracted with ethyl acetate; organic phase was washed with brine, dried over magnesium sulfate and concentrated under reduced pressure. The residue was separated by column chromatography in 20-40% acetone-hexane to provide Compound 204 as an off-white foam. Yield 105 mg (79%). ¹H-NMR (DMSO-d⁶), δ: 10.30 (s, 1H), 8.71 (s, 1H), 8.08 (d, 1H), 7.49 (d, 1H), 7.48 (s, 1H), 7.31 (d, 1H), 6.69 (d, 1H), 5.50 (m, 1H), 5.17-5.23 (dd and m, 2H), 5.09 (dd, 1H), 4.51 (d, 1H), 4.28 (d, 1H), 4.12 (dd, 1H), 4.08 (d, 1H), 3.94 (s, 3H), 3.16 (m, 1H), 2.92 (m, 1H), 2.59 (s, 3H), 2.18 (dd, 1H), 1.52 (dd, 1H), 1.41 (s, 3H), 1.35 (d, 6H), 1.27 (s, 9H), 1.05 (d, 3H), 1.00 (s, 9H), 0.91 (m, 2H). LC-MS (M+1)⁺: 881.3.

### Synthesis of Compound 205:

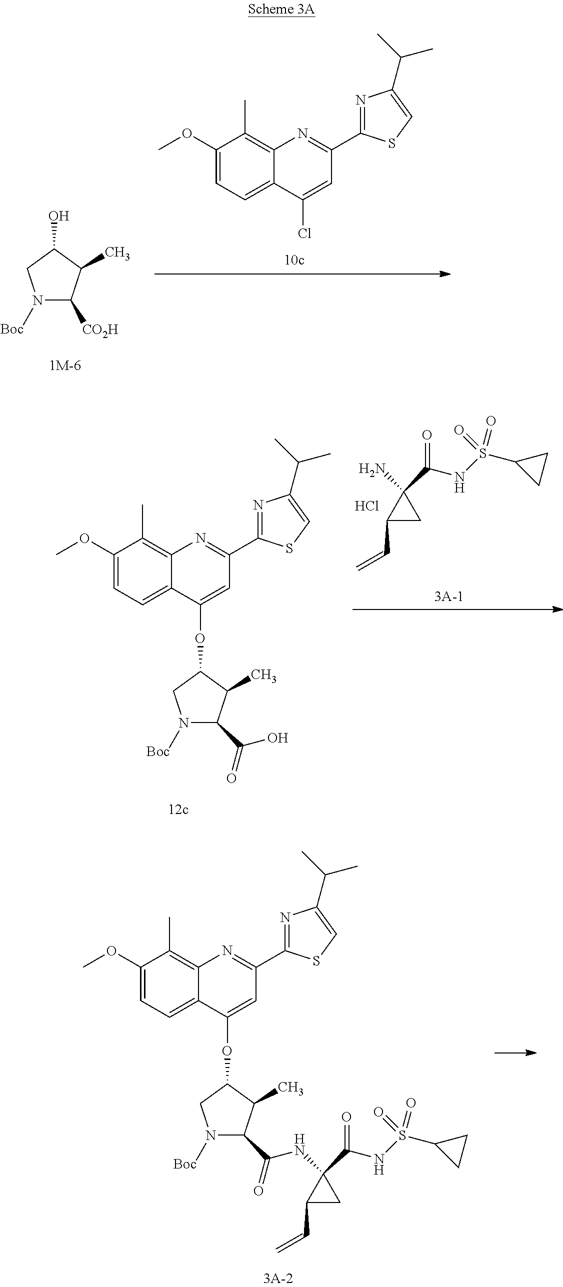

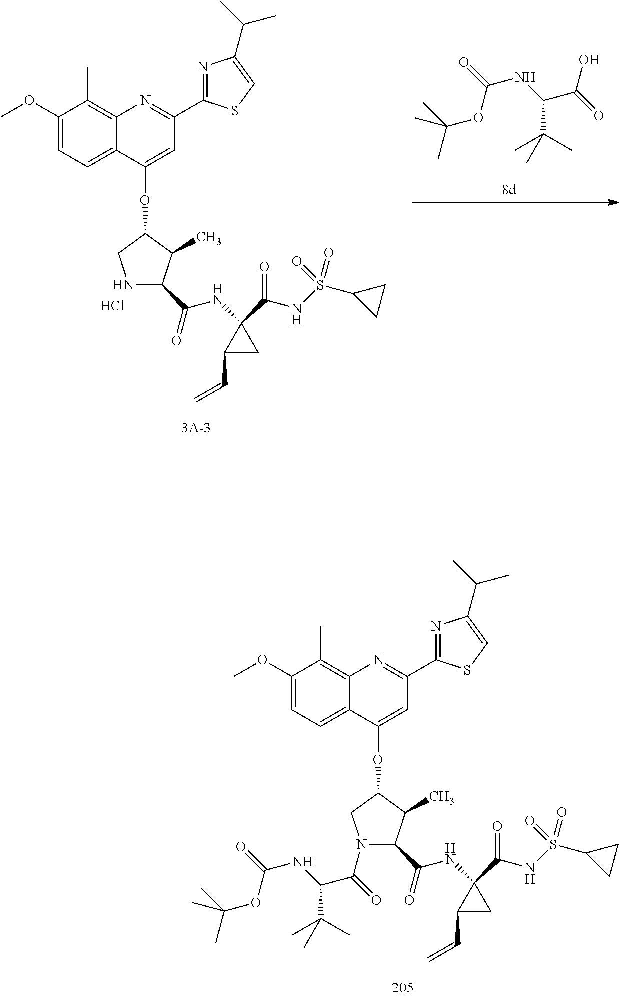

### Compound 3A-2

Compound 12c (540 mg, 1 mmol) was converted to Compound 3A-2 as described for the synthesis of Compound 12e. Yield 670 mg (89%). ¹H-NMR (chloroform-d, 60° C.) δ: 9.77 (s, 1H), 7.92 (d, 1H), 7.51 (s, 1H), 7.21 (d, 1H), 7.01 (s, 1H), 6.66 (br. s, 1H), 5.75-5.85 (m, 1H), 5.31 (d, 1H), 5.17 (d, 1H), 5.06 (m, 1H), 4.40 (d, 1H), 4.09-4.14 (m, 1H), 3.97 (s, 3H), 3.65-3.75 (m, 1H), 3.21 (m, 1H), 2.89-3.00 (m, 2H), 2.70 (s, 3H), 2.18 (dd, 1H), 1.97 (dd, 1H), 1.47 (s, 9H), 1.39 (d, 6H), 1.18 (d, 3H), 0.95-1.15 (m, 2H).

### Compound 3A-3:

Compound 3A-3 was synthesized from Compound 3A-2 (670 mg, 0.89 mmol) as described for the synthesis of Compound 12f using 4 N HCl-dioxane for BOC group cleavage. Yield 570 mg (92.9%). ¹H-NMR (DMSO-d⁶), δ: 11.73 (s, 1H), 10.95 (m, ex, 1H), 9.27 (s, 1H), 9.12 (m, ex., 1H), 8.19 (d, 1H), 7.50 (d, 1H), 7.48 (s, 1H), 7.47 (d, 1H), 5.47-5.56 (m, 1H), 5.43 (d, 1H), 5.26 (dd, 1H), 5.10 (dd, 1H), 4.64 (m, 1H), 3.98 (s, 3H), 3.61-3.65 (m, 1H), 3.11-3.20 (m, 2H), 2.90-3.00 (m, 1H), 2.60 (s, 3H), 2.28 (m, 1H), 1.85 (dd, 1H), 1.35 (d, 6H), 1.23 (dd, 1H), 0.97-1.12 (m, 4H), 0.94 (d, 3H).

### Compound 205:

Compound 205 was synthesized from Compound 3A-3 (200 mg, 0.29 mmol) as described for the synthesis of Compound 204. Yield 242 mg (100%). ¹H-NMR (DMSO-d⁶), δ: 10.40 (S, 1H), 8.76 (s, 1H), 8.07 (d, 1H), 7.49 (d, 1H), 7.48 (s, 1H), 7.32 (d, 1H), 6.70 (d, 1H), 5.51-5.62 (m, 1H), 5.19-5.24 (m, 2H), 5.09 (DD, 1H), 4.49 (d, 1H), 4.25-4.29 (m, 1H), 3.95 (s, 3H), 3.15 (m, 1H), 2.89-2.97 (m, 2H), 2.59 (s, 3H), 2.16 (dd, 1H), 1.17 (dd, 1H), 1.35 (d, 3H), 1.34 (d, 3H), 1.27 (s, 9H), 1.08-1.11 (m, 3H), 1.02 (d, 3H), 1.00 (s, 9H).

### Alternative Synthesis of Compounds 204 and 205:

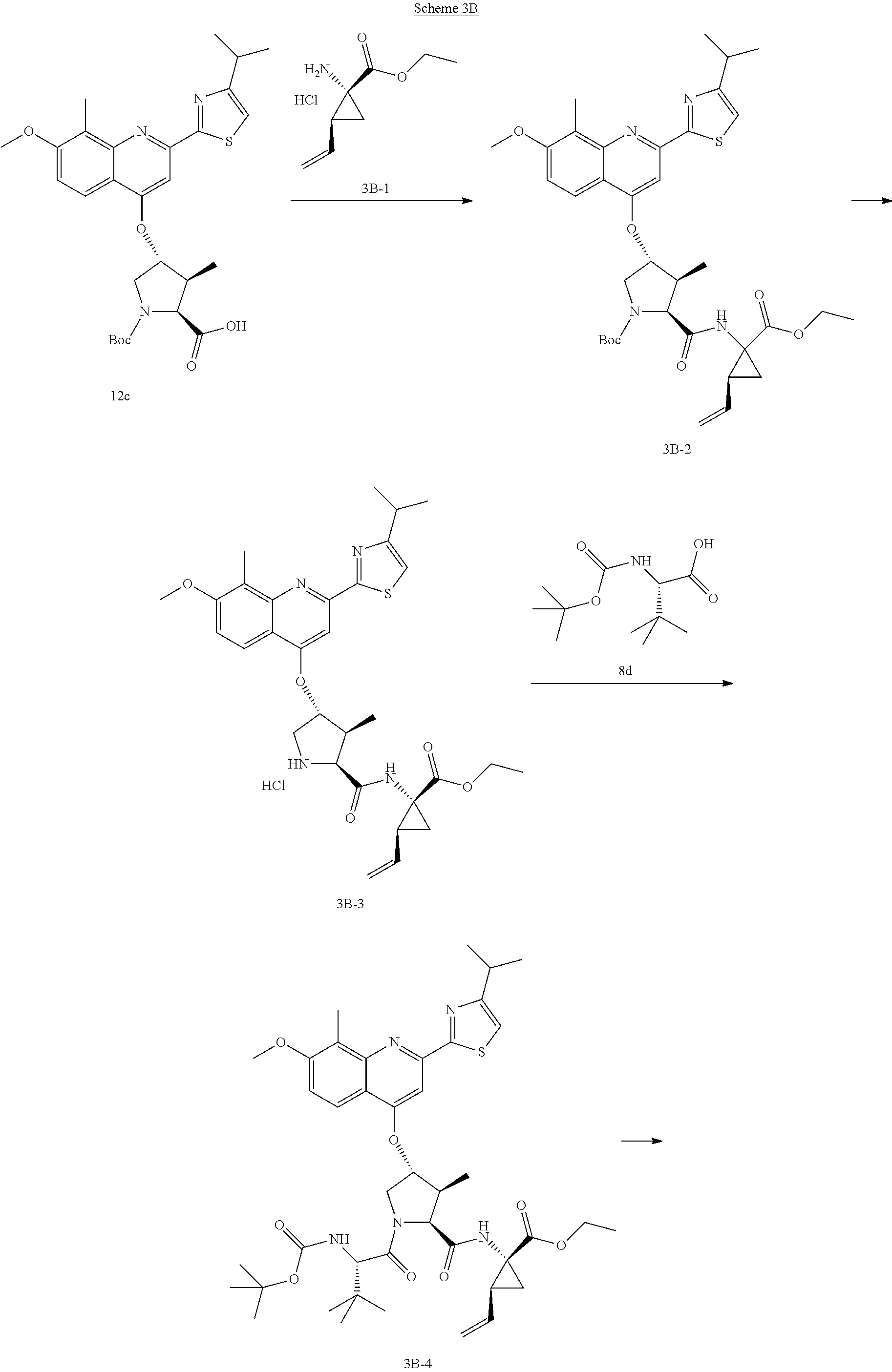

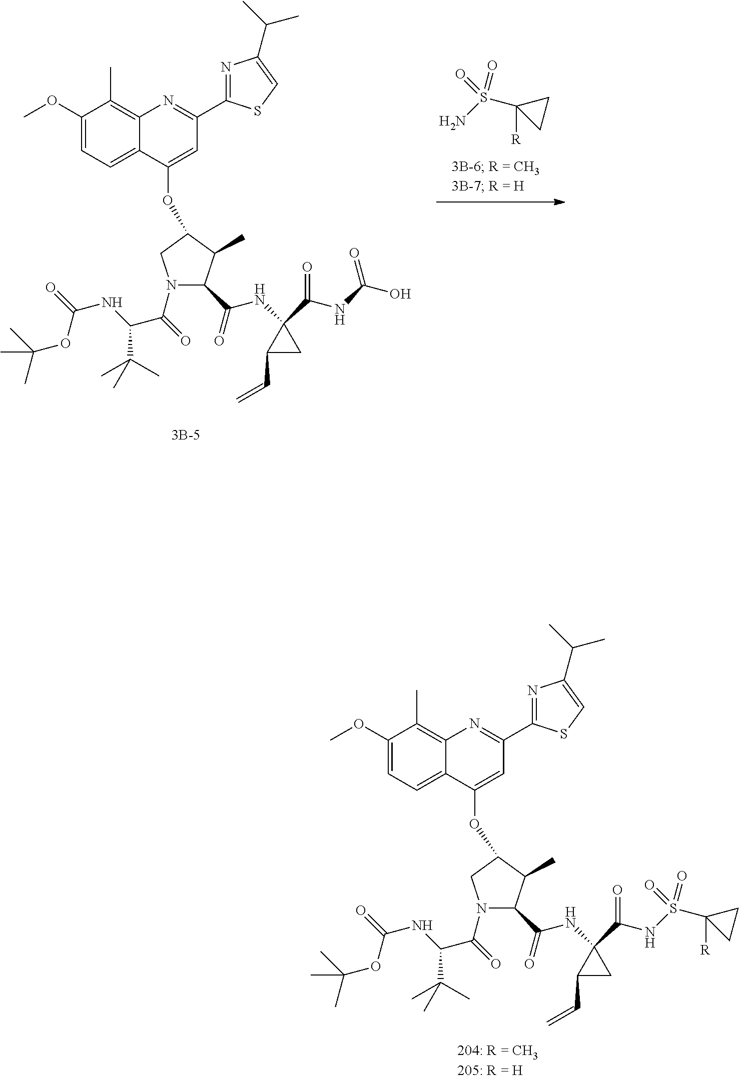

### Compound 3B-2:

To a stirred at 0° C. solution of Compound 12c (30.24 g, 55.8 mmol) and Compound 3B-1 (13 g, 90% pure, 61.4 mmol) in DMF (200 ml) was added DIPEA. After 5 min HATU (24.4 g, 64.2 mmol) was added and the reaction was allowed to proceed for 1 hour at room temperature. The reaction mixture was diluted with water and ethyl acetate and acidified with 2 N hydrochloric acid to pH 2 (85 ml). Organic phase was separated; water phase was back-extracted 3 times with ethyl acetate. Combined organic solution was washed with water, 5% sodium bicarbonate and brine. The resulted organic solution was dried over magnesium sulfate and the solvent was removed under reduced pressure to afford Compound 3B-2 which was used on the next step without any further purification. Yield 42.6 g (100%), ˜90% purity (NMR). ¹H-NMR (chloroform-d), δ: 7.95 (d, 1H), 7.50 (s, 1H), 7.21 (d, 1H), 7.02 (s, 1H), 6.69 (s, 1H), 5.78 (ddd, 1H), 5.32 (dd, 1H), 5.15 (d, 1H), 5.07 (m, 1H), 4.42 (d, 1H), 4.10-4.22 (m, 4H), 3.98 (s, 3H), 3.21 (m, 1H), 2.70 (s, 3H), 2.11 (dd, 1H), 1.92-1.96 (m, 1H), 1.58 (dd, 1H), 1.43 (s, 9H), 1.40 (d, 3H), 1.38 (d, 3H), 1.28 (d, 3H), 1.24 (t, 3H).

### Compound 3B-3:

To a solution of Compound 3B-2 (32.0 g, 47.1 mmol) in DCM (150 ml) was added 4 M HCl-dioxane (82 ml, 328 mmol). The reaction was stirred for one hour at room temperature and concentrated under reduced pressure. The residue was diluted with ethyl acetate with stirring and the resulted yellow solid was filtered off, washed with ethyl acetate and dried in vacuo to afford Compound 3B-3. Yield: 29.9 g (98%, 95% purity by HPLC). ¹H-NMR (DMSO-d⁶), δ: 10.98 (m, ex., 1H), 9.39 (s, 1H), 9.11 (m, ex., 1H), 8.22 (d, 1H), 7.50 (s, 1H), 7.49 (d, 1H), 7.48 (s, 1H), 5.66 (ddd, 1H), 5.45 (d, 1H), 5.26 (dd, 1H), 5.10 (dd, 1H), 4.48 (ddd, 1H), 4.06-4.13 (m, 2H), 4.00 (s, 3H), 3.92 (m, 1H) 3.62 (m, 1H), 3.15 (m, 1H), 2.60 (s, 3H), 2.19 (dd, 1H), 1.69 (dd, 1H), 1.35 (d, 6H), 1.15-1.19 (m, 3H), 1.05 (d, 3H).

### Compound 3B-4:

To a solution of Compound 3B-3 (28.0 g, 95% purity, 43.2 mmol) in DMF (250 ml) was added Boc-t-Leucine (8d, 11 g, 47.6 mmol). The solution was cooled to 0° C. and DIPEA (38 ml, 216 mmol) was added followed by addition of HATU (18.9 g, 49.7 mmol). The reaction was allowed to proceed for one hour at room temperature. The reaction mixture was diluted with water and ethyl acetate, acidified with 2 N aqueous hydrochloric acid to pH 2 and organic phase was separated. Aqueous phase was extracted with ethyl acetate; combined organic extract was washed with water and aqueous sodium bicarbonate. Organic solution was dried over magnesium sulfate and concentrated under reduced pressure. The residue was crystallized from ethanol to provide Compound 3B-4. Yield: 22.7 g (66%). ¹H-NMR (chloroform-d), δ: 7.97 (d, 1H), 7.54 (s, 1H), 7.19 (d, 1H), 7.01 (s, 1H), 6.42 (s, 1H), 5.80 (ddd, 1H), 5.29 (dd, 1H), 5.18 (m, 1H), 5.14 (dd, 1H), 4.27 (d, 1H), 4.15 (q, 1H), 3.98 (s, 3H), 3.72 (m, 1H), 3.18 (m, 1H), 2.91 (m, 1H), 2.70 (m, 1H), 2.18 (dd, 1H), 1.85 (dd, 1H), 1.51 (dd, 1H), 1.38-1.41 (m, 13H), 1.32 (d, 3H), 1.23 (t, 1H), 1.09 (s, 9H).

### Compound 3B-5:

To a stirred slurry of Compound 3B-4 (22.46 g, 28.3 mmol) in ethanol (600 ml) was added DCM (80 ml) and 2 N aqueous sodium hydroxide (140 ml, 70 mmol). The slurry was stirred at 45° C. and after approximately 1 hour the solid dissolved and after another hour product started to crystallize from the reaction mixture. The reaction was allowed to proceed for another two hours (total reaction time ˜4 h) and cooled to room temperature. The solid was filtered off and washed with water. The solid was slurried in water (300 ml) and the suspension was made acidic (pH ˜2) with 2 N aqueous hydrochloric acid. The product was extracted with DCM; organic solution was washed with water and dried over sodium sulfate. The solvent was removed under reduced pressure to afford Compound 3B-5 which was used on the next step without any further purification. Yield 20.7 g (96%). ¹H-NMR (DMSO-d⁶), δ: 12.40 (s, 1H), 8.64 (s, 1H), 7.50 (s, 1H), 7.48 (d, 1H), 7.35 (d, 1H), 6.68 (d, 1H), 5.71 (m, 1H), 5.04-5.21 (m, 4H), 4.50 (d, 1H), 4.21 (m, 1H), 4.06 (m, 1H), 3.95 (s, 3H), 3.15 (m, 1H), 2.83 (m, 1H), 2.58 (s, 3H), 2.01 (dd, 1H), 1.56 (dd, 1H), 1.35 (d, 6H), 1.28 (s, 9H), 1.10 (d, 3H), 0.99 (s, 9H).

### Compound 204:

To a stirred solution of Compound 3B-5 (10.0 g, 13.1 mmol) in anhydrous DCM (60 ml) was added CDI (2.55 g, 15.7 mmol) and the reaction was kept for 3 hours at room temperature. To this solution were added 1-methylcyclopropane-1-sulfonamide (3B-6, 2.30 g, 17 mmol) and DBU (2.54 ml, 17 mmol). The reaction was allowed to proceed overnight at 40° C. and then it was concentrated under reduced pressure. The residue was taken into water-ethyl acetate; the mixture was acidified to pH ˜2 with 2 N aqueous hydrochloric acid and aqueous phase was separated. The organic solution was washed with brine, dried over magnesium sulfate and the solvent was removed under vacuum. The residue was purified by chromatography in 20-30% acetone-hexane to provide Compound 204. Yield 10.5 g (93%). The analytical data was identical to data described herein.

### Compound 205:

Compound 205 was synthesized as described for the synthesis of Compound 104 from Compound 3B-5 (10.0 g, 13.1 mmol) and Compound 3B-7. Yield 10.3 g (90.7%). The analytical data was identical to data described herein.

### Synthesis of Compounds 203 and 207

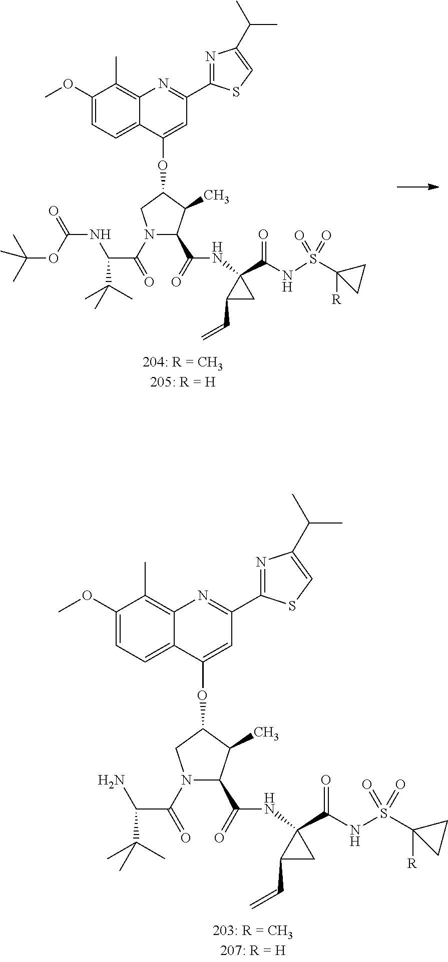

### Compound 203:

To a solution of Compound 204 (57 mg, 0.065 mmol) in DCM (1 ml) was added 4 M HCl-dioxane (0.2 ml, 0.8 mmol). The reaction mixture was stirred at room temperature for 3 hours. The yellow solid was filtered off and rinsed with ether to provide Compound 203. Yield 37 mg (70%). ¹H-NMR (DMSO-d⁶-D₂O, 70° C.), δ: 8.96 (s, 1H), 8.10 (d, 1H), 7.53 (s, 1H), 7.42 (d and s, 2H), 5.51 (m, 1H), 5.22 (d, 1H), 5.18 (m, 1H), 5.10 (d, 1H), 4.66 (d, 1H), 4.24 (dd, 1H), 3.93-4.06 (s and m, 4H), 2.60 (s, 3H), 2.19 (dd, 1H), 1.74 (dd, 1H), 1.42 (s, 3H), 1.34 (d, 6H), 1.04-1.10 (s and d, 12H), 0.89 (m, 2H).

### Compound 207:

Compound 207 was synthesized from Compound 205 as described for the synthesis of Compound 203. ¹H-NMR (DMSO-d⁶), δ: 10.57 (s, 1H), 8.93 (s, 1H), 8.15 (d, 1H), 8.11 (m, 3H), 7.42-7.51 (m, 3H), 5.49-5.56 (m, 1H), 5.24 (s, 1H), 5.21 (dd, 1H), 5.10 (dd, 1H), 4.63 (d, 1H), 4.05-4.10 (m, 3H), 3.98 (s, 3H), 3.15 (m, 1H), 2.91-2.98 (m, 2H), 2.60 (s, 3H), 2.19 (dd, 1H), 1.73 (dd, 1H), 1.35 (d, 6H), 0.99-1.12 (m, 15H).

### Compounds 205-207

Compounds 205-206 can be prepared from Compound 1e in a manner analogous to the synthesis of Compound 204 described above. Compound 207 can be prepared from Compound 205 in a manner analogous to the synthesis of Compound 203 described above.

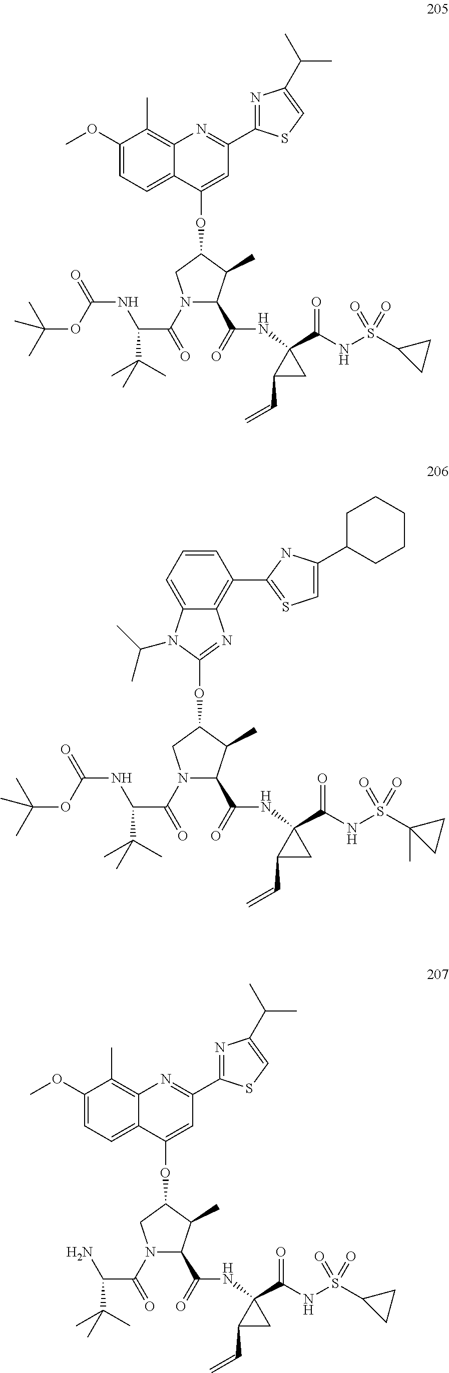

### Synthesis of Compound 206:

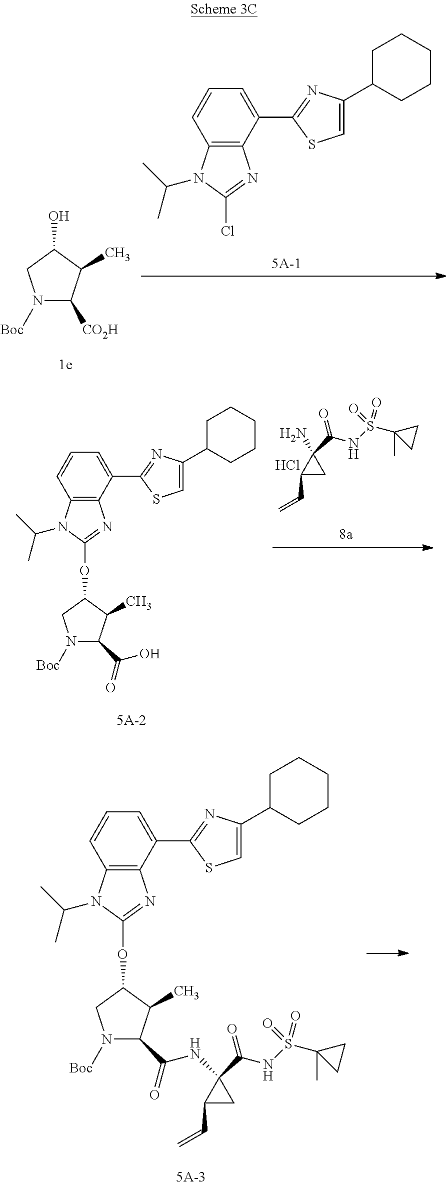

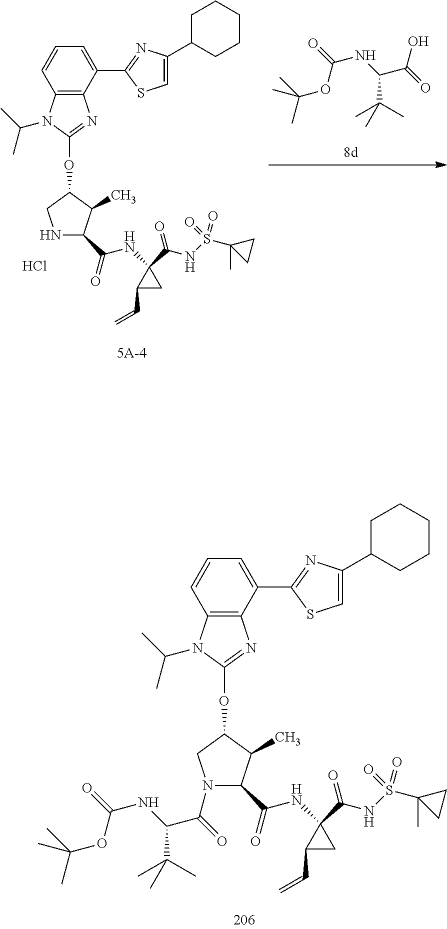

### Compound 5A-2

To a stirred solution of Compound 1e (140 mg, 0.57 mmol) and 2-(2-chloro-1-isopropyl-1H-benzo[d]imidazol-4-yl)-4-cyclohexylthiazole (5A-1, 206 mg, 0.57 mmol) in DMSO (3 ml) was added potassium tert-butoxide (147 mg, 1.31 mmol) and the reaction was allowed to stir for 2 hours at room temperature followed by stirring at 40° C. for 2 hours. The reaction was partitioned between ethyl acetate-water and acidified with 2 N aqueous hydrochloric acid to pH ˜2. The organic phase was separated, washed with water, dried over magnesium sulfate and concentrated under reduced pressure. The residue was purified by column chromatography in 0-10% methanol-DCM to afford Compound 5A-2. Yield 264 mg (81%). ¹H-NMR (DMSO-d⁶, 70° C.), δ: 12.52 (br. s, 1H), 7.97 (d, 1H), 7.51 (d, 1H), 7.28 (s, 1H), 7.20 dd, 1H), 5.27 (m, 1H), 4.72 (m, 1H), 4.34 (d, 1H), 4.05-4.27 (m, 1H), 3.58 (dd, 1H), 3.10 (m, 1H), 2.87-2.98 (m, 1H), 2.79 (m, 1H), 2.03-2.10 (m, 2H), 1.78-1.84 (m, 2H), 1.68-1.76 (m, 1H), 1.52 (d, 3H), 1.49 (d, 3H), 1.39 (s, 9H), 1.14 (d, 3H).

### Compound 5A-3:

To a stirred solution of Compound 5A-2 (200 mg, 0.35 mmol) and (1R,2S)-1-amino-N-((1-methylcyclopropyl)sulfonyl)-2-vinylcyclopropanecarboxamide hydrochloride (8a, 128 mg, 0.46 mmol) in DMF (3 ml) were added DIPEA (0.61 ml, 3.5 mmol) and HATU (175 mg, 0.46 mmol). The reaction was allowed to stir for 1 h, when it was taken into ethyl acetate-water and acidified to pH ˜3 with 2 N aqueous hydrochloric acid. The organic phase was separated, washed with water, dried over magnesium sulfate and the solvent was removed in vacuo. The residue was purified by column chromatography in 20-40% acetone-hexane to provide Compound 5A-3. Yield 265 mg (95%). ¹H-NMR (chloroform-d, 60° C.) δ: 9.61 (s, 1H), 8.14 (d, 1H), 7.17-7.25 (d, 1H), 7.21 (dd, 1H), 6.95 (s, 1H), 6.57 (s, 1H), 5.78 (ddd, 1H), 5.47 m, 1H), 5.32 (d, 1H), 5.18 (d, 1H), 4.59 (m, 1H), 4.39 (d, 1H), 4.10-4.20 (m, 1H), 3.81-3.85 (m, 1H), 2.98-3.05 (m, 1H), 2.88 (m, 1H), 2.14-2.19 (m, 2H), 1.94 (dd, 1H), 1.82-1.88 (m, 2H), 1.60-1.80 (m, 3H), 1.55 (d, 3H), 1.54 (d, 3H), 1.50 (s, 9H), 1.22-1.42 (m, 4H), 1.20 (d, 3H), 0.82 (m, 2H).

### Compound 5A-4:

To a stirred solution of Compound 5A-3 (248 mg, 0.31 mmol) in DCM (2 ml) was added 4 M HCl-dioxane (0.8 ml, 3.2 mmol) and the mixture was stirred for 1 hour a room temperature. The solvent was removed under reduced pressure. Compound 5A-4 was obtained by trituration of the residue with ethyl acetate. Yield 226 mg (100%). ¹H-NMR (DMSO-d⁶), δ: 11.45 (s, 1H), 10.56 (br. m, 1H), 9.26 (s, 1H), 9.12 (br. m, 1H), 7.98 (d, 1H), 7.61 (d, 1H), 7.33 (s, 1H), 7.22 (dd, 1H), 5.42-5.51 (m, 2H), 5.26 (d, 1H), 5.10 (d, 1H), 4.75 (m, 1H), 4.55 (ddd, 1H), 3.94-4.00 (m, 1H), 3.65-3.72 (m, 1H), 3.21 (m, 1H), 2.77 (m, 1H), 2.28 (dd, 1H), 2.03-2.06 (m, 2H), 1.84 (dd, 1H), 1.74-1.83 (m, 2H), 1.66-1.74 (m, 1H), 1.56 (d, 3H), 1.54 (d, 3H), 1.37-1.48 (m, 8H), 1.14-1.28 (m, 3H), 0.96 (d, 3H), 0.89 (m, 2H).

### Compound 206:

To a stirred solution of Compound 5A-4 (30 mg, 0.043 mmol) and Boc-t-Leucine (8d, 13 mg, 0.056 mmol) in DMF (1 ml) were added DIPEA (70 mcl, 0.4 mmol) and HATU (21 mg, 0.056 mmol). The reaction was allowed to proceed for 1 hour at room temperature, taken into ethyl acetate-water and acidified with 2 N aqueous hydrochloric acid to pH ˜3. The organic layer was separated, washed with water, dried over magnesium sulfate and concentrated under reduced pressure. The residue was purified by column chromatography in 20-30% acetone-hexane to afford Compound 206. Yield 21 mg (54%). ¹H-NMR (chloroform-d, 60° C.) δ: 9.58-9.75 (br. s, 1H), 8.15 (d, 1H), 7.26 (d, 1H), 7.19 (dd, 1H), 6.94 (s, 1H), 6.72-6.81 (br. s, 1H), 5.58-5.75 (m, 1H), 5.49 (m, 1H), 5.25 (d, 1H), 5.13 (d, 1H), 4.60 (m, 1H), 4.50 (m, 1H), 4.30 (m, 1H), 4.05-4.15 (m, 1H), 2.88-3.05 (m, 2H), 2.10-2.22 (m, 4H), 1.58-1.94 (m, 8H), 1.21-1.55 (m, 33H), 1.09 (s, 9H), 0.81 (m, 2H). LC-MS 920.4 (M+H)⁺.

### Synthesis of Compound 208

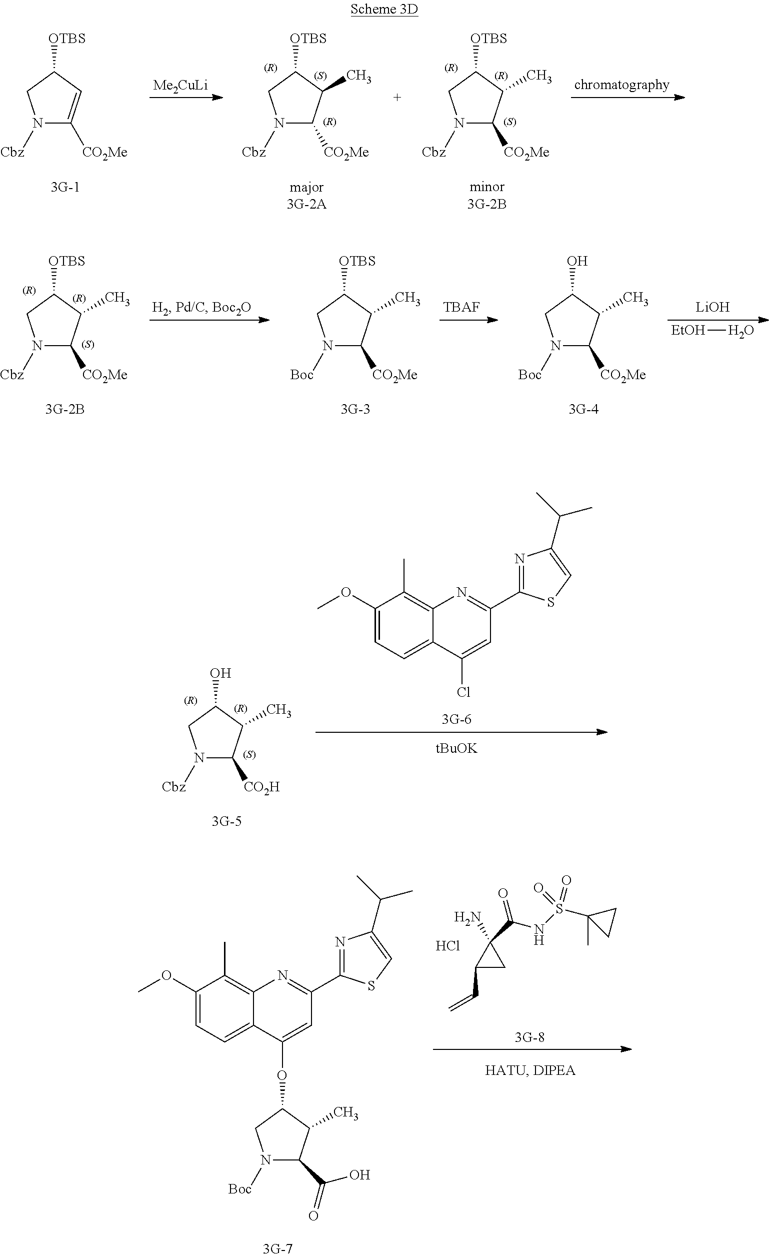

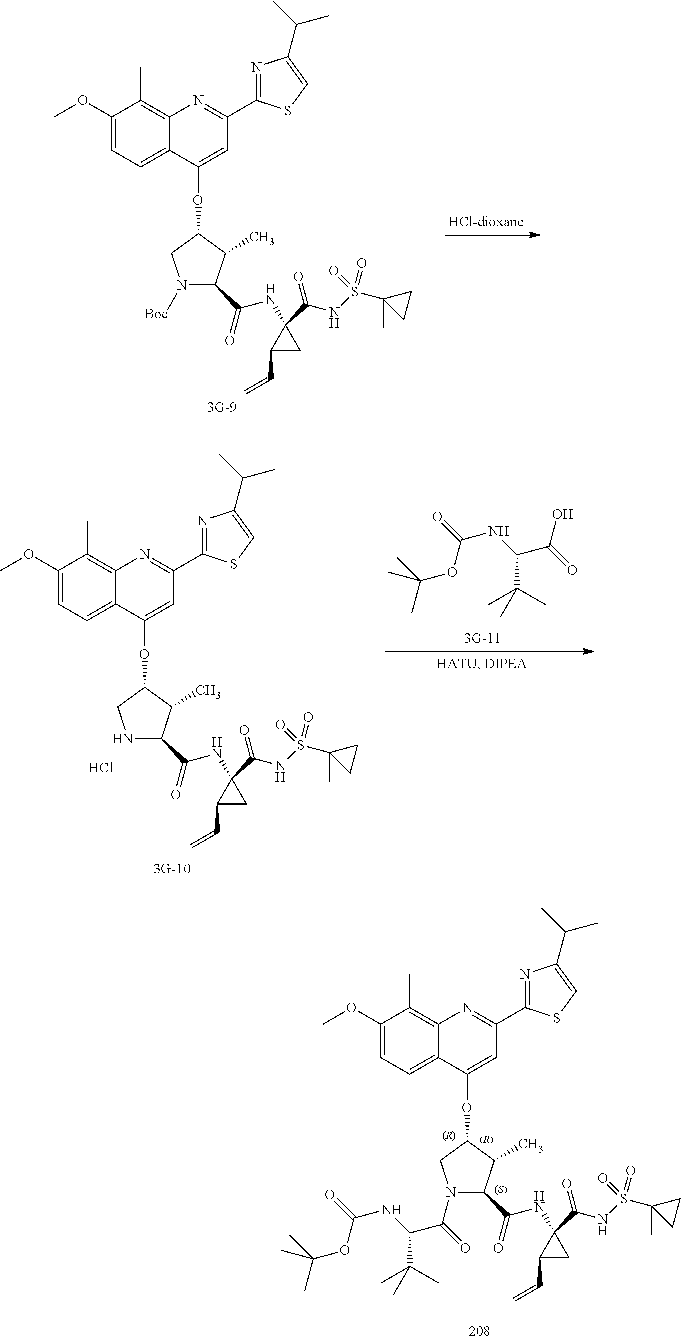

### Compound 3G-2B

The mother liquor after first crystallization of (2R,3S,4R)-diastereoisomer was concentrated under reduced pressure to give ˜36 g of an oily residue. Approximately 5 g of this oil was purified by column chromatography in 5-15% ethyl acetate-hexane to provide 3G-2B.

Yield 600 mg (approximately 6%). ¹H-NMR (chloroform-d), δ: (two rotamers) 7.28-7.34 (m, 5H), 5.01-5.22 (m, 2H), 4.15-4.18 (m, 1H), 4.03 and 3.97 (two d, !H), 3.77 (s, 1.4H), 3.56-3.55 (m, 2H), 3.53 (s, 1.6H) 2.20-2.26 (m, 1H), 1.11 and 1.08 (two d, 3H), 0.87 and 0.86 (two s, 9H), 0.06 (s, 6H).

### 6-2. (2S,3R,4R)-1-tert-butyl 2-methyl 4-((tert-butyldimethylsilyl)oxy)-3-methylpyrrolidine-1,2-dicarboxylate

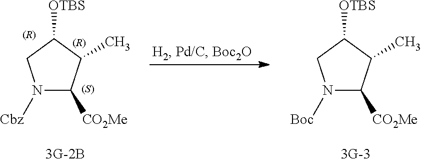

A solution of 3G-2B from the previous step (408 mg, 1 mmol) and Boc₂O (327 mg, 1.5 mmol) in THF (20 ml) was hydrogenated overnight in the presence of 10% Pd/C (50 mg). The catalyst was filtered off and the solvent was removed under reduced pressure. The residue was purified by column chromatography in 10-30% ethyl acetate-hexane to afford 3G-3 as an oil.

Yield 250 mg (67%). ¹H-NMR (chloroform-d), δ: (two rotamers) 4.14 (m, 1H), 3.97 and 3.88 (d and d, 1H), 3.76 and 3.74 (s and s, 3H), 3.42-3.54 (m, 2H), 2.18-2.25 (m, 1H), 1.45 and 1.41 (s and s, 9H), 1.10 and 1.08 (d and d, 3H), 0.88 (s, 9H), 0.06 (s, 6H).

### Compound 3G-4

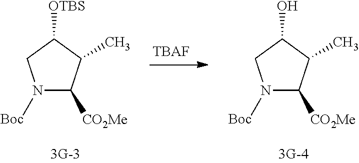

To a solution of 3G-3 from the previous step (250 mg, 0.67 mmol) in THF (3 ml) was added 1M solution of TBAF in THF (0.87 ml, 0.87 mmol). After 1 hour the reaction was quenched by addition of saturated aqueous sodium bicarbonate and then extracted with ethyl acetate. The solvent was removed under reduced pressure and the residue was purified by column chromatography in 30-70% ethyl acetate-hexane) to afford 3G-4 as a colorless oil.

Yield 177 mg (100%). ¹H-NMR (chloroform-d), δ: (two rotamers) 4.17 (m, 1H), 3.92 and 3.88 (d and d, 1H), 3.72 and 3.71 (s and s, 3H), 3.48-3.61 (m, 2H), 2.50-2.56 (br. s, 1H), 2.23 (m, 1H), 1.41 and 1.36 (s and s, 9H), 1.13 (d, 3H),

### Compound 3G-5

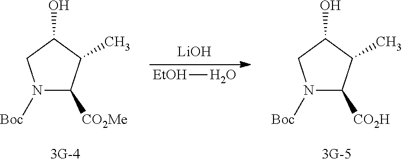

To a solution 3G-4 from the previous example (177 mg, 0.67 mmol) in ethanol (3 ml) was added 2N aqueous lithium hydroxide (3.4 ml, 6.8 mmol). The reaction mixture was stirred for one hour at 40° C. when it was acidified to pH ˜2 with 2N aqueous hydrochloric acid and extracted with ethyl acetate. The organic extract was dried over magnesium sulfate and the solvent was removed under reduced pressure to afford 3G-5 as an oil which was used on the next step without any further purification. Yield 160 mg (97%).

### Compound 3G-7

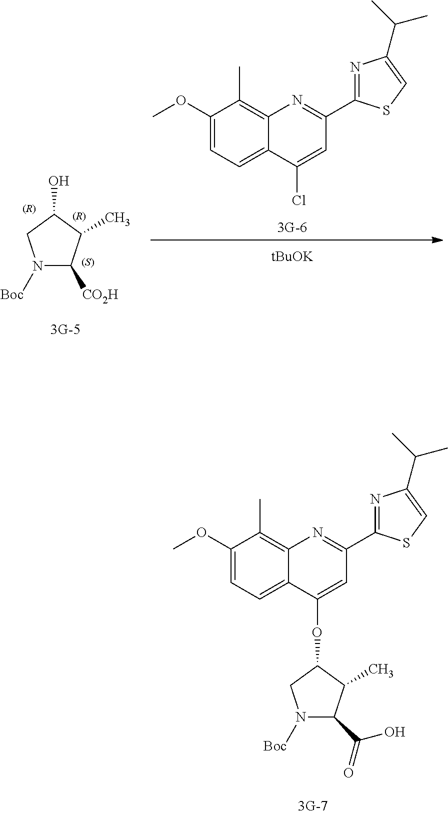

To a stirred solution of 3G-5 from the previous example (100 mg, 0.408 mmol) in DMSO (2 ml) was added 3G-6 (176 mg, 0.53 mmol) followed by addition of potassium tert-butoxide (105 mg, 0.94 mmol). The reaction was allowed to proceed overnight at room temperature, when it was quenched with water and acidified with aqueous hydrochloric acid to pH ˜2. The mixture was extracted with ethyl acetate; organic phase was washed with water, dried over magnesium sulfate and the solvent was removed under reduced pressure. The residue was purified by column chromatography in 0-15% methanol in DCM to afford 3G-7 as a yellow foam.

Yield: 196 mg (89%). ¹H-NMR (DMSO-d⁶, 70° C.), δ: 12.05-12.80 (br. s, 1H), 8.00 (d, 1H), 7.51 (s, 1H), 7.43 (d, 1H), 7.40 (s, 1H), 5.33 (m, 1H), 4.01 (d, 1H), 3.95 (s, 3H), 3.67-3.80 (m, 2H), 3.15 (m, 1H), 2.62-2.72 (m, 1H), 2.56 (s, 3H), 1.31-1.35 (m, 15H), 1.25 (d, 3H).

### Compound 3G-9

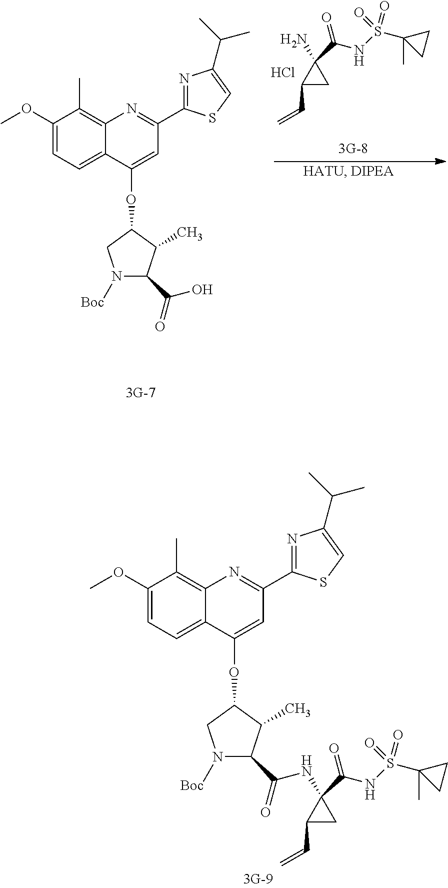

To a solution 3G-7 from the previous step (196 mg, 0.36 mmol) in DMF (3 ml) was added 3G-8 (132 mg, 0.47 mmol) followed by DIPEA (0.7 ml, 4 mmol) and HATU (179 mg, 0.47 mmol). The reaction was allowed to proceed for overnight. The reaction was quenched with water, acidified with aqueous hydrochloric acid to pH ˜2 and extracted with ethyl acetate. Organic extract was washed with water, dried over magnesium sulfate and the solvent was removed under reduced pressure. The residue was purified by column chromatography in 20-70% acetone-hexane to afford 3G-9 as a pale-yellow foam.

Yield 190 mg (69%). ¹H-NMR (chloroform-d), δ: 9.84 (s, 1H), 7.95 (d, 1H), 7.48 (s, 1H), 7.24 (d, 1H), 7.05 (s, 1H), 6.95 (s, 1H), 5.824 (s, 1H), 5.32 (d, 1H), 5.23 (m, 1H), 5.18 (d, 1H), 4.05 (d, 1H), 4.00 (s, 3H), 3.80-3.88 (m, 2H), 3.20 (m, 1H), 2.87 (m, 1H), 2.71 (s, 3H), 2.19 (dd, 1H), 2.03 (dd, 1H), 1.54-1.78 (m, 3H), 1.53 (s, 3H), 1.36-1.48 (m, 13H), 1.24-1.26 (m, 4H), 0.80-0.92 (m, 2H).

### Compound 3G-10

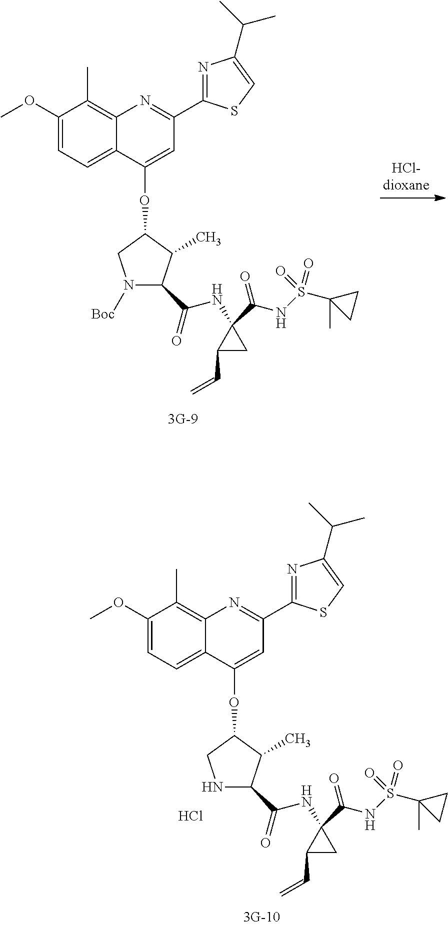

To a solution of 3G-9 from the previous example (190 mg, 0.247 mmol) in DCM (3 ml) was added 4M HCl-dioxane (0.62 ml, 2.5 mmol). The reaction was allowed to proceed for 1.5 h at room temperature and the solvent was removed in vacuo to afford 3G-10 as a yellow foam which was used on the next step without any further purification.

Yield 174 mg (100%). ¹H-NMR (DMSO-d⁶), δ: 11.40 (s, 1H), 10.10 (m, 1H), 9.65 (s, 1H), 9.13 (m, 1H), 8.16 (d, 1H), 7.52 (s, 1H), 7.49 (s, 1H), 7.46 (d, 1H), 5.52-5.62 (m, 2H), 5.36 (d, 1H), 5.16 (d, 1H), 4.28 (m, 1H), 3.99 (s, 3H), 3.69-3.87 (m, 1H), 3.59-3.64 (m, 1H), 3.15 (m, 1H), 2.56-2.65 (s and m, 4H), 2.38 (dd, 1H), 1.86 (dd, 1H), 1.38-1.42 (s and m, 5H), 1.34 (d, 6H), 1.20 (d, 3H), 0.86-0.96 (m, 2H).

### Compound 208

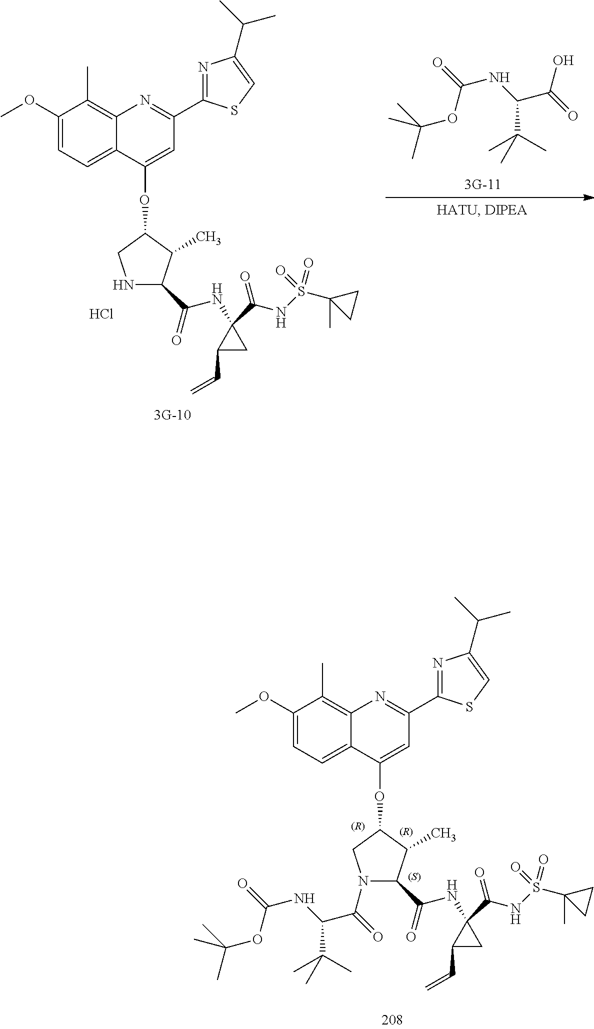

To a solution of 3G-10 from the previous example (100 mg, 0.14 mmol) in DMF (2 ml) was added 3G-11 (43 mg, 0.185 mmol) followed by DIPEA (0.24 ml, 1.4 mmol) and HATU (70 mg, 0.185 mmol). The reaction was allowed to proceed for 2 h at room temperature, when it was quenched with water. The reaction mixture was acidified with aqueous hydrochloric acid to ph ˜2 and extracted with ethyl acetate. The organic phase was washed with water, dried over magnesium sulfate and concentrated under reduced pressure. The residue was purified by column chromatography in 20-40% acetone-hexane to afford the target compound 208 as a pale-yellow foam.

Yield 107 mg (87%). ¹H-NMR (chloroform-d), δ: 9.85 (s, 1H), 7.92 (d, 1H), 7.51 (s, 1H), 7.31 (s, 1H), 7.18 (d, 1H), 7.03 (s, 1H), 5.75 (m, 1H), 5.28-5.33 (m, 2H), 5.12-5.18 (m, 2H), 4.36 (d, 1H), 4.25 (d, 1H), 4.15 (d, 1H), 4.05-4.11 (m, 1H), 3.97 (s, 3H), 3.21 (m, 1H), 2.89 (m, 1H), 2.69 (s, 3H), 2.19 (dd, 1H), 1.98 (dd, 1H), 1.62-1.73 (m, 2H), 1.53 (s, 3H), 1.47 (dd, 1H), 1.40 and 1.39 (d and d, 6H), 1.25 (d, 3H), 1.15 (s, 9H), 1.01 (s, 9H), 0.82-0.92 (m, 2H).

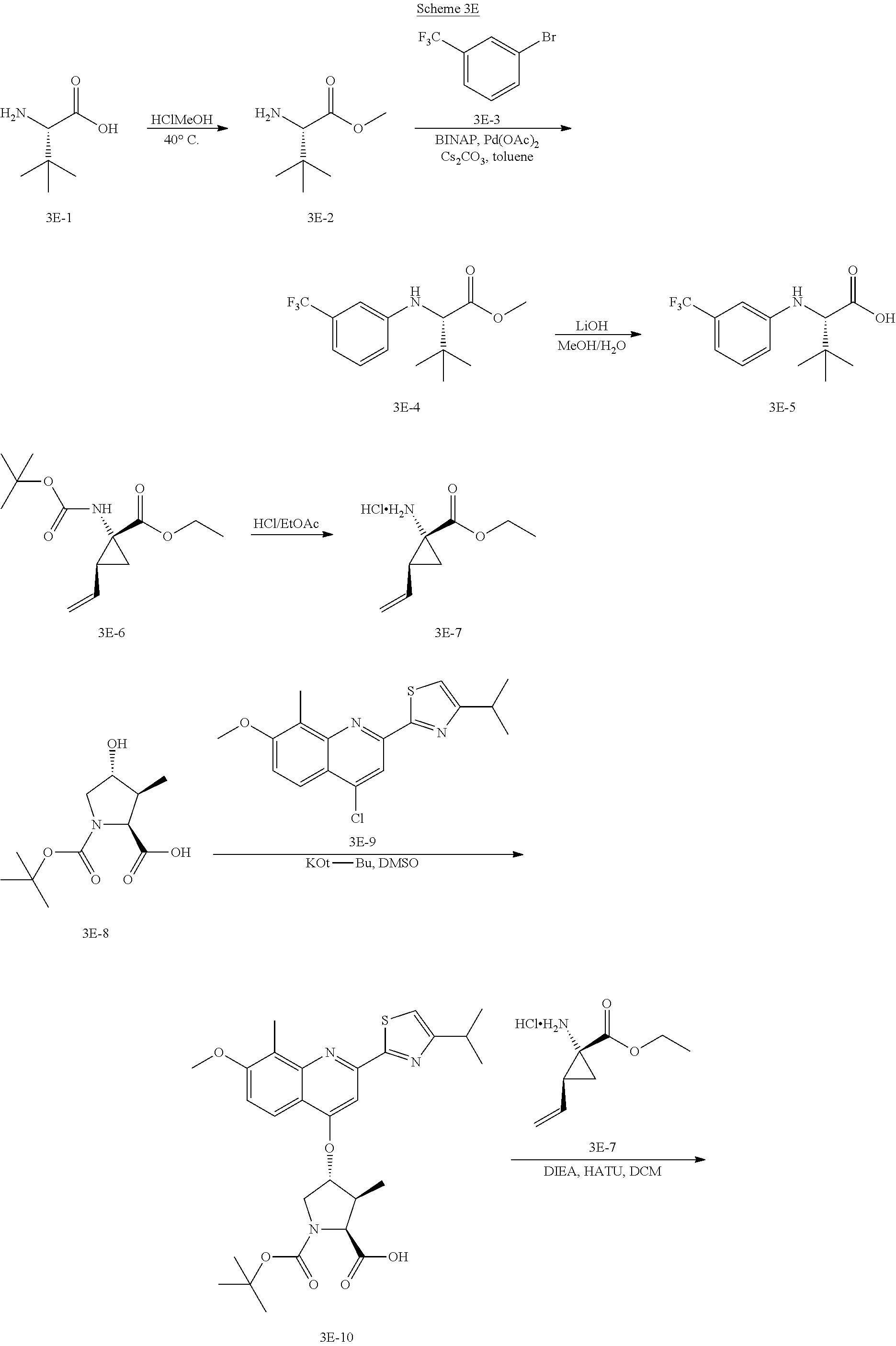

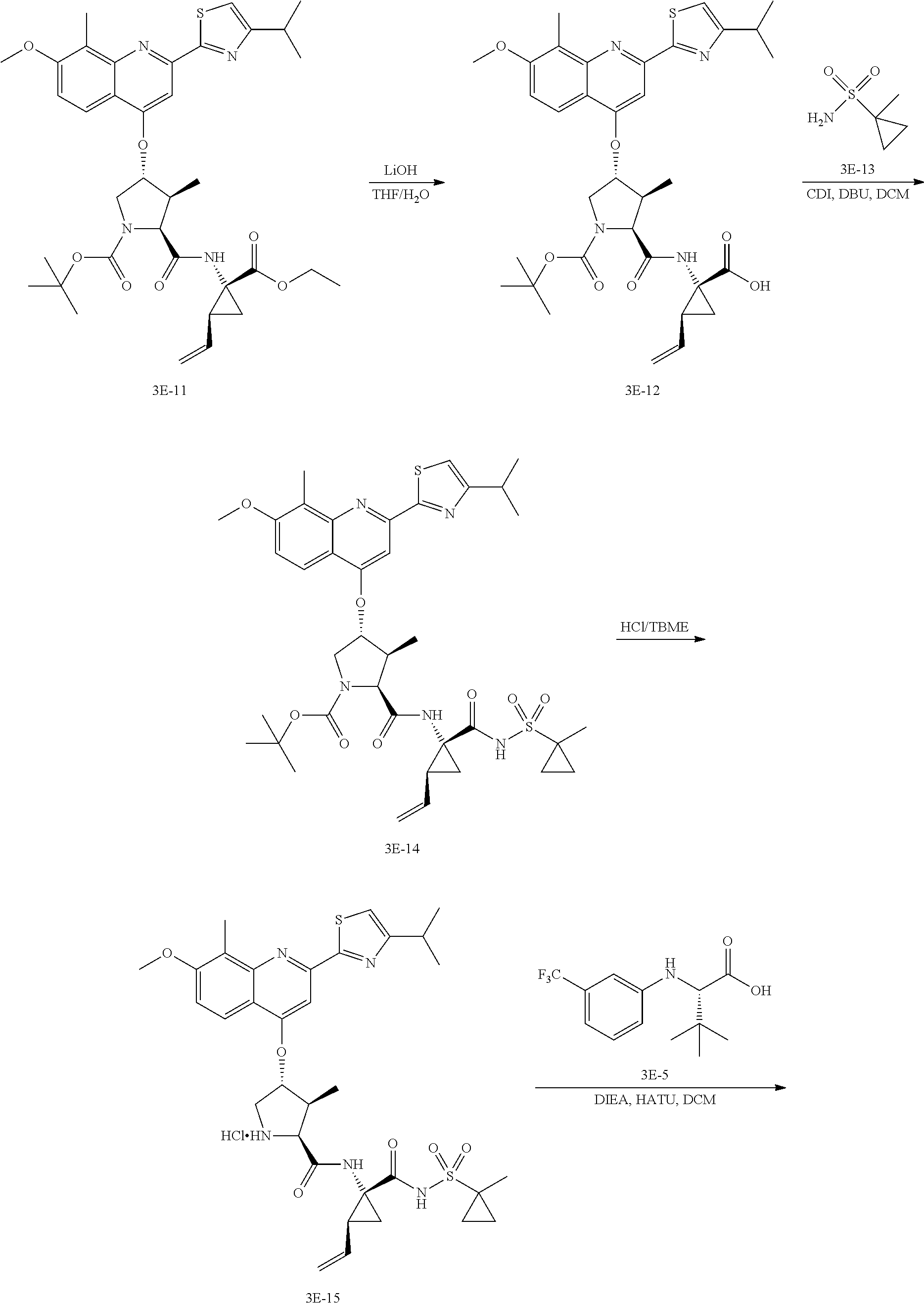

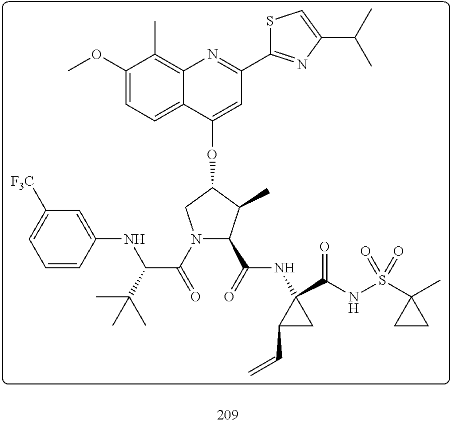

### Compound 3E-2

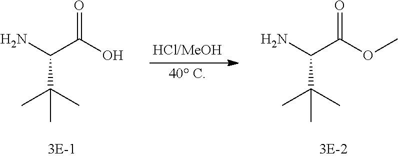

The solution of compound 3E-1 (3 g, 22.8 mmol) in 30 mL of MeOH/HCl was heated at 40° C. for 20 h. The solvent was removed under reduced pressure. The residual was diluted with water, neutralized with saturated aqueous NaHCO₃, extracted with EtOAc (100 mL×3). The combined organic layer was washed with brine, dried over anhydrous sodium sulfate, filtered and concentrated under reduced pressure. The residual was used directly for the next step (2.5 g, crude yield 76%). ¹H NMR (400 MHz, CDCl₃): δ 3.68 (s, 3H), 3.14 (s, 1H), 0.94 (s, 9H).

### Compound 3E-4

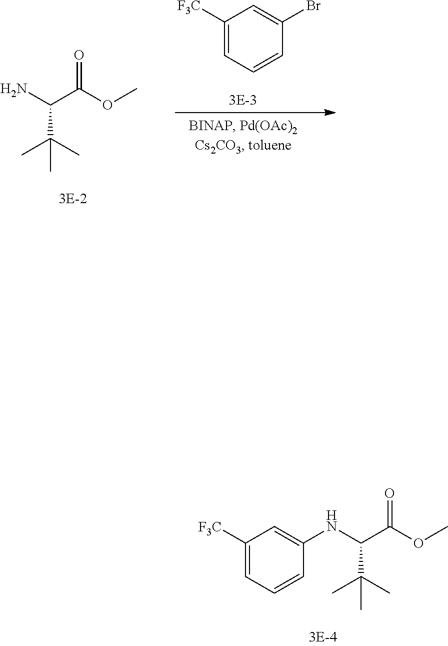

A flask were charged with compound 3E-2 (1.36 g, 9.3, mmol, 1.05 eq), compound 3E-3 (2 g, 8.89 mmol, 1 eq), BINAP (1.11 g, 1.78 mmol, 0.2 eq), Pd(OAc)₂ (0.43 g, 1.78 mmol, 0.2 eq), Cs₂CO₃ (5.8 g, 17.8 mmol, 2 eq) and toluene. The flask was flushed with nitrogen for three times. The mixture was heated to reflux for 18 hrs. LCMS analysis showed the reaction completed. The solvent was removed under reduced pressure. The residual was diluted with water, extracted with EA (100 mL×3). The combined organic layer was washed with brine, dried over anhydrous sodium sulfate, filtered and concentrated under reduced pressure. Purified by column chromatography (PE:EA=50:1-10:1) to give compound 3E-4 as yellow solid (1 g, yield 40%). MS (ESI) m/z (M+H)⁺ 289.9.

### Compound 3E-5

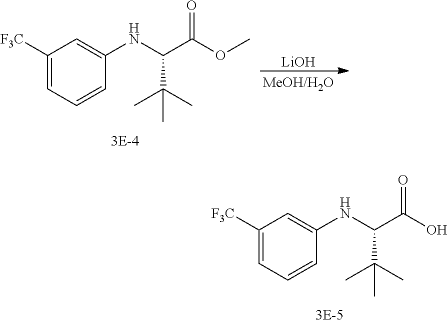

To a solution of compound 3E-4 (500 mg, 1.73 mmol, 1 eq) in 10 mL of MeOH was added LiOH.H₂O (727 mg, 17.3 mmol, 10 eq) in 3 mL of H₂O. The mixture was heated at 40° C. and stirred for 18 hrs. LCMS analysis showed the reaction completed. All the volatiles were removed under reduced pressure. The residual was diluted with water, adjusted to pH=5-6 with aq. HCl (2 N), extracted with EA (50 mL×3). The combined organic layer was washed with brine, dried over anhydrous sodium sulfate, filtered and concentrated under reduced pressure to give crude compound 3E-5 (430 mg, yield 90%). MS (ESI) m/z (M+H)⁺ 275.9.

### Compound 3E

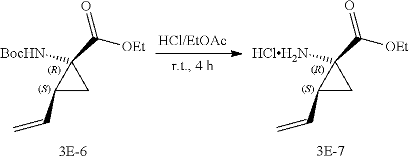

Compound 3E-6 (2.5 g, 9.8 mmol) was taken up with a solution of HCl (g) in EtOAc (4 M, 30 mL). The mixture was stirred at ambient temperature for 12 hrs. After that, the reaction mixture was concentrated under reduced pressure to afford compound 3E-7 (1.9 g, yield 99%) as a brown oil.

### Compound 3E-10

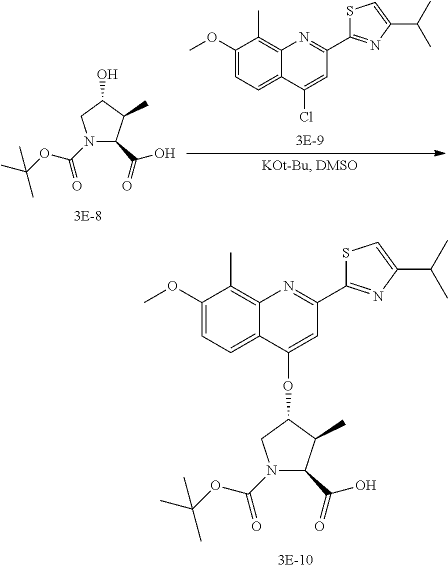

To a suspension of compound 3E-8 (650 mg, 2.65 mmol, 1 eq) in 20 mL of DMSO was added KOt-Bu (890 mg, 7.95 mmol, 3 eq) at 0° C. The generated mixture was stirred for 15 min and then the compound 3E-9 (880 mg, 2.65 mmol, 1 eq) was added in one portion. Then the reaction was stirred at r.t. overnight. The reaction mixture was quenched with ice-water (20 mL), acidified to pH=5-6 with aq. citric acid (5%). extracted with ethyl acetate, the combined organic layer was washed with brine, dried over Na₂SO₄, concentrated to give crude compound 3E-10 (1.4 g, crude 100%). MS (ESI) m/z (M+H)⁺ 542.0.

### Compound 3E-11

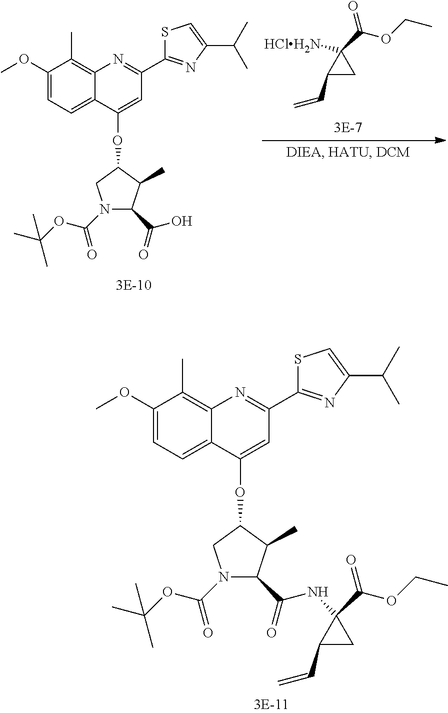

To a solution of compound 3E-10 (1 g, 1.85 mmol, 1 eq) in 30 mL of DCM was added HATU (1.05 g, 2.8 mmol, 1.5 eq), DIEA (0.95 g, 7.4 mmol, 4 eq), and compound 3E-7 (0.71 g, 3.7 mmol, 2 eq). The mixture was stirred for 18 h at room temperature. LCMS analysis showed the reaction completed. All the volatiles were removed under reduced pressure. The residual was diluted with water, extracted with EA (100 mL×3). The combined organic layer was washed with brine, dried over anhydrous sodium sulfate, filtered and concentrated under reduced pressure. Purified by column chromatography (PE:EA=50:1˜2:1) to afford compound 3E-11 as yellow solid (0.9 g, yield 72%). MS (ESI) m/z (M+H)⁺ 679.2.

### Compound 3E-12

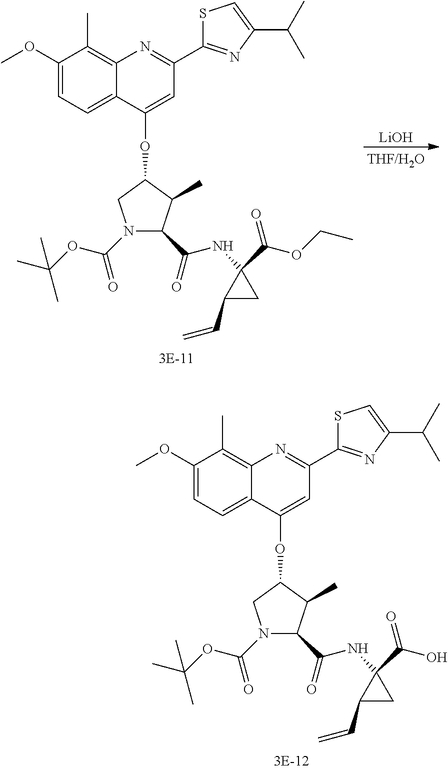

To a solution of compound 3E-11 (0.98 g, 1.44 mmol, 1 eq) in 20 mL of THF was added LiOH.H₂O 0.61 g, 14.4 mmol, 10 eq) in 5 mL of H₂O. The mixture was stirred at r.t. for 18 hrs. LCMS analysis showed the reaction completed. All the volatiles were removed under reduced pressure. The residual was diluted with water, adjusted to pH=5-6 with aq.HCl (2 N), extracted with EA (100 mL×3). The combined organic layer was washed with brine, dried over anhydrous sodium sulfate, filtered and concentrated under reduced pressure. The residual was dried and used directly for the next step without purification (0.9 g, crude yield 96%). MS (ESI) m/z (M+H)⁺ 651.1.

### Compound 3E-14

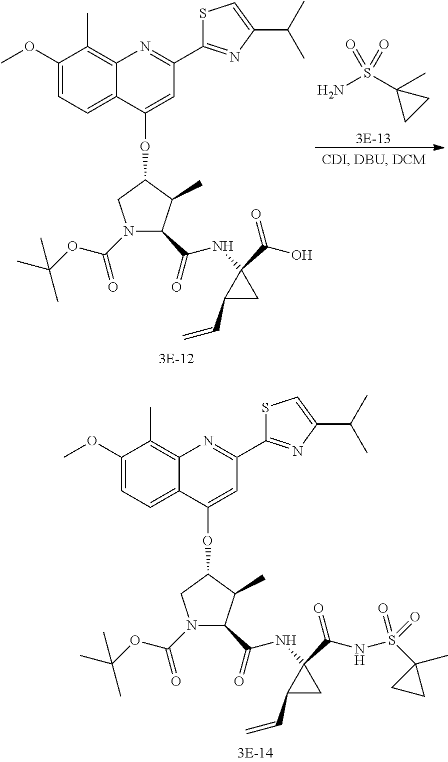

To a solution of 3E-12 (300 mg, 0.46 mmol, 1 eq) in 5 mL of DCM was added CDI (301 mg, 1.84 mmol, 4 eq). The solution was heated to reflux for 2 hrs. After that, DBU (559 mg, 3.68 mmol, 8 eq), and compound 3E-13 (311 mg, 2.3 mmol, 5 eq) were added into the flask. The mixture was stirred for 18 hrs at 30° C. LCMS analysis showed the reaction completed. All the volatiles were removed under reduced pressure. The residual was diluted with water, extracted with EA (50 mL×3). The combined organic layer was washed with brine, dried over anhydrous sodium sulfate, filtered and concentrated under reduced pressure. The residue was purified by prep-HPLC to provide compound 3E-14 as white solid (200 mg, yield 56%). MS (ESI) m/z (M+H)⁺ 767.9.

### Compound 3E-15

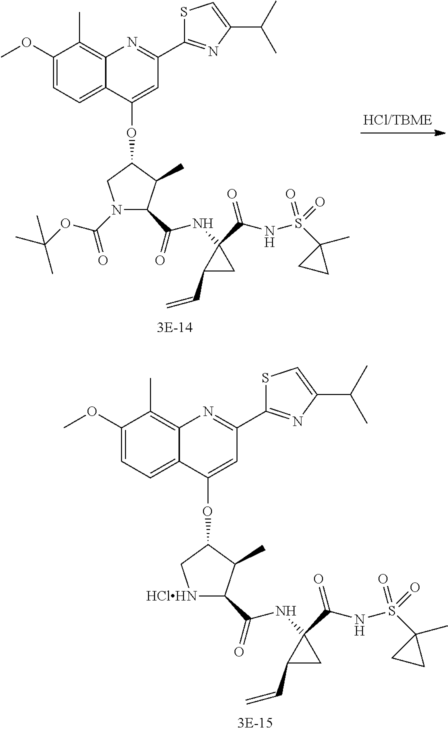

The mixture of compound 3E-14 (200 mg, 0.26 mmol) in 10 mL of solution of HCl (g) in TBME (saturated) was stirred at r.t. for 2 hrs. LCMS analysis showed the reaction completed. The solvent was removed under reduced pressure. The residual was dried and used directly for the next step without purification (170 mg, crude yield 98%). MS (ESI) m/z (M+H)⁺ 668.1.

### Compound 209

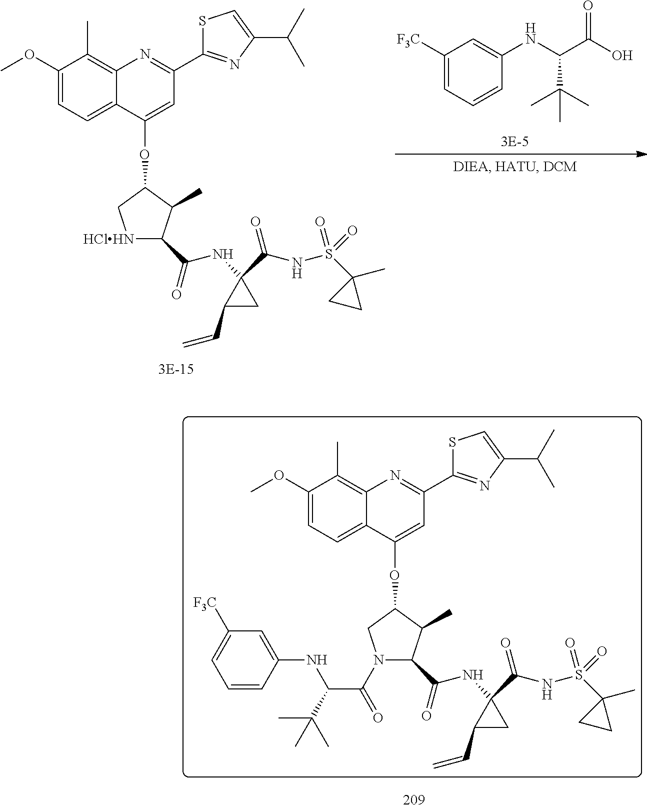

To a solution of compound 3E-5 (117 mg, 0.43 mmol, 1.5 eq) in 6 mL of DCM was added HATU (162 mg, 0.43 mmol, 1.5 eq), DIEA (145 mg, 1.12 mmol, 4 eq), and compound 3E-15 (200 mg, 0.28 mmol, 1 eq). The mixture was stirred for 18 h at room temperature. TLC (DCM:Methanol=40:1) analysis showed the reaction completed. All the volatiles were removed under reduced pressure. The residual was diluted with water, extracted with EA (50 mL×3). The combined organic layer was washed with brine, dried over anhydrous sodium sulfate, filtered and concentrated under reduced pressure. Purification by prep-TLC gave Compound 209 as white solid (132 mg, yield 51%). MS (ESI) m/z (M+H)⁺ 925.3.

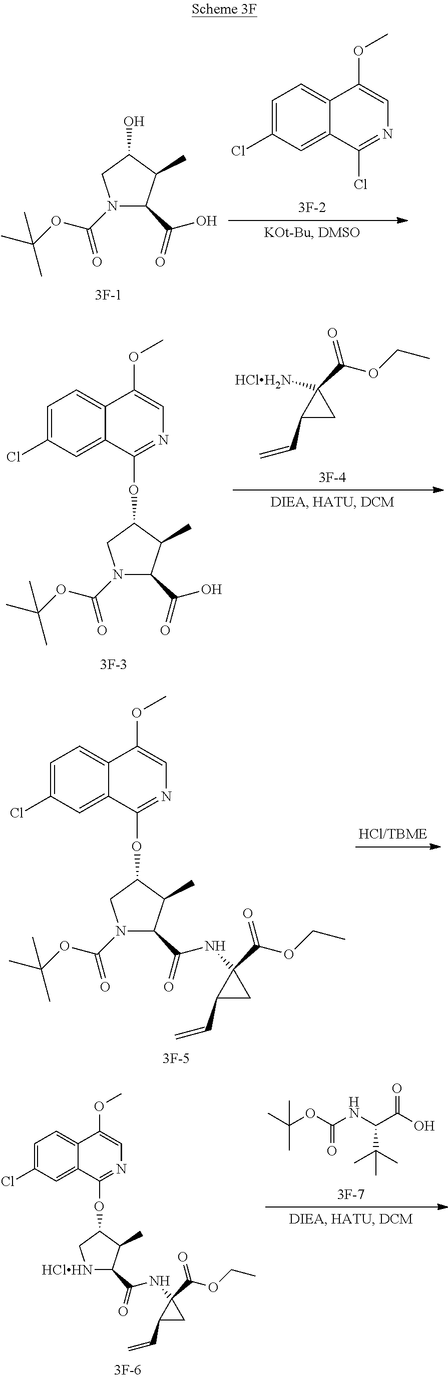

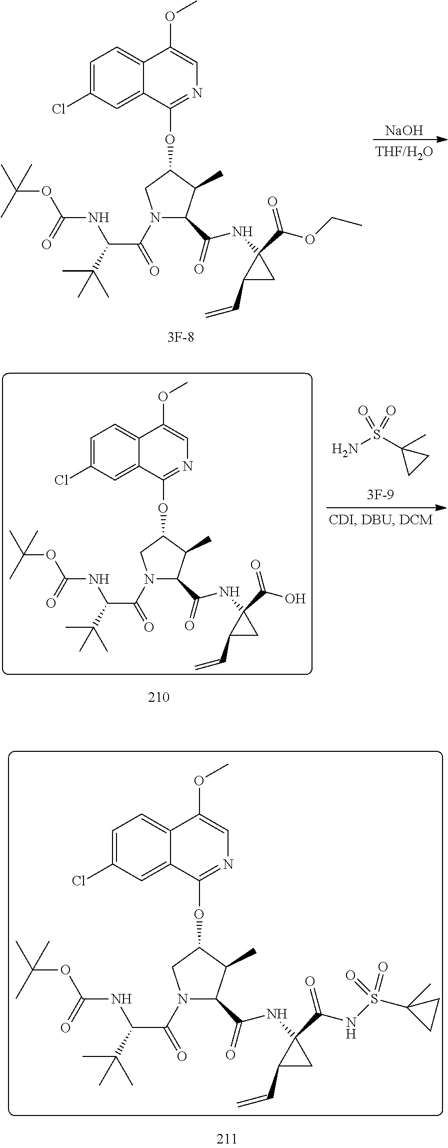

### Compound 3F-3

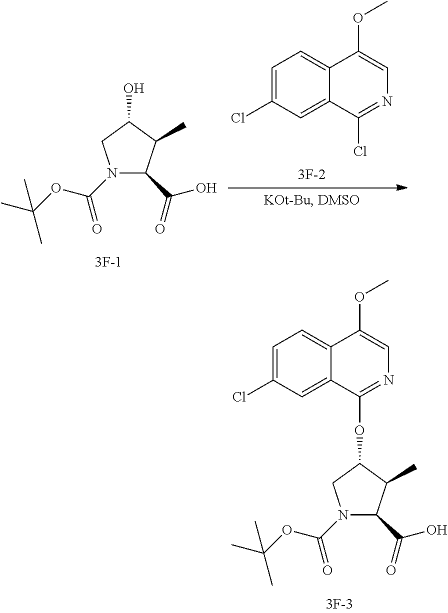

To a solution of compound 3F-1 (500 mg, 2.03 mmol, 1.05 eq) in 10 mL of DMSO was added KOt-Bu (683 mg, 6.09 mmol, 3.15 eq) at 0° C. The suspension was stirred for 15 min at 0° C. After that, compound 3F-2 (439 mg, 1.93 mmol, 1 eq) was added into the flask. The resulting mixture was stirred for another 18 h. TLC analysis showed the reaction completed. The reaction mixture was diluted with water, adjusted to pH=5-6 with aq. HCl (2 N), extracted with EA (50 mL×3). The combined organic layer was washed with brine, dried over anhydrous sodium sulfate, filtered and concentrated under reduced pressure. Purification by prep-TLC (DCM:MeOH=13:1) gave compound 3F-3 (690 mg, yield 82%). MS (ESI) m/z (M+H)⁺ 437.1.

### Compound 3F-5

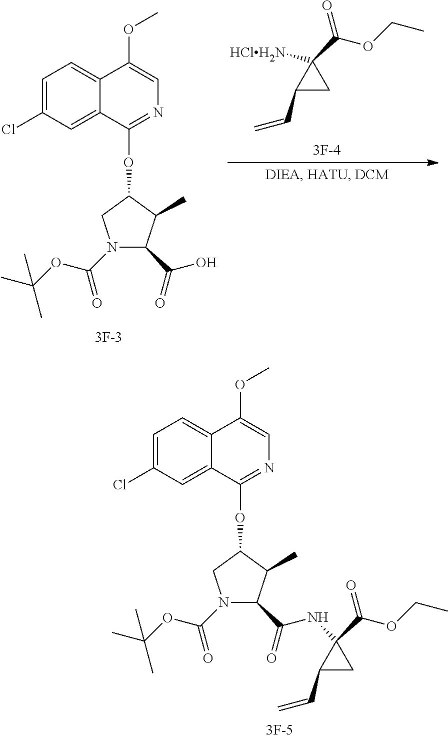

To a solution of compound 3F-3 (690 mg, 1.58 mmol, 1 eq) in 30 mL of DCM was added HATU (902 mg, 2.37 mmol, 1.5 eq), DIEA (815 mg, 6.32 mmol, 4 eq), and compound 3F-4 (604 mg, 3.16 mmol, 2 eq). The mixture was stirred for 18 hrs at room temperature. TLC (PE:EA=2:1) analysis showed the reaction completed. All the volatiles were removed under reduced pressure. The residual was diluted with water, extracted with EA (50 mL×3). The combined organic layer was washed with brine, dried over anhydrous sodium sulfate, filtered and concentrated under reduced pressure. Purification by column chromatography (PE:EA=2:1) gave compound 3F-5 as yellow solid (700 mg, yield 77%). MS (ESI) m/z (M+H)⁺ 574.1.

### Compound 3F-6

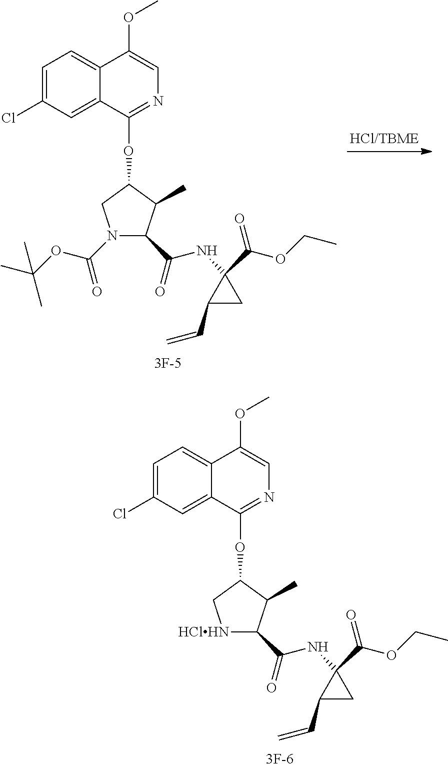

The mixture of compound 3F-5 (465 mg, 0.81 mmol) in 10 mL of solution of HCl (g) in TBME (saturated) was stirred at r.t. for 2 hrs. LCMS analysis showed the reaction completed. The solvent was removed under reduced pressure. The residual was dried and used directly for the next step without purification (400 mg, yield 97%). MS (ESI) m/z (M+H)⁺ 474.0.

### Compound 3F-8

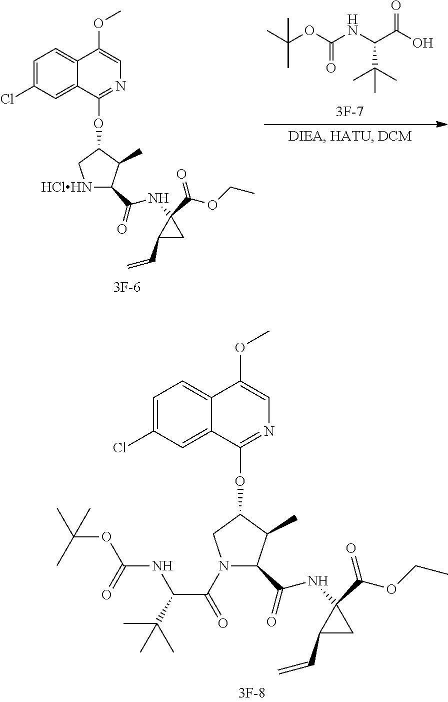

To a solution of compound 3F-7 (88 mg, 0.38 mmol, 1.3 eq) in 6 mL of DCM was added HATU (145 mg, 0.38 mmol, 1.3 eq), DIEA (150 mg, 1.26 mmol, 4 eq), and compound 3F-6 (150 mg, 0.29 mmol, 1 eq). The mixture was stirred for 18 h at room temperature. TLC analysis showed the reaction completed. All the volatiles were removed under reduced pressure. The residual was diluted with water, extracted with EA (50 mL×3). The combined organic layer was washed with brine, dried over anhydrous sodium sulfate, filtered and concentrated under reduced pressure. Purification by prep-TLC (PE:EA=3:1) gave compound 3F-8 as light yellow solid (150 mg, yield 75%). MS (ESI) m/z (M+H)⁺ 687.3.

### Compound 210

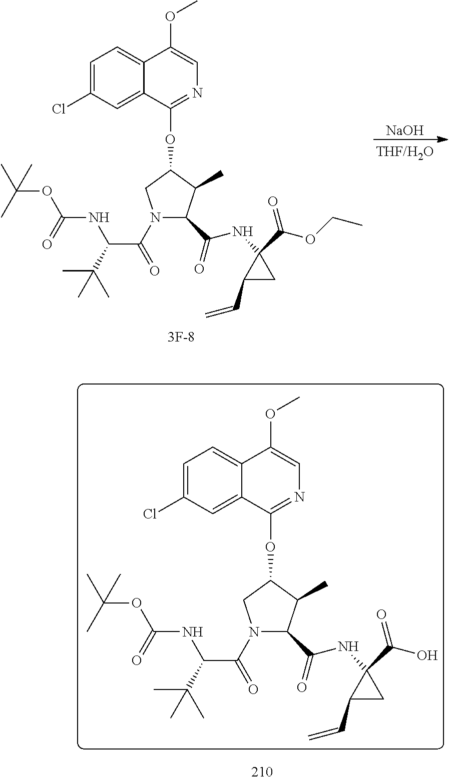

To a solution of compound 3F-8 (80 mg, 0.12 mmol, 1 eq) in 6 mL of THF was added NaOH (46 mg, 1.2 mmol, 10 eq) in 2 mL of H₂O. The mixture was stirred at r.t. for 18 hrs. LCMS analysis showed the reaction completed. All the volatiles were removed under reduced pressure. The residual was diluted with water, adjusted to pH=5-6 with aq. HCl (2 N), extracted with EA (50 mL×3). The combined organic layer was washed with brine, dried over anhydrous sodium sulfate, filtered and concentrated under reduced pressure to give Compound 210 (80 mg, crude yield 100%). MS (ESI) m/z (M+H)⁺ 659.2.

### Compound 211

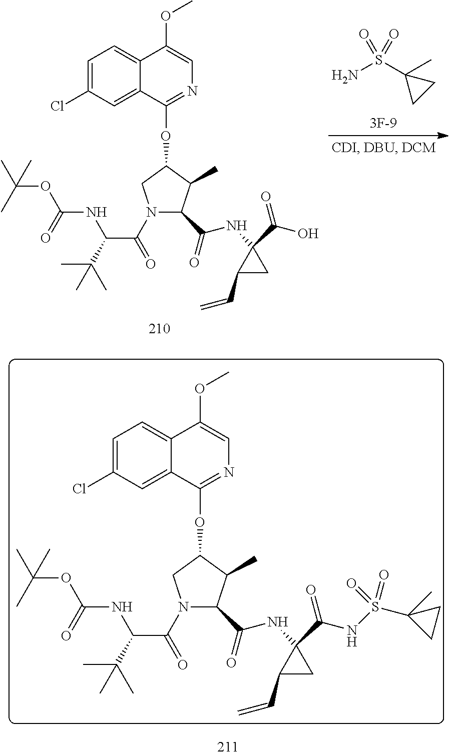

To a solution of Compound 210 (100 mg, 0.15 mmol, 1 eq) in 5 mL of DCM was added CDI (99 mg, 0.60 mmol, 4 eq). The solution was heated to reflux for 2 hrs. After that, DBU (182 mg, 1.2 mmol, 8 eq), and compound 3F-9 (101 mg, 0.75 mmol, 5 eq) were added into the flask. The mixture was stirred for 18 hrs at 30° C. LCMS analysis showed the reaction completed. All the volatiles were removed under reduced pressure. The residual was diluted with water, extracted with EA (50 mL×3). The combined organic layer was washed with brine, dried over anhydrous sodium sulfate, filtered and concentrated under reduced pressure. Purification by prep-HPLC gave Compound 211 as white solid (16.1 mg, yield 14%). MS (ESI) m/z (M+H)⁺ 776.1.

### Compound 212

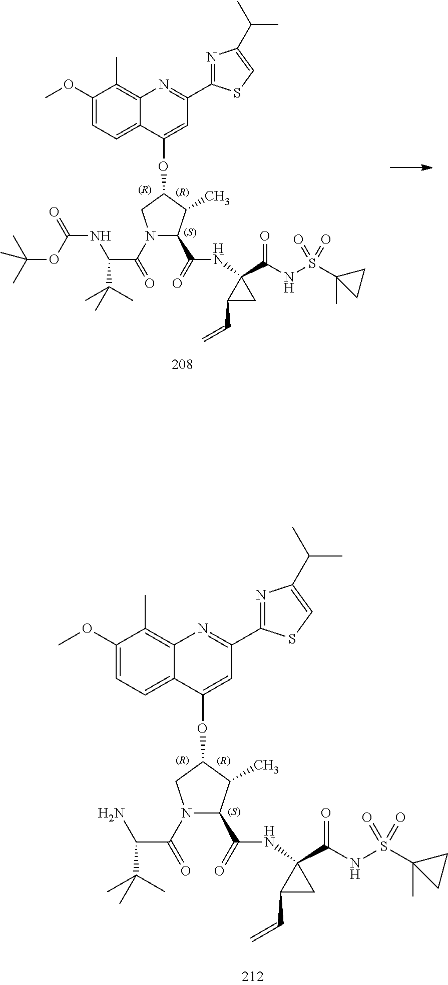

4 M HCl-dioxane is added to Compound 208 in DCM. The reaction mixture is stirred at room temperature for 3 hours and filtered to provide Compound 212.

### Example 9

### Salts

Sodium and potassium salts of Compounds 102, 103, and 205 and the potassium salt of Compound 204 were prepared according to the following general procedure:

To a warmed (˜45° C.) and magnetically stirred solution of the corresponding NH acid in ethanol or methanol (˜8-10 ml/mmol or ˜10 ml/g) was added portion wise either 2N aqueous sodium hydroxide (for sodium salts) or 1N aqueous potassium hydroxide (for potassium salts) until pH of the reaction mixture reached 9. pH was checked using Panpena pH paper (Sigma-Aldrich) which gives unambiguous color change at pH 9. Reaction mixture was carefully diluted with water to reach final alcohol-water ratio about 4:1 to 3:1 v/v while keeping warm (˜45° C.). With gentle stirring the reaction was seeded and the left for crystallization for several hours at room temperature, stirred occasionally. Finally, the reaction mixture was left in a refrigerator overnight.

The solid was filtered off and rinsed with ice-cold water. The product was dried on the air and then under vacuum, first at room temperature and finally at 45° C. overnight until constant weight.

### Example 10

### NS3-NS4 Activity

NS3-NS4 inhibition activity can be determined using known assay methods. For example, NS3/NS4 complexes may be formed and inhibitory concentrations of test compounds determined as described in U.S. Patent Application Publication Number 2007/0054842 paragraph numbers 1497-1509, which is incorporated herein by reference in its entirety. Similarly, hepatitis C replicon EC₅₀ may be determined using known assay methods such as described in U.S. Patent Application Publication Number 2007/0054842 paragraph numbers 1510-1515, which is incorporated herein by reference in its entirety. Assays may be conducted at ambient temperature (23° C.) in assay buffer containing 50 mM Tris-HCl, pH 7.5, 15% glycerol, 0.6 mM Lauryldimethylamine Oxide (LDAO), 25 μM NS4A peptide, and 10 mM Dithiothreitol (DTT).

Examples of inhibition of NS3/NS4 activity is presented in Table 1.

TABLE 1
Examples NS3-NS4 activity.

| Compound | EC₅₀ (nM) | IC₅₀ (nM) |
|---|---|---|
| 101 | A | B |
| 102 | C | C |
| 103 | C | C |
| 104 | B | C |
| 105 | C | C |
| 106 | A | B |
| 107 | B | C |
| 109 | B | C |
| 110 | A | C |
| 111 | C | C |
| 202 | A | A |
| 203 | B | C |
| 204 | C | C |
| 205 | C | C |
| 206 | B | C |
| 207 | A | C |
| 208 | A | B |
| 209 | B | C |
| 210 | A | B |
| 211 | C | C |
| 212 | | A |

A indicates an EC₅₀ or IC₅₀ > 100 nM
B indicates an EC₅₀ or IC₅₀ from 10 to 100 nM
C indicates an EC₅₀ or IC₅₀ < 10 nM

1. A compound having a formula I: 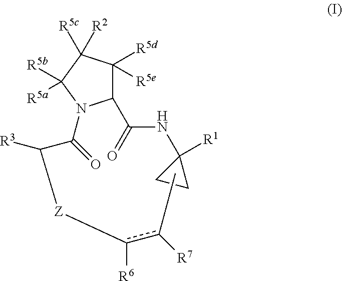
or a pharmaceutically acceptable salt or prodrug thereof, wherein:
(a) R¹ is —C(O)NHS(O)₂R^{1a}, —C(O)NHS(O)₂NR^{1b}R^{1c}, —C(O)NHS(O)R^{1a}, —C(O)NHS(O)NR^{1b}R^{1c}, —C(O)NHC(O)R^{1a}, —C(O)NHOR^{1d}, —C(O)NR^{1b}R^{1c}, —C(O)R^{1a}, —C(O)OR^{1d}, or —C(O)C(O)NR^{1b}R^{1c}, —C(O)C(O)OH, or —P(O)R¹ⁱR^{1j};
R^{1a} is selected from the group consisting of —H, —C(O)NHO(CH₂)ₘR^{1e}, optionally substituted C₁₋₆ alkyl, optionally substituted —(CH₂)ₘC₃₋₇ cycloalkyl, optionally substituted —(CH₂)ₘaryl, optionally substituted —(CH₂)ₘheterocyclyl, and optionally substituted —(CH₂)ₘheteroaryl;
R^{1b}, R^{1c}, and R^{1d} are independently selected from the group consisting of —H, optionally substituted C₁₋₆ alkyl, optionally substituted —(CH₂)ₘC₃₋₇ cycloalkyl, optionally substituted —(CH₂)ₘaryl, optionally substituted —(CH₂)ₘheterocyclyl, and optionally substituted —(CH₂)ₘheteroaryl;
or R^{1b} and R^{1c} are taken together with the nitrogen to which they are attached to form optionally substituted heteroaryl or heterocyclyl, each optionally substituted with 1-3 R^{1f};
R^{1e} is selected from the group consisting of C₁₋₆ alkyl, —(CH₂)ₘC₃₋₇ cycloalkyl, optionally substituted aryl, and optionally substituted heteroaryl;
R^{1f} is each independently selected from the group consisting of halo, cyano, amido, phenyl, heteroaryl, heterocyclyl, C₁₋₆ alkyl, C₃₋₇ cycloalkyl, C₁₋₆ alkoxy, C₃₋₇ cycloalkyloxy, —NO₂, —N(R^{1g})₂, —NHC(O)R^{1g}, —NHC(O)NHR^{1g}, and —NHC(O)OR^{1h};
R^{1g} is —H, C₁₋₆ alkyl, or C₃₋₇ cycloalkyl;
R^{1h} is C₁₋₆ alkyl or C₃₋₇ cycloalkyl;
R¹ⁱ and R^{1j} are each separately selected from the group consisting of hydroxy, —(O)ₜ—C₁₋₆ alkyl, —(O)ₜ—(CH₂)ₘC₃₋₇cycloalkyl, —(O)ₜ-aryl, and —(O)ₜ-heteroaryl, each optionally substituted with one or more substituents each independently selected from the group consisting of halo, cyano, nitro, hydroxy, —C(O)OH, C₁₋₆ alkyl, —(CH₂)ₘC₃₋₇cycloalkyl, C₂₋₆ alkenyl, C₁₋₆ alkoxy, hydroxy-C₁₋₆ alkyl, C₁₋₆ alkyl optionally substituted with up to 5 fluoro, and C₁₋₆ alkoxy optionally substituted with up to 5 fluoro;
(b) R² is 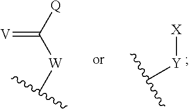
V is selected from O, S, or NH; when V is O or S, W is O, —NR^{2k}—, or —CR^{2k}—; when V is NH, W is —NR^{2k}— or —CR^{2k}—; where R^{2k} is —H, optionally substituted C₁₋₆ alkyl or optionally substituted C₃₋₇ cycloalkyl;
Y is —O—, —S—, —S(O)—, —S(O)₂—, —OCH₂—, —CH₂O—, or a bond;
X is —(CH₂)ₚR^{2b};
Q is —(CH₂)ₚR^{2b} or —O(CH₂)ₚR^{2b};
R^{2b} is selected from the group consisting hydrogen, alkyl, aryl, heterocyclyl, or heteroaryl; each optionally substituted with one or more substituents selected from the group consisting of halo, cyano, nitro, hydroxy, cyanoamino, optionally substituted C₁₋₆ alkyl, optionally substituted C₃₋₇ cycloalkyl, alkylcycloalkyl, C₂₋₆ alkenyl, C₂₋₆ alkynyl, optionally substituted heterocyclyl, optionally substituted C₁₋₆ alkoxy, optionally substituted aryl, optionally substituted heteroaryl, arylthio, ester, sulfonamide, urea, thiourea, amido, thioamide, carboxyl, carbamyl, carbamate, sulfide, sulfoxide, sulfonyl, amino, alkoxyamino, aminoalkoxy, aminoalkylthio, aminoalkyl, C₁₋₆ alkylthio, alkoxyheterocyclyl, alkylamino, hydroxyalkylamino, alkylcarboxy, carbonyl, spirocyclic cyclopropyl, spirocyclic cyclobutyl, spirocyclic cyclopentyl, spirocyclic cyclohexyl, and —NR^{2c}R^{2d};
R^{2c} and R^{2d} are each independently —H, or independently selected from the group consisting of optionally substituted C₁₋₆ alkyl, optionally substituted C₃₋₇ cycloalkyl, and optionally substituted phenyl; or R^{2c} and R^{2d} are taken together with the nitrogen to which they are attached to form heterocyclyl or heteroaryl;
(c) R³ is —NR^{3a}R^{3b} or optionally substituted aryl;
R^{3a} is selected from the group consisting of —H, optionally substituted C₁₋₆ alkyl, optionally substituted C₃₋₇ cycloalkyl, optionally substituted C₄₋₁₀ alkylcycloalkyl, aryl, heteroaryl, arylalkyl, heteroarylalkyl; wherein said aryl, said heteroaryl, said arylalkyl, and said heteroarylalkyl are each optionally substituted with one or more substituents selected from the group consisting of halo, —CF₃, nitro, cyano, hydroxy, cyanoamino, —SH, optionally substituted C₁₋₆ alkyl, optionally substituted C₃₋₇ cycloalkyl, optionally substituted C₁₋₆ alkoxy, optionally substituted C₂₋₆ alkenyl, optionally substituted C₂₋₆ alkynyl, optionally substituted aryl, optionally substituted heteroaryl, optionally substituted heterocycl, aryloxy, arylthio, C₁₋₆ alkylthio, —N[(CH₂)_{q}OH][(CH₂)_{q}OH], —S(O)₂NR^{3c}R^{3d}, —NHC(O)NR^{3c}R^{3d}, —NHC(S)NR^{3c}R^{3d}, —C(O)NR^{3c}R^{3d}, —NR^{3c}R^{3d}, —C(O)R^{3e}, —C(O)OR^{3e}, —NHC(O)R^{3e}, —NHC(O)OR^{3e}, —S(O)ₘR^{3e}, —NHS(O)₂R^{3e}, —NR^{3e}[(CH₂)_{q}OH], —O[(CH₂)_{q}NR^{3c}R^{3d}], and —S[(CH₂)_{q}NR^{3c}R^{3d}];
R^{3b} is selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted aryl, optionally substituted heteroaryl, optionally substituted heterocyclyl, —C(O)R^{3e}, —C(O)OR^{3e}, —C(O)NR^{3c}R^{3d}, —C(S)NR^{3c}R^{3d}, —S(O)ₘR^{3e}, —S(O)₂OR^{3e}, —S(O)₂NR^{3c}R^{3d}, —C(O)CHR^{3f}(CH₂)ₙC(O)R^{3g}, —C(O)CHR^{3f}NHC(O)R^{3g};
or R^{3a} and R^{3b} are taken together with the nitrogen to which they are attached to form optionally substituted heterocyclyl or optionally substituted heteroaryl;
R^{3c} and R^{3d} are each independently selected from the group consisting of —H, optionally substituted C₁₋₆ alkyl, optionally substituted C₁₋₆ alkoxy, C₂₋₆ alkenyl, C₂₋₆ alkynyl, carboxyl, halo, hydroxyl, amino, amido, —OC(O)—C₁₋₆ alkyl, optionally substituted C₃₋₇ cycloalkyl, optionally substituted C₃₋₇ cycloalkyloxy, optionally substituted C₄₋₁₀ alkylcycloalkyl, optionally substituted C₄₋₁₀ cycloalkyl-alkyl, optionally substituted aryl, optionally substituted C₇₋₁₀ arylalkyl, optionally substituted heteroaryl, optionally substituted C₆₋₁₂ heteroarylalkyl and optionally substituted heterocyclyl; or R^{3c} and R^{3d} are taken together with the nitrogen to which they are attached to form optionally substituted heterocyclyl or optionally substituted heteroaryl;
R^{3e} is selected from the group consisting of optionally substituted C₁₋₆ alkyl, optionally substituted C₃₋₇ cycloalkyl, optionally substituted C₂₋₆ alkenyl, optionally substituted C₂₋₆ alkynyl, optionally substituted C₆₋₁₀ aryl, optionally substituted heterocyclyl, optionally substituted heteroaryl, and optionally substituted bicycloalkyl;
R^{3f} is optionally substituted C₁₋₆ alkyl, optionally substituted C₃₋₇ cycloalkyl, optionally substituted C₆₋₁₀ aryl, optionally substituted heteroaryl, optionally substituted heterocyclyl, aryloxy and heteroaryloxy;
R^{3g} is C₁₋₆ alkyl, C₃₋₇ cycloalkyl, or C₄₋₁₀ alkylcycloalkyl, which are all optionally substituted from one to three times with halo, cyano, nitro, hydroxy, C₁₋₆ alkyl optionally substituted with up to 5 fluoro, or phenyl;
(d) R^{5a}, R^{5b}, R^{5c}, R^{5d}, and R^{5e} are each independently selected from —H, optionally substituted C₁₋₆ alkyl, optionally substituted C₂₋₆ alkenyl, optionally substituted C₂₋₆ alkynyl, or optionally substituted C₁₋₆ alkoxy;
provided that at least one of R^{5c}, R^{5d}, and R^{5e} is not —H or at least one of R^{5a} and R^{5b} is methyl;
or R^{5a} and R^{5b} together with the carbon atom to which they are attached to form a C₃₋₆ cycloalkyl or C₃₋₆ cycloalkoxy, and R^{5c}, R^{5d}, and R^{5e} are —H;
or R^{5d} and R^{5e} together with the carbon atom to which they are attached to form a C₃₋₆ cycloalkyl or C₃₋₆ cycloalkoxy, and R^{5a}, R^{5b}, and R^{5c} are —H;
(e) R⁶ and R⁷ are each independently hydrogen, halo, or together with the carbon atoms to which they are attached to form an optionally substituted cycloalkyl;
(f) Z is C₃₋₆ alkyl or three- to seven-membered heteroalkyl containing 1-2 heteroatoms selected from O or N, wherein each said alkylene and said heteroalkylene is optionally substituted by 1-3 R⁸;
wherein R⁸ is —OH, —F, C₁₋₆ alkyl optionally substituted with up to 5 fluoro, or —SOₘR^{8a};
R^{8a} is selected from the group consisting of C₁₋₆ alkyl, C₃₋₇ cycloalkyl, and C₆₋₁₀ aryl, each optionally substituted with one or more substituents each independently selected from the group consisting of halo, cyano, nitro, hydroxy, optionally substituted C₁₋₆ alkyl, C₂₋₆ alkenyl, C₁₋₆ alkoxy, and phenyl;
(g) each m is independently 0, 1 or 2;
(h) each n is independently 1, 2, or 3;
(i) each p is independently 0, 1, 2, 3, 4, 5, or 6;
(j) each q is independently 1, 2, 3, 4, 5, or 6;
(k) each t is independently 0 or 1; and
(l) the dashed line represents an optional double bond.

2. The compound of claim 1, further represented by a formula Ia:

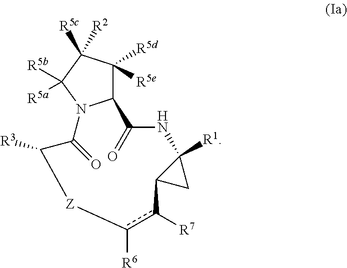

3. The compound of claim 1, wherein:
R² is 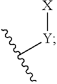
Y is —O— or a bond;
X is 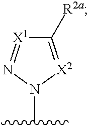
X¹ and X² are each independently selected from —CR^{2e}— or —N—;
R^{2a} and R^{2e} are each selected from the group consisting of —H, halo, optionally substituted aryl, optionally substituted heteroaryl; or R^{2a} and R^{2e} together form an aryl ring optionally substituted by 1-3 R^{2f};
R^{2f} is selected from the group consisting of halo, —C(O)OR^{2g}, —C(O)NR^{2h}R²ⁱ, —NR^{2h}R²ⁱ, —NHC(O)NR^{2h}R²ⁱ, —NHC(O)OR^{2g}, —NHS(O)₂R^{2g}, C₁₋₆ alkyl optionally substituted with up to 5 fluoro, C₂₋₆ alkenyl, C₃₋₇ cycloalkyl, optionally substituted C₁₋₆ alkoxy, optionally substituted aryl, optionally substituted heteroaryl, and optionally substituted heterocyclyl;
R^{2g} is selected from the group consisting of —H, C₁₋₆ alkoxy, C₁₋₆ alkyl, C₃₋₇ cycloalkyl, aryl, arylalkyl heteroaryl, optionally substituted heterocyclyl; and
R^{2h} and R²ⁱ are each independently selected from the group consisting of —H, optionally substituted C₁₋₆ alkyl, optionally substituted C₂₋₆ alkenyl, optionally substituted aryl, optionally substituted arylalkyl optionally substituted heteroaryl, and optionally substituted heterocyclyl.

4. The compound of claim 1, wherein
R² is 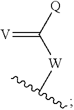
V and W are each 0; and
Q is selected from the group consisting of: 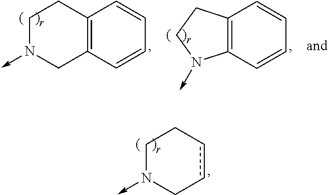
each is optionally substituted with one or more substituents selected from the group consisting of halo, cyano, nitro, hydroxy, cyanoamino, optionally substituted C₁₋₆ alkyl, optionally substituted C₃₋₇ cycloalkyl, C₂₋₆ alkenyl, C₂₋₆ alkynyl, optionally substituted heterocyclyl, optionally substituted C₁₋₆ alkoxy, optionally substituted aryl, optionally substituted heteroaryl, arylthio, ester, sulfonamide, urea, thiourea, amido, thioamide, carboxyl, carbamyl, carbamate, sulfide, sulfoxide, sulfonyl, amino, alkoxyamino, aminoalkoxy, aminoalkylthio, aminoalkyl, C₁₋₆ alkylthio, alkoxyheterocyclyl, alkylamino, hydroxyalkylamino, alkylcarboxy, carbonyl, spirocyclic cyclopropyl, spirocyclic cyclobutyl, spirocyclic cyclopentyl, and spirocyclic cyclohexyl; and wherein r=0 or 1.

5. (canceled)

6. (canceled)

7. The compound of claim 1, wherein
R² is 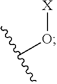
X is selected from 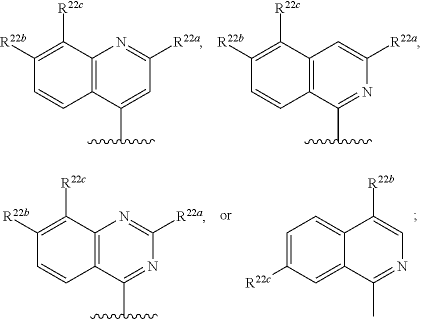
R^{22a} is selected from the group consisting of aryl, heterocyclyl and heteroaryl, each substituted with R^{22e};
R^{22b} is selected from the group consisting of —H, halo, C₁₋₆ alkoxy, C₃₋₇ cycloalkyloxy, and hydroxy;
R^{22c} is —H, optionally substituted C₁₋₆ alkyl or halo;
R^{22e} is selected from the group consisting of —H, C₁₋₆ alkyl, C₃₋₇ cycloalkyl, and —NR^{22f}R^{22g}; wherein R^{22f} and R^{22g} are each independently —H, C₁₋₆ alkyl, or C₃₋₇ cycloalkyl;
R³ is —NR^{3a}R^{3b} or aryl optionally substituted with 1-3 substituents independently selected from halo or C₁₋₆ haloalkyl;
R^{3a} is selected from the group consisting of —H, C₁₋₆ alkyl, and C₃₋₇ cycloalkyl; and
R^{3b} is selected from the group consisting of —H, —C(O)OR^{3e}, —C(O)NR^{3c}R^{3d}, and aryl optionally substituted with 1-3 substituents selected from the group consisting of halo, —CF₃, hydroxy, nitro, amino, optionally substituted C₁₋₆ alkyl, optionally substituted C₁₋₆ alkoxy, optionally substituted heterocyclyl, and optionally substituted heteroaryl.

8. (canceled)

9. (canceled)

10. (canceled)

11. (canceled)

12. The compound of claim 1, wherein
R³ is —NR^{3a}R^{3b} or aryl optionally substituted with 1-3 substituents independently selected from halo or C₁₋₆ haloalkyl;
R^{3a} is selected from the group consisting of —H, C₁₋₆ alkyl, and C₃₋₇ cycloalkyl;
R^{3b} is selected from the group consisting of —H, —C(O)OR^{3e}, —C(O)NR^{3c}R^{3d}, heteroaryl, and aryl, wherein the heteroaryl or aryl of R^{3b} is optionally substituted with halo or C₁₋₆ haloalkyl; and
R^{3c} and R^{3d} are taken together with the nitrogen to which they are attached to form optionally substituted heterocyclyl or optionally substituted heteroaryl; and
R^{3e} is C₁₋₆ alkyl, C₃₋₇ cycloalkyl, or heterocyclyl; each optionally substituted with one or more substituents each independently selected from the group consisting of halo, cyano, nitro, hydroxy, C₁₋₆ alkyl optionally substituted with up to 5 fluoro, C₂₋₆ alkenyl, —(CH₂)ₚC₃₋₇ cycloalkyl, C₁₋₆ alkoxy optionally substituted with up to 5 fluoro, phenyl, and hydroxy-C₁₋₆ alkyl.

13. (canceled)

14. (canceled)

15. (canceled)

16. (canceled)

17. (canceled)

18. (canceled)

19. (canceled)

20. A compound having a formula II: 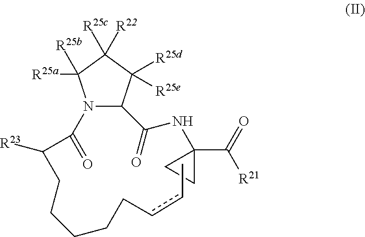
or a pharmaceutically acceptable salt or prodrug thereof, wherein:
(a) R²¹ is selected from hydroxy, —NHS(O)₂R^{21a}, —NHS(O)₂R^{21b}R^{21c}, or —NR^{21b}R^{21c}; wherein
R^{21a} is selected from the group consisting of optionally substituted C₁₋₆ alkyl, optionally substituted C₃₋₇ cycloalkyl, optionally substituted aryl, and optionally substituted heterocyclyl;
R^{21b} and R^{21c} are each independently selected from —H, optionally substituted C₁₋₆ alkyl, optionally substituted C₃₋₇ cycloalkyl, optionally substituted aryl, optionally substituted heterocyclyl, and arylalkyl; or R^{21b} and R^{21c} together with the nitrogen to which they are attached to form an optionally substituted 3-7 membered heterocyclyl ring;
(b) R²² is selected from 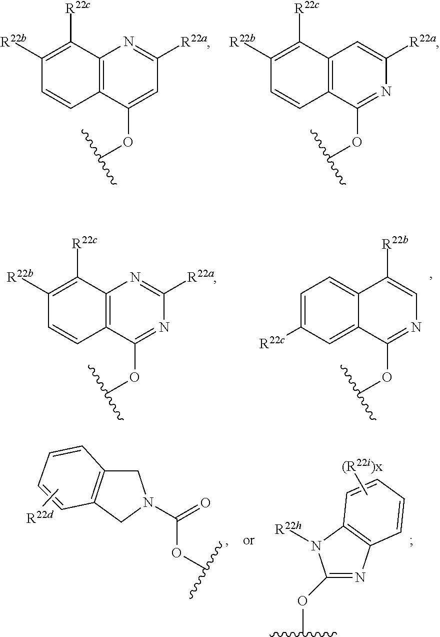
wherein
R^{22a} is selected from the group consisting of —H, halo, C₁₋₆ alkyl, C₃₋₇ cycloalkyl, C₁₋₆ haloalkyl, C₁₋₆ thioalkyl, C₁₋₆ alkoxy, C₃₋₇ cycloalkyloxy, C₂₋₇ alkoxyalkyl, aryl, heterocyclyl, and heteroaryl; wherein said C₃₋₇ cycloalkyl, said aryl, said heterocyclyl and said heteroaryl are each substituted with 1-3 R^{22e};
R^{22b} is selected from the group consisting of —H, halo, C₁₋₆ alkyl, C₃₋₇ cycloalkyl, C₁₋₆ haloalkyl, C₁₋₆ alkoxy, C₃₋₇ cycloalkyloxy, hydroxy, phenyl, heterocyclyl, heteroaryl, and —NR^{22f};
R^{22c} is —H, optionally substituted C₁₋₆ alkyl, optionally substituted C₁₋₆ alkoxy, C₁₋₆ alkylamino or halo;
R^{22d} is selected from the group consisting of —H, halo, cyano, hydroxy, optionally substituted C₁₋₆ alkyl, and optionally substituted C₁₋₆ alkoxy;
R^{22e} is selected from the group consisting of —H, halo, C₁₋₆ alkyl, C₃₋₇ cycloalkyl, C₁₋₆ alkoxy, C₃₋₇ cycloalkyloxy, —NO₂, —NR^{22f}R^{22g}, —NHC(O)R^{22f}, —NHC(O)OR^{22g}, and —NHC(O)NHR^{22f};
R^{22f} and R^{22g} are each independently —H, C₁₋₆ alkyl, or C₃₋₇ cycloalkyl;
R^{22h} is —H or C₁₋₆ alkyl optionally substituted with up to 5 fluoro;
R²²ⁱ is selected from the group consisting of halo, cyano, nitro, hydroxy, cyanoamino, —SH, optionally substituted C₁₋₆ alkyl, optionally substituted C₁₋₆ alkoxy, C₂₋₆ alkenyl, C₂₋₆ alkynyl, heterocyclyl, optionally substituted aryl, optionally substituted heteroaryl, aryloxy, arylthio, C₁₋₆ alkylthio, —N[(CH₂)_{q}OH][(CH₂)_{q}OH], —S(O)₂NR^{22j}R^{22k}, —NHC(O)NR^{22j}R^{22k}, —NHC(S)NR^{22j}R^{22k}, —C(O)NR^{22j}R^{22k}, —NR^{22j}R^{22k}, —C(O)R^{22l}, —C(O)OR^{22l}, —NHC(O)R^{22l}, —NHC(O)OR^{22l}, —SOₘR^{22l}, —NHS(O)₂R^{22l}, —NR^{22l}[(CH₂)_{q}OH], —O[(CH₂)_{q}NR^{22m}R²²ⁿ], —S[(CH₂)_{q}NR^{22m}R²²ⁿ], —(CH₂)_{q}NR^{22m}R²²ⁿ, —(CH₂)_{q}R^{22p} and —O(CH₂)ₚR^{22p};
R^{22j} and R^{22k} are each separately a —H, or separately selected from the group consisting of C₁₋₆ alkyl, —(CH₂)ₘC₃₋₇ cycloalkyl, and phenyl, each optionally substituted with one or more substituents each independently selected from the group consisting of halo, cyano, nitro, hydroxy, C₁₋₆ alkyl, C₃₋₇ cycloalkyl, C₄₋₁₀ alkylcycloalkyl, C₂₋₆ alkenyl, hydroxy-C₁₋₆ alkyl, C₁₋₆ alkyl optionally substituted with up to 5 fluoro, and C₁₋₆ alkoxy optionally substituted with up to 5 fluoro; or R²²ⁱ and R^{22k} are taken together with the nitrogen to which they are attached to form a heterocyclyl;
R^{22l} is selected from the group consisting of C₁₋₆ alkyl, C₃₋₇ cycloalkyl, heterocyclyl, and C₆₋₁₀ aryl, each optionally substituted with one or more substituents each independently selected from the group consisting of halo, cyano, nitro, hydroxy, C₁₋₆ alkyl, C₂₋₆ alkenyl, —(CH₂)ₘC₃₋₇ cycloalkyl, C₁₋₆ alkoxy, phenyl, and hydroxy-C₁₋₆ alkyl;
R^{22m} and R²²ⁿ are each separately —H or C₁₋₆ alkyl; or R^{22m} and R²²ⁿ are taken together with the nitrogen to which they are attached to form a heterocyclyl;
each R^{22p} is heteroaryl;
each p is separately 0, 1, 2, 3, 4, 5, or 6;
each q is separately 1, 2, 3, 4, 5, or 6;
each m is separately 0, 1 or 2;
x is 0, 1, 2 or 3;
(c) R²³ is —NR^{23a}R^{23b} or aryl optionally substituted with 1-3 substituents independently selected from halo, C₁₋₆ alkyl, or C₁₋₆ haloalkyl;
R^{23a} is selected from the group consisting of —H, optionally substituted C₁₋₆ alkyl, optionally substituted C₃₋₇ cycloalkyl, optionally substituted C₄₋₁₀ alkylcycloalkyl, aryl, heteroaryl, arylalkyl, heteroarylalkyl; wherein said aryl, said heteroaryl, said arylalkyl, and said heteroarylalkyl are each optionally substituted with one or more substituents selected from the group consisting of halo, nitro, cyano, hydroxy, cyanoamino, —SH, optionally substituted C₁₋₆ alkyl, optionally substituted C₃₋₇ cycloalkyl, optionally substituted C₁₋₆ alkoxy, optionally substituted C₂₋₆ alkenyl, optionally substituted C₂₋₆ alkynyl, optionally substituted aryl, optionally substituted heteroaryl, aryloxy, arylthio, C₁₋₆ alkylthio, —N[(CH₂)_{q}OH][(CH₂)_{q}OH], —S(O)₂NR^{23c}R^{23d}, —NHC(O)NR^{23c}R^{23d}; —NHC(S)NR^{23c}R^{23d}; —C(O)NR^{23c}R^{23d}, —NR^{23c}R^{23d}, —C(O)R^{23e}, —C(O)OR^{23e}, —NHC(O)R^{23e}, —NHC(O)OR^{23e}, —S(O)ₘR^{23e}, —NHS(O)₂R^{23e}, —NR^{23e}[(CH₂)_{q}OH], —O[(CH₂)_{q}NR^{23c}R^{23d}], and —S[(CH₂)_{q}NR^{23c}R^{23d}];
R^{23b} is selected from the group consisting of —H, optionally substituted C₁₋₆ alkyl, optionally substituted C₂₋₆ alkenyl, optionally substituted C₂₋₆ alkynyl, optionally substituted C₃₋₇ cycloalkyl, optionally substituted aryl, optionally substituted heteroaryl, —C(O)R^{23e}, —C(O)OR^{23e}, —C(O)NR^{23c}R^{23d}, —C(S)NR^{23c}R^{23d}, —S(O)ₘR^{23e}, —S(O)₂OR^{23e}, —S(O)₂NR^{3c}R^{3d}, —C(O)CHR^{23f}(CH₂)ₙC(O)R^{23g}, —C(O)CHR^{23f}NHC(O)R^{23g};
R^{23c} and R^{23d} are each independently selected from the group consisting of —H, optionally substituted C₁₋₆ alkyl, optionally substituted C₁₋₆ alkoxy, C₂₋₆ alkenyl, C₂₋₆ alkynyl, carboxyl, halo, hydroxyl, amino, amido, —OC(O)—C₁₋₆ alkyl, optionally substituted C₃₋₇ cycloalkyl, optionally substituted C₃₋₇ cycloalkyloxy, optionally substituted C₄₋₁₀ alkylcycloalkyl, optionally substituted C₄₋₁₀ cycloalkyl-alkyl, optionally substituted aryl, optionally substituted C₇₋₁₀ arylalkyl, optionally substituted heteroaryl, optionally substituted C₆₋₁₂ heteroarylalkyl and optionally substituted heterocyclyl; or R^{23c} and R^{23d} are taken together with the nitrogen to which they are attached to form optionally substituted heterocyclyl or optionally substituted heteroaryl;
R^{23e} is selected from the group consisting of optionally substituted C₁₋₆ alkyl, optionally substituted C₃₋₇ cycloalkyl, optionally substituted C₂₋₆ alkenyl, optionally substituted C₂₋₆ alkynyl, optionally substituted C₆₋₁₀ aryl, and optionally substituted heterocyclyl;
R^{23f} is optionally substituted C₁₋₆ alkyl, optionally substituted C₃₋₇ cycloalkyl, optionally substituted C₆₋₁₀ aryl, optionally substituted heteroaryl, optionally substituted heterocyclyl, aryloxy and heteroaryloxy;
R^{23g} is C₁₋₆ alkyl, C₃₋₇ cycloalkyl, or C₄₋₁₀ alkylcycloalkyl, which are all optionally substituted from one to three times with halo, cyano, nitro, hydroxy, C₁₋₆ alkyl optionally substituted with up to 5 fluoro, or phenyl;
(d) R^{25a}, R^{25b}, R^{25c}, R^{25d} and R^{25e} are each independently selected from —H, optionally substituted C₁₋₆ alkyl, optionally substituted C₂₋₆ alkenyl, optionally substituted C₂₋₆ alkynyl or optionally substituted C₁₋₆ alkoxy;
provided that at least one of R^{25c}, R^{25d}, and R^{25e} is not —H or at least one of R^{25a} and R^{25b} is methyl;
or R^{25a} and R^{25b} together with the carbon atom to which they are attached to form a C₃₋₇ cycloalkyl, and R^{25c}, R^{25d} and R^{25e} are —H;
or R^{25d} and R^{25e} together with the carbon atom to which they are attached to form a C₃₋₇ cycloalkyl, and R^{25a}, R^{25b}, and R^{25c} are —H; and
(e) the dashed line represents an optional double bond.

21. The compound of claim 20, further represented by a formula IIa:

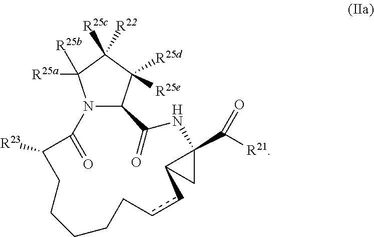

22. The compound of claim 20, wherein
R²² is 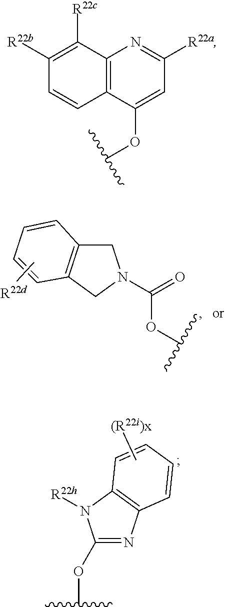
R^{22a} is thiazole optionally substituted by C₁₋₆ alkyl or thiazole optionally substituted by —NH—C₁₋₆ alkyl;
R^{22b} is C₁₋₆ alkoxy or C₃₋₇ cycloalkyloxy; and
R²³ is —NHR^{23b} or C₆₋₁₀ aryl optionally substituted with 1-3 substituents independently selected from halo, C₁₋₆ alkyl, or C₁₋₆ haloalkyl; where R^{23b} is selected from the group consisting of —H, —C(O)OR^{23c}, —C(O)R^{23c}, or —C(O)NR^{23c}R^{23d}.

23. The compound of claim 22, wherein R^{22b} is C₁₋₆ alkoxy; and R^{22c} is —H, —Br, or optionally substituted C₁₋₆ alkyl.

24. The compound of claim 23, wherein
R^{22a} is thiazole optionally substituted by propyl or thiazole optionally substituted with —NH-propyl;
R^{22b} is methoxy; and
R^{22c} is —H or methyl.

25. The compound of claim 20, wherein R²¹ is hydroxyl, —NHS(O)₂R^{21a} or —NR^{21b}R^{21c}; wherein R^{21a} is optionally substituted C₁₋₆ alkyl or optionally substituted C₃₋₇cycloalkyl; and R^{21b} and R^{21c} are each independently optionally substituted C₁₋₆ alkyl or optionally substituted C₃₋₇ cycloalkyl.

26. The compound of claim 20, wherein
R²² is 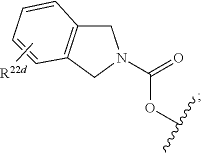
R^{22d} is —H, halo, or C₁₋₆ haloalkyl;
R²³ is —NHR^{23b} or C₆₋₁₀ aryl optionally substituted with 1-3 substituents independently selected from halo, C₁₋₆ alkyl, or C₁₋₆ haloalkyl; where R^{23b} is selected from the group consisting of —H, —C(O)OR^{23e}, —C(O)R^{23e}, or —C(O)NR^{23c}R^{23d}.

27. The compound of claim 20, wherein R²³ is —NHR^{23b} or phenyl optionally substituted with 1-3 substituents independently selected from halo or C₁₋₆ haloalkyl; where R^{23b} is selected from the group consisting of —H, —C(O)OR^{23e}, or —C(O)NR^{23c}R^{23d}, where R^{23c} and R^{23d} are taken together with the nitrogen to which they are attached to form optionally substituted heterocyclyl.

28. (canceled)

29. (canceled)

30. (canceled)

31. (canceled)

32. (canceled)

33. (canceled)

34. (canceled)

35. (canceled)

36. The compound of claim 1, selected from the group consisting of: 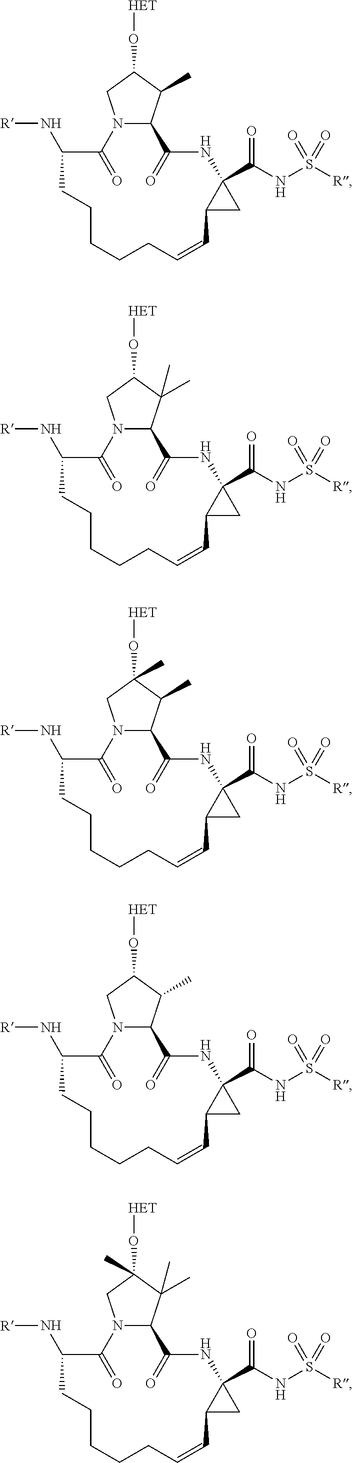 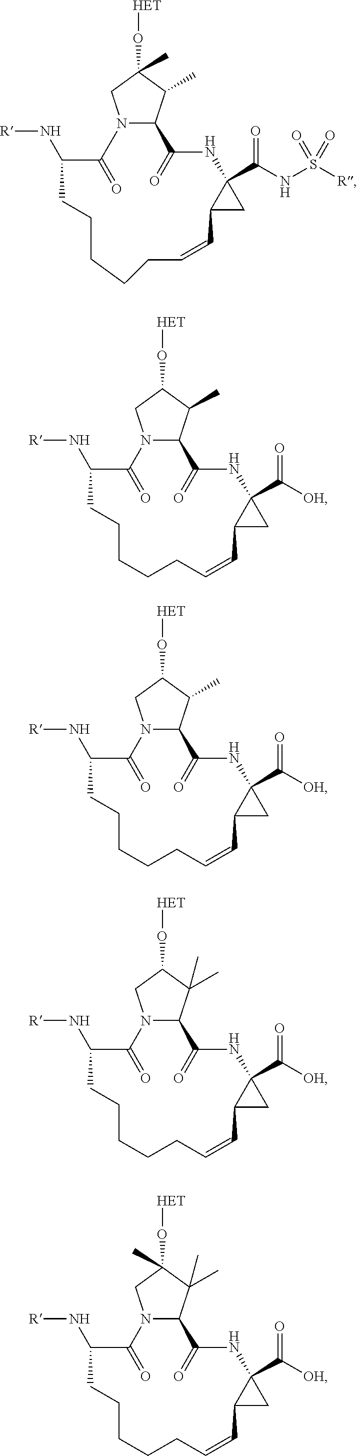 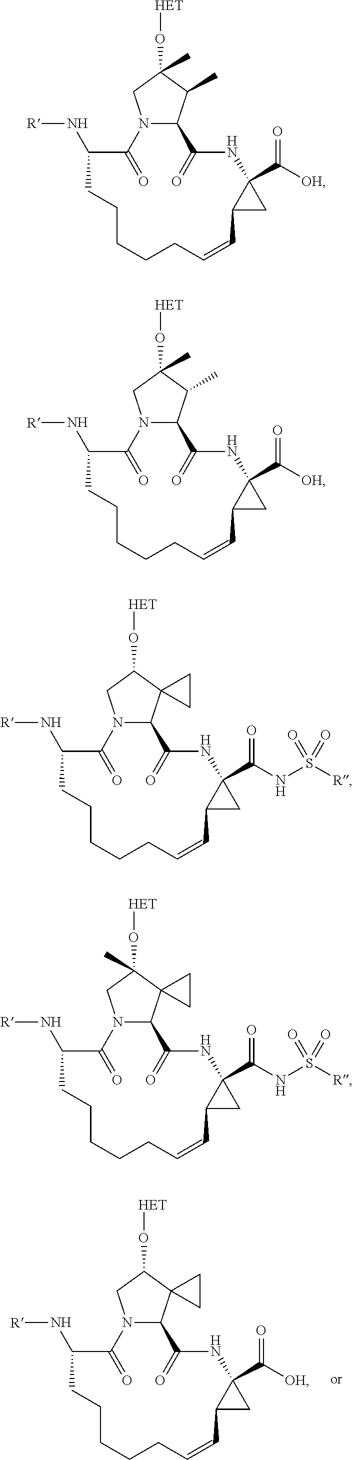 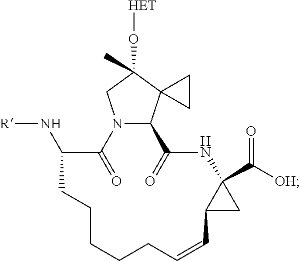
wherein R′ is hydrogen, 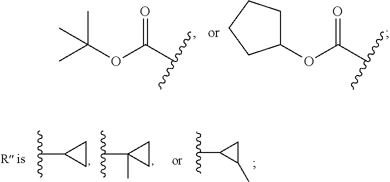
and HET is:

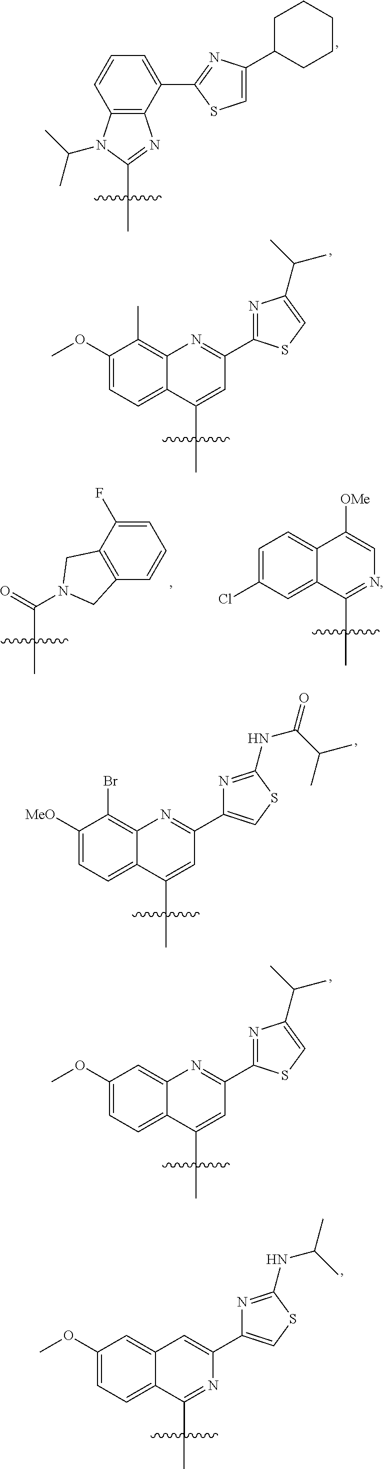

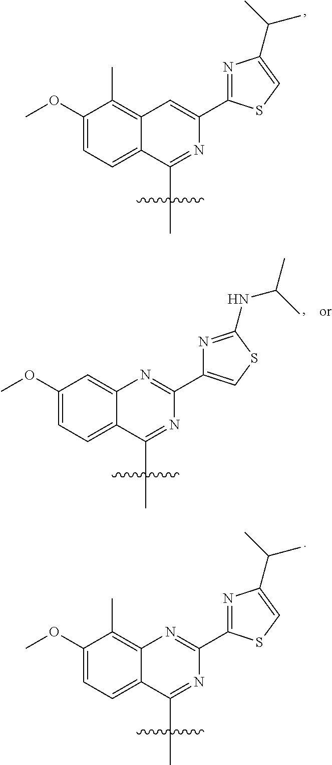

37. The compound of claim 1, selected from the group consisting of:

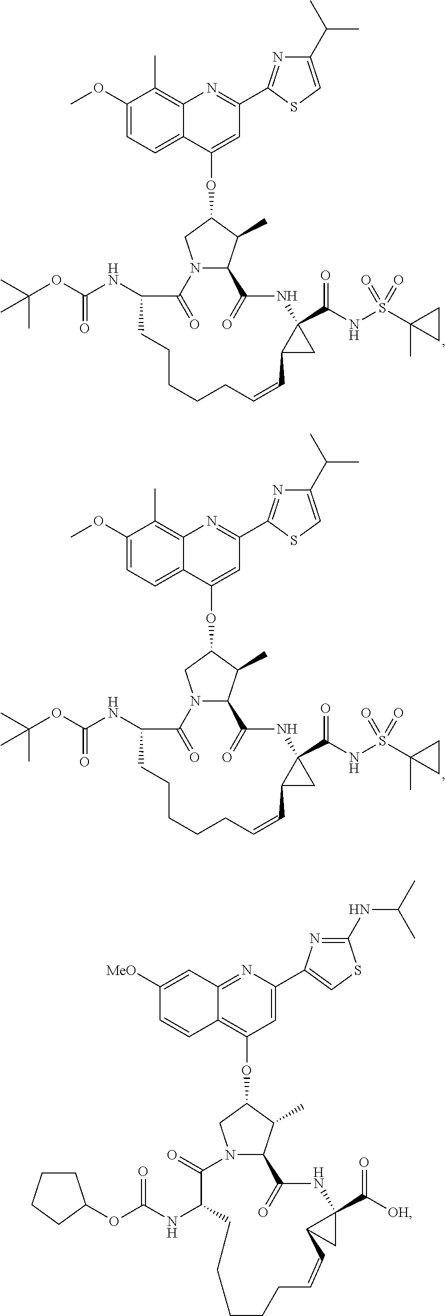

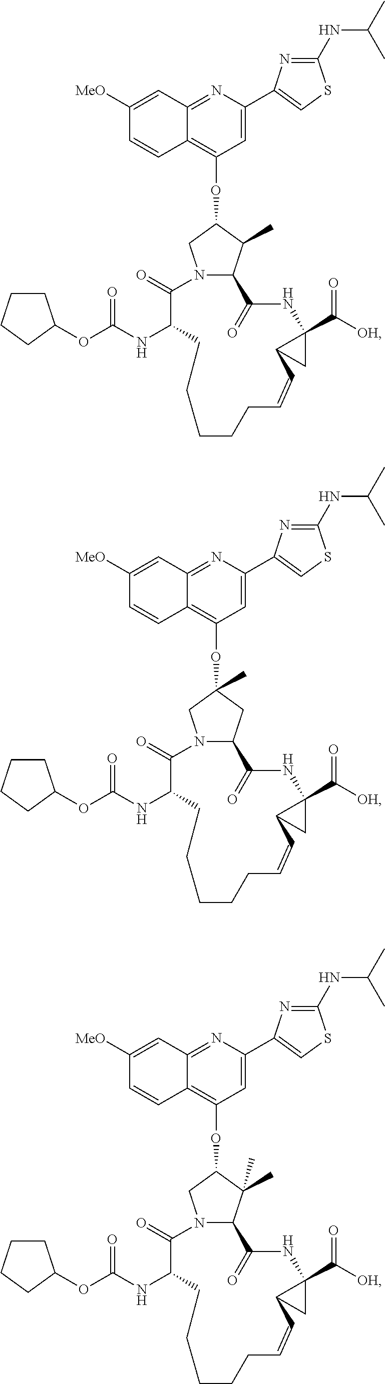

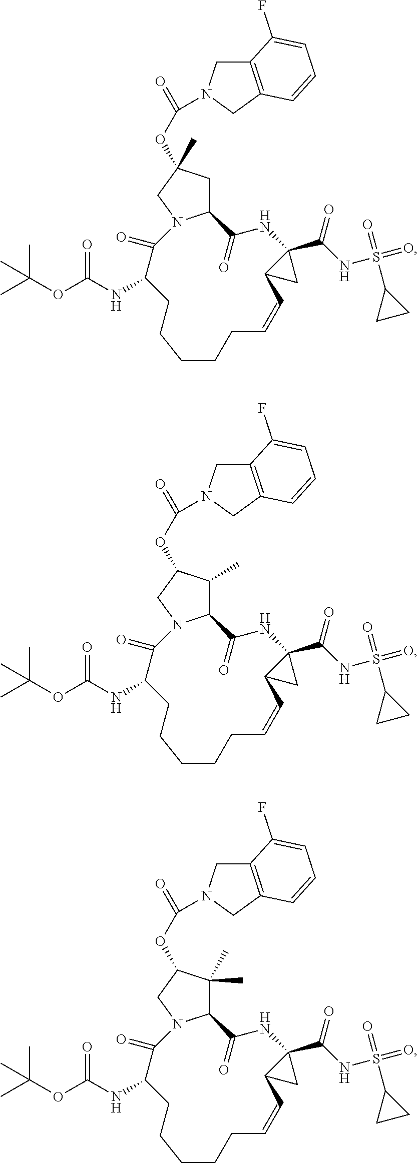

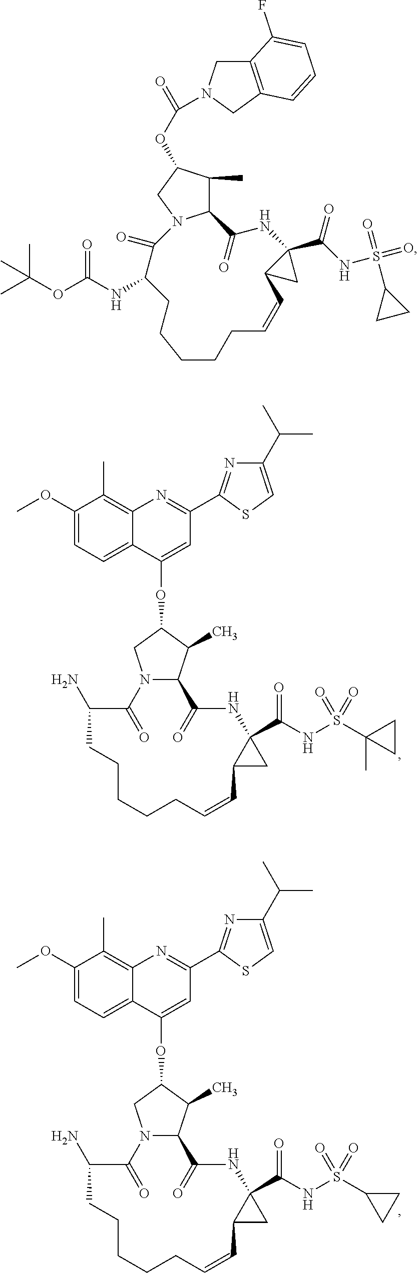

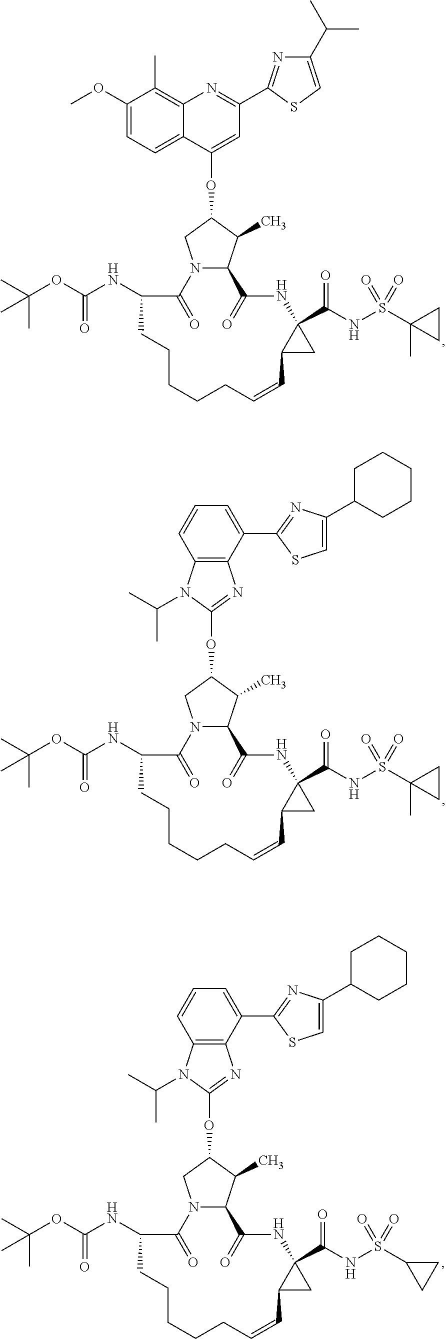

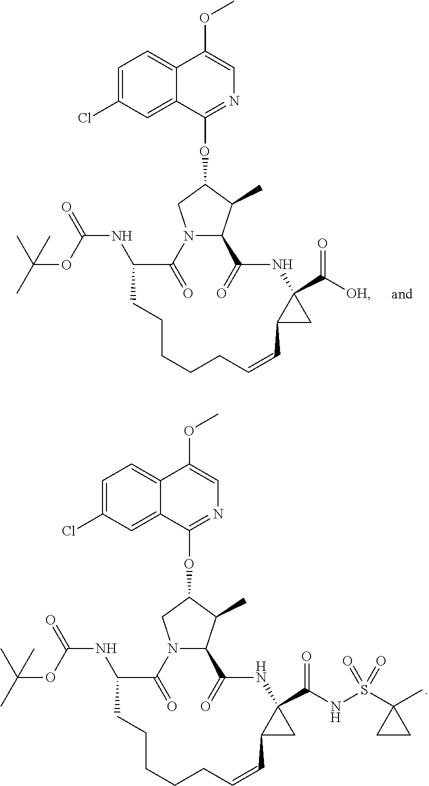

38. A compound having a Formula III: 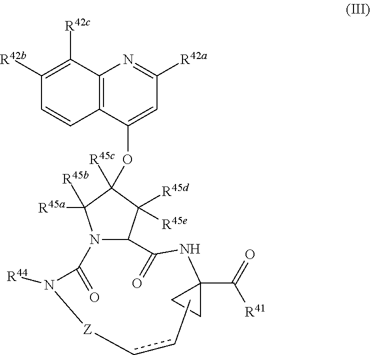
or a pharmaceutically acceptable salt or prodrug thereof, wherein:
(a) R⁴¹ is selected from or —OR^{41a}, —NHS(O)₂R^{41b}, —NHS(O)₂NR^{41c}R^{41d}, or —NR^{41c}R^{41d}; wherein
R^{41a} is selected from the group consisting of —H, optionally substituted C₁₋₆ alkyl, optionally substituted C₃₋₇ cycloalkyl, optionally substituted aryl, optionally substituted heteroaryl, and optionally substituted heterocyclyl;
R^{41b} is selected from the group consisting of optionally substituted C₁₋₆ alkyl, optionally substituted C₃₋₇ cycloalkyl, optionally substituted aryl, optionally substituted heteroaryl, and optionally substituted heterocyclyl;
R^{41c} and R^{41d} are each independently selected from hydrogen, optionally substituted C₁₋₆ alkyl, optionally substituted C₃₋₇ cycloalkyl, optionally substituted aryl, optionally substituted heterocyclyl, and arylalkyl; or R^{41c} and R^{41d} together with the N to which they are attached to form an optionally substituted 3-7 membered heterocyclyl ring;
(b) R^{42a} is selected from the group consisting of —H, halo, C₁₋₆ alkyl, C₃₋₇ cycloalkyl, C₁₋₆ haloalkyl, C₁₋₆ thioalkyl, C₁₋₆ alkoxy, C₃₋₇ cycloalkyloxy, C₂₋₇ alkoxyalkyl, C₆₋₁₀ aryl, heterocyclyl, and heteroaryl; wherein said cycloalkyl, aryl, heterocyclyl and heteroaryl are each substituted with R^{42d};
R^{42d} is selected from the group consisting of —H, halo, C₁₋₆ alkyl, C₃₋₇ cycloalkyl, C₁₋₆ alkoxy, C₃₋₇ cycloalkyloxy, —NO₂, —NR^{42e}R^{42f}, —NHC(O)R^{42e}, —NHC(O)OR^{42f}, and —NHC(O)NHR^{42e}; wherein R^{42e} and R^{42f} are each independently —H, C₁₋₆ alkyl, or C₃₋₇ cycloalkyl;
(c) R^{42b} is selected from the group consisting of —H, halo, C₁₋₆ alkyl, hydroxy, C₁₋₆ alkoxy, C₃₋₇ cycloalkyl, C₁₋₆ haloalkyl, C₃₋₇ cycloalkyloxy, phenyl, heterocyclyl, heteroaryl, and NR^{42e};
(d) R^{42c} is selected from —H, halo, optionally substituted C₁₋₆ alkyl, optionally substituted C₁₋₆ alkoxy, C₁₋₆ alkylamino;
(e) R⁴⁴ is selected from —H, optionally substituted C₁₋₆ alkyl, optionally substituted C₁₋₆ alkoxy-C₁₋₆ alkyl, or optionally substituted C₃₋₇ cycloalkyl;
(f) R^{45a}, R^{45b}, R^{45c}, R^{45d}, and R^{45e} are each independently selected from —H, optionally substituted C₁₋₆ alkyl, optionally substituted C₂₋₆ alkenyl, optionally substituted C₂₋₆ alkynyl or optionally substituted C₁₋₆ alkoxy; and
(g) the dashed line represents an optional double bond.

39. The compound of claim 38 having the formula IIIa:

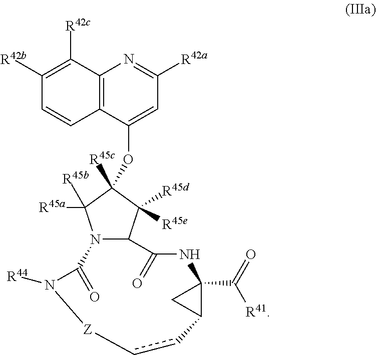

40. (canceled)

41. (canceled)

42. The compound of claim 38, wherein
R⁴¹ is hydroxy, —NHS(O)₂R^{41b}, or —NR^{41c}R^{41d}.
R^{42a} is thiazole optionally substituted by C₁₋₆ alkyl or thiazole optionally substituted by —NH—C₁₋₆ alkyl;
R^{42b} is C₁₋₆ alkoxy;
R^{42c} is —H, optionally substituted C₁₋₆ alkyl or —Br; and
R⁴⁴ is optionally substituted C₁₋₆ alkyl.

43. (canceled)

44. (canceled)

45. (canceled)

46. (canceled)

47. (canceled)

48. (canceled)

49. (canceled)

50. (canceled)

51. The compound of claim 38, wherein the compound is selected from the group consisting of:

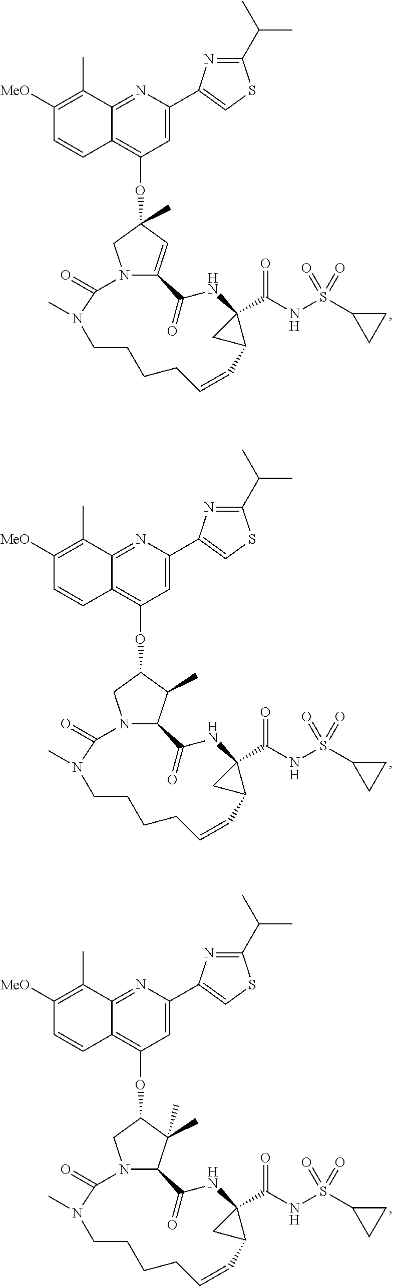

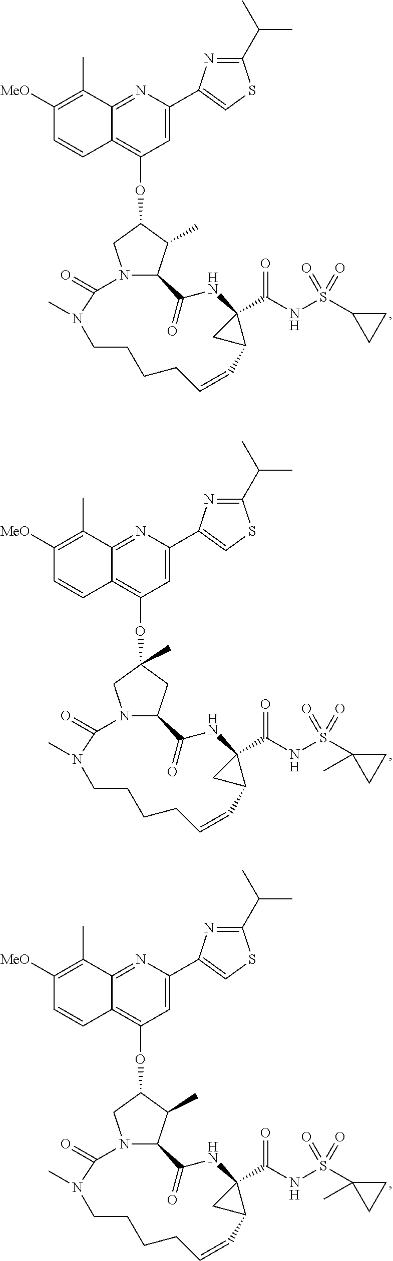

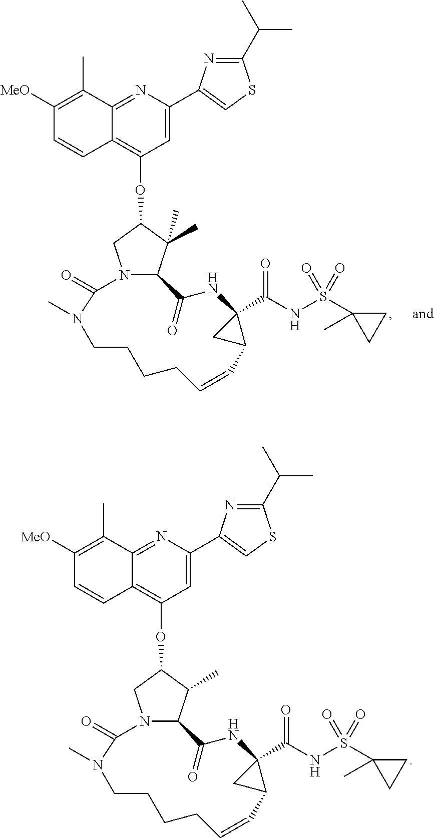

52-297. (canceled)
